(19) United States
(12) Patent Application Publication
(10) Pub. No.: US 2024/0270832 A1
(43) Pub. Date: Aug. 15, 2024

(54) ACUTE TREATMENT AND RAPID TREATMENT OF HEADACHE USING ANTI-CGRP ANTIBODIES

(71) Applicant: H. LUNDBECK A/S, Valby (DK)

(72) Inventors: Roger K. Cady, Bothell, WA (US); Jeffrey T.L. Smith, Dublin (IE); Joseph Hirman, Bothell, WA (US); Barbara Schaeffler, Bothell, WA (US); Lahar Mehta, Valby (DK)

(21) Appl. No.: 18/609,223

(22) Filed: Mar. 19, 2024

Related U.S. Application Data
(62) Division of application No. 18/173,116, filed on Feb. 23, 2023.
Application No. 18/173,116 is a division of application No. 16/736,925, filed on Jan. 8, 2020, now Pat. No. 11,639,380.
(60) Provisional application No. 62/789,828, filed on Jan. 8, 2019.
(60) Provisional application No. 62/872,989, filed on Jul. 11, 2019.
(60) Provisional application No. 62/842,162, filed on May 2, 2019.

(51) Int. Cl. C07K 16/18; A61K 9/00; A61K 31/4172; A61K 38/00; A61K 39/00; A61K 39/395; A61K 45/06; A61K 47/26; A61P 25/06

(52) U.S. Cl. CPC C07K 16/18; A61K 9/0019; A61K 31/4172; A61K 39/3955; A61K 45/06; A61K 47/26; A61P 25/06; A61K 38/00; A61K 2039/505; A61K 2039/545; C07K 2317/24

(57) ABSTRACT

Methods for rapid treatment of chronic migraine are provided. Exemplary methods provide relief from migraine within 24 hours of administration. Also provided are methods for acute treatment of migraine. Exemplary methods comprise administration of an anti-CGRP antagonist antibody to a patient in need thereof.

### RELATED APPLICATIONS

The instant application claims priority to U.S. Provisional Application No. 62/789,828, filed Jan. 8, 2019 (Attorney Docket No. 1143257.008600); U.S. Provisional Application No. 62/872,989, filed Jul. 11, 2019 (Attorney Docket No. 1143257.009001); and U.S. Provisional Application No. 62/842,162, filed May 2, 2019 (Attorney Docket No. 1143257.009000), all of which applications are incorporated by reference in their entireties herein.

### SEQUENCE LISTING DISCLOSURE

The instant application contains a Sequence Listing which has been submitted in ASCII format via EFS-Web and is hereby incorporated by reference in its entirety. Said ASCII copy, created on Jan. 7, 2020, is named “1143257o008601.txt” and is 357,512 bytes in size.

### BACKGROUND

### Field

This invention pertains to methods of acute treatment of headache disorders, such as migraine, using antibodies and fragments thereof (including Fab fragments) that specifically bind to human Calcitonin Gene Related Peptide (hereinafter “CGRP”). The invention also pertains to rapid treatment of headache, e.g., chronic migraine, using antibodies and fragments thereof (including Fab fragments) that specifically bind to human Calcitonin Gene Related Peptide (hereinafter “CGRP”).

### Description of Related Art

Calcitonin Gene Related Peptide (CGRP) is produced as a multifunctional neuropeptide of 37 amino acids in length. Two forms of CGRP, the CGRP-alpha and CGRP-beta forms, exist in humans and have similar activities. CGRP-alpha and CGRP-beta differ by three amino acids in humans, and are derived from different genes. CGRP is released from numerous tissues such as trigeminal nerves, which when activated release neuropeptides within the meninges, mediating neurogenic inflammation that is characterized by vasodilation, vessel leakage, and mast-cell degradation. Durham, P. L., New Eng. J. Med., 350 (11):1073-75 (2004). Biological effects of CGRP are mediated via the CGRP receptor (CGRP-R), which consists of a seven-transmembrane component, in conjunction with receptor-associated membrane protein (RAMP). CGRP-R further requires the activity of the receptor component protein (RCP), which is essential for an efficient coupling to adenylate cyclase through G proteins and the production of cAMP. Doods, H., Curr. Op. Invest. Drugs, 2(9):1261-68 (2001).

Migraines are neurovascular disorder affecting approximately 10% of the adult population in the U.S., and are typically accompanied by intense headaches. CGRP is believed to play a prominent role in the development of migraines. In fact several companies, i.e., Amgen, Eli Lilly, Teva and Alder Biopharmaceuticals (recently acquired by Lundbeck A/S) have developed anti-CGRP and anti-CGRP-R antibodies for use in treating or preventing migraine headaches. The present assignee has previously filed patent applications related to anti-CGRP antibodies and uses thereof including published PCT Application WO/2012/162243 filed May 21, 2012 entitled “ANTI-CGRP COMPOSITIONS AND USE THEREOF”, published PCT Application WO/2012/162257 filed May 21, 2012, entitled “USE OF ANTI-CGRP ANTIBODIES AND ANTIBODY FRAGMENTS TO PREVENT OR INHIBIT PHOTOPHOBIA OR LIGHT AVERSION IN SUBJECTS IN NEED THEREOF, ESPECIALLY MIGRAINE SUFFERERS” published PCT Application WO/2012/162253, filed May 21, 2012, entitled “USE OF ANTI-CGRP OR ANTI-CGRP-R ANTIBODIES OR ANTIBODY FRAGMENTS TO TREAT OR PREVENT CHRONIC AND ACUTE FORMS OF DIARRHEA” and published PCT Application WO/2015/003122, filed Jul. 3, 2014, entitled “REGULATION OF GLUCOSE METABOLISM USING ANTI-CGRP ANTIBODIES” all of which applications are incorporated by reference in their entirety.

### BRIEF SUMMARY

The present disclosure provides methods of acute treatment of headache, comprising administering to a patient in need an effective amount of at least one anti-CGRP antibody or antibody fragment or an anti-CGRP-R antibody or antibody fragment or one or more formulations comprising said antibody or antibody fragment as disclosed herein. Said antibody may be administered while said patient has a headache. Said antibody administration may be initiated within 1-6 hours of the onset of said headache. Said headache may comprise migraine, e.g., episodic migraine or chronic migraine. Said headache may comprise medication overuse headache. Said anti-CGRP antibody or antibody fragment optionally comprises any one of Ab1-Ab14 or a Fab fragment thereof, such as Ab6 or a Fab fragment thereof, e.g., having the light chain CDR 1, 2, and 3 polypeptide sequences of SEQ ID NO: 224; SEQ ID NO: 226; and SEQ ID NO: 228, respectively and the heavy chain CDR 1, 2, and 3 polypeptide sequences of SEQ ID NO: 204; SEQ ID NO: 206; and SEQ ID NO: 208; or having the light chain CDR 1, 2, and 3 polypeptide sequences encoded by SEQ ID NO: 234; SEQ ID NO: 236; and SEQ ID NO: 238, respectively and heavy chain CDR 1, 2, and 3 polypeptide sequences encoded by SEQ ID NO: 214; SEQ ID NO: 216; and SEQ ID NO: 218, respectively. Said anti-CGRP antibody may comprise the variable light chain polypeptide of SEQ ID NO: 222 and the variable heavy chain polypeptide of SEQ ID NO: 202. Said anti-CGRP antibody may comprise the variable light chain polypeptide encoded by SEQ ID NO: 232 and the variable heavy chain polypeptide encoded by SEQ ID NO: 212. Said anti-CGRP antibody may comprise the light chain polypeptide of SEQ ID NO: 221 and the heavy chain polypeptide of SEQ ID NO: 201 or SEQ ID NO: 566. Said anti-CGRP antibody may comprise the light chain polypeptide encoded by SEQ ID NO: 231 and the heavy chain polypeptide encoded by SEQ ID NO: 211 or SEQ ID NO: 567. Said anti-CGRP antibody may comprise the antibody expression product isolated from recombinant cells which express nucleic acid sequences encoding the variable light chain polypeptide of SEQ ID NO: 222 and the variable heavy chain polypeptide of SEQ ID NO: 202, which polypeptides optionally are respectively linked to human light and heavy constant region polypeptides, e.g., human IgG1, IgG2, IgG3 or IgG4 constant regions, which constant regions optionally may be modified to alter glycosylation or proteolysis, wherein said recombinant cells optionally comprise yeast or mammalian cells, e.g., Pichia pastoris or CHO cells. Said anti-CGRP antibody may comprise the antibody expression product isolated from recombinant cells which express nucleic acid sequences encoding the light chain of SEQ ID NO: 221 and the heavy chain polypeptide of SEQ ID NO: 201 or SEQ ID NO: 566, wherein said recombinant cells optionally comprise yeast or mammalian cells, e.g., Pichia pastoris or CHO cells, wherein the constant regions thereof optionally may be modified to alter glycosylation or proteolysis or other effector functions. Any of the aforementioned anti-CGRP antibodies or antibody fragments, preferably Ab6, may be optionally comprised in a formulation as disclosed herein, e.g., comprising histidine (L-histidine), sorbitol, polysorbate 80, such as, per 1 mL volume, about 100 mg anti-CGRP antibody, about 3.1 mg L-Histidine, about 40.5 mg Sorbitol, and about 0.15 mg Polysorbate 80, having a pH of about 5.8. The administered dosage of said antibody may be between about 100 mg and about 300 mg, such as about 100 mg, about 300 mg, 100 mg, or 300 mg. The dosage may be administered by different means, e.g., intravenously, e.g., in a saline solution such as 0.9% sodium chloride in a suitable volume, such as 100 mL.

Said patient may exhibit less than 25 headache days per month, less than 20 headache days per month, less than 15 headache days per month, or less than 10 headache days per month. For example, said patient may exhibit less than 14 headache days, less than headache 13 days, less than headache 12 days, less than headache 11 days, less than 10 headache days, less than 9 headache days, less than 8 headache days, less than 7 headache days, or less than 6 headache days per month. Said patient may exhibit between 2-15 headache days, e.g., 3-14 headache days, 4-13 headache days, 5-12 headache days, 6-11 headache days, or 7-10 headache days/month.

Said patient may exhibit less than 10 migraines per month, such as between 1-9 migraines per month, such as between 2-8 migraines per month, between 3-7 migraine per month, between 4-6 migraine per month, or about 5 migraines per month. Said patient may exhibit fewer than 1 migraine per month on average, e.g., on average one migraine every 2 months, one every 3 months, one every 4 or 6 months, or intermediate values such as 2 every 3 months, etc. Said migraine may be diagnosed in accord with the ICHD-3 guidelines.

In exemplary embodiments, said headache may comprise medication overuse headache. Said medication overuse headache may be determined based on meeting the following criteria: (a) headache occurring on 15 or more days/month in a patient with a pre-existing headache disorder; and (b) overuse for more than 3 months of one or more drugs that can be taken for acute and/or symptomatic treatment of headache.

Said overuse may comprise use of an ergot alkoid (e.g., ergotamine) on 10 or more days/month, use of a triptan on 10 or more days/month, use of one or more non-opioid analgesics (such as paracetamol (acetaminophen), acetylsalicylic acid (aspirin), another NSAID, or another non-opioid analgesic) on 15 or more days/month, use of one or more combination-analgesics (as further described below) on 10 or more days/month, use of one or more opioids on 10 or more days/month, or use of a combination of two or more drug classes (as further described below) on 10 or more days/month.

In the methods herein, said triptan may include, without limitation thereto, any one of or any combination of triptans such as sumatriptan, zolmitriptan, naratriptan, rizatriptan, eletriptan, almotriptan, and frovatriptan, among others.

Said medication overuse headache may comprise ergotamine-overuse headache, triptan-overuse headache, non-opioid analgesic-overuse headache, opioid-overuse headache, combination-analgesic-overuse headache, medication-overuse headache attributed to multiple drug classes not individually overused, medication-overuse headache attributed to unspecified or unverified overuse of multiple drug classes, or medication-overuse headache attributed to other medication.

Said non-opioid analgesic-overuse headache may comprise paracetamol (acetaminophen)-overuse headache, non-steroidal anti-inflammatory drug (NSAID)-overuse headache such as acetylsalicylic acid (aspirin)-overuse headache or ibuprofen-overuse headache, or another non-opioid analgesic-overuse headache.

Said ergotamine-overuse headache may comprise headache occurring on 15 or more days/month in a patient with a pre-existing primary headache and developing as a consequence of regular use of an ergot alkaloid such as ergotamine on 10 or more days/month for more than 3 months.

In the methods herein, said ergot alkaloid may comprise ergotamine, nicergoline, methysergide, or dihydroergotamine, or may comprise an ergot derivative.

Said triptan-overuse headache may comprise headache occurring on 15 or more days/month in a patient with a pre-existing primary headache and developing as a consequence of regular use of one or more triptans on 10 or more days/month for more than 3 months.

Said non-opioid analgesic-overuse headache may comprise headache occurring on 15 or more days/month in a patient with a pre-existing primary headache and developing as a consequence of regular use of one or more non-opioid analgesics (such as paracetamol (acetaminophen), acetylsalicylic acid (aspirin), ibuprofen, another NSAID, or another non-opioid analgesic) on 15 or more days/month for more than 3 months.

In the methods herein, said NSAID may comprise any NSAID or combination thereof, including without limitation thereto, ibuprofen, naproxen, or indomethacin.

Said combination-analgesic-overuse headache may comprise headache occurring on 15 or more days/month developing as a consequence of regular use of one or more combination-analgesics on 10 or more days/month for more than 3 months. In the context of medication overuse headache, the term combination-analgesic refers to formulations combining drugs of two or more classes, each with analgesic effects (for example, paracetamol and codeine) or analgesics in combination with agents acting as adjuvants (for example, caffeine). Commonly overused combination-analgesics combine non-opioid analgesics with at least one opioid, barbiturate such as butalbital and/or caffeine. In exemplary embodiments, the combination-analgesic overuse-headache is due to the combination of acetaminophen, aspirin, and caffeine, e.g., EXCEDRIN® or EXCEDRIN MIGRAINE®. Other known combination analgesics comprise an analgesic in combination with at least one non-analgesic, e.g., with a vasoconstrictor drug such as pseudoephedrine for sinus-related preparations, antihistamine drug used to treat allergy sufferers, etc.

Said opioid-overuse headache may comprise headache occurring on 15 or more days/month in a patient with a pre-existing primary headache and developing as a consequence of regular use of one or more opioids 10 or more days/month for more than 3 months.

Said medication-overuse headache attributed to multiple drug classes not individually overused may comprise headache occurring on 15 or more days/month in a patient with a pre-existing primary headache and developing as a result of regular intake of any combination of ergotamine, triptans, non-opioid analgesics and/or opioids on a total of at least 10 days/month for more than 3 months without overuse of any single drug or drug class alone.

In the methods herein, said opioid may be any one or any combination of opioid drugs, including without limitation thereto, oxycodone, tramadol, butorphanol, morphine, codeine, hydrocodone, thebaine, oripavine, mixed opium alkaloids such as papaveretum, diacetylmorphine, nicomorphine, dipropanoylmorphine, diacetyldihydromorphine, acetylpropionylmorphine, desomorphine, methyldesorphine, dibenzoylmorphine, ethylmorphine, heterocodeine, buprenorphine, etorphine, hydromorphone, oxymorphone, fentanyl, alphamethylfentanyl, alfentanil, sufentanil, remifentanil, carfentanyl, ohmefentanyl, pethidine (meperidine), ketobemidone, MPPP, allylprodine, prodine, PEPAP, promedol, diphenylpropylamine, propoxyphene, dextropropoxyphene, dextromoramide, bezitramide, piritramide, among others.

Said medication-overuse headache attributed to unspecified or unverified overuse of multiple drug classes may comprise headache occurring on 15 or more days/month in a patient with a pre-existing primary headache and developing as a result of regular intake of any combination of ergotamine, triptans, non-opioid analgesics and/or opioids on at least 10 days/month for more than 3 months, wherein the identity, quantity and/or pattern of use or overuse of these classes of drug is not reliably established.

Said medication-overuse headache attributed to other medication may comprise headache occurring on 15 or more days/month in a patient with a pre-existing primary headache and developing as a result of regular intake of one or more medications other than those described above, taken for acute or symptomatic treatment of headache, on at least 10 days/month for more than 3 months.

The amount and duration of medication use may be determined utilizing known methods, such as the usage reported by the patient or a relative, a diary, medical records, drug purchase history, prescription fulfilment, biomarkers of medication use, incidence of medication toxicity, incidence of medication overdose, and/or other indicators of a patient's medication use.

The present disclosure provides methods of treating or preventing probable medication overuse headache, comprising administering to a patient in need an effective amount of an anti-CGRP antibody or anti-CGRP antibody fragment or one or more formulations comprising said anti-CGRP antibody or anti-CGRP antibody fragment as disclosed herein. Said anti-CGRP antibody optionally comprises any one of Ab1-Ab14, such as Ab6, e.g., having the light chain CDR 1, 2, and 3 polypeptide sequences of SEQ ID NO: 224; SEQ ID NO: 226; and SEQ ID NO: 228, respectively and the heavy chain CDR 1, 2, and 3 polypeptide sequences of SEQ ID NO: 204; SEQ ID NO: 206; and SEQ ID NO: 208; or having the light chain CDR 1, 2, and 3 polypeptide sequences encoded by SEQ ID NO: 234; SEQ ID NO: 236; and SEQ ID NO: 238, respectively and heavy chain CDR 1, 2, and 3 polypeptide sequences encoded by SEQ ID NO: 214; SEQ ID NO: 216; and SEQ ID NO: 218, respectively. Said anti-CGRP antibody may comprise the variable light chain polypeptide of SEQ ID NO: 222 and the variable heavy chain polypeptide of SEQ ID NO: 202. Said anti-CGRP antibody may comprise the variable light chain polypeptide encoded by SEQ ID NO: 232 and the variable heavy chain polypeptide encoded by SEQ ID NO: 212. Said anti-CGRP antibody may comprise the light chain polypeptide of SEQ ID NO: 221 and the heavy chain polypeptide of SEQ ID NO: 201 or SEQ ID NO: 566. Said anti-CGRP antibody may comprise the light chain polypeptide encoded by SEQ ID NO: 231 and the heavy chain polypeptide encoded by SEQ ID NO: 211 or SEQ ID NO: 567. Said anti-CGRP antibody may comprise the antibody expression product isolated from recombinant cells which express nucleic acid sequences encoding the variable light chain polypeptide of SEQ ID NO: 222 and the variable heavy chain polypeptide of SEQ ID NO: 202, which polypeptides optionally are respectively linked to human light and heavy constant region polypeptides, e.g., human IgG1, IgG2, IgG3 or IgG4 constant regions, which constant regions optionally may be modified to alter glycosylation or proteolysis, wherein said recombinant cells optionally comprise yeast or mammalian cells, e.g., Pichia pastoris or CHO cells. Said anti-CGRP antibody may comprise the antibody expression product isolated from recombinant cells which express nucleic acid sequences encoding the light chain of SEQ ID NO: 221 and the heavy chain polypeptide of SEQ ID NO: 201 or SEQ ID NO: 566, wherein said recombinant cells optionally comprise yeast or mammalian cells, e.g., Pichia pastoris or CHO cells, wherein the constant regions thereof optionally may be modified to alter glycosylation or proteolysis or other effector functions. Any of the aforementioned anti-CGRP antibodies or antibody fragments, preferably Ab6, may be optionally comprised in a formulation as disclosed herein, e.g., comprising histidine (L-histidine), sorbitol, polysorbate 80, such as, per 1 mL volume, about 100 mg anti-CGRP antibody, about 3.1 mg L-Histidine, about 40.5 mg Sorbitol, and about 0.15 mg Polysorbate 80, having a pH of about 5.8. The administered dosage of said antibody may be between about 100 mg and about 300 mg, such as about 100 mg, about 300 mg, 100 mg, or 300 mg. The dosage may be administered by different means, e.g., intravenously, e.g., in a saline solution such as 0.9% sodium chloride in a suitable volume, such as 100 mL. Probable medication overuse headache refers to criteria (a) and (b) not being entirely fulfilled, e.g., having at least 80% or at least 90% of the specified number of headache days and/or medication use days per month, and/or over a shorter time period such as at least 2 months, optionally in the absence of another ICHD-3 diagnosis.

Said medication-overuse headache (such as ergotamine-overuse headache, triptan-overuse headache, non-opioid analgesic-overuse headache, opioid-overuse headache, combination-analgesic-overuse headache, medication-overuse headache attributed to multiple drug classes not individually overused, medication-overuse headache attributed to unspecified or unverified overuse of multiple drug classes, or medication-overuse headache attributed to other medication) may be diagnosed according to the third edition of the International Classification of Headache Disorders (ICHD-3). See Headache Classification Committee of the International Headache Society (IHS), The International Classification of Headache Disorders, 3rd edition, Cephalalgia. 2018 January; 38(1):1-211, which is hereby incorporated by reference in its entirety.

Herein, the criterion that a headache occurs “as a consequence of” over use of a medication or medications refers to the apparent association between the medication(s) overuse and the headache, e.g., that the medication(s) overuse and headache are present at the above-specified frequency such that causation may be presumed.

The present disclosure also provides methods of treating chronic migraine, comprising intravenously administering to a patient in need thereof a first dosage comprising between about 100 mg and about 300 mg of an anti-CGRP antibody, wherein said anti-CGRP antibody preferably comprises the light chain polypeptide of SEQ ID NO: 221 and the heavy chain polypeptide of SEQ ID NO: 201 or 566, wherein in the first 24 hours after administration of said first dosage the patient exhibits at least a 50% reduction in migraine prevalence.

In another aspect, the disclosure provides methods of treating chronic migraine, comprising intravenously administering to a patient in need thereof a first dosage comprising between about 100 mg and about 300 mg of an anti-CGRP antibody, wherein said anti-CGRP antibody preferably comprises the light chain polypeptide of SEQ ID NO: 221 and the heavy chain polypeptide of SEQ ID NO: 201 or 566, wherein on the first day following the day of administration the patient exhibits at least a 50% reduction in migraine prevalence.

In some exemplary embodiments the dosage, e.g., the first dosage, of said anti-CGRP antibody may be 100 mg.

In other exemplary embodiments the dosage, e.g., the first dosage, of said anti-CGRP antibody may be 300 mg.

The method may further comprise intravenously administering 100 mg of said anti-CGRP antibody every 10⁻¹⁴ weeks, preferably every 11-13 weeks, more preferably every 12 weeks.

The method may further comprise intravenously administering 300 mg of said anti-CGRP antibody every 10⁻¹⁴ weeks, preferably every 11-13 weeks, more preferably every 12 weeks.

The antibody may be provided or administered in a formulation as disclosed herein, e.g., comprising histidine (L-histidine), sorbitol, polysorbate 80, such as, per 1 mL volume, about 100 mg anti-CGRP antibody, about 3.1 mg L-Histidine, about 40.5 mg Sorbitol, and about 0.15 mg Polysorbate 80, having a pH of about 5.8.

Prior to said first dosage, the patient may exhibit between about 10 and about 22 migraine days per month, such as between about 13 and about 19 migraine days per month, such as about 16 migraine days per month.

Prior to said first dosage, the patient may exhibit between about 14 and about 27 headache days per month, such as between about 17 and about 24 headache days per month, such as about 20 or about 21 headache days per month.

Said patient may have been diagnosed with migraine at least 10 years prior to said first dosage, such as at least 15 years prior to said first dosage, such as at least 18 or at least 19 years prior to said first dosage.

Said patient may have been diagnosed with chronic migraine at least 5 years prior to said first dosage, such as at least 8 years prior to said first dosage, such as at least 11 or at least 12 years prior to said first dosage.

The patient may have a headache when administered said first dosage.

The patient may have a migraine, such as a migraine with aura, when administered said first dosage.

Said patient may have a reduction in the number of migraine days by at least 50% in the one month period after being administered said first dose relative to the baseline number of migraine days experienced by that patient prior to said first dose.

Said patient may have a reduction in the number of migraine days by at least 75% in the one month period after being administered said first dose relative to the baseline number of migraine days experienced by that patient prior to said first dose.

Said patient may have a reduction in the number of migraine days by 100% in the one month period after being administered said first dose relative to the baseline number of migraine days experienced by that patient prior to said first dose.

Said patient may have a reduction in the number of migraine days by at least 50% in the 12 week period after being administered said first dose relative to the baseline number of migraine days experienced by that patient prior to said first dose.

Said patient may have a reduction in the number of migraine days by at least 75% in the 12 week period after being administered said first dose relative to the baseline number of migraine days experienced by that patient prior to said first dose.

Said patient may have a reduction in the number of migraine days by 100% in the 12 week period after being administered said first dose relative to the baseline number of migraine days experienced by that patient prior to said first dose.

The method may further comprise administering, e.g., intravenously, a second dose of said anti-CGRP antibody to said patient within about 10⁻¹⁴ weeks, preferably 11-13 weeks, more preferably about 12 weeks or about 3 months, after said first dose.

Said first dose may comprise about 100 mg, about 125 mg, about 150 mg, about 175 mg, about 200 mg, about 225 mg, about 250 mg, about 275 mg, or about 300 mg of said anti-CGRP antibody.

Said patient may be a chronic migraine patient or episodic migraine patient at risk of developing medication overuse headache. Said patient may use acute headache medication on at least 1, 2, 3, 4, 5, 6, 7, 8, 9, or 10 day(s) per month. Said patient may use acute headache medication on at least 10 days per month. Optionally said acute medication use is determined over a baseline period of at least 28 days. Said acute medication use may be reported by the patient, a caregiver, or based on records. Said acute medication may comprise use of ergot alkaloids, triptans, non-opioid analgesics, acetaminophen, aspirin, NSAIDs, non-opioid analgesics, combination-analgesics, or opioids.

Prior to said administration, the patient may exhibit between about 15 and about 30 migraine days per month, such as between about 16 and about 28 migraine days per month, such as between about 17 and about 26 migraine days per month, such as about 16 migraine days per month.

Prior to said administration, the patient may exhibit between about 15 and about 27 headache days per month, such as between about 17 and about 24 headache days per month, such as about 20 or about 21 headache days per month.

Said patient may have been diagnosed with migraine at least 10 years prior to said administration, such as at least 15 years prior to said administration, such as at least 18 or at least 19 years prior to said administration.

Said patient may have been diagnosed with chronic migraine at least 5 years prior to said administration, such as at least 8 years prior to said administration, such as at least 11 or at least 12 years prior to said administration.

Said patient may have a reduction in the number of migraine days by at least 50% in the one month period after being administered said antibody relative to the baseline number of migraine days experienced by that patient prior to said administration.

Said patient may have a reduction in the number of migraine days by at least 75% in the one month period after being administered said antibody relative to the baseline number of migraine days experienced by that patient prior to said administration.

Said patient may have a reduction in the number of migraine days by 100% in the one month period after being administered said antibody relative to the baseline number of migraine days experienced by that patient prior to said administration.

Said patient may have a reduction in the number of migraine days by at least 50% in the 12 week period after being administered said antibody relative to the baseline number of migraine days experienced by that patient prior to said administration.

Said patient may have a reduction in the number of migraine days by at least 75% in the 12 week period after being administered said antibody relative to the baseline number of migraine days experienced by that patient prior to said administration.

Said patient may have a reduction in the number of migraine days by 100% in the 12 week period after being administered said antibody relative to the baseline number of migraine days experienced by that patient prior to said administration.

The method may further comprise administering, e.g., intravenously, a second dose of said anti-CGRP antibody to said patient within about 10⁻¹⁴ weeks, preferably 11-13 weeks, more preferably about 12 weeks or about 3 months, after said administration.

Said administration may comprise about 100 mg, about 125 mg, about 150 mg, about 175 mg, about 200 mg, about 225 mg, about 250 mg, about 275 mg, or about 300 mg of said anti-CGRP antibody.

Said anti-CGRP antibody may be aglycosylated or if glycosylated only may contain only mannose residues.

Said anti-CGRP antibody may consist of the light chain polypeptide of SEQ ID NO: 221 and the heavy chain polypeptide of SEQ ID NO: 201 or SEQ ID NO: 566. Said anti-CGRP antibody may consist of the light chain polypeptide encoded by SEQ ID NO: 231 and the heavy chain polypeptide encoded by SEQ ID NO: 211 or SEQ ID NO: 567.

In some embodiments, said anti-human CGRP antibody or antibody fragment comprises the variable light chain of SEQ ID NO: 222 and/or the variable heavy chain of SEQ ID NO: 202. In some embodiments, said anti-human CGRP antibody or antibody fragment comprises the variable light chain encoded by SEQ ID NO: 232 and/or the variable heavy chain encoded by SEQ ID NO: 212.

In some embodiments, said anti-human CGRP antibody or antibody fragment comprises the light chain of SEQ ID NO: 221 and/or the heavy chain of SEQ ID NO: 201 or SEQ ID NO: 566. In some embodiments, said anti-human CGRP antibody or antibody fragment comprises the light chain encoded by SEQ ID NO: 231 and/or the heavy chain encoded by SEQ ID NO: 211 or SEQ ID NO: 567.

In some embodiments, said anti-CGRP antibody may comprise the antibody expression product isolated from recombinant cells which express nucleic acid sequences encoding the VL polypeptide of SEQ ID NO: 222 and the VH polypeptide of SEQ ID NO: 202, which polypeptides optionally are respectively linked to human light and heavy constant region polypeptides, e.g., human IgG1, IgG2, IgG3 or IgG4 constant regions, which constant regions optionally may be modified to alter glycosylation or proteolysis, wherein said recombinant cells optionally comprise yeast or mammalian cells, e.g., Pichia pastoris or CHO cells.

In some embodiments, said anti-CGRP antibody may comprise the antibody expression product isolated from recombinant cells which express nucleic acid sequences encoding the light chain of SEQ ID NO: 221 and the heavy chain polypeptide of SEQ ID NO: 201 or SEQ ID NO: 566, wherein said recombinant cells optionally comprise yeast or mammalian cells, e.g., Pichia pastoris or CHO cells, wherein the constant regions thereof optionally may be modified to alter glycosylation or proteolysis or other effector functions.

In some embodiments any of the aforementioned anti-CGRP antibodies or antibody fragments may be comprised in a formulation as disclosed herein, e.g., comprising histidine (L-histidine), sorbitol, polysorbate 80, such as, per 1 mL volume, about 100 mg anti-CGRP antibody, about 3.1 mg L-Histidine, about 40.5 mg Sorbitol, and about 0.15 mg Polysorbate 80, having a pH of about 5.8. The antibody or fragment may be administered by different means, e.g., intravenously, e.g., in a saline solution such as 0.9% sodium chloride in a suitable volume, such as 100 mL.

In some embodiments, about 100 mg, about 125 mg, about 150 mg, about 175 mg, about 200 mg, about 225 mg, about 250 mg, about 275 mg, or about 300 mg of said anti-CGRP antibody or antibody fragment is administered, e.g., intravenously.

In other embodiments, about 100 mg of said anti-CGRP antibody or antibody fragment is administered.

In other embodiments, about 300 mg of said anti-CGRP antibody or antibody fragment is administered, e.g., intravenously.

In exemplary embodiments, the anti-human CGRP antibody or antibody fragment is administered, e.g., intravenously at a frequency which is at most every 10⁻¹⁴ weeks, preferably every 11-13 weeks, more preferably every 3 months or every 12 weeks, wherein the antibody dosage is administered in a single formulation or divided into different formulations which are administered at a frequency of approximately every 10⁻¹⁴ weeks, preferably every 11-13 weeks, more preferably every 3 months or every 12 weeks. The phrase “the antibody dosage is administered in a single formulation or divided into different formulations” refers to the administration of the recited amount of antibody within a relatively short period of time, e.g., within a period of several hours, e.g., 1 to 8 hours, about one day, within about two days, or within about one week, which may be by the same or different routes (e.g., i.v., i.m., and/or s.c.), sites of administration. The term “different formulations” in this context refers to antibody dosages that are administered at different times and/or at different sites and/or different routes, irrespective of whether the dosages are the same or different with respect to the chemical composition of the pharmaceutical formulation in with each dosage is administered; for example, the concentration, excipients, carriers, pH, and the like may be the same or different between the different administered dosages.

In other exemplary embodiments, the anti-human CGRP antibody or antibody fragment dosage is administered in a single formulation or divided into different formulations which are administered at a frequency of approximately every 8 weeks or every 2 months.

In other exemplary embodiments, the anti-human CGRP antibody or antibody fragment dosage is administered in a single formulation or divided into different formulations which are administered at a frequency of approximately every 10⁻¹⁴ weeks, preferably every 11-13 weeks, more preferably every 12 weeks or every 3 months.

In other exemplary embodiments, the anti-human CGRP antibody or antibody fragment dosage is administered in a single formulation or divided into different formulations which are administered at a frequency of approximately every 16 weeks or every 4 months.

In other exemplary embodiments, the anti-human CGRP antibody or antibody fragment dosage is administered in a single formulation or divided into different formulations which are administered at a frequency of approximately every 20 weeks or every 5 months.

In other exemplary embodiments, the anti-human CGRP antibody or antibody fragment dosage is administered in a single formulation or divided into different formulations which are administered at a frequency of approximately every 24 weeks or every 6 months.

In other exemplary embodiments, the anti-human CGRP antibody or antibody fragment dosage is administered in a single formulation or divided into different formulations which are administered at a frequency of approximately every 28 weeks or every 7 months.

In other exemplary embodiments, the anti-human CGRP antibody or antibody fragment dosage is administered in a single formulation or divided into different formulations which are administered at a frequency of approximately every 32 weeks or every 8 months.

In other exemplary embodiments, the anti-human CGRP antibody or antibody fragment dosage is administered in a single formulation or divided into different formulations which are administered at a frequency of approximately every 36 weeks or every 9 months.

In other exemplary embodiments, the anti-human CGRP antibody or antibody fragment dosage is administered in a single formulation or divided into different formulations which are administered at a frequency of approximately every 40 weeks or every 8 months.

In other exemplary embodiments, the anti-human CGRP antibody or antibody fragment dosage is administered in a single formulation or divided into different formulations which are administered at a frequency of approximately every 44 weeks or every 9 months.

In other exemplary embodiments, the anti-human CGRP antibody or antibody fragment dosage is administered in a single formulation or divided into different formulations which are administered at a frequency of approximately every 48 weeks or every 10 months.

In other exemplary embodiments, the anti-human CGRP antibody or antibody fragment dosage is administered in a single formulation or divided into different formulations which are administered at a frequency of approximately every 52 weeks or every 11 months.

In other exemplary embodiments, the anti-human CGRP antibody or antibody fragment dosage is administered in a single formulation or divided into different formulations which are administered at a frequency of approximately every 56 weeks or every 12 months.

In other exemplary embodiments, the anti-human CGRP antibody or antibody fragment dosage is administered in a single formulation or divided into different formulations which are administered at a frequency of approximately every 15-18 months.

In other exemplary embodiments, the anti-human CGRP antibody or antibody fragment dosage is administered in a single formulation or divided into different formulations which are administered at a frequency of approximately every 18-21 months.

In other exemplary embodiments, the anti-human CGRP antibody dosage or antibody fragment used in the afore-mentioned methods is administered in a single formulation or divided into different formulations which are administered at a frequency of approximately every 2 years.

In other exemplary embodiments, the anti-human CGRP antibody used in the afore-mentioned methods is administered systemically.

In other exemplary embodiments, the anti-human CGRP antibody or antibody fragment used in the afore-mentioned methods is administered by a mode of administration is selected from intravenous, intramuscular, intravenous, intrathecal, intracranial, topical, intranasal, and oral. In a preferred embodiment, the anti-human CGRP antibody or antibody fragment used in the afore-mentioned methods is administered intravenously.

In other exemplary embodiments, the anti-human CGRP antibody used in the afore-mentioned methods has an in vivo half-life of at least 10 days.

In other exemplary embodiments, the anti-human CGRP antibody has an in vivo half-life of at least 15 days.

In other exemplary embodiments, the anti-human CGRP antibody used in the afore-mentioned methods has an in vivo half-life of at least 20 days.

In other exemplary embodiments, the anti-human CGRP antibody used in the afore-mentioned methods has an in vivo half-life of at least 20-30 days.

In other exemplary embodiments, the anti-human CGRP antibody is administered at a dosage of between about 100 mg and about 300 mg has an in vivo half-life of ±20% of at least about (284±44 hours).

In other exemplary embodiments, the anti-human CGRP antibody used in the afore-mentioned methods binds to human α- and β-CGRP.

In other exemplary embodiments, the administered anti-human CGRP antibody dosage results in the inhibition of vasodilation induced by topically applied capsaicin at least 30 days after antibody administration.

In other exemplary embodiments, the administered anti-human CGRP antibody dosage results in the inhibition of vasodilation induced by topically applied capsaicin at least 60 days after antibody administration.

In other exemplary embodiments, the administered anti-human CGRP antibody dosage results in inhibition of vasodilation induced by topically applied capsaicin at least 90 days after antibody administration.

In other exemplary embodiments, the administered anti-human CGRP antibody dosage results in the inhibition of vasodilation induced by topically applied capsaicin at least 120 days after antibody administration.

In other exemplary embodiments, the administered anti-human CGRP antibody dosage results in the inhibition of vasodilation induced by topically applied capsaicin at least 150 days after antibody administration.

In other exemplary embodiments, the administered anti-human CGRP antibody dosage results in the inhibition of vasodilation induced by topically applied capsaicin at least 180 days after antibody administration.

In other exemplary embodiments, the administered anti-human CGRP antibody dosage results in the inhibition of vasodilation induced by topically applied capsaicin more than 180 days after antibody administration.

In other exemplary embodiments, the administered anti-human CGRP antibody dosage results in sustained pharmacodynamic (PK) activity, within 5% of the maximal response (Imax) (as compared to lower antibody doses).

In other exemplary embodiments, the administered anti-human CGRP antibody dosage results in sustained pharmacodynamic (PK) activity which is maintained for at least 2-3 months after antibody administration, wherein PK analysis of the anti-human CGRP antibody is derived from plasma concentrations.

In other exemplary embodiments, the administered anti-human CGRP antibody dosage is between about 100 mg and about 300 mg or more which is administered no more frequently than every 2 months.

The present invention is additionally directed to the use of specific antibodies and fragments thereof having binding specificity for CGRP, in particular antibodies having desired epitopic specificity, high affinity or avidity and/or functional properties. A preferred embodiment of the invention is directed to usage of chimeric or humanized antibodies and fragments thereof (including Fab fragments) capable of binding to CGRP and/or inhibiting the biological activities mediated by the binding of CGRP to the CGRP receptor (“CGRP-R”) e.g., wherein such antibodies optionally are derived from recombinant cells engineered to express same, optionally yeast or mammalian cells, further optionally Pichia pastoris and CHO cells.

In another preferred embodiment of the invention, full length antibodies and Fab fragments thereof are contemplated that inhibit the CGRP-alpha-, CGRP-beta-, and rat CGRP-driven production of cAMP. In a further preferred embodiment of the invention, full length and Fab fragments thereof are contemplated that reduce vasodilation in a recipient following administration.

The invention also contemplates usage of conjugates of anti-CGRP antibodies and binding fragments thereof conjugated to one or more functional or detectable moieties. The invention also contemplates usage of chimeric or humanized anti-CGRP or anti-CGRP/CGRP-R complex antibodies and binding fragments thereof. In one embodiment, binding fragments include, but are not limited to, Fab, Fab′, F(ab′)₂, Fv, scFv fragments, SMIPs (small molecule immunopharmaceuticals), camelbodies, nanobodies, and IgNAR.

### BRIEF DESCRIPTION OF THE SEVERAL VIEWS OF THE DRAWINGS

FIGS. 1A-IF provide the polypeptide sequences of the full-length heavy chain for antibodies Ab1-Ab14 with their framework regions (FR), complementarity determining regions (CDRs), and constant region sequences delimited.

FIGS. 2A-2D provide the polypeptide sequences of the full-length light chain for antibodies Ab1-Ab14 with their framework regions (FR), complementarity determining regions (CDRs), and constant region sequences delimited.

FIGS. 3A-3P provide exemplary polynucleotide sequences encoding the full-length heavy chain for antibodies Ab1-Ab14 with their framework regions (FR), complementarity determining regions (CDRs), and constant region coding sequences delimited.

FIGS. 4A-4I provide exemplary polynucleotide sequences encoding the full-length light chain for antibodies Ab1-Ab14 with their framework regions (FR), complementarity determining regions (CDRs), and constant region coding sequences delimited.

FIG. 5 provides the polypeptide sequence coordinates within the full-length heavy chain polypeptide sequences of antibodies Ab1-Ab14 of sequence features including the variable region and complementarity determining regions (CDRs), and the SEQ ID NO of each individual feature.

FIG. 6 provides the polypeptide sequence coordinates within the full-length heavy chain polypeptide sequences of antibodies Ab1-Ab14 of sequence features including the framework regions (FRs) and constant region, and the SEQ ID NO of each individual feature.

FIG. 7 provides the polypeptide sequence coordinates within the full-length light chain polypeptide sequences of antibodies Ab1-Ab14 of sequence features including the variable region and complementarity determining regions (CDRs), and the SEQ ID NO of each individual feature.

FIG. 8 provides the polypeptide sequence coordinates within the full-length light chain polypeptide sequences of antibodies Ab1-Ab14 of sequence features including the framework regions (FRs) and constant region, and the SEQ ID NO of each individual feature.

FIG. 9 provides the polynucleotide sequence coordinates within the exemplary polynucleotide sequences encoding the full-length heavy chain polypeptide sequences of antibodies Ab1-Ab14 of sequence features including the variable region and complementarity determining regions (CDRs), and the SEQ ID NO of each individual feature.

FIG. 10 provides the polynucleotide sequence coordinates within the exemplary polynucleotide sequences encoding the full-length heavy chain polypeptide sequences of antibodies Ab1-Ab14 of sequence features including the framework regions (FRs) and constant region, and the SEQ ID NO of each individual feature.

FIG. 11 provides the polynucleotide sequence coordinates within the exemplary polynucleotide sequences encoding the full-length light chain polypeptide sequences of antibodies Ab1-Ab14 of sequence features including the variable region and complementarity determining regions (CDRs), and the SEQ ID NO of each individual feature.

FIG. 12 provides the polynucleotide sequence coordinates within the exemplary polynucleotide sequences encoding the full-length light chain polypeptide sequences of antibodies Ab1-Ab14 of sequence features including the framework regions (FRs) and constant region, and the SEQ ID NO of each individual feature.

FIG. 13 shows the number of subjects in a human clinical trial described in Example 2 who were either treated with Ab6 (treatment group) or placebo groups who showed a 50, 75 or 100% reduction in migraines at each monitoring point throughout the period. The right bar in each group corresponds to patients receiving 1000 mg Ab6 and the left bar in each group corresponds to matched placebo controls. In each response rate group the patients receiving Ab6 had a significantly greater response rate than placebo-treated controls, with p values of 0.0155, 0.0034, and 0.0006 in each respective group as indicated. The administered antibody was produced in P. pastoris and consisted of the light chain polypeptide of SEQ ID NO: 221 and the heavy chain polypeptide of SEQ ID NO: 201.

FIG. 14 shows the median (±QR) % change from baseline in the number of migraine days per month in the placebo and Ab6-treated group over the 12 weeks post-treatment. (p=0.0078). The upper (red) line and lower (blue) line show results for placebo-treated controls and patients administered 1000 mg Ab6, respectively.

FIG. 15 shows the median (±QR) % change from baseline in the number of migraine episodes per month in the placebo and Ab6-treated group over the 12 weeks post-treatment. The upper (red) line and lower (blue) line show results for placebo-treated controls and patients administered 1000 mg Ab6, respectively.

FIG. 16 shows the median (±QR) % change from baseline in the number of migraine hours per month in the placebo and Ab6-treated group over the 12 weeks post-treatment. The upper (red) line and lower (blue) line show results for placebo-treated controls and patients administered 1000 mg Ab6, respectively.

FIG. 17 summarizes the screening of patients, allocation into the treatment and control groups, and loss of patients through follow-up.

FIG. 18 compares the HIT-6 responder analysis for the Ab6-treated and placebo groups at baseline, week 4 after treatment, week 8 after treatment and week 12 after treatment.

FIG. 19 shows the percentage of patients for whom the HIT-6 analysis indicated that the effect of headaches was only “some” or “little/none” at baseline and after Ab6 administration. At baseline most patients had either “substantial” or “severe” impact from migraines. At each subsequent time point, a significantly greater percentage of patients administered 1000 mg Ab6 had only “some” or “little/none” HIT-6 impact (left bar in each group, colored blue) as compared to placebo controls (right bar in each group, colored red).

FIG. 20 contains the pharmacokinetic (PK) profile for Ab6 administered intravenously at a single dosage of 1000 mg.

FIG. 21 contains plasma-free pharmacokinetic (PK) parameters N (number of patients), mean, and standard deviation (SD) for a single 1000 mg intravenous dosage of Ab6. The parameters shown in the table and the units are Cₘₐₓ (μg/mL), AUC_{0-∞} (mg*hr/mL), half-life (days), V_{z} (L) and C_{L}(mL/hr).

FIG. 22 shows the change (mean+−SEM) change from baseline in migraine days per month for Ab6 (1000 mg i.v.) versus placebo as a single dose for the study described in Example 2.

FIG. 23 shows the average migraine days (+/−SD) over time for the full analysis population for the study described in Example 2. Normalization was applied to visit intervals where eDiaries were completed for 21-27 days by multiplying the observed frequency by the inverse of the completion rate.

FIG. 24 shows the distribution of migraine days actual and change for the Ab6 treatment group during weeks 1-4 for the study described in Example 2.

FIG. 25 shows the distribution of migraine days actual and change for the placebo group during weeks 1-4 for the study described in Example 2.

FIG. 26 shows the distribution of migraine days actual and change for the Ab6 treatment group during weeks 5-8 for the study described in Example 2.

FIG. 27 shows the distribution of migraine days actual and change for the placebo group during weeks 5-8 for the study described in Example 2.

FIG. 28 shows the distribution of migraine days actual and change for the Ab6 treatment group during weeks 9-12 for the study described in Example 2.

FIG. 29 shows the distribution of migraine days actual and change for the placebo group during weeks 9-12 for the study described in Example 2.

FIG. 30 shows the 50% responder rate for the Ab6 and placebo treatment groups for the study described in Example 2. Subjects with ≥50% reduction in migraine frequency were considered to be a 50% responder. Normalization was applied to visit intervals where eDiary was completed for 21-27 days by multiplying the observed frequency by the inverse of the completion rate.

FIG. 31 shows the 75% responder rate for the Ab6 and placebo treatment groups for the study described in Example 2. Subjects with ≥75% reduction in migraine frequency were considered to be a 75% responder. Normalization was applied as described with FIG. 30.

FIG. 32 shows the 100% responder rate for the Ab6 and placebo treatment group for the study described in Example 2. Subjects with 100% reduction in migraine frequency were considered to be a 100% responder. Normalization was applied as described with FIG. 30.

FIG. 33 shows the mean migraine severity over time for the full analysis population for the study described in Example 2. On the scale used, a mean migraine score of 3 represents “moderate pain.”

FIG. 34 summarizes the change from baseline in measured attributes for the placebo and treatment groups in the study described in Example 2.

FIG. 35 shows the percentages of patients with migraine in the 300 mg, 100 mg, and placebo treatment groups at days 1, 7, 14, 21, and 28 in the clinical trial described in Example 3. The uppermost line shows results for placebo, the lowest line shows results for the 300 mg dosage, and the middle line shows results for the 100 mg dosage.

FIG. 36 show the percentage of patients in the 300 mg and 100 mg treatment groups achieving a 50% reduction in migraine days in month 1, over months 1-3 (after the 1st infusion), and over months 4-5 (after the 2nd infusion) in the clinical trial described in Example 3. In each graph, the data bars, from left to right, show results for the 100 mg, 300 mg, and placebo groups. Statistical significance is as shown. ++ indicates a statistically significant difference from placebo; + indicates a statistically significant difference from placebo (unadjusted); and § indicates a statistically significant difference from placebo (post hoc).

FIG. 37 show the percentage of patients in the 300 mg and 100 mg treatment groups achieving a 75% reduction in migraine days in month 1, over months 1-3 (after the 1st infusion), and over months 4-5 (after the 2nd infusion) in the clinical trial described in Example 3. Data order and statistical significance labels are as indicated with FIG. 36.

FIG. 38 show the percentage of patients in the 300 mg and 100 mg treatment groups achieving a 100% reduction in migraine days in month 1, over months 1-3 (after the 1st infusion), and over months 4-5 (after the 2nd infusion) in the clinical trial described in Example 3. Data order and statistical significance labels are as indicated with FIG. 36.

FIG. 39 summarizes the characteristics of patients in each treatment group in the clinical trial described in Example 3. * According to the American Academy of Neurology/American Headache Society guidelines for migraine preventative treatment (medications identified by clinical review of coded medical data); SD, standard deviation; BMI, body mass index.

FIG. 40. Difference from placebo in change from baseline in mean migraine days (MMD) over months 1-3 by baseline subgroup for a human clinical trial of chronic migraine patients. In the graph, the data point refers to the mean value and the line shows the 95% confidence interval (CI) of the change from placebo for the 100 mg (upper line) or 300 mg (lower line) treatment group, for each subgroup as labeled at the far left.

FIG. 41. Difference from placebo in change from baseline in mean migraine days (MMD) over months 1-3 by baseline subgroup for a human clinical trial of episodic migraine patients. The graph is labeled as in FIG. 40.

FIG. 42. Change from baseline in mean migraine days (MMDs) across 2 dose intervals in chronic migraine patients with at least 1 day of acute medication use per month at baseline. Triangle: placebo (n=366). Circle: 100 mg Ab6 per dose (n=356). Square: 300 mg Ab6 per dose (n=350).

FIG. 43. Mean days with acute medication use in chronic migraine patients with at least one day per month of acute medication use at baseline. Triangle: placebo (n=366). Circle: 100 mg Ab6 per dose (n=356). Square: 300 mg Ab6 per dose (n=350).

FIG. 44. Change from baseline in acute medication use by subgroups of chronic migraine patients with differing baseline days of acute medication use. Solid lines: patients with 10 or more days of acute medication use per month at baseline. Dashed lines: patients with at least 1 and less than 10 days of acute medication use per month at baseline. Triangle: placebo. Circle: 100 mg Ab6 per dose. Square: 300 mg Ab6 per dose.

FIG. 45. Summary of Acute Medication Days by Subgroups of Chronic Migraine Patients with Baseline Acute Medication Use.

FIG. 46. Change from baseline in mean migraine days (MMDs) across 2 dose intervals in episodic migraine patients with at least 1 day of acute medication use per month at baseline. Triangle: placebo (n=222). Circle: 100 mg Ab6 per dose (n=221). Square: 300 mg Ab6 per dose (n=222).

FIG. 47. Mean days with acute medication use in episodic migraine patients with at least one day per month of acute medication use at baseline. Triangle: placebo (n=222). Circle: 100 mg Ab6 per dose (n=221). Square: 300 mg Ab6 per dose (n=222).

FIG. 48. Change from baseline in acute medication use by subgroups of episodic migraine patients with differing baseline days of acute medication use. Solid lines: patients with 10 or more days of acute medication use per month at baseline. Dashed lines: patients with at least 1 and less than 10 days of acute medication use per month at baseline. Triangle: placebo. Circle: 100 mg Ab6 per dose. Square: 300 mg Ab6 per dose.

FIG. 49. Summary of Acute Medication Days by Subgroups of Episodic Migraine Patients with Baseline Acute Medication Use.

### DETAILED DESCRIPTION

Use of anti-CGRP antibodies for treatment of medication overuse headache is described herein. Additionally, anti-CGRP antibodies are demonstrated herein to be effective for treatment of chronic migraine. The treatment was shown to have a very rapid onset of efficacy, with relief from migraine observed on the first day following administration.

### Definitions

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims. As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

As used herein, the term “medication overuse headache” refers to a headache that meets the criteria for that condition specified in ICHD-3 (Headache Classification Committee of the International Headache Society (IHS), The International Classification of Headache Disorders, 3rd edition, Cephalalgia. 2018 January; 38(1):1-211). The term includes subtypes of medication overuse headache, as defined in the ICHD-3, such as triptan-overuse headache, non-opioid analgesic overuse headache, opioid overuse headache, etc.

As used herein, the term “reduction in migraine prevalence” refers to a reduction (e.g., a stated percentage reduction, such as 50%) in the likelihood of a patient having a migraine in the stated period, such as the 18 hour, 20 hour, 24 hour, 28 hour, or 30 hour period, preferably the 24 hour period, after a first dosage of an antibody, or on the first day following the day of antibody administration (i.e., on the first full day following the day on which the antibody administration is completed). It is to be understood that a given patient may or may not have a migraine during that period, as the reduction in likelihood may be observable over a large number of patients irrespective of the outcome for an individual patient.

As used herein, the term “chronic migraine” refers to a condition wherein a patient exhibits, on average, at least 15 migraine days and/or headache per month. The term “episodic migraine” refers to a condition wherein a patient exhibits, on average, less than 15 headache and/or migraine days per month.

As used herein, the term “diagnosed with chronic migraine” refers to a patient meeting the clinical criteria for chronic migraine, whether or not a formal diagnosis of that patient was performed.

As used herein, the term “intravenously administering” refers to a mode of administration wherein a substance, e.g., an antibody, is introduced directly into the circulation of that patient, most typically into the venous circulation. The substance may be introduced in a carrier fluid, such as an aqueous solution, e.g., normal saline. The substance may be administered in a single formulation or in multiple formulations, as long as the administration is completed over a short period of time (e.g., within 1 day, preferably within 12 hours, more preferably within 6 hours, and most preferably within 1-2 hours).

As used herein, the term “the baseline number of migraine days” refers to the number of migraine days exhibited by a patient in a specified time period, e.g., prior to treatment. For example, the baseline number of migraine days may be determined over a period of one month, or longer, e.g., by recording each day whether or not a migraine occurred.

As used herein, the term “migraine days per month” refers to the number of days per month on which a patient has a migraine, i.e., at any time during that day, the patient has symptoms that meet the clinical definition of migraine. The number of migraine days per month may be determined by recording each day whether or not a migraine occurred.

As used herein, the term “headache days per month” refers to the number of days per month on which a patient has a headache, i.e., at any time during that day, the patient has symptoms that meet the clinical definition of a headache. The number of headache days per month may be determined by recording each day whether or not a headache occurred.

Calcitonin Gene Related Peptide (CGRP): As used herein, CGRP encompasses not only the following Homo sapiens CGRP-alpha and Homo sapiens CGRP-beta amino acid sequences available from American Peptides (Sunnyvale CA) and Bachem (Torrance, CA):
CGRP-alpha: ACDTATCVTHRLAGLLSRSGGVVKNNFVPTNVGSKAF-NH₂ (SEQ ID NO: 561), wherein the terminal phenylalanine is amidated;
CGRP-beta: ACNTATCVTHRLAGLLSRSGGMVKSNFVPTNVGSKAF-NH₂ (SEQ ID NO: 562), wherein the terminal phenylalanine is amidated; but also any membrane-bound forms of these CGRP amino acid sequences, as well as mutants (mutiens), splice variants, isoforms, orthologs, homologues and variants of this sequence.

Expression Vector: These DNA vectors contain elements that facilitate manipulation for the expression of a foreign protein within the target host cell, e.g., a yeast or mammalian cell such as Pichia pastoris or CHO cells. Conveniently, manipulation of sequences and production of DNA for transformation is first performed in a bacterial host, e.g. E. coli, and usually vectors will include sequences to facilitate such manipulations, including a bacterial origin of replication and appropriate bacterial selection marker. Selection markers encode proteins necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, (b) complement auxotrophic deficiencies, or (c) supply critical nutrients not available from complex media. Exemplary vectors and methods for transformation of yeast are described, for example, in Burke, D., Dawson, D., & Steams, T. (2000). Methods in yeast genetics: a Cold Spring Harbor Laboratory course manual. Plainview, N.Y.: Cold Spring Harbor Laboratory Press.

Expression vectors for use in yeast or mammalian cells will generally further include yeast or mammalian specific sequences, including a selectable auxotrophic or drug marker for identifying transformed yeast strains or transformed mammalian cells. A drug marker may further be used to amplify copy number of the vector in the host cell.

The polypeptide coding sequence of interest is operably linked to transcriptional and translational regulatory sequences that provide for expression of the polypeptide in host cells, e.g., Pichia pastoris or CHO cells. These vector components may include, but are not limited to, one or more of the following: an enhancer element, a promoter, and a transcription termination sequence. Sequences for the secretion of the polypeptide may also be included, e.g. a signal sequence, and the like. A yeast or mammalian origin of replication is optional, as expression vectors are often integrated into the host cell genome. In one embodiment of the invention, the polypeptide of interest is operably linked, or fused, to sequences providing for optimized secretion of the polypeptide from yeast diploid cells.

Nucleic acids are “operably linked” when placed into a functional relationship with another nucleic acid sequence. For example, DNA for a signal sequence is operably linked to DNA for a polypeptide if it is expressed as a preprotein that participates in the secretion of the polypeptide; a promoter or enhancer is operably linked to a coding sequence if it affects the transcription of the sequence. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites or alternatively via a PCR/recombination method familiar to those skilled in the art (Gateway® Technology; Invitrogen, Carlsbad California). If such sites do not exist, the synthetic oligonucleotide adapters or linkers are used in accordance with conventional practice.

Promoters are untranslated sequences located upstream (5′) to the start codon of a structural gene (generally within about 100 to 1000 bp) that control the transcription and translation of particular nucleic acid sequences to which they are operably linked. Such promoters fall into several classes: inducible, constitutive, and repressible promoters (that increase levels of transcription in response to absence of a repressor). Inducible promoters may initiate increased levels of transcription from DNA under their control in response to some change in culture conditions, e.g., the presence or absence of a nutrient or a change in temperature.

The promoter fragment may also serve as the site for homologous recombination and integration of the expression vector into the same site in the host genome; alternatively a selectable marker is used as the site for homologous recombination. Examples of suitable promoters from Pichia include the AOX1 and promoter (Cregg et al. (1989) Mol. Cell. Biol. 9:1316-1323); ICL1 promoter (Menendez et al. (2003) Yeast 20(13):1097-108); glyceraldehyde-3-phosphate dehydrogenase promoter (GAP) (Waterham et al. (1997) Gene 186(1):37-44); and FLD1 promoter (Shen et al. (1998) Gene 216(1):93-102). The GAP promoter is a strong constitutive promoter and the AOX and FLD1 promoters are inducible.

Other yeast promoters include ADH1, alcohol dehydrogenase II, GAL4, PHO3, PHO5, Pyk, and chimeric promoters derived therefrom. Additionally, non-yeast promoters may be used in the invention such as mammalian, insect, plant, reptile, amphibian, viral, and avian promoters. Most typically the promoter will comprise a mammalian promoter (potentially endogenous to the expressed genes) or will comprise a yeast or viral promoter that provides for efficient transcription in yeast systems.

Examples of mammalian promoters include cytomegalovirus (CMV) derived promoters, chicken 3-actin (CBM) derived promoters, adenomatous polyposis coli (APC) derived promoters, leucine-rich repeat containing G protein-coupled receptor 5 (LGR5) promoters, CAG promoter, Beta actin promoter, elongation factor-1 (EF1) promoter, early growth response 1 (EGR-1) promoter, eukaryotic initiation factor 4A (EIF4A1) promoter, simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter, among others. Combinations of two or more of the foregoing promoters may also be used. Further, inducible promoters may be used. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The polypeptides of interest may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the polypeptide coding sequence that is inserted into the vector. The heterologous signal sequence selected preferably is one that is recognized and processed through one of the standard pathways available within the host cell. The S. cerevisiae alpha factor pre-pro signal has proven effective in the secretion of a variety of recombinant proteins from P. pastoris. Other yeast signal sequences include the alpha mating factor signal sequence, the invertase signal sequence, and signal sequences derived from other secreted yeast polypeptides. Additionally, these signal peptide sequences may be engineered to provide for enhanced secretion in diploid yeast expression systems. Secretion signals for use in mammalian as well as yeast cells include mammalian signal sequences, which may be heterologous to the protein being secreted, or may be a native sequence for the protein being secreted. Signal sequences include pre-peptide sequences, and in some instances may include propeptide sequences. Many such signal sequences are known in the art, including the signal sequences found on immunoglobulin chains, e.g., K28 preprotoxin sequence, PHA-E, FACE, human MCP-1, human serum albumin signal sequences, human Ig heavy chain, human Ig light chain, and the like. For example, see Hashimoto et. al. Protein Eng 11(2) 75 (1998); and Kobayashi et. al. Therapeutic Apheresis 2(4) 257 (1998).

Transcription may be increased by inserting a transcriptional activator sequence into the vector. These activators are cis-acting elements of DNA, usually about from 10 to 300 bp, which act on a promoter to increase its transcription. Transcriptional enhancers are relatively orientation and position independent, having been found 5′ and 3′ to the transcription unit, within an intron, as well as within the coding sequence itself. The enhancer may be spliced into the expression vector at a position 5′ or 3′ to the coding sequence, but is preferably located at a site 5′ from the promoter.

Expression vectors used in eukaryotic host cells may also contain sequences necessary for the termination of transcription and for stabilizing the mRNA. Such sequences are commonly available from 3′ to the translation termination codon, in untranslated regions of eukaryotic or viral DNAs or cDNAs. These regions contain nucleotide segments transcribed as polyadenylated fragments in the untranslated portion of the mRNA.

Construction of suitable vectors containing one or more of the above-listed components employs standard ligation techniques or PCR/recombination methods. Isolated plasmids or DNA fragments are cleaved, tailored, and re-ligated in the form desired to generate the plasmids required or via recombination methods. For analysis to confirm correct sequences in plasmids constructed, the ligation mixtures are used to transform host cells, and successful transformants selected by antibiotic resistance (e.g. ampicillin or Zeocin) where appropriate. Plasmids from the transformants are prepared, analyzed by restriction endonuclease digestion and/or sequenced.

As an alternative to restriction and ligation of fragments, recombination methods based on att sites and recombination enzymes may be used to insert DNA sequences into a vector. Such methods are described, for example, by Landy (1989) Ann. Rev. Biochem. 58:913-949; and are known to those of skill in the art. Such methods utilize intermolecular DNA recombination that is mediated by a mixture of lambda and E. coli-encoded recombination proteins. Recombination occurs between specific attachment (att) sites on the interacting DNA molecules. For a description of att sites see Weisberg and Landy (1983) Site-Specific Recombination in Phage Lambda, in Lambda II, Weisberg, ed. (Cold Spring Harbor, NY: Cold Spring Harbor Press), pp. 211-250. The DNA segments flanking the recombination sites are switched, such that after recombination, the att sites are hybrid sequences comprised of sequences donated by each parental vector. The recombination can occur between DNAs of any topology.

Att sites may be introduced into a sequence of interest by ligating the sequence of interest into an appropriate vector; generating a PCR product containing att B sites through the use of specific primers; generating a cDNA library cloned into an appropriate vector containing att sites; and the like.

Folding, as used herein, refers to the three-dimensional structure of polypeptides and proteins, where interactions between amino acid residues act to stabilize the structure. Proper folding is typically the arrangement of a polypeptide that results in optimal biological activity, and in the case of antibodies can conveniently be monitored by assays for activity, e.g. antigen binding.

The expression host may be further modified by the introduction of sequences encoding one or more enzymes that enhance folding and disulfide bond formation, i.e. foldases, chaperonins, etc. Such sequences may be constitutively or inducibly expressed in the yeast host cell, using vectors, markers, etc. as known in the art. Preferably the sequences, including transcriptional regulatory elements sufficient for the desired pattern of expression, are stably integrated in the yeast genome through a targeted methodology.

For example, the eukaryotic PDI is not only an efficient catalyst of protein cysteine oxidation and disulfide bond isomerization, but also exhibits chaperone activity. Co-expression of PDI can facilitate the production of active proteins having multiple disulfide bonds. Also of interest is the expression of BIP (immunoglobulin heavy chain binding protein); cyclophilin; and the like. In one embodiment of the invention, each of the haploid parental strains expresses a distinct folding enzyme, e.g. one strain may express BIP, and the other strain may express PDI or combinations thereof.

The terms “desired protein” or “desired antibody” are used interchangeably and refer generally to a parent antibody specific to a target, i.e., CGRP or a chimeric or humanized antibody or a binding portion thereof derived therefrom as described herein. The term “antibody” is intended to include any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammals, chicken, other avians, etc., are considered to be “antibodies.” A preferred source for producing antibodies useful as starting material according to the invention is rabbits. Numerous antibody coding sequences have been described; and others may be raised by methods well-known in the art. Examples thereof include chimeric antibodies, human antibodies and other non-human mammalian antibodies, humanized antibodies, single chain antibodies (such as scFvs), camelbodies, nanobodies, IgNAR (single-chain antibodies derived from sharks), small-modular immunopharmaceuticals (SMIPs), and antibody fragments such as Fabs, Fab′, F(ab′)₂ and the like. See Streltsov V A, et al., Structure of a shark IgNAR antibody variable domain and modeling of an early-developmental isotype, Protein Sci. 2005 November; 14(11):2901-9. Epub 2005 Sep. 30; Greenberg A S, et al., A new antigen receptor gene family that undergoes rearrangement and extensive somatic diversification in sharks, Nature. 1995 Mar. 9; 374(6518):168-73; Nuttall S D, et al., Isolation of the new antigen receptor from wobbegong sharks, and use as a scaffold for the display of protein loop libraries, Mol Immunol. 2001 August; 38(4):313-26; Hamers-Casterman C, et al., Naturally occurring antibodies devoid of light chains, Nature. 1993 Jun. 3; 363(6428):446-8; Gill D S, et al., Biopharmaceutical drug discovery using novel protein scaffolds, Curr Opin Biotechnol. 2006 December; 17(6):653-8. Epub 2006 Oct. 19.

For example, antibodies or antigen binding fragments may be produced by genetic engineering. In this technique, as with other methods, antibody-producing cells are sensitized to the desired antigen or immunogen. The messenger RNA isolated from antibody producing cells is used as a template to make cDNA using PCR amplification. A library of vectors, each containing one heavy chain gene and one light chain gene retaining the initial antigen specificity, is produced by insertion of appropriate sections of the amplified immunoglobulin cDNA into the expression vectors. A combinatorial library is constructed by combining the heavy chain gene library with the light chain gene library. This results in a library of clones which co-express a heavy and light chain (resembling the Fab fragment or antigen binding fragment of an antibody molecule). The vectors that carry these genes are co-transfected into a host cell. When antibody gene synthesis is induced in the transfected host, the heavy and light chain proteins self-assemble to produce active antibodies that can be detected by screening with the antigen or immunogen.

Antibody coding sequences of interest include those encoded by native sequences, as well as nucleic acids that, by virtue of the degeneracy of the genetic code, are not identical in sequence to the disclosed nucleic acids, and variants thereof. Variant polypeptides can include amino acid (aa) substitutions, additions or deletions. The amino acid substitutions can be conservative amino acid substitutions or substitutions to eliminate non-essential amino acids, such as to alter a glycosylation site, or to minimize misfolding by substitution or deletion of one or more cysteine residues that are not necessary for function. Variants can be designed so as to retain or have enhanced biological activity of a particular region of the protein (e.g., a functional domain, catalytic amino acid residues, etc). Variants also include fragments of the polypeptides disclosed herein, particularly biologically active fragments and/or fragments corresponding to functional domains. Techniques for in vitro mutagenesis of cloned genes are known. Also included in the subject invention are polypeptides that have been modified using ordinary molecular biological techniques so as to improve their resistance to proteolytic degradation or to optimize solubility properties or to render them more suitable as a therapeutic agent.

Chimeric antibodies may be made by recombinant means by combining the variable light and heavy chain regions (V_{L} and V_{H}), obtained from antibody producing cells of one species with the constant light and heavy chain regions from another. Typically chimeric antibodies utilize rodent or rabbit variable regions and human constant regions, in order to produce an antibody with predominantly human domains. The production of such chimeric antibodies is well known in the art, and may be achieved by standard means (as described, e.g., in U.S. Pat. No. 5,624,659, incorporated herein by reference in its entirety). It is further contemplated that the human constant regions of chimeric antibodies of the invention may be selected from IgG1, IgG2, IgG3, and IgG4 constant regions.

Humanized antibodies are engineered to contain even more human-like immunoglobulin domains, and incorporate only the complementarity-determining regions of the animal-derived antibody. This is accomplished by carefully examining the sequence of the hyper-variable loops of the variable regions of the monoclonal antibody, and fitting them to the structure of the human antibody chains. Although facially complex, the process is straightforward in practice. See, e.g., U.S. Pat. No. 6,187,287, incorporated fully herein by reference.

In addition to entire immunoglobulins (or their recombinant counterparts), immunoglobulin fragments comprising the epitope binding site (e.g., Fab′, F(ab′)₂, or other fragments) may be synthesized. “Fragment,” or minimal immunoglobulins may be designed utilizing recombinant immunoglobulin techniques. For instance “Fv” immunoglobulins for use in the present invention may be produced by synthesizing a fused variable light chain region and a variable heavy chain region. Combinations of antibodies are also of interest, e.g. diabodies, which comprise two distinct Fv specificities. In another embodiment of the invention, SMIPs (small molecule immunopharmaceuticals), camelbodies, nanobodies, and IgNAR are encompassed by immunoglobulin fragments.

Immunoglobulins and fragments thereof may be modified post-translationally, e.g. to add effector moieties such as chemical linkers, detectable moieties, such as fluorescent dyes, enzymes, toxins, substrates, bioluminescent materials, radioactive materials, chemiluminescent moieties and the like, or specific binding moieties, such as streptavidin, avidin, or biotin, and the like may be utilized in the methods and compositions of the present invention. Examples of additional effector molecules are provided infra.

A polynucleotide sequence “corresponds” to a polypeptide sequence if translation of the polynucleotide sequence in accordance with the genetic code yields the polypeptide sequence (i.e., the polynucleotide sequence “encodes” the polypeptide sequence), one polynucleotide sequence “corresponds” to another polynucleotide sequence if the two sequences encode the same polypeptide sequence.

A “heterologous” region or domain of a DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous region is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A “coding sequence” is an in-frame sequence of codons that (in view of the genetic code) correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide. A polyadenylation signal and transcription termination sequence will usually be located 3′ to the coding sequence. A “promoter sequence” is a DNA regulatory region capable of binding RNA polymerase in a cell and initiating transcription of a downstream (3′ direction) coding sequence. Promoter sequences typically contain additional sites for binding of regulatory molecules (e.g., transcription factors) which affect the transcription of the coding sequence. A coding sequence is “under the control” of the promoter sequence or “operatively linked” to the promoter when RNA polymerase binds the promoter sequence in a cell and transcribes the coding sequence into mRNA, which is then in turn translated into the protein encoded by the coding sequence.

Vectors are used to introduce a foreign substance, such as DNA, RNA or protein, into an organism or host cell. Typical vectors include recombinant viruses (for polynucleotides) and liposomes (for polypeptides). A “DNA vector” is a replicon, such as plasmid, phage or cosmid, to which another polynucleotide segment may be attached so as to bring about the replication of the attached segment. An “expression vector” is a DNA vector which contains regulatory sequences which will direct polypeptide synthesis by an appropriate host cell. This usually means a promoter to bind RNA polymerase and initiate transcription of mRNA, as well as ribosome binding sites and initiation signals to direct translation of the mRNA into a polypeptide(s). Incorporation of a polynucleotide sequence into an expression vector at the proper site and in correct reading frame, followed by transformation of an appropriate host cell by the vector, enables the production of a polypeptide encoded by said polynucleotide sequence.

“Amplification” of polynucleotide sequences is the in vitro production of multiple copies of a particular nucleic acid sequence. The amplified sequence is usually in the form of DNA. A variety of techniques for carrying out such amplification are described in a review article by Van Brunt (1990, Bio/Technol., 8(4):291-294). Polymerase chain reaction or PCR is a prototype of nucleic acid amplification, and use of PCR herein should be considered exemplary of other suitable amplification techniques.

The general structure of antibodies in vertebrates now is well understood (Edelman, G. M., Ann. N.Y. Acad. Sci., 190: 5 (1971)). Antibodies consist of two identical light polypeptide chains of molecular weight approximately 23,000 daltons (the “light chain”), and two identical heavy chains of molecular weight 53,000-70,000 (the “heavy chain”). The four chains are joined by disulfide bonds in a “Y” configuration wherein the light chains bracket the heavy chains starting at the mouth of the “Y” configuration. The “branch” portion of the “Y” configuration is designated the F_{ab} region; the stem portion of the “Y” configuration is designated the Fc region. The amino acid sequence orientation runs from the N-terminal end at the top of the “Y” configuration to the C-terminal end at the bottom of each chain. The N-terminal end possesses the variable region having specificity for the antigen that elicited it, and is approximately 100 amino acids in length, there being slight variations between light and heavy chain and from antibody to antibody.

The variable region is linked in each chain to a constant region that extends the remaining length of the chain and that within a particular class of antibody does not vary with the specificity of the antibody (i.e., the antigen eliciting it). There are five known major classes of constant regions that determine the class of the immunoglobulin molecule (IgG, IgM, IgA, IgD, and IgE corresponding to γ, μ, α, δ, and ε (gamma, mu, alpha, delta, or epsilon) heavy chain constant regions). The constant region or class determines subsequent effector function of the antibody, including activation of complement (Kabat, E. A., Structural Concepts in Immunology and Immunochemistry, 2nd Ed., p. 413-436, Holt, Rinehart, Winston (1976)), and other cellular responses (Andrews, D. W., et al., Clinical Immunobiology, pp 1-18, W. B. Sanders (1980); Kohl, S., et al., Immunology, 48: 187 (1983)); while the variable region determines the antigen with which it will react. Light chains are classified as either κ (kappa) or λ (lambda). Each heavy chain class can be prepared with either kappa or lambda light chain. The light and heavy chains are covalently bonded to each other, and the “tail” portions of the two heavy chains are bonded to each other by covalent disulfide linkages when the immunoglobulins are generated either by hybridomas or by B cells.

The expression “variable region” or “VR” refers to the domains within each pair of light and heavy chains in an antibody that are involved directly in binding the antibody to the antigen. Each heavy chain has at one end a variable domain (VH) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

The expressions “complementarity determining region,” “hypervariable region,” or “CDR” refer to one or more of the hyper-variable or complementarity determining regions (CDRs) found in the variable regions of light or heavy chains of an antibody (See Kabat, E. A. et al., Sequences of Proteins of Immunological Interest, National Institutes of Health, Bethesda, Md., (1987)). These expressions include the hypervariable regions as defined by Kabat et al. (“Sequences of Proteins of Immunological Interest,” Kabat E., et al., US Dept. of Health and Human Services, 1983) or the hypervariable loops in 3-dimensional structures of antibodies (Chothia and Lesk, J Mol. Biol. 196 901-917 (1987)). The CDRs in each chain are held in close proximity by framework regions and, with the CDRs from the other chain, contribute to the formation of the antigen binding site. Within the CDRs there are select amino acids that have been described as the selectivity determining regions (SDRs) which represent the critical contact residues used by the CDR in the antibody-antigen interaction (Kashmiri, S., Methods, 36:25-34 (2005)). In the present invention when specific antibody amino acid or nucleic acid residues are referenced by number this generally refers to its position within a specified amino acid or nucleic acid sequence (i.e., particular sequence identifier) and/or in accordance with Kabat et al numbering.

The expressions “framework region” or “FR” refer to one or more of the framework regions within the variable regions of the light and heavy chains of an antibody (See Kabat, E. A. et al., Sequences of Proteins of Immunological Interest, National Institutes of Health, Bethesda, Md., (1987)). These expressions include those amino acid sequence regions interposed between the CDRs within the variable regions of the light and heavy chains of an antibody.

“Cmax” refers to the maximum (or peak) concentration that an antibody or other compound achieves in tested area (e.g., in the serum or another compartment such as cerebrospinal fluid) after the drug has been administered. For example, serum Cmax may be measured from serum, e.g., prepared by collecting a blood sample, allowing it to clot and separating solid components by centrifugation or other means to yield serum (blood containing neither blood cells nor clotting factors), and then detecting the concentration of the analyte in the serum by ELISA or other means known in the art.

“AUC” refers to the area under the concentration-time curve which is expressed in units of mg/mL*hr (or equivalently mg*hr/ml) unless otherwise specified. “AUC₀₋ₜ” refers to the area under the concentration-time curve from time=0 to last quantifiable concentration. “AUC_{0-inf}” refers to the area under the concentration-time curve from time=0 extrapolated to infinity.

“Iₘₐₓ” refers to the maximal pharmacodynamic response elicited by an anti-CGRP antibody dosage, preferably a dosage of 350 mg or more, more typically at least 750 or 1000 mg, as compared to the response elicited by a lower anti-CGRP antibody doses, e.g., wherein such response may be detected by the inhibition of vasodilation after topical application of capsaicin.

### Anti-CGRP Antibodies and Binding Fragments Thereof Having Binding Specificity for CGRP

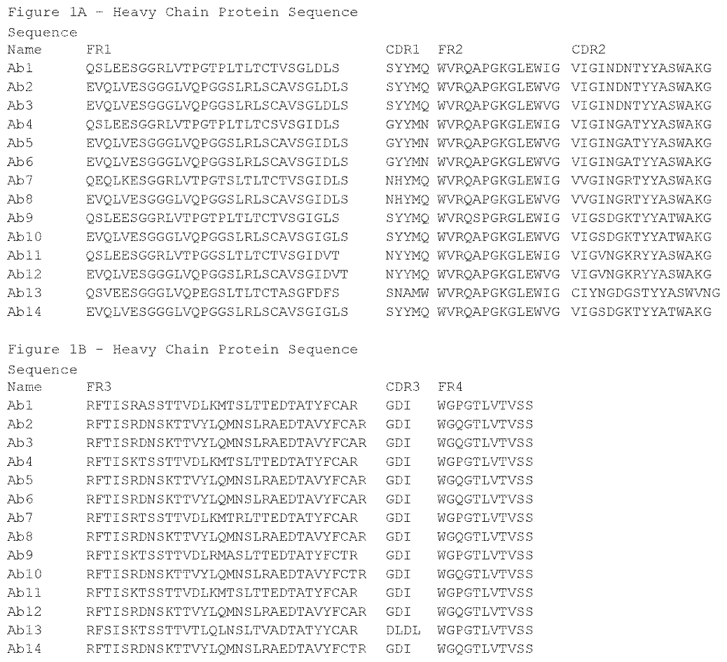

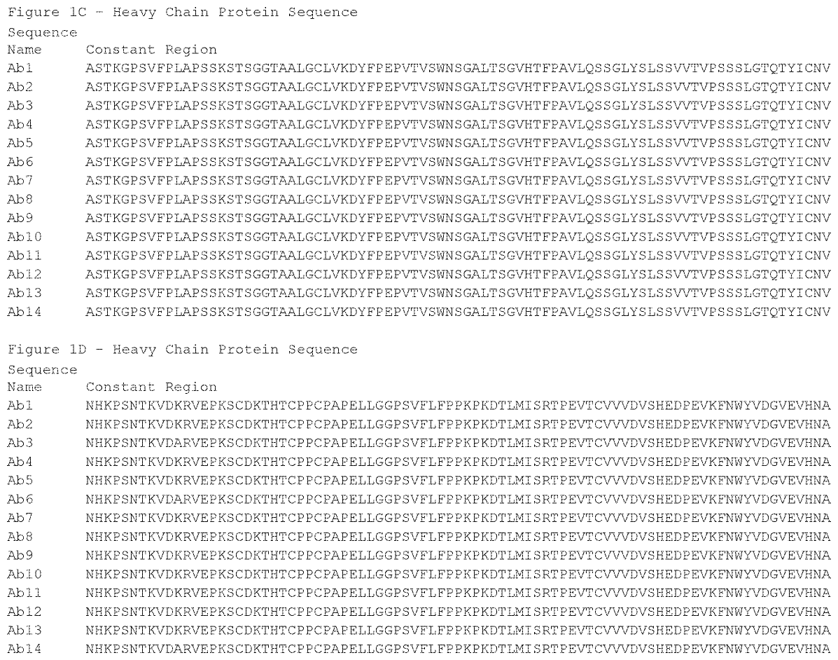

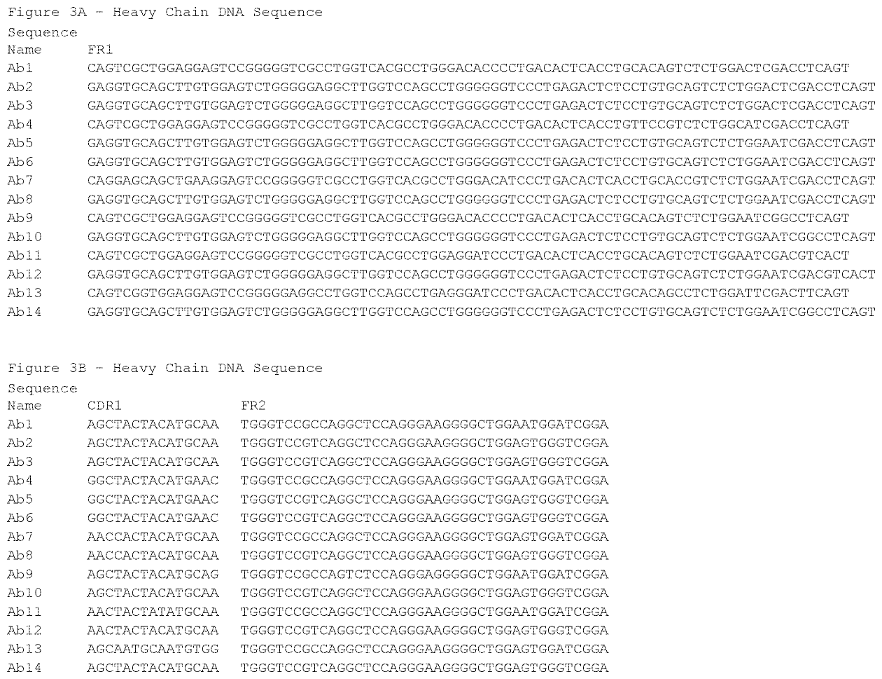

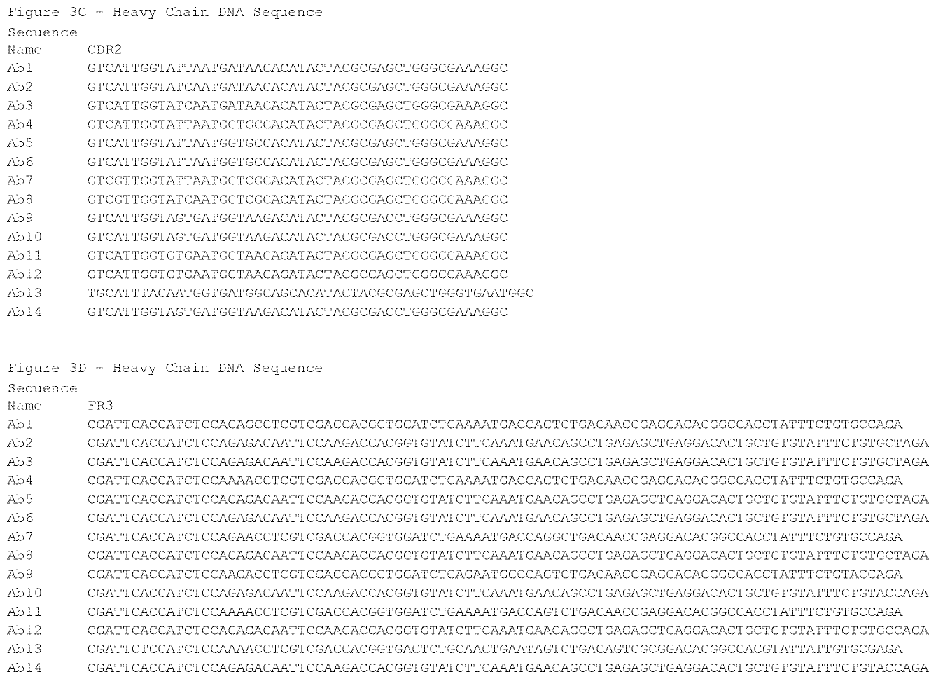

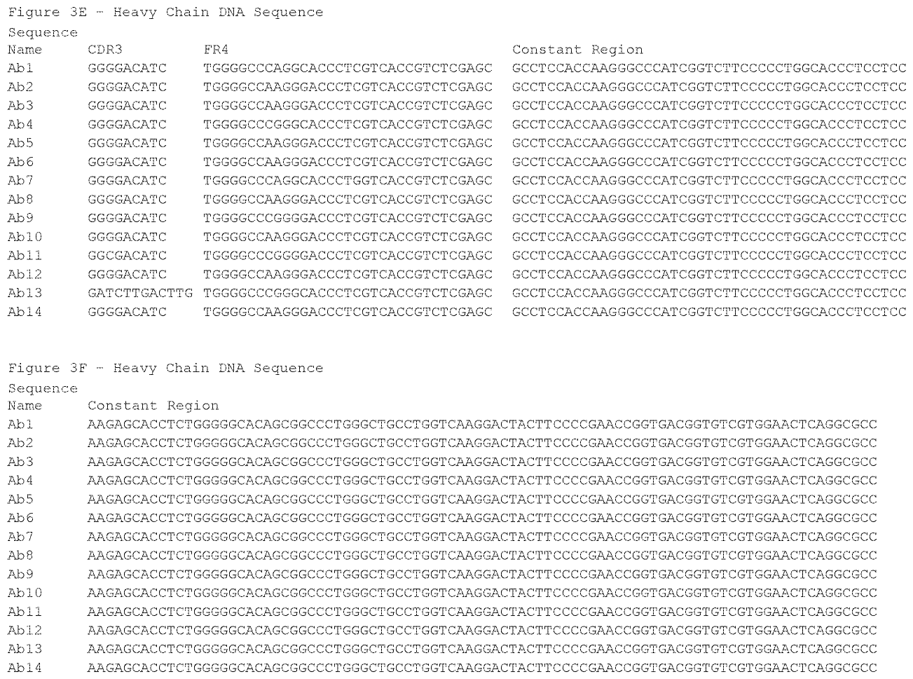

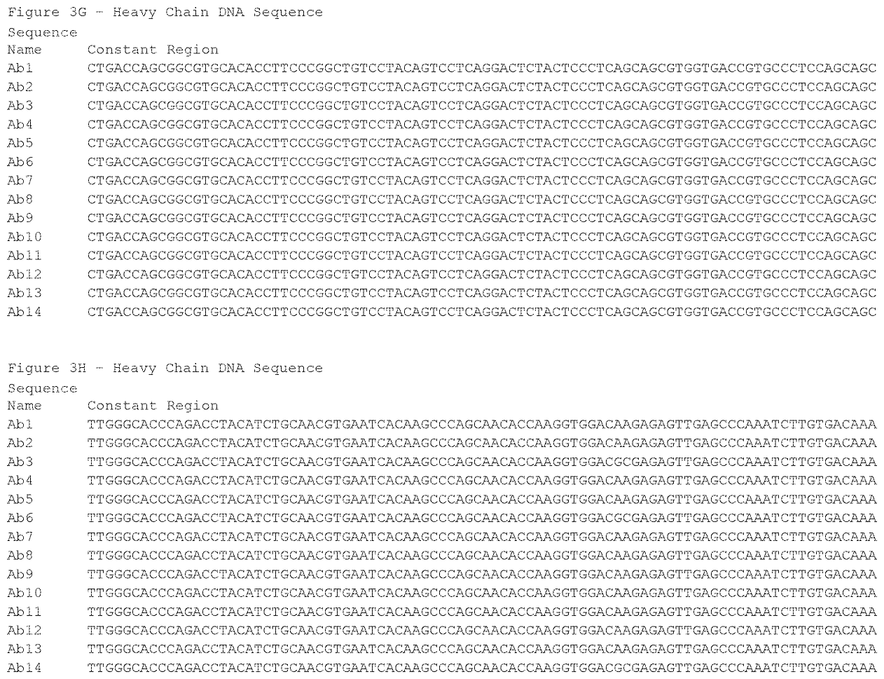

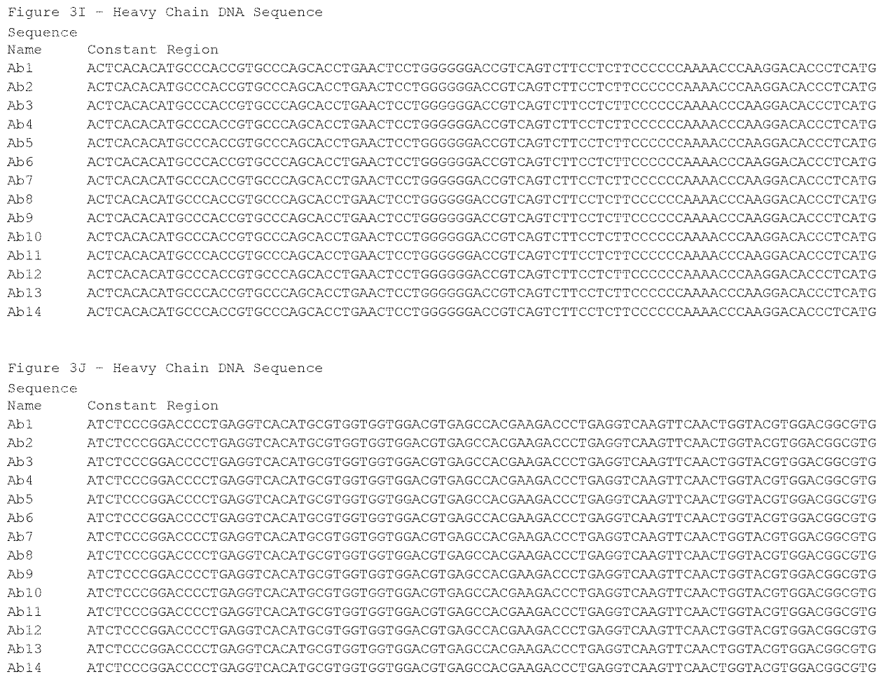

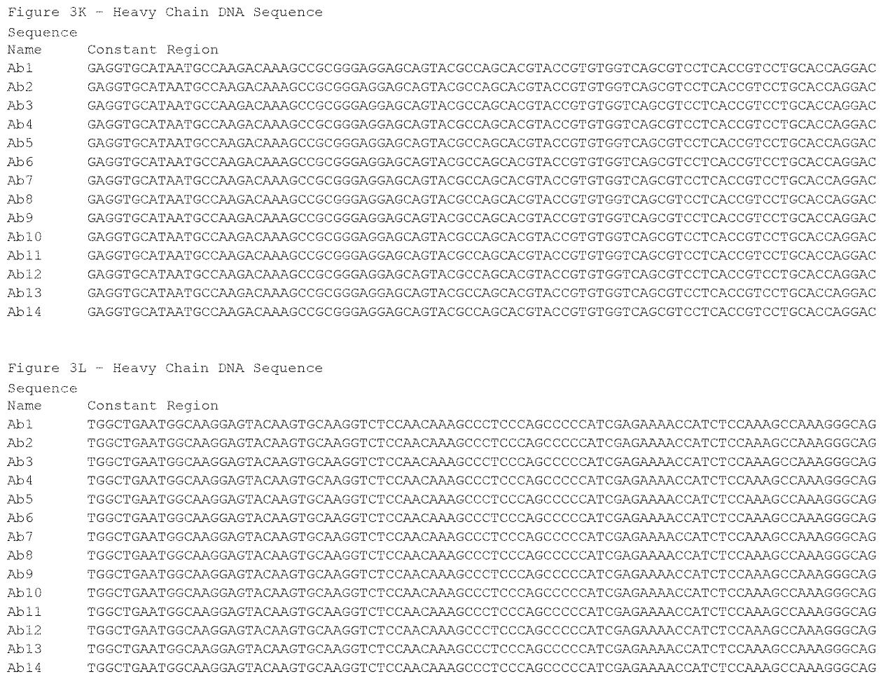

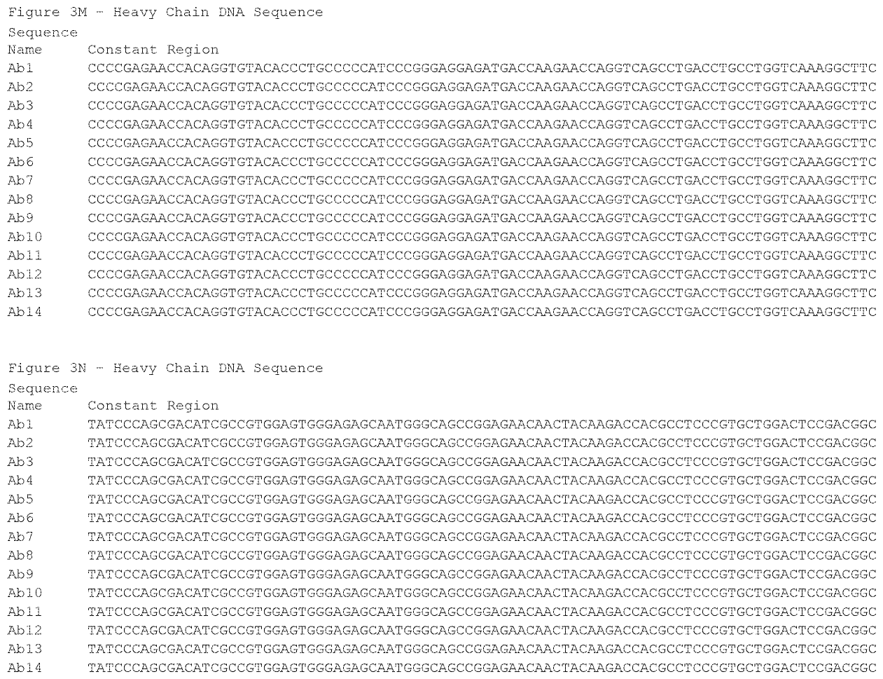

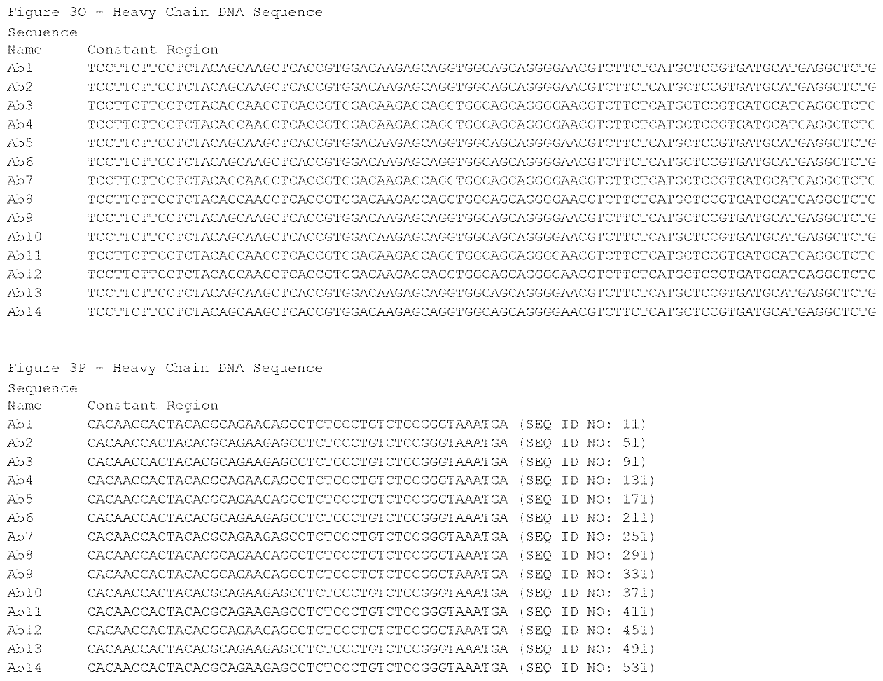

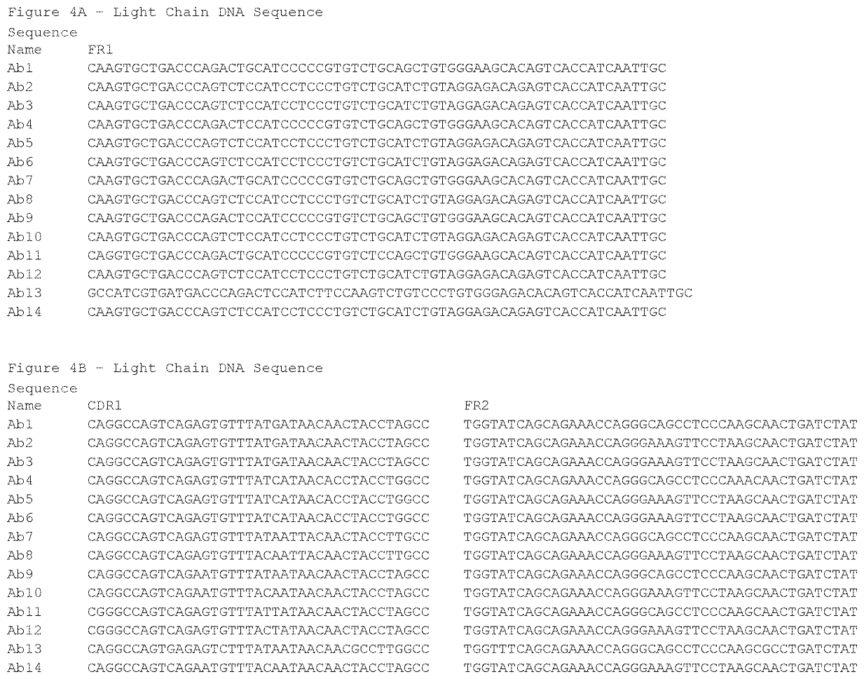

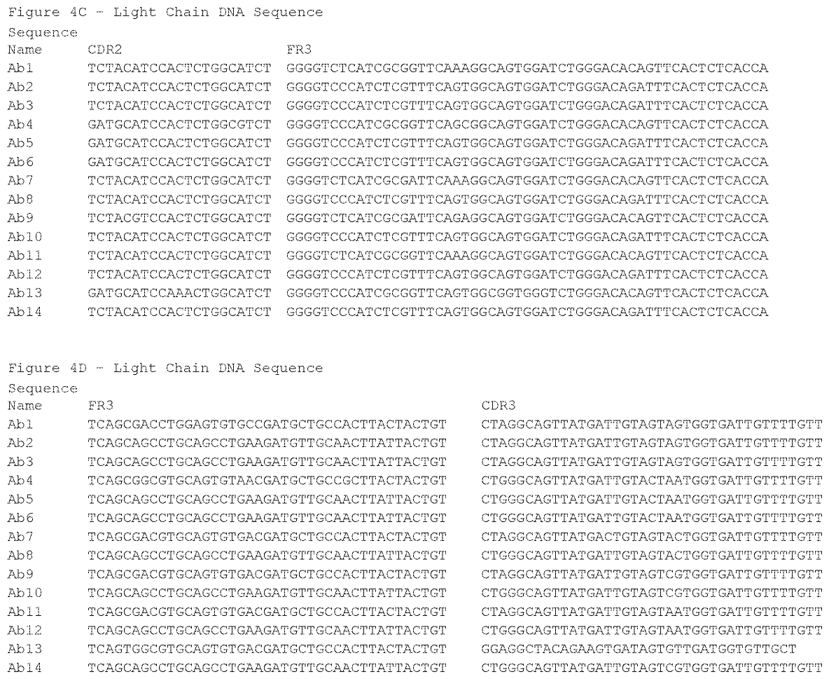

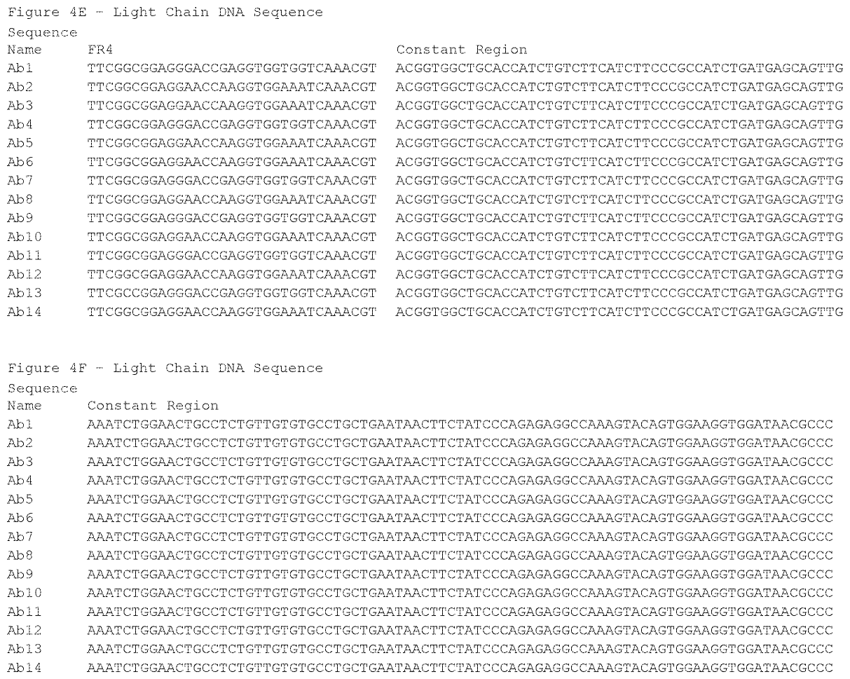

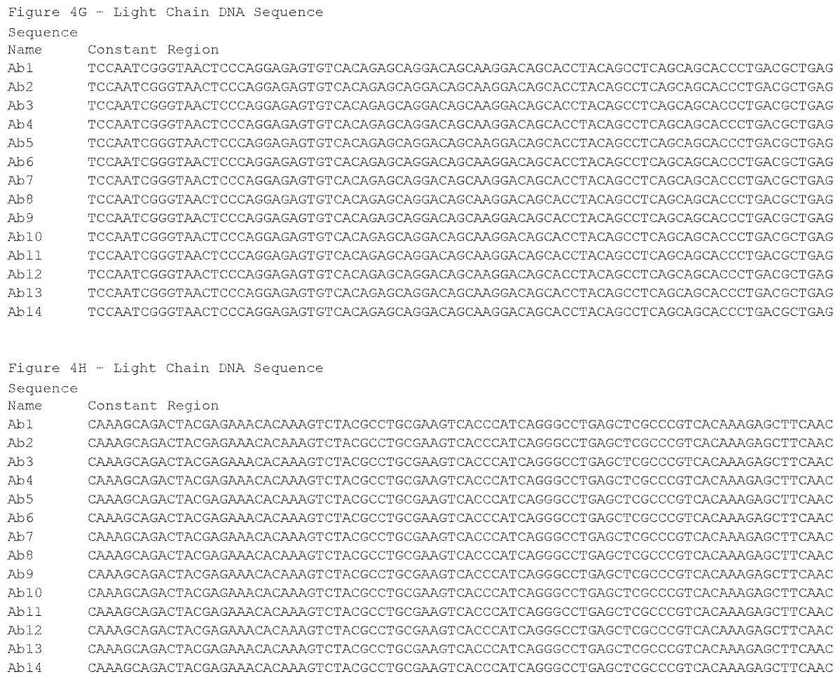

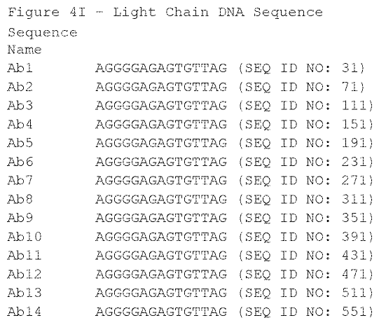

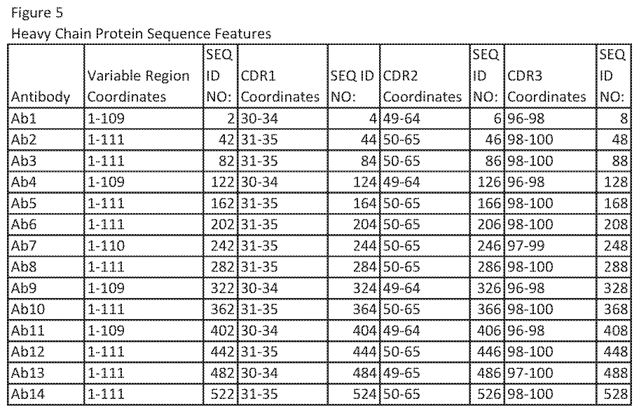

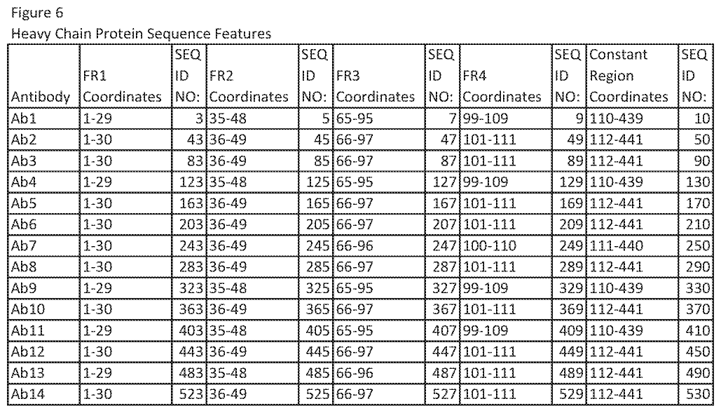

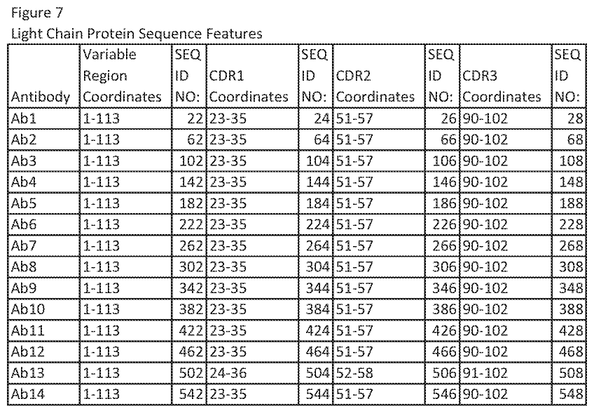

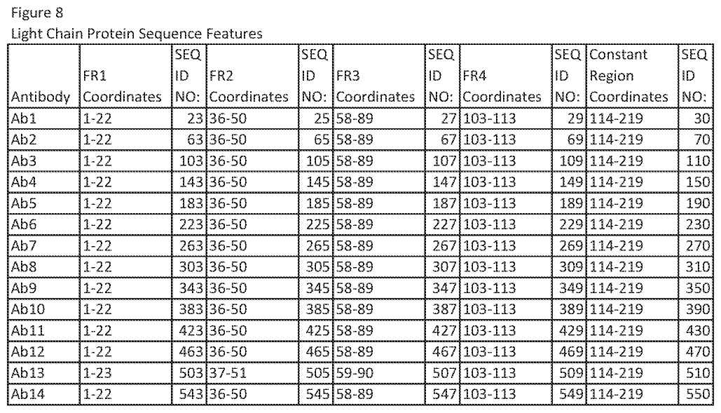

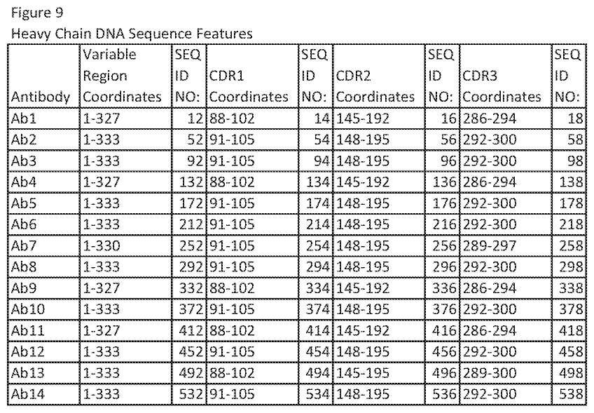

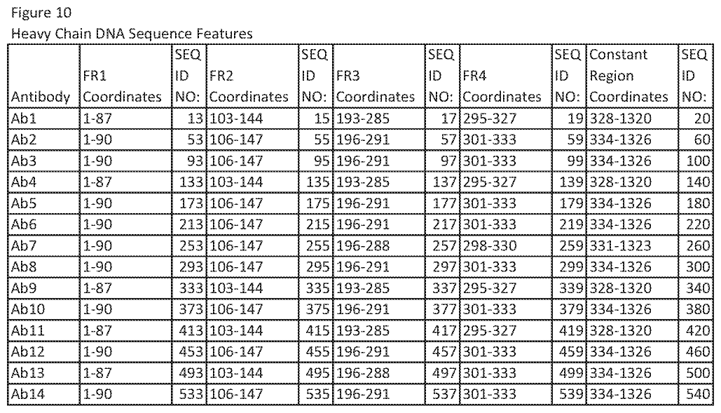

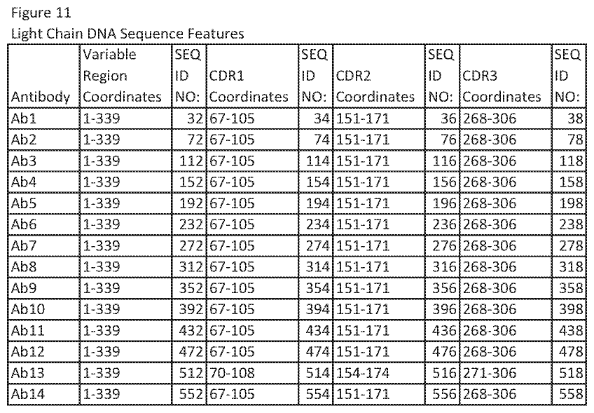

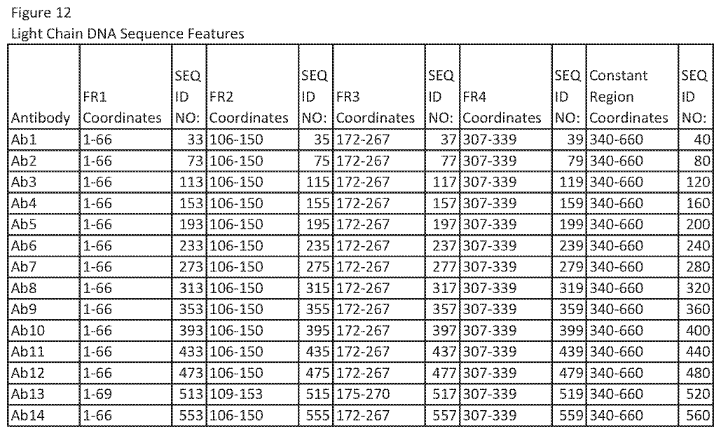

The invention specifically includes the use of specific anti-CGRP antibodies and antibody fragments referred to herein as Ab1-Ab14 which comprise or consist of the CDR, VL, VH, CL, CH polypeptides sequences identified in FIGS. 1A-12. The polypeptides comprised in an especially preferred anti-CGRP antibody, Ab6 is further described below.

### Antibody Ab6

In a preferred exemplary embodiment, the invention includes humanized antibodies having binding specificity to CGRP and possessing a variable light chain sequence comprising the sequence set forth below:

(SEQ ID NO: 222)
QVLTQSPSSLSASVGDRVTINCQASQSVYHNTYLAWYQQKPGKVPKQLIY
DASTLASGVPSRFSGSGSGTDFTLTISSLQPEDVATYYCLGSYDCTNGDC
FVFGGGTKVEIKR.

The invention also includes humanized antibodies having binding specificity to CGRP and possessing a light chain sequence comprising the sequence set forth below:

(SEQ ID NO: 221)
QVLTQSPSSLSASVGDRVTINCQASQSVYHNTYLAWYQQKPGKVPKQLIY
DASTLASGVPSRFSGSGSGTDFTLTISSLQPEDVATYYCLGSYDCTNGDC
FVFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

The invention further includes humanized antibodies having binding specificity to CGRP and possessing a variable heavy chain sequence comprising the sequence set forth below:

(SEQ ID NO: 202)
EVQLVESGGGLVQPGGSLRLSCAVSGIDLSGYYMNWVRQAPGKGLEWVGV
IGINGATYYASWAKGRFTISRDNSKTTVYLQMNSLRAEDTAVYFCARGDI
WGQGTLVTVSS.

The invention also includes humanized antibodies having binding specificity to CGRP and possessing a heavy chain sequence comprising the sequence set forth below:

(SEQ ID NO: 201)
EVQLVESGGGLVQPGGSLRLSCAVSGIDLSGYYMNWVRQAPGKGLEWVGV
IGINGATYYASWAKGRFTISRDNSKTTVYLQMNSLRAEDTAVYFCARGDI
WGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDARVEPKSCDKTHTCPPCPAPELLGGPSVFLFPPKPKDTLMISRT
PEVTCVVVDVSHEDPEVKFNWYVDGVEVHNAKTKPREEQYASTYRVVSVL
TVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFL
YSKLTVDKSRWQQGNVFSCSVMHEALHNHYTQKSLSLSPGK.

Alternatively, the heavy chain of Ab6 may lack the C-terminal lysine of SEQ ID NO: 201, i.e., a heavy chain sequence comprising the sequence set forth below:

(SEQ ID NO: 566)
EVQLVESGGGLVQPGGSLRLSCAVSGIDLSGYYMNWVRQAPGKGLEWVGV
IGINGATYYASWAKGRFTISRDNSKTTVYLQMNSLRAEDTAVYFCARGDI
WGQGTLVTVSSASTKGPSVFPLAPSSKSTSGGTAALGCLVKDYFPEPVTV
SWNSGALTSGVHTFPAVLQSSGLYSLSSVVTVPSSSLGTQTYICNVNHKP
SNTKVDARVEPKSCDKTHTCPPCPAPELLGGPSVFLFPPKPKDTLMISRT
PEVTCVVVDVSHEDPEVKFNWYVDGVEVHNAKTKPREEQYASTYRVVSVL
TVLHQDWLNGKEYKCKVSNKALPAPIEKTISKAKGQPREPQVYTLPPSRE
EMTKNQVSLTCLVKGFYPSDIAVEWESNGQPENNYKTTPPVLDSDGSFFL
YSKLTVDKSRWQQGNVFSCSVMHEALHNHYTQKSLSLSPG.

The invention further contemplates antibodies comprising one or more of the polypeptide sequences of SEQ ID NO: 224; SEQ ID NO: 226; and SEQ ID NO: 228 which correspond to the complementarity-determining regions (CDRs, or hypervariable regions) of the variable light chain sequence of SEQ ID NO: 222 or the light chain sequence of SEQ ID NO: 221, and/or one or more of the polypeptide sequences of SEQ ID NO: 204; SEQ ID NO: 206; and SEQ ID NO: 208 which correspond to the complementarity-determining regions (CDRs, or hypervariable regions) of the variable heavy chain sequence of SEQ ID NO: 202 or the heavy chain sequence of SEQ ID NO: 201 or SEQ ID NO: 566, or combinations of these polypeptide sequences. In another embodiment of the invention, the antibodies of the invention or fragments thereof comprise, or alternatively consist of, combinations of one or more of the CDRs, the variable heavy and variable light chain sequences, and the heavy and light chain sequences set forth above, including all of them.

The invention also contemplates fragments of the antibody having binding specificity to CGRP. In one embodiment of the invention, antibody fragments of the invention comprise, or alternatively consist of, the polypeptide sequence of SEQ ID NO: 222 or SEQ ID NO: 221. In another embodiment of the invention, antibody fragments of the invention comprise, or alternatively consist of, the polypeptide sequence of SEQ ID NO: 202 or SEQ ID NO: 201 or SEQ ID NO: 566.

In a further embodiment of the invention, fragments of the antibody having binding specificity to CGRP comprise, or alternatively consist of, one or more of the polypeptide sequences of SEQ ID NO: 224; SEQ ID NO: 226; and SEQ ID NO: 228 which correspond to the complementarity-determining regions (CDRs, or hypervariable regions) of the variable light chain sequence of SEQ ID NO: 222 or the light chain sequence of SEQ ID NO: 221.

In a further embodiment of the invention, fragments of the antibody having binding specificity to CGRP comprise, or alternatively consist of, one or more of the polypeptide sequences of SEQ ID NO: 204; SEQ ID NO: 206; and SEQ ID NO: 208 which correspond to the complementarity-determining regions (CDRs, or hypervariable regions) of the variable heavy chain sequence of SEQ ID NO: 202 or the heavy chain sequence of SEQ ID NO: 201 or SEQ ID NO: 566.

The invention also contemplates antibody fragments which include one or more of the antibody fragments described herein. In one embodiment of the invention, fragments of the antibodies having binding specificity to CGRP comprise, or alternatively consist of, one, two, three or more, including all of the following antibody fragments: the variable light chain region of SEQ ID NO: 222; the variable heavy chain region of SEQ ID NO: 202; the complementarity-determining regions (SEQ ID NO: 224; SEQ ID NO: 226; and SEQ ID NO: 228) of the variable light chain region of SEQ ID NO: 222; and the complementarity-determining regions (SEQ ID NO: 204; SEQ ID NO: 206; and SEQ ID NO: 208) of the variable heavy chain region of SEQ ID NO: 202.

In a particularly preferred embodiment of the invention, the humanized anti-CGRP antibody is Ab6, comprising, or alternatively consisting of, SEQ ID NO: 221 and SEQ ID NO: 201 or SEQ ID NO: 566, and having at least one of the biological activities set forth herein.

In a further particularly preferred embodiment of the invention, antibody fragments comprise, or alternatively consist of, Fab (fragment antigen binding) fragments having binding specificity for CGRP. With respect to antibody Ab6, the Fab fragment includes the variable light chain sequence of SEQ ID NO: 222 and the variable heavy chain sequence of SEQ ID NO: 202. This embodiment of the invention further contemplates additions, deletions, and variants of SEQ ID NO: 222 and/or SEQ ID NO: 202 in said Fab while retaining binding specificity for CGRP.

In another particularly preferred embodiment of the invention, said anti-CGRP antibody may comprise the antibody expression product isolated from recombinant cells which express nucleic acid sequences encoding the variable light chain polypeptide of SEQ ID NO: 222 and the variable heavy chain polypeptide of SEQ ID NO: 202, which polypeptides optionally are respectively linked to human light and heavy constant region polypeptides, e.g., human IgG1, IgG2, IgG3 or IgG4 constant regions, which constant regions optionally may be modified to alter glycosylation or proteolysis, wherein said recombinant cells optionally comprise yeast or mammalian cells, e.g., Pichia pastoris or CHO cells.

In another particularly preferred embodiment of the invention, said anti-CGRP antibody may comprise the antibody expression product isolated from recombinant cells which express nucleic acid sequences encoding the light chain of SEQ ID NO: 221 and the heavy chain polypeptide of SEQ ID NO: 201 or SEQ ID NO: 566, wherein said recombinant cells optionally comprise yeast or mammalian cells, e.g., Pichia pastoris or CHO cells, wherein the constant regions thereof optionally may be modified to alter glycosylation or proteolysis or other effector functions.

In another particularly preferred embodiment of the invention, any of the aforementioned anti-CGRP antibodies or antibody fragments may be optionally comprised in a formulation as disclosed herein, e.g., comprising histidine (L-histidine), sorbitol, polysorbate 80, such as, per 1 mL volume, about 100 mg anti-CGRP antibody, about 3.1 mg L-Histidine, about 40.5 mg Sorbitol, and about 0.15 mg Polysorbate 80, having a pH of about 5.8.

In one embodiment of the invention described herein (infra), Fab fragments may be produced by enzymatic digestion (e.g., papain) of Ab6. In another embodiment of the invention, anti-CGRP antibodies such as Ab6 or Fab fragments thereof may be produced via expression in mammalian cells such as CHO, NSO or HEK 293 cells, fungal, insect, or microbial systems such as yeast cells (for example diploid yeast such as diploid Pichia) and other yeast strains. Suitable Pichia species include, but are not limited to, Pichia pastoris.

In another embodiment, antibody fragments may be present in one or more of the following non-limiting forms: Fab, Fab′, F(ab′)₂, Fv and single chain Fv antibody forms. In a preferred embodiment, the anti-CGRP antibodies described herein further comprises the kappa constant light chain sequence comprising the sequence set forth below:

(SEQ ID NO: 563)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC.

In another preferred embodiment, the anti-CGRP antibodies described herein further comprises the gamma-1 constant heavy chain polypeptide sequence comprising the sequence set forth below or the same sequence lacking the carboxy terminal lysine residue (SEQ ID NO: 564 and SEQ ID NO: 565, respectively):

(SEQ ID NO: 564)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYASTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK.
(SEQ ID NO: 565)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYASTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG.

For clarity, any antibody disclosed herein is intended to include any variant of the disclosed constant region variant sequences, e.g., Ab6 may comprise the constant region of SEQ ID NO: 564 containing the C-terminal lysine or may comprise the constant region of SEQ ID NO: 565 lacking the C-terminal lysine. Thus, every disclosure herein of the heavy chain of SEQ ID NO: 201 also includes a variant lacking the C-terminal lysine residue thereof, i.e., having the heavy chain variable region sequence of Ab6 (SEQ ID NO: 202) and the constant region sequence of SEQ ID NO: 565. For example, the sequence encoding an antibody comprising a C-terminal lysine in the heavy chain may, when expressed in cell lines such as CHO cells, produce an antibody lacking said C-terminal lysine due to proteolysis, or a mixture of heavy chains containing or lacking said C-terminal lysine.

In another embodiment, the invention contemplates use of an isolated anti-CGRP antibody comprising a VH polypeptide sequence selected from: SEQ ID NO: 2, SEQ ID NO: 42, SEQ ID NO: 82, SEQ ID NO: 122, SEQ ID NO: 162, SEQ ID NO: 202, SEQ ID NO: 242, SEQ ID NO: 282, SEQ ID NO: 322, SEQ ID NO: 362, SEQ ID NO: 402, SEQ ID NO: 442, SEQ ID NO: 482, or SEQ ID NO: 522, or a variant thereof; and further comprising a V_{L} polypeptide sequence selected from: SEQ ID NO: 22, SEQ ID NO: 62, SEQ ID NO: 102, SEQ ID NO: 142, SEQ ID NO: 182, SEQ ID NO: 222, SEQ ID NO: 262, SEQ ID NO: 302, SEQ ID NO: 342, SEQ ID NO: 382, SEQ ID NO: 422, SEQ ID NO: 462, SEQ ID NO: 502, or SEQ ID NO: 542, or a variant thereof, wherein one or more of the framework residues (FR residues) in said V_{H} or V_{L} polypeptide has been substituted with another amino acid residue resulting in an anti-CGRP antibody that specifically binds CGRP. The invention contemplates humanized and chimeric forms of these antibodies. The chimeric antibodies may include an Fe derived from IgG1, IgG2, IgG3, or IgG4 constant regions.

In one embodiment of the invention, the antibodies or VH or V_{L} polypeptides originate or are selected from one or more rabbit B cell populations prior to initiation of the humanization process referenced herein.

In another embodiment of the invention, the anti-CGRP antibodies and fragments thereof do not have binding specificity for CGRP-R. In a further embodiment of the invention, the anti-CGRP antibodies and fragments thereof inhibit the association of CGRP with CGRP-R. In another embodiment of the invention, the anti-CGRP antibodies and fragments thereof inhibit the association of CGRP with CGRP-R and/or additional proteins and/or multimers thereof, and/or antagonizes the biological effects thereof.

As stated herein, antibodies and fragments thereof may be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

Antibodies or fragments thereof may also be chemically modified to provide additional advantages such as increased solubility, stability and circulating time (in vivo half-life) of the polypeptide, or decreased immunogenicity (See U.S. Pat. No. 4,179,337). The chemical moieties for derivatization may be selected from water soluble polymers such as polyethylene glycol, ethylene glycol/propylene glycol copolymers, carboxymethylcellulose, dextran, polyvinyl alcohol and the like. The antibodies and fragments thereof may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

There are a number of attachment methods available to those skilled in the art, See e.g., EP 0 401 384, herein incorporated by reference (coupling PEG to G-CSF), See also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. For example, polyethylene glycol can be linked to polypeptides via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) or to more than one type of amino acid residue (e.g., lysine, histidine, aspartic acid, glutamic acid, cysteine and combinations thereof).

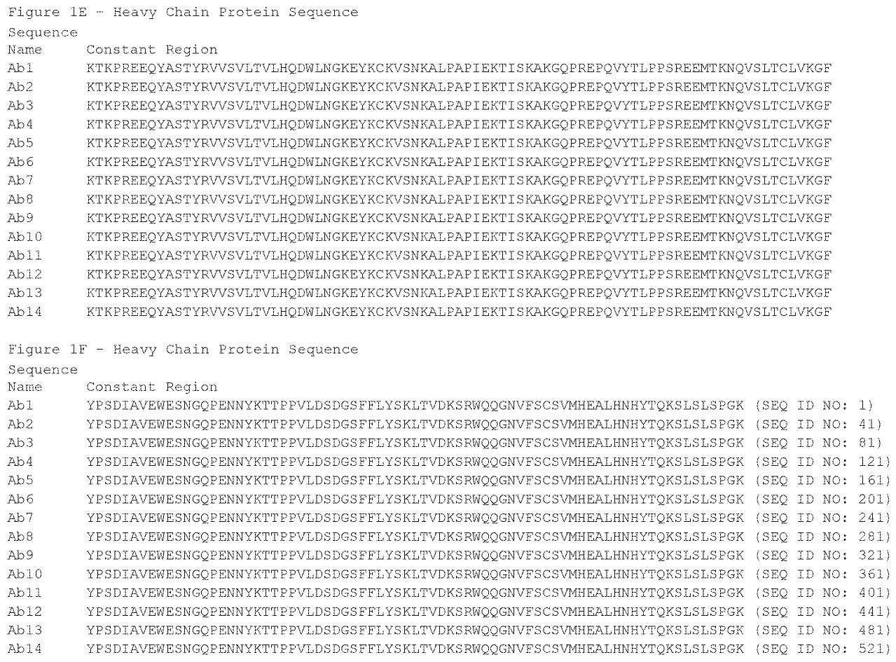

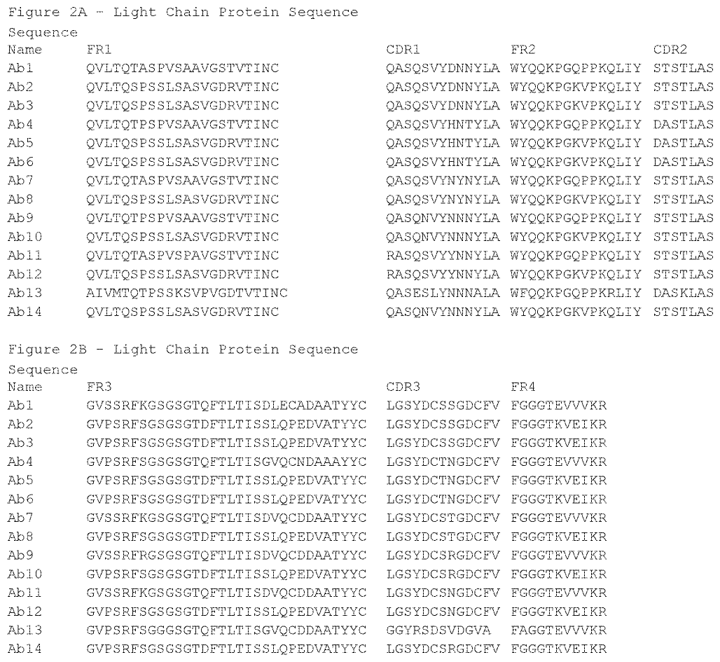

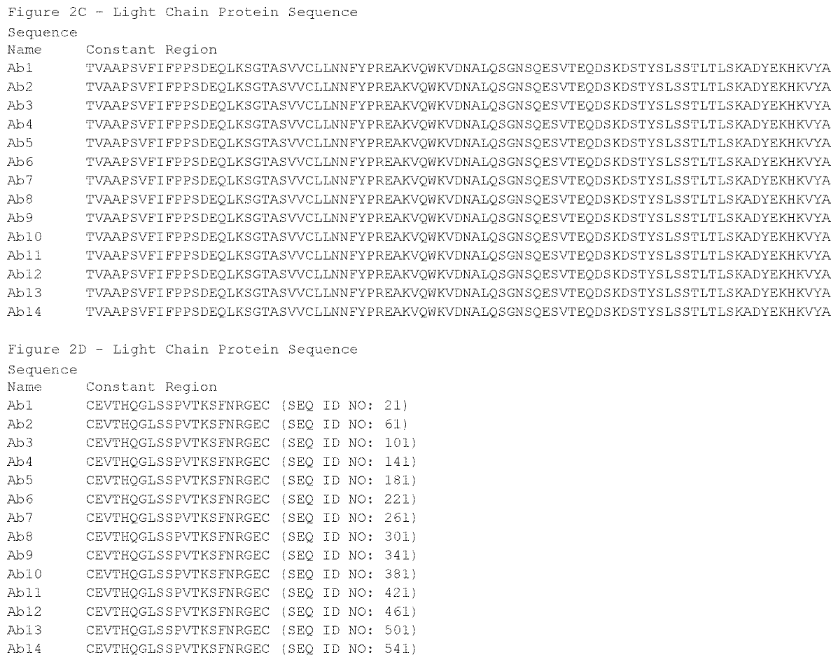

Alternatively, antibodies or fragments thereof may have increased in vivo half-lives via fusion with albumin (including but not limited to recombinant human serum albumin or fragments or variants thereof (See, e.g., U.S. Pat. No. 5,876,969, issued Mar. 2, 1999, EP Patent 0 413 622, and U.S. Pat. No. 5,766,883, issued Jun. 16, 1998, herein incorporated by reference in their entirety)) or other circulating blood proteins such as transferrin or ferritin. In a preferred embodiment, polypeptides and/or antibodies of the present invention (including fragments or variants thereof) are fused with the mature form of human serum albumin (i.e., amino acids 1-585 of human serum albumin as shown in FIGS. 1 and 2 of EP Patent 0 322 094) which is herein incorporated by reference in its entirety. Polynucleotides encoding fusion proteins of the invention are also encompassed by the invention.

Regarding detectable moieties, further exemplary enzymes include, but are not limited to, horseradish peroxidase, acetylcholinesterase, alkaline phosphatase, beta-galactosidase and luciferase. Further exemplary fluorescent materials include, but are not limited to, rhodamine, fluorescein, fluorescein isothiocyanate, umbelliferone, dichlorotriazinylamine, phycoerythrin and dansyl chloride. Further exemplary chemiluminescent moieties include, but are not limited to, luminol. Further exemplary bioluminescent materials include, but are not limited to, luciferin and aequorin. Further exemplary radioactive materials include, but are not limited to, Iodine 125 (¹²⁵I), Carbon 14 (¹⁴C), Sulfur 35 (³⁵S), Tritium (³H) and Phosphorus 32 (³²P).

Regarding functional moieties, exemplary cytotoxic agents include, but are not limited to, methotrexate, aminopterin, 6-mercaptopurine, 6-thioguanine, cytarabine, 5-fluorouracil decarbazine; alkylating agents such as mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP) cisplatin and carboplatin (paraplatin); anthracyclines include daunorubicin (formerly daunomycin), doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, and anthramycin (AMC); and antimytotic agents such as the vinca alkaloids, vincristine and vinblastine. Other cytotoxic agents include paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, asparaginase, corticosteroids, mytotane (O,P′-(DDD)), interferons, and mixtures of these cytotoxic agents.

Further cytotoxic agents include, but are not limited to, chemotherapeutic agents such as carboplatin, cisplatin, paclitaxel, gemcitabine, calicheamicin, doxorubicin, 5-fluorouracil, mitomycin C, actinomycin D, cyclophosphamide, vincristine and bleomycin. Toxic enzymes from plants and bacteria such as ricin, diphtheria toxin and Pseudomonas toxin may be conjugated to the humanized or chimeric antibodies, or binding fragments thereof, to generate cell-type-specific-killing reagents (Youle, et al., Proc. Nat'l Acad. Sci. USA 77:5483 (1980); Gilliland, et al., Proc. Nat'l Acad. Sci. USA 77:4539 (1980); Krolick, et al., Proc. Nat'l Acad. Sci. USA 77:5419 (1980)).

Other cytotoxic agents include cytotoxic ribonucleases as described by Goldenberg in U.S. Pat. No. 6,653,104. Embodiments of the invention also relate to radioimmunoconjugates where a radionuclide that emits alpha or beta particles is stably coupled to the antibody, or binding fragments thereof, with or without the use of a complex-forming agent. Such radionuclides include beta-emitters such as Phosphorus-32 (³²P), Scandium-47 (⁴⁷Sc), Copper-67 (⁶⁷Cu), Gallium-67 (⁶⁷Ga), Yttrium-88 (⁸⁸Y), Yttrium-90 (⁹⁰Y), Iodine-125 (¹²⁵I), Iodine-131 (¹³¹I), Samarium-153 (¹⁵³Sm), Lutetium-177 (¹⁷⁷Lu), Rhenium-186 (¹⁸⁶Re) or Rhenium-188 (¹⁸⁸Re), and alpha-emitters such as Astatine-211 (²¹¹At), Lead-212 (²¹²Pb), Bismuth-212 (²¹²Bi) or -213 (²¹³Bi) or Actinium-225 (²²⁵Ac).

Methods are known in the art for conjugating an antibody or binding fragment thereof to a detectable moiety and the like, such as for example those methods described by Hunter et al, Nature 144:945 (1962); David et al, Biochemistry 13:1014 (1974); Pain et al, J. Immunol. Meth. 40:219 (1981); and Nygren, J., Histochem. and Cytochem. 30:407 (1982).

Embodiments described herein further include variants and equivalents that are substantially homologous to the antibodies, antibody fragments, diabodies, SMIPs, camelbodies, nanobodies, IgNAR, polypeptides, variable regions and CDRs set forth herein. These may contain, e.g., conservative substitution mutations, (i.e., the substitution of one or more amino acids by similar amino acids). For example, conservative substitution refers to the substitution of an amino acid with another within the same general class, e.g., one acidic amino acid with another acidic amino acid, one basic amino acid with another basic amino acid, or one neutral amino acid by another neutral amino acid. What is intended by a conservative amino acid substitution is well known in the art.

In another embodiment, the invention contemplates polypeptide sequences having at least 90% or greater sequence homology to any one or more of the polypeptide sequences of antibody fragments, variable regions and CDRs set forth herein. More preferably, the invention contemplates polypeptide sequences having at least 95% or greater sequence homology, even more preferably at least 98% or greater sequence homology, and still more preferably at least 99% or greater sequence homology to any one or more of the polypeptide sequences of antibody fragments, variable regions and CDRs set forth herein. Methods for determining homology between nucleic acid and amino acid sequences are well known to those of ordinary skill in the art.

In another embodiment, the invention further contemplates the above-recited polypeptide homologs of the antibody fragments, variable regions and CDRs set forth herein further having anti-CGRP activity. Non-limiting examples of anti-CGRP activity are set forth herein.

The present invention also contemplates anti-CGRP antibodies comprising any of the polypeptide or polynucleotide sequences described herein substituted for any of the other polynucleotide sequences described herein. For example, without limitation thereto, the present invention contemplates antibodies comprising the combination of any of the variable light chain and variable heavy chain sequences described herein, and further contemplates antibodies resulting from substitution of any of the CDR sequences described herein for any of the other CDR sequences described herein.

### Additional Exemplary Embodiments of the Invention

In another embodiment, the invention contemplates treatment methods using one or more anti-human CGRP antibodies or antibody fragments thereof which specifically bind to the same overlapping linear or conformational epitope(s) and/or competes for binding to the same overlapping linear or conformational epitope(s) on an intact human CGRP polypeptide or fragment thereof as an anti-human CGRP antibody selected from Ab1, Ab2, Ab3, Ab4, Ab5, Ab6, Ab7, Ab8, Ab9, Ab10, Ab11, Ab12, Ab13, or Ab14. In a preferred embodiment, the anti-human CGRP antibody or fragment thereof specifically binds to the same overlapping linear or conformational epitope(s) and/or competes for binding to the same overlapping linear or conformational epitope(s) on an intact human CGRP polypeptide or a fragment thereof as Ab3, Ab6, Ab13, or Ab14.

A preferred embodiment of the invention is directed to treatment methods using chimeric or humanized antibodies and fragments thereof (including Fab fragments) having binding specificity for CGRP and inhibiting biological activities mediated by the binding of CGRP to the CGRP receptor. In a particularly preferred embodiment of the invention, the chimeric or humanized anti-CGRP antibodies are selected from Ab3, Ab6, Ab13, or Ab14.

In another embodiment of the invention, the anti-human CGRP antibody used in the described treatment methods is an antibody which specifically binds to the same overlapping linear or conformational epitopes on an intact CGRP polypeptide or fragment thereof that is (are) specifically bound by Ab3, Ab6, Ab13, or Ab14 as ascertained by epitopic mapping using overlapping linear peptide fragments which span the full length of the native human CGRP polypeptide.

The invention is also directed to treatment methods using an anti-CGRP antibody that binds with the same CGRP epitope and/or competes with an anti-CGRP antibody for binding to CGRP as an antibody or antibody fragment disclosed herein, including but not limited to an anti-CGRP antibody selected from Ab1, Ab2, Ab3, Ab4, Ab5, Ab6, Ab7, Ab8, Ab9, Ab10, Ab11, Ab12, Ab13, or Ab14.

In another embodiment, the invention is also directed to treatment methods using an isolated anti-CGRP antibody or antibody fragment comprising one or more of the CDRs contained in the VH polypeptide sequences selected from: 3, 13, 23, 33, 43, 53, 63, 73, 83, 93, 103, 113, 123, or 133, or a variant thereof, and/or one or more of the CDRs contained in the V_{L} polypeptide sequences selected from: 1, 11, 21, 31, 41, 51, 61, 71, 81, 91, 101, 111, 121, or 131, or a variant thereof.

In one embodiment of the invention, the anti-human CGRP antibody discussed in the two prior paragraphs comprises at least 2 complementarity determining regions (CDRs) in each the variable light and the variable heavy regions which are identical to those contained in an anti-human CGRP antibody selected from Ab1, Ab2, Ab3, Ab4, Ab5, Ab6, Ab7, Ab8, Ab9, Ab10, Ab11, Ab12, Ab13, or Ab14.

In a preferred embodiment, the anti-human CGRP antibody used in the described treatment methods comprises at least 2 complementarity determining regions (CDRs) in each the variable light and the variable heavy regions which are identical to those contained in Ab3 or Ab6. In another embodiment, all of the CDRs of the anti-human CGRP antibody discussed above are identical to the CDRs contained in an anti-human CGRP antibody selected from Ab1, Ab2, Ab3, Ab4, Ab5, Ab6, Ab7, Ab8, Ab9, Ab10, Ab11, Ab12, Ab13, or Ab14. In a preferred embodiment of the invention, all of the CDRs of the anti-human CGRP antibody discussed above are identical to the CDRs contained in an anti-human CGRP antibody selected from Ab3 or Ab6.

The invention further contemplates treatment methods wherein the one or more anti-human CGRP antibodies discussed above are aglycosylated or if glycosylated are only mannosylated; that contain an Fc region that has been modified to alter effector function, half-life, proteolysis, and/or glycosylation; are human, humanized, single chain or chimeric; and are a humanized antibody derived from a rabbit (parent) anti-human CGRP antibody. An exemplary mutation which impairs glycosylation comprises the mutation of the Asn residue at position 297 of an IgG heavy chain constant region such as IgG1 to another amino acid, such as Ala as described in U.S. Pat. No. 5,624,821, which is incorporated by reference in its entirety.

The invention further contemplates one or more anti-human CGRP antibodies wherein the framework regions (FRs) in the variable light region and the variable heavy regions of said antibody respectively are human FRs which are unmodified or which have been modified by the substitution of one or more human FR residues in the variable light or heavy chain region with the corresponding FR residues of the parent rabbit antibody, and wherein said human FRs have been derived from human variable heavy and light chain antibody sequences which have been selected from a library of human germline antibody sequences based on their high level of homology to the corresponding rabbit variable heavy or light chain regions relative to other human germline antibody sequences contained in the library.

The invention also contemplates a method of treating or preventing medication overuse headache, e.g., associated with the overuse of anti-migraine drugs and/or associated with triptan and/or ergot and/or analgesic overuse, comprising administering to a patient exhibiting medication overuse headache or at risk of developing medication overuse headache a therapeutically effective amount of at least one anti-human CGRP antibody or fragment described herein. The invention also contemplates that the treatment method may involve the administration of two or more anti-CGRP antibodies or fragments thereof and disclosed herein. If more than one antibody is administered to the patient, the multiple antibodies may be administered simultaneously or concurrently, or may be staggered in their administration. The anti-CGRP activity of the anti-CGRP antibodies of the present invention, and fragments thereof having binding specificity to CGRP, may also be described by their strength of binding or their affinity for CGRP. In one embodiment of the invention, the anti-CGRP antibodies of the present invention, and fragments thereof having binding specificity to CGRP, bind to CGRP with a dissociation constant (K_{D}) of less than or equal to 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, or 10⁻¹³ M. Preferably, the anti-CGRP antibodies and fragments thereof bind CGRP with a dissociation constant of less than or equal to 10⁻¹¹ M, 5×10⁻¹² M, or 10⁻¹² M. In another embodiment of the invention, the anti-CGRP antibodies of the present invention, and fragments thereof having binding specificity to CGRP, bind to a linear or conformational CGRP epitope.

In another embodiment of the invention, the anti-CGRP activity of the anti-CGRP antibodies of the present invention, and fragments thereof having binding specificity to CGRP, bind to CGRP with an off-rate of less than or equal to 10⁻⁴ S⁻¹, 5×10⁻⁵ S⁻¹, 10⁻⁵ S⁻¹, 5×10⁻⁶ S⁻¹, 10⁻⁶ S⁻¹, 5×10⁻⁷ S⁻¹, or 10⁻⁷ S⁻¹.

In a further embodiment of the invention, the anti-CGRP activity of the anti-CGRP antibodies of the present invention, and fragments thereof having binding specificity to CGRP, exhibit anti-CGRP activity by preventing, ameliorating or reducing the symptoms of, or alternatively treating, diseases and disorders associated with CGRP. Non-limiting examples of diseases and disorders associated with CGRP are set forth herein and include headache and migraine disorders.

### Polynucleotides Encoding Anti-CGRP Antibody Polypeptides

As aforementioned the invention specifically includes the use of specific anti-CGRP antibodies and antibody fragments referred to herein as Ab1-Ab14 which comprise or consist of the CDR, VL, VH, CL, and CH polypeptides having the sequences identified in FIGS. 1A-12. The nucleic acid sequences encoding the foregoing VL, VH, CL, and CH polypeptides comprised in Ab1-Ab14 are also comprised in FIGS. 1A-12. The nucleic acid sequences which encode the CDR, VL, VH, CL, and CH polypeptides of an especially preferred anti-CGRP antibody, Ab6, are further described below.

### Antibody Ab6

The invention is further directed to polynucleotides encoding antibody polypeptides having binding specificity to CGRP. In one embodiment of the invention, polynucleotides of the invention comprise, or alternatively consist of, the following polynucleotide sequence encoding the variable light chain polypeptide sequence of SEQ ID NO: 222:

(SEQ ID NO: 232)
CAAGTGCTGacccagtctccatcctccctgtctgcatctgtaggagacag
agtcaccatcAATtgcCAGGCCAGTCAGAGTGTTTATCATAACACCTACC
TGGCCtggtatcagcagaaaccagggaaagttcctaagCAActgatctat
GATGCATCCACTCTGGCATCTggggtcccatctcgtttcagtggcagtgg
atctgggacagatttcactctcaccatcagcagcctgcagcctgaagatg
ttgcaacttattactgtCTGGGCAGTTATGATTGTACTAATGGTGATTGT
TTTGTTttcggcggaggaaccaaggtggaaatcaaacgt.

In one embodiment of the invention, polynucleotides of the invention comprise, or alternatively consist of, the following polynucleotide sequence encoding the light chain polypeptide sequence of SEQ ID NO: 221:

(SEQ ID NO: 231)
CAAGTGCTGacccagtctccatcctccctgtctgcatctgtaggagacag
agtcaccatcAATtgcCAGGCCAGTCAGAGTGTTTATCATAACACCTACC
TGGCCtggtatcagcagaaaccagggaaagttcctaagCAActgatctat
GATGCATCCACTCTGGCATCTggggtcccatctcgtttcagtggcagtgg
atctgggacagatttcactctcaccatcagcagcctgcagcctgaagatg
ttgcaacttattactgtCTGGGCAGTTATGATTGTACTAATGGTGATTGT
TTTGTTttcggcggaggaaccaaggtggaaatcaaacgtACGGTGGCTGC
ACCATCTGTCTTCATCTTCCCGCCATCTGATGAGCAGTTGAAATCTGGAA
CTGCCTCTGTTGTGTGCCTGCTGAATAACTTCTATCCCAGAGAGGCCAAA
GTACAGTGGAAGGTGGATAACGCCCTCCAATCGGGTAACTCCCAGGAGAG
TGTCACAGAGCAGGACAGCAAGGACAGCACCTACAGCCTCAGCAGCACCC
TGACGCTGAGCAAAGCAGACTACGAGAAACACAAAGTCTACGCCTGCGAA
GTCACCCATCAGGGCCTGAGCTCGCCCGTCACAAAGAGCTTCAACAGGGG
AGAGTGTTAG.

In another embodiment of the invention, polynucleotides of the invention comprise, or alternatively consist of, the following polynucleotide sequence encoding the variable heavy chain polypeptide sequence of SEQ ID NO: 202:

(SEQ ID NO: 212)
gaggtgcagctTgtggagtctgggggaggcttggtccagcctggggggtc
cctgagactctcctgtgcaGTCtctggaATCGACCTCagtGGCTACTACA
TGAACtgggtccgtcaggctccagggaaggggctggagtgggtcGGAGTC
ATTGGTATTAATGGTGCCACATACTACGCGAGCTGGGCGAAAGGCcgatt
caccatctccagagacaattccaagACCACGGTGtatcttcaaatgaaca
gcctgagagctgaggacactgctgtgtatTTCtgtGCTAGAGGGGACATC
tggggccaagggaccctcgtcaccgtcTCGAGC.

In one embodiment of the invention, polynucleotides of the invention comprise, or alternatively consist of, the following polynucleotide sequence encoding the heavy chain polypeptide sequence of SEQ ID NO: 201:

(SEQ ID NO: 211)
gaggtgcagctTgtggagtctgggggaggcttggtccagcctggggggtc
cctgagactctcctgtgcaGTCtctggaATCGACCTCagtGGCTACTACA
TGAACtgggtccgtcaggctccagggaaggggctggagtgggtcGGAGTC
ATTGGTATTAATGGTGCCACATACTACGCGAGCTGGGCGAAAGGCcgatt
caccatctccagagacaattccaagACCACGGTGtatcttcaaatgaaca
gcctgagagctgaggacactgctgtgtatTTCtgtGCTAGAGGGGACATC
tggggccaagggaccctcgtcaccgtcTCGAGCGCCTCCACCAAGGGCCC
ATCGGTCTTCCCCCTGGCAcCCTCCTCCaAGAGCACCTCTGGGGGCACAG
CGGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGT
CCTACAGTCCTCAGGACTCTACTCCCTCAGCAGCGTGGTGACCGTGCCCT
CCAGCAGCTTGGGCACCCAGACCTACATCTGCAACGTGAATCACAAGCCC
AGCAACACCAAGGTGGACGCGAGAGTTGAGCCCAAATCTTGTGACAAAAC
TCACACATGCCCACCGTGCCCAGCACCTGAACTCCTGGGGGGACCGTCAG
TCTTCCTCTTCCCCCCAAAACCCAAGGACACCCTCATGaTCTCCCgGACC
CCTGAGGTCACATGCGTGGTGGTGGACGTGAGCCACGAAGACCCTGAGGT
CAAGTTCAACTGGTACGTGGACGGCGTGGAGGTGCATAATGCCAAGACAA
AGCCGCGGGAGGAGCAGTACGCCAGCACGTACCGTGTGGTCAGCGTCCTC
ACCGTCCTGCACCAGGACTGGCTGAATGGCAAGGAGTACAAGTGCAAGGT
CTCCAACAAAGCCCTCCCAGCCCCCATCGAGAAAACCATCTCCAAAGCCA
AAGGGCAGCCCCGAGAACCACAGGTGTACACCCTGCCCCCATCCCGGGAG
GAGATGACCAAGAACCAGGTCAGCCTGACCTGCCTGGTCAAAGGCTTCTA
TCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTC
TACAGCAAGCTCACCGTGGACAAGAGCAGGTGGCAGCAGGGGAACGTCTT
CTCATGCTCCGTGATGCATGAGGCTCTGCACAACCACTACACGCAGAAGA
GCCTCTCCCTGTCTCCGGGTAAATGA.

In one embodiment of the invention, polynucleotides of the invention comprise, or alternatively consist of, the following polynucleotide sequence encoding the heavy chain polypeptide sequence of SEQ ID NO: 566:

(SEQ ID NO: 567)
gaggtgcagctTgtggagtctgggggaggcttggtccagcctggggggtc
cctgagactctcctgtgcaGTCtctggaATCGACCTCagtGGCTACTACA
TGAACtgggtccgtcaggctccagggaaggggctggagtgggtcGGAGTC
ATTGGTATTAATGGTGCCACATACTACGCGAGCTGGGCGAAAGGCcgatt
caccatctccagagacaattccaagACCACGGTGtatcttcaaatgaaca
gcctgagagctgaggacactgctgtgtatTTCtgtGCTAGAGGGGACATC
tggggccaagggaccctcgtcaccgtcTCGAGCGCCTCCACCAAGGGCCC
ATCGGTCTTCCCCCTGGCAcCCTCCTCCaAGAGCACCTCTGGGGGCACAG
CGGCCCTGGGCTGCCTGGTCAAGGACTACTTCCCCGAACCGGTGACGGTG
TCGTGGAACTCAGGCGCCCTGACCAGCGGCGTGCACACCTTCCCGGCTGT
CCTACAGTCCTCAGGACTCTACTCCCTCAGCAGCGTGGTGACCGTGCCCT
CCAGCAGCTTGGGCACCCAGACCTACATCTGCAACGTGAATCACAAGCCC
AGCAACACCAAGGTGGACGCGAGAGTTGAGCCCAAATCTTGTGACAAAAC
TCACACATGCCCACCGTGCCCAGCACCTGAACTCCTGGGGGGACCGTCAG
TCTTCCTCTTCCCCCCAAAACCCAAGGACACCCTCATGaTCTCCCgGACC
CCTGAGGTCACATGCGTGGTGGTGGACGTGAGCCACGAAGACCCTGAGGT
CAAGTTCAACTGGTACGTGGACGGCGTGGAGGTGCATAATGCCAAGACAA
AGCCGCGGGAGGAGCAGTACGCCAGCACGTACCGTGTGGTCAGCGTCCTC
ACCGTCCTGCACCAGGACTGGCTGAATGGCAAGGAGTACAAGTGCAAGGT
CTCCAACAAAGCCCTCCCAGCCCCCATCGAGAAAACCATCTCCAAAGCCA
AAGGGCAGCCCCGAGAACCACAGGTGTACACCCTGCCCCCATCCCGGGAG
GAGATGACCAAGAACCAGGTCAGCCTGACCTGCCTGGTCAAAGGCTTCTA
TCCCAGCGACATCGCCGTGGAGTGGGAGAGCAATGGGCAGCCGGAGAACA
ACTACAAGACCACGCCTCCCGTGCTGGACTCCGACGGCTCCTTCTTCCTC
TACAGCAAGCTCACCGTGGACAAGAGCAGGTGGCAGCAGGGGAACGTCTT
CTCATGCTCCGTGATGCATGAGGCTCTGCACAACCACTACACGCAGAAGA
GCCTCTCCCTGTCTCCGGGTTGA.

In a further embodiment of the invention, polynucleotides encoding antibody fragments having binding specificity to CGRP comprise, or alternatively consist of, one or more of the polynucleotide sequences of SEQ ID NO: 234; SEQ ID NO: 236; and SEQ ID NO: 238 which correspond to polynucleotides encoding the complementarity-determining regions (CDRs, or hypervariable regions) of the light chain variable sequence of SEQ ID NO: 222 or the light chain sequence of SEQ ID NO: 221.

In a further embodiment of the invention, polynucleotides encoding antibody fragments having binding specificity to CGRP comprise, or alternatively consist of, one or more of the polynucleotide sequences of SEQ ID NO: 214; SEQ ID NO: 216; and SEQ ID NO: 218 which correspond to polynucleotides encoding the complementarity-determining regions (CDRs, or hypervariable regions) of the heavy chain variable sequence of SEQ ID NO: 202 or the heavy chain sequence of SEQ ID NO: 201 or SEQ ID NO: 566.

The invention also contemplates polynucleotide sequences including one or more of the polynucleotide sequences encoding antibody fragments described herein. In one embodiment of the invention, polynucleotides encoding antibody fragments having binding specificity to CGRP comprise, or alternatively consist of, one, two, three or more, including all of the following polynucleotides encoding antibody fragments: the polynucleotide SEQ ID NO: 232 encoding the light chain variable sequence of SEQ ID NO: 222; the polynucleotide SEQ ID NO: 231 encoding the light chain sequence of SEQ ID NO: 221; the polynucleotide SEQ ID NO: 212 encoding the heavy chain variable sequence of SEQ ID NO: 202; the polynucleotide SEQ ID NO: 211 encoding the heavy chain sequence of SEQ ID NO: 201; the polynucleotide SEQ ID NO: 567 encoding the heavy chain sequence of SEQ ID NO: 566; polynucleotides encoding the complementarity-determining regions (SEQ ID NO: 234; SEQ ID NO: 236; and SEQ ID NO: 238) of the light chain variable sequence of SEQ ID NO: 222 or the light chain sequence of SEQ ID NO: 221; and polynucleotides encoding the complementarity-determining regions (SEQ ID NO: 214; SEQ ID NO: 216; and SEQ ID NO: 218) of the heavy chain variable sequence of SEQ ID NO: 202 or the heavy chain sequence of SEQ ID NO: 201 or SEQ ID NO: 566.

In a preferred embodiment of the invention, polynucleotides of the invention comprise, or alternatively consist of, polynucleotides encoding Fab (fragment antigen binding) fragments having binding specificity for CGRP. With respect to antibody Ab6, the polynucleotides encoding the full length Ab6 antibody comprise, or alternatively consist of, the polynucleotide SEQ ID NO: 231 encoding the light chain sequence of SEQ ID NO: 221 and the polynucleotide SEQ ID NO: 211 encoding the heavy chain sequence of SEQ ID NO: 201 or the polynucleotide SEQ ID NO: 567 encoding the heavy chain sequence of SEQ ID NO: 566.

Another embodiment of the invention contemplates these polynucleotides incorporated into an expression vector for expression in mammalian cells such as CHO, NSO, HEK-293, or in fungal, insect, or microbial systems such as yeast cells such as the yeast Pichia. Suitable Pichia species include, but are not limited to, Pichia pastoris. In one embodiment of the invention described herein (infra), Fab fragments may be produced by enzymatic digestion (e.g., papain) of Ab6 following expression of the full-length polynucleotides in a suitable host. In another embodiment of the invention, anti-CGRP antibodies such as Ab6 or Fab fragments thereof may be produced via expression of Ab6 polynucleotides in mammalian cells such as CHO, NSO or HEK 293 cells, fungal, insect, or microbial systems such as yeast cells (for example diploid yeast such as diploid Pichia) and other yeast strains. Suitable Pichia species include, but are not limited to, Pichia pastoris.

In one embodiment, the invention is directed to an isolated polynucleotide comprising a polynucleotide encoding an anti-CGRP VH antibody amino acid sequence selected from SEQ ID NO: 2, SEQ ID NO: 42, SEQ ID NO: 82, SEQ ID NO: 122, SEQ ID NO: 162, SEQ ID NO: 202, SEQ ID NO: 242, SEQ ID NO: 282, SEQ ID NO: 322, SEQ ID NO: 362, SEQ ID NO: 402, SEQ ID NO: 442, SEQ ID NO: 482, or SEQ ID NO: 522 or encoding a variant thereof wherein at least one framework residue (FR residue) has been substituted with an amino acid present at the corresponding position in a rabbit anti-CGRP antibody V_{H} polypeptide or a conservative amino acid substitution.

In another embodiment, the invention is directed to an isolated polynucleotide comprising the polynucleotide sequence encoding an anti-CGRP V_{L} antibody amino acid sequence of SEQ ID NO: 22, SEQ ID NO: 62, SEQ ID NO: 102, SEQ ID NO: 142, SEQ ID NO: 182, SEQ ID NO: 222, SEQ ID NO: 262, SEQ ID NO: 302, SEQ ID NO: 342, SEQ ID NO: 382, SEQ ID NO: 422, SEQ ID NO: 462, SEQ ID NO: 502, or SEQ ID NO: 542, or encoding a variant thereof wherein at least one framework residue (FR residue) has been substituted with an amino acid present at the corresponding position in a rabbit anti-CGRP antibody V_{L} polypeptide or a conservative amino acid substitution.

In yet another embodiment, the invention is directed to one or more heterologous polynucleotides comprising a sequence encoding the polypeptides contained in SEQ ID NO: 22 and SEQ ID NO: 2; SEQ ID NO: 62 and SEQ ID NO: 42; SEQ ID NO: 102 and SEQ ID NO: 82; SEQ ID NO: 142 and SEQ ID NO: 122; SEQ ID NO: 182 and SEQ ID NO: 162; SEQ ID NO: 222 and SEQ ID NO: 202; SEQ ID NO: 262 and SEQ ID NO: 242; SEQ ID NO: 302 and SEQ ID NO: 282; SEQ ID NO: 342 and SEQ ID NO: 322; SEQ ID NO: 382 and SEQ ID NO: 362; SEQ ID NO: 422 and SEQ ID NO: 402; SEQ ID NO: 462 and SEQ ID NO: 442; SEQ ID NO: 502 and SEQ ID NO: 482; or SEQ ID NO: 542 and SEQ ID NO: 522.

In another embodiment, the invention is directed to an isolated polynucleotide that expresses a polypeptide containing at least one CDR polypeptide derived from an anti-CGRP antibody wherein said expressed polypeptide alone specifically binds CGRP or specifically binds CGRP when expressed in association with another polynucleotide sequence that expresses a polypeptide containing at least one CDR polypeptide derived from an anti-CGRP antibody wherein said at least one CDR is selected from those contained in the V_{L} or VH polypeptides of SEQ ID NO: 22, SEQ ID NO: 2, SEQ ID NO: 62, SEQ ID NO: 42, SEQ ID NO: 102, SEQ ID NO: 82, SEQ ID NO: 142, SEQ ID NO: 122, SEQ ID NO: 182, SEQ ID NO: 162, SEQ ID NO: 222, SEQ ID NO: 202, SEQ ID NO: 262, SEQ ID NO: 242, SEQ ID NO: 302, SEQ ID NO: 282, SEQ ID NO: 342, SEQ ID NO: 322, SEQ ID NO: 382, SEQ ID NO: 362, SEQ ID NO: 422, SEQ ID NO: 402, SEQ ID NO: 462, SEQ ID NO: 442, SEQ ID NO: 502, SEQ ID NO: 482, SEQ ID NO: 542, or SEQ ID NO: 522.

Host cells and vectors comprising said polynucleotides are also contemplated.

The invention further contemplates vectors comprising the polynucleotide sequences encoding the variable heavy and light chain polypeptide sequences, as well as the individual complementarity-determining regions (CDRs, or hypervariable regions), as set forth herein, as well as host cells comprising said vector sequences. In one embodiment of the invention, the host cell is a yeast cell. In another embodiment of the invention, the yeast host cell belongs to the genus Pichia.

Methods of Producing Antibodies and Fragments thereof

In another embodiment, the present invention contemplates methods for producing anti-CGRP antibodies and fragments thereof. Methods for producing antibodies and fragments thereof secreted from polyploidal, preferably diploid or tetraploid strains of mating competent yeast are taught, for example, in U.S. patent application publication no. US 2009/0022659 to Olson et al., and in U.S. Pat. No. 7,935,340 to Garcia-Martinez et al., the disclosures of each of which are herein incorporated by reference in their entireties. Methods for producing antibodies and fragments thereof in mammalian cells, e.g., CHO cells are further well known in the art.

Other methods of producing antibodies are also well known to those of ordinary skill in the art. For example, methods of producing chimeric antibodies are now well known in the art (See, for example, U.S. Pat. No. 4,816,567 to Cabilly et al.; Morrison et al., P.N.A.S. USA, 81:8651-55 (1984); Neuberger, M. S. et al., Nature, 314:268-270 (1985); Boulianne, G. L. et al., Nature, 312:643-46 (1984), the disclosures of each of which are herein incorporated by reference in their entireties).

Likewise, other methods of producing humanized antibodies are now well known in the art (See, for example, U.S. Pat. Nos. 5,530,101, 5,585,089, 5,693,762, and 6,180,370 to Queen et al; U.S. Pat. Nos. 5,225,539 and 6,548,640 to Winter; U.S. Pat. Nos. 6,054,297, 6,407,213 and 6,639,055 to Carter et al; U.S. Pat. No. 6,632,927 to Adair; Jones, P. T. et al, Nature, 321:522-525 (1986); Reichmann, L., et al, Nature, 332:323-327 (1988); Verhoeyen, M, et al, Science, 239:1534-36 (1988), the disclosures of each of which are herein incorporated by reference in their entireties).

The term “opioid analgesic” herein refers to all drugs, natural or synthetic, with morphine-like actions. The synthetic and semi-synthetic opioid analgesics are derivatives of five chemical classes of compound: phenanthrenes; phenylheptylamines; phenylpiperidines; morphinans; and benzomorphans, all of which are within the scope of the term. Exemplary opioid analgesics include codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine or pharmaceutically acceptable salts thereof.

The term “NSAID” refers to a non-steroidal anti-inflammatory compound. NSAIDs are categorized by virtue of their ability to inhibit cyclooxygenase. Cyclooxygenase 1 and cyclooxygenase 2 are two major isoforms of cyclooxygenase and most standard NSAIDs are mixed inhibitors of the two isoforms. Most standard NSAIDs fall within one of the following five structural categories: (1) propionic acid derivatives, such as ibuprofen, naproxen, naprosyn, diclofenac, and ketoprofen; (2) acetic acid derivatives, such as tolmetin and slindac; (3) fenamic acid derivatives, such as mefenamic acid and meclofenamic acid; (4) biphenylcarboxylic acid derivatives, such as diflunisal and flufenisal; and (5) oxicams, such as piroxim, sudoxicam, and isoxicam. Another class of NSAID has been described which selectively inhibit cyclooxygenase 2. Cox-2 inhibitors have been described, e.g., in U.S. Pat. Nos. 5,616,601; 5,604,260; 5,593,994; 5,550,142; 5,536,752; 5,521,213; 5,475,995; 5,639,780; 5,604,253; 5,552,422; 5,510,368; 5,436,265; 5,409,944; and 5,130,311, all of which are hereby incorporated by reference. Certain exemplary COX-2 inhibitors include celecoxib (SC-58635), DUP-697, flosulide (CGP-28238), meloxicam, 6-methoxy-2 naphthylacetic acid (6-MNA), rofecoxib, MK-966, nabumetone (prodrug for 6-MNA), nimesulide, NS-398, SC-5766, SC-58215, T-614; or combinations thereof.

In some embodiments, aspirin and/or acetaminophen may be taken in conjunction with the subject CGRP antibody or fragment. Aspirin is another type of non-steroidal anti-inflammatory compound.

The subject to which the pharmaceutical formulation is administered can be, e.g., any human or non-human animal that is in need of such treatment, prevention and/or amelioration, or who would otherwise benefit from the inhibition or attenuation of medication overuse headache. For example, the subject can be an individual that is diagnosed with, or who is deemed to be at risk of being afflicted by medication overuse headache. The present invention further includes the use of any of the pharmaceutical formulations disclosed herein in the manufacture of a medicament for the treatment, prevention and/or amelioration of medication overuse headache.

### Administration

In one embodiment of the invention, the anti-CGRP antibodies described herein, or CGRP binding fragments thereof, as well as combinations of said antibodies or antibody fragments, are administered to a subject at a concentration of between about 0.1 and 100.0 mg/kg of body weight of recipient subject. In a preferred embodiment of the invention, the anti-CGRP antibodies described herein, or CGRP binding fragments thereof, as well as combinations of said antibodies or antibody fragments, are administered to a subject at a concentration of about 0.4 mg/kg of body weight of recipient subject and/or at a dosage of 100 or 300 mg. In a preferred embodiment of the invention, the anti-CGRP antibodies described herein, or CGRP binding fragments thereof, as well as combinations of said antibodies or antibody fragments, are administered to a recipient subject with a frequency of once every twenty-six weeks or six months or less, such as once every sixteen weeks or four months or less, once every eight weeks or two months or less, once every four weeks or monthly or less, once every two weeks or bimonthly or less, once every week or less, or once daily or less. In general the administration of sequential doses may vary by plus or minus a few days from the aforementioned schedule, e.g., administration every 3 months or every 12 weeks includes administration of a dose varying from the schedule day by plus or minus 1, 2, 3, 4, 5, 5, or 7 days.

Fab fragments may be administered every two weeks or less, every week or less, once daily or less, multiple times per day, and/or every few hours. In one embodiment of the invention, a patient receives Fab fragments of 0.1 mg/kg to 40 mg/kg per day given in divided doses of 1 to 6 times a day, or in a sustained release form, effective to obtain desired results.

It is to be understood that the concentration of the antibody or Fab administered to a given patient may be greater or lower than the exemplary administration concentrations set forth above.

A person of skill in the art would be able to determine an effective dosage and frequency of administration through routine experimentation, for example guided by the disclosure herein and the teachings in Goodman, L. S., Gilman, A., Brunton, L. L., Lazo, J. S., & Parker, K. L. (2006). Goodman & Gilman's the pharmacological basis of therapeutics. New York: McGraw-Hill; Howland, R. D., Mycek, M. J., Harvey, R. A., Champe, P. C., & Mycek, M. J. (2006). Pharmacology. Lippincott's illustrated reviews. Philadelphia: Lippincott Williams & Wilkins; and Golan, D. E. (2008). Principles of pharmacology: the pathophysiologic basis of drug therapy. Philadelphia, Pa., [etc.]: Lippincott Williams & Wilkins.

In another embodiment of the invention, the anti-CGRP antibodies described herein, or CGRP binding fragments thereof, as well as combinations of said antibodies or antibody fragments, are administered to a subject in a pharmaceutical formulation.

A “pharmaceutical composition” refers to a chemical or biological composition suitable for administration to a mammal. Such compositions may be specifically formulated for administration via one or more of a number of routes, including but not limited to buccal, epicutaneous, epidural, inhalation, intraarterial, intracardial, intracerebroventricular, intradermal, intramuscular, intranasal, intraocular, intraperitoneal, intraspinal, intrathecal, intravenous, oral, parenteral, rectally via an enema or suppository, subcutaneous, subdermal, sublingual, transdermal, and transmucosal, preferably intravenous. In addition, administration can occur by means of injection, powder, liquid, gel, drops, or other means of administration.

A “pharmaceutical excipient” or a “pharmaceutically acceptable excipient” is a carrier, usually a liquid, in which an active therapeutic agent is formulated. In one embodiment of the invention, the active therapeutic agent is a humanized antibody described herein, or one or more fragments thereof. The excipient generally does not provide any pharmacological activity to the formulation, though it may provide chemical and/or biological stability, and release characteristics. Exemplary formulations can be found, for example, in Remington's Pharmaceutical Sciences, 19^{th} Ed., Grennaro, A., Ed., 1995 which is incorporated by reference.

As used herein “pharmaceutically acceptable carrier” or “excipient” includes any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, or sublingual administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions typically must be sterile and stable under the conditions of manufacture and storage. The invention contemplates that the pharmaceutical composition is present in lyophilized form. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol), and suitable mixtures thereof. The invention further contemplates the inclusion of a stabilizer in the pharmaceutical composition. The proper fluidity can be maintained, for example, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the alkaline polypeptide can be formulated in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

An exemplary composition comprises, consists essentially of, or consists of an anti-CGRP antibody or fragment thereof (e.g., Ab6), an excipient such as histidine, an isotonic agent such as sorbitol, and a surfactant such as polysorbate 80 in an aqueous solution. For example, the composition may comprise, consist essentially of, or consist of histidine (L-histidine), sorbitol, polysorbate 80, such as, per 1 mL volume, about 100 mg anti-CGRP antibody (e.g., Ab6), about 3.1 mg L-Histidine, about 40.5 mg Sorbitol, and about 0.15 mg Polysorbate 80, having a pH of about 5.8, or approximately that constitution, e.g., within 10% of those values, within 5% of those values, within 1% of those values, within 0.5% of those values, or within 0.10% of those values, and water. For example, the pH value may be within 10% of 5.8, i.e., between 5.22 and 6.38. The Ab6 antibody may comprise or consist of the variable light and heavy chain polypeptides of SEQ ID NO: 222 and SEQ ID NO: 202 respectively, or the light and heavy chain polypeptides of SEQ ID NO: 221 and SEQ ID NO: 201 respectively, or the light and heavy chain polypeptides of SEQ ID NO: 221 and SEQ ID NO: 566 respectively. The composition may be in the form of an aqueous solution, or a concentrate (e.g., lyophilized) which when reconstituted, e.g., by addition of water, yields the aforementioned constitution. An exemplary composition consists of, per mL, 100 mg of the light and heavy chain polypeptides of SEQ ID NO: 221 and SEQ ID NO: 201 respectively, about 3.1 mg L-Histidine, about 40.5 mg Sorbitol, and about 0.15 mg Polysorbate 80, and water Q.S, or approximately that constitution, e.g., within 10% of those quantities, within 5% of those quantities, within 1% of those quantities, within 0.5% of those quantities, or within 0.1% of those quantities. Another exemplary composition consists of, per mL, 100 mg of the light and heavy chain polypeptides of SEQ ID NO: 221 and SEQ ID NO: 566 respectively, about 3.1 mg L-Histidine, about 40.5 mg Sorbitol, and about 0.15 mg Polysorbate 80, and water Q.S, or approximately that constitution, e.g., within 10% of those quantities, within 5% of those quantities, within 1% of those quantities, within 0.5% of those quantities, or within 0.10% of those quantities. The composition may be suitable for intravenous or subcutaneous administration, preferably intravenous administration. For example, the composition may be suitable for mixing with an intravenous solution (such as 0.9% sodium chloride) at an amount of between about 100 mg and about 300 mg antibody added to 100 mL of intravenous solution. Preferably the composition may be shelf-stable for at least 1, 3, 6, 12, 18, or 24 months, e.g., showing formation of aggregates of no more than 5% or no more than 10% of the antibody or fragment after storage at room temperature or when refrigerated at 4° C. for the specified duration, or in an accelerated aging test that simulates storage for that duration.

For each of the recited embodiments, the compounds can be administered by a variety of dosage forms. Any biologically-acceptable dosage form known to persons of ordinary skill in the art, and combinations thereof, are contemplated. Examples of such dosage forms include, without limitation, reconstitutable powders, elixirs, liquids, solutions, suspensions, emulsions, powders, granules, particles, microparticles, dispersible granules, cachets, inhalants, aerosol inhalants, patches, particle inhalants, implants, depot implants, injectables (including subcutaneous, intramuscular, intravenous, and intradermal, preferably intravenous), infusions, and combinations thereof.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

These and other changes can be made to the invention in light of the above detailed description. In general, in the following claims, the terms used should not be construed to limit the invention to the specific embodiments disclosed in the specification and the claims. Accordingly, the invention is not limited by the disclosure, but instead the scope of the invention is to be determined entirely by the following claims.

The invention may be practiced in ways other than those particularly described in the foregoing description and examples. Numerous modifications and variations of the invention are possible in light of the above teachings and, therefore, are within the scope of the appended claims.

Certain CGRP antibody polynucleotides and polypeptides are disclosed in the sequence listing accompanying this patent application filing, and the disclosure of said sequence listing is herein incorporated by reference in its entirety.

The entire disclosure of each document cited (including patents, patent applications, journal articles, abstracts, manuals, books, or other disclosures) in the Background of the Invention, Detailed Description, and Examples is herein incorporated by reference in their entireties.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Additional Exemplary Embodiments

Additional exemplary embodiments of the invention are provided as follows:
S1. Use of an anti-CGRP antibody for the manufacturing of a medicament for treating acute migraine, comprising administering to a patient an anti-CGRP antibody while the patient has a headache and within 30 hours of headache onset, preferably within 24 hours of headache onset.
S2. Use of the anti-CGRP antibody of embodiment 1, wherein the medicament is for administration within 18 hours, within 12 hours, within 6 hours, within 5 hours, within 4 hours, within 3 hours, within 2 hours, or within 1 hour of headache onset, such as between 1-6 hours from headache onset.
S3. Use of the anti-CGRP antibody of any one of the foregoing embodiments, wherein said medicament is for intravenous or subcutaneous infusion.
S4. Use of the anti-CGRP antibody of any one of the foregoing embodiments, wherein said medicament is for intravenous infusion.
S5. Use of the anti-CGRP antibody of any one of the foregoing embodiments, wherein said patient is headache free 2 hours post-completion of administration or infusion.
S6. Use of the anti-CGRP antibody of any one of the foregoing embodiments, wherein said patient is free from the most bothersome symptom (MBS) at 2 hours post-completion of administration or infusion.
S7. Use of the anti-CGRP antibody of any one of the foregoing embodiments, wherein the administration of said medicament improves one or more of the following: time to pain relief; time to pain freedom; headache pain freedom at 2 hours with sustained headache pain freedom for 24 and 48 hours post completion of infusion; use of rescue medication by 24 hours and by 48 hours post completion of infusion; absence of photophobia at 2 hours post completion of infusion; absence of phonophobia at 2 hours post completion of infusion; absence of nausea at 2 hours post completion of infusion; change from baseline in Headache Impact Test (HIT-6) at Week 4; or change from baseline in Migraine Treatment Optimization Questionnaire-6 (MTOQ-6) at Week 4.
S8. Use of the anti-CGRP antibody of any one of the foregoing embodiments, wherein said anti-CGRP antibody comprises any one of Ab1-Ab14.
S9. Use of the anti-CGRP antibody of any one of the foregoing embodiments, wherein said anti-CGRP antibody comprises Ab6.
S10. Use of the anti-CGRP antibody of any one of the foregoing embodiments, wherein said anti-CGRP antibody comprises the light chain complementarity-determining region (CDR) 1, 2, and 3 polypeptide sequences of SEQ ID NO: 224; SEQ ID NO: 226; and SEQ ID NO: 228, respectively.
S11. Use of the anti-CGRP antibody of any one of the foregoing embodiments, wherein said anti-CGRP antibody comprises the light chain CDR 1, 2, and 3 polypeptide sequences encoded by SEQ ID NO: 234; SEQ ID NO: 236; and SEQ ID NO: 238, respectively.
S12. Use of the anti-CGRP antibody of any one of the foregoing embodiments, wherein said anti-CGRP antibody comprises the heavy chain CDR 1, 2, and 3 polypeptide sequences of SEQ ID NO: 204; SEQ ID NO: 206; and SEQ ID NO: 208, respectively.
S13. Use of the anti-CGRP antibody of any one of the foregoing embodiments, wherein said anti-CGRP antibody comprises the heavy chain CDR 1, 2, and 3 polypeptide sequences encoded by SEQ ID NO: 214; SEQ ID NO: 216; and SEQ ID NO: 218, respectively.
S14. Use of the anti-CGRP antibody of any one of the foregoing embodiments, wherein said anti-CGRP antibody comprises the light chain CDR 1, 2, and 3 polypeptide sequences of SEQ ID NO: 224; SEQ ID NO: 226; and SEQ ID NO: 228, respectively and heavy chain CDR 1, 2, and 3 polypeptide sequences of SEQ ID NO: 204; SEQ ID NO: 206; and SEQ ID NO: 208, respectively.
S15. Use of the anti-CGRP antibody of any one of the foregoing embodiments, wherein said anti-CGRP antibody comprises the light chain CDR 1, 2, and 3 polypeptide sequences encoded by SEQ ID NO: 234; SEQ ID NO: 236; and SEQ ID NO: 238, respectively and heavy chain CDR 1, 2, and 3 polypeptide sequences encoded by SEQ ID NO: 214; SEQ ID NO: 216; and SEQ ID NO: 218, respectively.
S16. Use of the anti-CGRP antibody of any one of the foregoing embodiments, wherein said anti-CGRP antibody comprises the variable light chain polypeptide of SEQ ID NO: 222.
S17. Use of the anti-CGRP antibody of any one of the foregoing embodiments, wherein said anti-CGRP antibody comprises the variable light chain polypeptide encoded by SEQ ID NO: 232.
S18. Use of the anti-CGRP antibody of any one of the foregoing embodiments, wherein said anti-CGRP antibody comprises the variable heavy chain polypeptide of SEQ ID NO: 202.
S19. Use of the anti-CGRP antibody of any one of the foregoing embodiments, wherein said anti-CGRP antibody comprises the variable heavy chain polypeptide encoded by SEQ ID NO: 212.
S20. Use of the anti-CGRP antibody of any one of the foregoing embodiments, wherein said anti-CGRP antibody comprises the variable light chain polypeptide of SEQ ID NO: 222 and the variable heavy chain polypeptide of SEQ ID NO: 202.
S21. Use of the anti-CGRP antibody of any one of the foregoing embodiments, wherein said anti-CGRP antibody comprises the variable light chain polypeptide encoded by SEQ ID NO: 232 and the variable heavy chain polypeptide encoded by SEQ ID NO: 212.
S22. Use of the anti-CGRP antibody of any one of the foregoing embodiments, wherein said anti-CGRP antibody comprises the light chain polypeptide of SEQ ID NO: 221.
S23. Use of the anti-CGRP antibody of any one of the foregoing embodiments, wherein said anti-CGRP antibody comprises the light chain polypeptide encoded by SEQ ID NO: 231.
S24. Use of the anti-CGRP antibody of any one of the foregoing embodiments, wherein said anti-CGRP antibody comprises the heavy chain polypeptide of SEQ ID NO: 201 or SEQ ID NO: 566.
S25. Use of the anti-CGRP antibody of any one of the foregoing embodiments, wherein said anti-CGRP antibody comprises the heavy chain polypeptide encoded by SEQ ID NO: 211 or SEQ ID NO: 567.
S26. Use of the anti-CGRP antibody of any one of the foregoing embodiments, wherein said anti-CGRP antibody comprises the light chain polypeptide of SEQ ID NO: 221 and the heavy chain polypeptide of SEQ ID NO: 201 or SEQ ID NO: 566.
S27. Use of the anti-CGRP antibody of any one of the foregoing embodiments, wherein said anti-CGRP antibody comprises the light chain polypeptide encoded by SEQ ID NO: 231 and the heavy chain polypeptide encoded by SEQ ID NO: 211 or SEQ ID NO: 567.
S28. Use of the anti-CGRP antibody of any one of the foregoing embodiments, wherein the administered amount of said anti-CGRP antibody is between about 100 mg and about 300 mg, or is about 100 mg, or is about 300 mg.
S29. Use of the anti-CGRP antibody of any one of the foregoing embodiments, wherein the administered amount of said anti-CGRP antibody is 100 mg.
S30. Use of the anti-CGRP antibody of any one of the foregoing embodiments, wherein said medicament is for intravenous administration in a dosage of 100 mg of said anti-CGRP antibody every 10⁻¹⁴ weeks, preferably every 11-13 weeks, more preferably every 12 weeks.
S31. Use of the anti-CGRP antibody of any one of embodiments S1-S29, wherein said medicament is for intravenous administration in a dosage of 300 mg of said anti-CGRP antibody every 10⁻¹⁴ weeks, preferably every 11-13 weeks, more preferably every 12 weeks.
S32. Use of the anti-CGRP antibody of any one of the foregoing embodiments, wherein said patient suffers from medication overuse headache.
S33. Use of the anti-CGRP antibody of any one of the foregoing embodiments wherein, prior to administration of said medicament, the patient exhibits between 1-10 migraine attacks per month in the month or in the 3 months prior to administration.
S34. Use of the anti-CGRP antibody of any one of the foregoing embodiments wherein, prior to administration of said medicament, the patient exhibits between 2-8 migraine attacks per month in the month or in the 3 months prior to administration.
S35. Use of the anti-CGRP antibody of any one of the foregoing embodiments wherein, prior to administration of said medicament, the patient exhibits between 3-7 migraine attacks per month in the month or in the 3 months prior to administration.
S36. Use of the anti-CGRP antibody of any one of the foregoing embodiments wherein, prior to administration of said medicament, the patient exhibits less than 25 headache days per month in the month or in the 3 months prior to administration.
S37. Use of the anti-CGRP antibody of any one of the foregoing embodiments wherein, prior to administration of said medicament, the patient exhibits less than 20 headache days per month in the month or in the 3 months prior to administration.
S38. Use of the anti-CGRP antibody of any one of the foregoing embodiments wherein, prior to administration of said medicament, the patient exhibits less than 15 headache days per month in the month or in the 3 months prior to administration.
S39. Use of the anti-CGRP antibody of any one of the foregoing embodiments wherein, prior to administration of said medicament, the patient exhibits less than 10 headache days per month in the month or in the 3 months prior to administration.
S40. Use of the anti-CGRP antibody of any one of the foregoing embodiments wherein said patient was diagnosed with migraine at least 10 years prior to administration of said medicament.
S41. Use of the anti-CGRP antibody of any one of the foregoing embodiments wherein said patient was diagnosed with migraine at least 15 years prior to administration of said medicament.
S42. Use of the anti-CGRP antibody of any one of the foregoing embodiments wherein said patient was diagnosed with migraine at least 18 or at least 19 years prior to administration of said medicament.
S43. Use of the anti-CGRP antibody of any one of the foregoing embodiments, wherein said patient has a reduction in the number of migraine days by at least 50% in the one month period after being administered said antibody relative to the baseline number of migraine days experienced by that patient prior to administration of said medicament.
S44. Use of the anti-CGRP antibody of any one of the foregoing embodiments, wherein said patient has a reduction in the number of migraine days by at least 75% in the one month period after being administered said antibody relative to the baseline number of migraine days experienced by that patient prior to administration of said medicament.
S45. Use of the anti-CGRP antibody of any one of the foregoing embodiments, wherein said patient has a reduction in the number of migraine days by 100% in the one month period after being administered said antibody relative to the baseline number of migraine days experienced by that patient prior to administration of said medicament.
S46. Use of the anti-CGRP antibody of any one of the foregoing embodiments, wherein said patient has a reduction in the number of migraine days by at least 50% in the 12 week period after being administered said antibody relative to the baseline number of migraine days experienced by that patient prior to administration of said medicament.
S47. Use of the anti-CGRP antibody of any one of the foregoing embodiments, wherein said patient has a reduction in the number of migraine days by at least 75% in the 12 week period after being administered said antibody relative to the baseline number of migraine days experienced by that patient prior to administration of said medicament.
S48. Use of the anti-CGRP antibody of any one of the foregoing embodiments, wherein said patient has a reduction in the number of migraine days by 100% in the 12 week period after being administered said antibody relative to the baseline number of migraine days experienced by that patient prior to administration of said medicament.
S49. Use of the anti-CGRP antibody of any one of the foregoing embodiments, wherein said medicament is further for administration in a second dose of said anti-CGRP antibody about 10⁻¹⁴ weeks, preferably 11-13 weeks, more preferably about 12 weeks or about 3 months after administration of said medicament.
S50. Use of the anti-CGRP antibody of any one of the foregoing embodiments, wherein said medicament comprises about 100 mg, about 125 mg, about 150 mg, about 175 mg, about 200 mg, about 225 mg, about 250 mg, about 275 mg, or about 300 mg of said anti-CGRP antibody.
S51. Use of the anti-CGRP antibody of any one of the foregoing embodiments, wherein said anti-CGRP antibody is aglycosylated or if glycosylated only contains only mannose residues.
S52. Use of the anti-CGRP antibody of any one of the foregoing embodiments, wherein said anti-CGRP antibody consists of the light chain polypeptide of SEQ ID NO: 221 and the heavy chain polypeptide of SEQ ID NO: 201 or SEQ ID NO: 566.
S53. Use of the anti-CGRP antibody of any one of the foregoing embodiments, wherein said anti-CGRP antibody consists of the light chain polypeptide encoded by SEQ ID NO: 231 and the heavy chain polypeptide encoded by SEQ ID NO: 211 or SEQ ID NO: 567.
S54. Use of the anti-CGRP antibody of any one of embodiments S7-S53, wherein said rescue medication comprises one or more of a triptan, non-opioid analgesics (such as paracetamol (acetaminophen), acetylsalicylic acid (aspirin), another NSAID, or another non-opioid analgesic), a combination of two or more drug classes, wherein said triptan use optionally comprises use of one or more of sumatriptan, zolmitriptan, naratriptan, rizatriptan, eletriptan, almotriptan, and frovatriptan, and/or wherein said opioid use optionally comprises use of one or more of oxycodone, tramadol, butorphanol, morphine, codeine, and hydrocodone, and/or wherein said combination of two or more drug class optionally comprises two drugs with analgesic effects (for example, paracetamol and codeine) or acting as adjuvants (for example, caffeine), optionally wherein said combination-analgesics combine non-opioid analgesic includes at least one opioid (such as tramadol, butorphanol, morphine, codeine, hydrocodone, or any combination thereof), barbiturate such as butalbital and/or caffeine.
S55. Use of the anti-CGRP antibody of any one of the foregoing embodiments, wherein said headache or said migraine is diagnosed according to the third edition of the International Classification of Headache Disorders.
S56. Use of the anti-CGRP antibody of any of any one of the foregoing embodiments, wherein said anti-CGRP antibody is expressed in or obtained by expression in Pichia pastoris.
S57. Use of the anti-CGRP antibody of any of any one of embodiments S1-S55, wherein said anti-CGRP antibody is expressed in or obtained by expression in CHO cells.
S58. Use of the anti-CGRP antibody of any one of the foregoing embodiments, wherein said anti-CGRP antibody or anti-CGRP antibody fragment is comprised in a formulation comprising or consisting of histidine (L-histidine), sorbitol, polysorbate 80, and water.
S59. Use of the anti-CGRP antibody of embodiment S58, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−10% of said values, and having a pH of 5.8 or within +/−10% of said value.
S60. Use of the anti-CGRP antibody of embodiment S58, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−5% of said values, and/or having a pH of 5.8 or within +/−5% of said value.
S61. Use of the anti-CGRP antibody of embodiment S58, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−1% of said values, and/or having a pH of 5.8 or within +/−1% of said value.
S62. Use of the anti-CGRP antibody of embodiment S58, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−0.5% of said values, and/or having a pH of 5.8 or within +/−0.5% of said value.
S63. Use of the anti-CGRP antibody of embodiment S58, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−0.1% of said values, and/or having a pH of 5.8 or within +/−0.1% of said value.
S64. Use of an anti-CGRP antibody for the manufacture of a medicament for treating chronic migraine, wherein said medicament is for intravenous administration in a first dosage comprising between about 100 mg and about 300 mg of an anti-CGRP antibody, wherein said anti-CGRP antibody comprises the light chain polypeptide of SEQ ID NO: 221 and the heavy chain polypeptide of SEQ ID NO: 201 or SEQ ID NO: 566, wherein in the first 24 hours after administration of said first dosage the patient exhibits at least a 50% reduction in migraine prevalence.
S65. Use of an anti-CGRP antibody for the manufacture of a medicament for treating chronic migraine, wherein said medicament is for intravenous administration in a first dosage comprising between about 100 mg and about 300 mg of an anti-CGRP antibody, wherein said anti-CGRP antibody comprises the light chain polypeptide of SEQ ID NO: 221 and the heavy chain polypeptide of SEQ ID NO: 201 or SEQ ID NO: 566, wherein on the first day following the day of administration the patient exhibits at least a 50% reduction in migraine prevalence.
S66. Use of the anti-CGRP antibody of embodiment S64 or embodiment S65, wherein the first dosage of said anti-CGRP antibody is 100 mg.
S67. Use of the anti-CGRP antibody of embodiment S64 or embodiment S65, wherein the first dosage of said anti-CGRP antibody is 300 mg.
S68. Use of the anti-CGRP antibody of embodiment S64, S65, or S66, wherein said medicament is for intravenous administration in a dosage of 100 mg of said anti-CGRP antibody every 10⁻¹⁴ weeks, preferably 11-13 weeks, more preferably every 12 weeks.
S69. Use of the anti-CGRP antibody of embodiment S64, S65, or S67, wherein said medicament is for intravenous administration in a dosage of 300 mg of said anti-CGRP antibody every 10⁻¹⁴ weeks, preferably 11-13 weeks, more preferably every 12 weeks.
S70. Use of the anti-CGRP antibody of any one of embodiments S64-S69 wherein, prior to said first dosage, the patient exhibits between about 10 and about 22 migraine days per month.
S71. Use of the anti-CGRP antibody of any one of embodiments S64-S70 wherein, prior to said first dosage, the patient exhibits between about 13 and about 19 migraine days per month.
S72. Use of the anti-CGRP antibody of any one of embodiments S64-S71 wherein, prior to said first dosage, the patient exhibits about 16 migraine days per month.
S73. Use of the anti-CGRP antibody of any one of embodiments S64-S72 wherein, prior to said first dosage, the patient exhibits between about 14 and about 27 headache days per month.
S74. Use of the anti-CGRP antibody of any one of embodiments S64-S73 wherein, prior to said first dosage, the patient exhibits between about 17 and about 24 headache days per month.
S75. Use of the anti-CGRP antibody of any one of embodiments S64-S74 wherein, prior to said first dosage, the patient exhibits about 20 or about 21 headache days per month.
S76. Use of the anti-CGRP antibody of any one of embodiments S64-S75 wherein said patient was diagnosed with migraine at least 10 years prior to said first dosage.
S77. Use of the anti-CGRP antibody of any one of embodiments S64-S76 wherein said patient was diagnosed with migraine at least 15 years prior to said first dosage.
S78. Use of the anti-CGRP antibody of any one of embodiments S64-S77 wherein said patient was diagnosed with migraine at least 18 or at least 19 years prior to said first dosage.
S79. Use of the anti-CGRP antibody of any one of embodiments S64-S78 wherein said patient was diagnosed with chronic migraine at least 5 years prior to said first dosage.
S80. Use of the anti-CGRP antibody of any one of embodiments S64-S79 wherein said patient was diagnosed with chronic migraine at least 8 years prior to said first dosage.
S81. Use of the anti-CGRP antibody of any one of embodiments S64-S80 wherein said patient was diagnosed with chronic migraine at least 11 or at least 12 years prior to said first dosage.
S82. Use of the anti-CGRP antibody of any one of embodiments S64-S81 wherein said medicament is for administration of said first dosage when the patient has a headache.
S83. Use of the anti-CGRP antibody of any one of embodiments S64-S82 wherein said medicament is for administration of said first dosage when the patient has a migraine.
S84. Use of the anti-CGRP antibody of any one of embodiments S64-S83 wherein said medicament is for administration of said first dosage when the patient has a migraine with aura.
S85. Use of the anti-CGRP antibody of any one of embodiments S64-S84, wherein said patient has a reduction in the number of migraine days by at least 50% in the one month period after being administered said first dose relative to the baseline number of migraine days experienced by that patient prior to said first dose.
S86. Use of the anti-CGRP antibody of any one of embodiments S64-S85, wherein said patient has a reduction in the number of migraine days by at least 75% in the one month period after being administered said first dose relative to the baseline number of migraine days experienced by that patient prior to said first dose.
S87. Use of the anti-CGRP antibody of any one of embodiments S64-S86, wherein said patient has a reduction in the number of migraine days by 100% in the one month period after being administered said first dose relative to the baseline number of migraine days experienced by that patient prior to said first dose.
S88. Use of the anti-CGRP antibody of any one of embodiments S64-S87, wherein said patient has a reduction in the number of migraine days by at least 50% in the 12 week period after being administered said first dose relative to the baseline number of migraine days experienced by that patient prior to said first dose.
S89. Use of the anti-CGRP antibody of any one of embodiments S64-S88, wherein said patient has a reduction in the number of migraine days by at least 75% in the 12 week period after being administered said first dose relative to the baseline number of migraine days experienced by that patient prior to said first dose.
S90. Use of the anti-CGRP antibody of any one of embodiments S64-S89, wherein said patient has a reduction in the number of migraine days by 100% in the 12 week period after being administered said first dose relative to the baseline number of migraine days experienced by that patient prior to said first dose.
S91. Use of the anti-CGRP antibody of any one of embodiments S64-S90, further comprising administering a second dose of said anti-CGRP antibody to said patient about 12 weeks or about 3 months after said first dose.
S92. Use of the anti-CGRP antibody of any one of embodiments S64-S91, wherein said first dose comprises about 100 mg, about 125 mg, about 150 mg, about 175 mg, about 200 mg, about 225 mg, about 250 mg, about 275 mg, or about 300 mg of said anti-CGRP antibody.
S93. Use of the anti-CGRP antibody of any one of embodiments S64-S92, wherein said anti-CGRP antibody or antibody fragment is aglycosylated or if glycosylated only contains only mannose residues.
S94. Use of the anti-CGRP antibody of any one of embodiments S64-S93, wherein said anti-CGRP antibody consists of the light chain polypeptide of the light chain polypeptide of SEQ ID NO: 221 and the heavy chain polypeptide of SEQ ID NO: 201 or SEQ ID NO: 566.
S95. Use of the anti-CGRP antibody of any of any one of embodiments S64-S94, wherein said anti-CGRP antibody is expressed in or obtained by expression in Pichia pastoris.
S96. Use of the anti-CGRP antibody of any of any one of embodiments S64-S94, wherein said anti-CGRP antibody is expressed in or obtained by expression in CHO cells.
S97. Use of the anti-CGRP antibody of any one of embodiments S64-S96, wherein said anti-CGRP antibody or anti-CGRP antibody fragment is comprised in a formulation comprising or consisting of histidine (L-histidine), sorbitol, polysorbate 80, and water.
S98. Use of the anti-CGRP antibody of embodiment S97, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−10% of said values, and having a pH of 5.8 or within +/−10% of said value.
S99. Use of the anti-CGRP antibody of embodiment S97, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−5% of said values, and/or having a pH of 5.8 or within +/−5% of said value.
S100. Use of the anti-CGRP antibody of embodiment S97, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−1% of said values, and/or having a pH of 5.8 or within +/−1% of said value.
S101. Use of the anti-CGRP antibody of embodiment S97, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−0.5% of said values, and/or having a pH of 5.8 or within +/−0.5% of said value.
S102. Use of the anti-CGRP antibody of embodiment S97, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−0.1% of said values, and/or having a pH of 5.8 or within +/−0.1% of said value.

### Further Exemplary Embodiments

Further exemplary embodiments of the invention are provided as follows:
E1. An anti-CGRP antibody for use in treating acute migraine while the patient has a headache and within 30 hours of headache onset, preferably within 24 hours of headache onset.
E2. The anti-CGRP antibody for use of embodiment E1, wherein the anti-CGRP antibody is for administration within 18 hours, within 12 hours, within 6 hours, within 5 hours, within 4 hours, within 3 hours, within 2 hours, or within 1 hour of headache onset, such as between 1-6 hours from headache onset.
E3. The anti-CGRP antibody for use of any one of the foregoing embodiments, wherein said anti-CGRP antibody is for intravenous or subcutaneous infusion.
E4. The anti-CGRP antibody for use of any one of the foregoing embodiments, wherein said anti-CGRP antibody is for intravenous infusion.
E5. The anti-CGRP antibody for use of any one of the foregoing embodiments, wherein said patient is headache free 2 hours post-completion of administration or infusion.
E6. The anti-CGRP antibody for use of any one of the foregoing embodiments, wherein said patient is free from the most bothersome symptom (MBS) at 2 hours post-completion of administration or infusion.
E7. The anti-CGRP antibody for use of any one of the foregoing embodiments, wherein the administration of said anti-CGRP antibody improves one or more of the following: time to pain relief, time to pain freedom; headache pain freedom at 2 hours with sustained headache pain freedom for 24 and 48 hours post completion of infusion; use of rescue medication by 24 hours and by 48 hours post completion of infusion; absence of photophobia at 2 hours post completion of infusion; absence of phonophobia at 2 hours post completion of infusion; absence of nausea at 2 hours post completion of infusion; change from baseline in Headache Impact Test (HIT-6) at Week 4; or change from baseline in Migraine Treatment Optimization Questionnaire-6 (MTOQ-6) at Week 4.
E8. The anti-CGRP antibody for use of any one of the foregoing embodiments, wherein said anti-CGRP antibody comprises any one of Ab1-Ab14.
E9. The anti-CGRP antibody for use of any one of the foregoing embodiments, wherein said anti-CGRP antibody comprises Ab6.
E10. The anti-CGRP antibody for use of any one of the foregoing embodiments, wherein said anti-CGRP antibody comprises the light chain complementarity-determining region (CDR) 1, 2, and 3 polypeptide sequences of SEQ ID NO: 224; SEQ ID NO: 226; and SEQ ID NO: 228, respectively.
E11. The anti-CGRP antibody for use of any one of the foregoing embodiments, wherein said anti-CGRP antibody comprises the light chain CDR 1, 2, and 3 polypeptide sequences encoded by SEQ ID NO: 234; SEQ ID NO: 236; and SEQ ID NO: 238, respectively.
E12. The anti-CGRP antibody for use of any one of the foregoing embodiments, wherein said anti-CGRP antibody comprises the heavy chain CDR 1, 2, and 3 polypeptide sequences of SEQ ID NO: 204; SEQ ID NO: 206; and SEQ ID NO: 208, respectively.
E13. The anti-CGRP antibody for use of any one of the foregoing embodiments, wherein said anti-CGRP antibody comprises the heavy chain CDR 1, 2, and 3 polypeptide sequences encoded by SEQ ID NO: 214; SEQ ID NO: 216; and SEQ ID NO: 218, respectively.
E14. The anti-CGRP antibody for use of any one of the foregoing embodiments, wherein said anti-CGRP antibody comprises the light chain CDR 1, 2, and 3 polypeptide sequences of SEQ ID NO: 224; SEQ ID NO: 226; and SEQ ID NO: 228, respectively and heavy chain CDR 1, 2, and 3 polypeptide sequences of SEQ ID NO: 204; SEQ ID NO: 206; and SEQ ID NO: 208, respectively.
E15. The anti-CGRP antibody for use of any one of the foregoing embodiments, wherein said anti-CGRP antibody comprises the light chain CDR 1, 2, and 3 polypeptide sequences encoded by SEQ ID NO: 234; SEQ ID NO: 236; and SEQ ID NO: 238, respectively and heavy chain CDR 1, 2, and 3 polypeptide sequences encoded by SEQ ID NO: 214; SEQ ID NO: 216; and SEQ ID NO: 218, respectively.
E16. The anti-CGRP antibody for use of any one of the foregoing embodiments, wherein said anti-CGRP antibody comprises the variable light chain polypeptide of SEQ ID NO: 222.
E17. The anti-CGRP antibody for use of any one of the foregoing embodiments, wherein said anti-CGRP antibody comprises the variable light chain polypeptide encoded by SEQ ID NO: 232.
E18. The anti-CGRP antibody for use of any one of the foregoing embodiments, wherein said anti-CGRP antibody comprises the variable heavy chain polypeptide of SEQ ID NO: 202.
E19. The anti-CGRP antibody for use of any one of the foregoing embodiments, wherein said anti-CGRP antibody comprises the variable heavy chain polypeptide encoded by SEQ ID NO: 212.
E20. The anti-CGRP antibody for use of any one of the foregoing embodiments, wherein said anti-CGRP antibody comprises the variable light chain polypeptide of SEQ ID NO: 222 and the variable heavy chain polypeptide of SEQ ID NO: 202.
E21. The anti-CGRP antibody for use of any one of the foregoing embodiments, wherein said anti-CGRP antibody comprises the variable light chain polypeptide encoded by SEQ ID NO: 232 and the variable heavy chain polypeptide encoded by SEQ ID NO: 212.
E22. The anti-CGRP antibody for use of any one of the foregoing embodiments, wherein said anti-CGRP antibody comprises the light chain polypeptide of SEQ ID NO: 221.
E23. The anti-CGRP antibody for use of any one of the foregoing embodiments, wherein said anti-CGRP antibody comprises the light chain polypeptide encoded by SEQ ID NO: 231.
E24. The anti-CGRP antibody for use of any one of the foregoing embodiments, wherein said anti-CGRP antibody comprises the heavy chain polypeptide of SEQ ID NO: 201 or SEQ ID NO: 566.
E25. The anti-CGRP antibody for use of any one of the foregoing embodiments, wherein said anti-CGRP antibody comprises the heavy chain polypeptide encoded by SEQ ID NO: 211 or SEQ ID NO: 567.
E26. The anti-CGRP antibody for use of any one of the foregoing embodiments, wherein said anti-CGRP antibody comprises the light chain polypeptide of SEQ ID NO: 221 and the heavy chain polypeptide of SEQ ID NO: 201 or SEQ ID NO: 566.
E27. The anti-CGRP antibody for use of any one of the foregoing embodiments, wherein said anti-CGRP antibody comprises the light chain polypeptide encoded by SEQ ID NO: 231 and the heavy chain polypeptide encoded by SEQ ID NO: 211 or SEQ ID NO: 567.
E28. The anti-CGRP antibody for use of any one of the foregoing embodiments, wherein the administered amount of said anti-CGRP antibody is between about 100 mg and about 300 mg, or is about 100 mg, or is about 300 mg.
E29. The anti-CGRP antibody for use of any one of the foregoing embodiments, wherein the administered amount of said anti-CGRP antibody is 100 mg.
E30. The anti-CGRP antibody for use of any one of the foregoing embodiments, wherein said anti-CGRP antibody is for intravenous administration in a dosage of 100 mg of said anti-CGRP antibody every 10⁻¹⁴ weeks, preferably every 11-13 weeks, more preferably every 12 weeks.
E31. The anti-CGRP antibody for use of any one of embodiments E1-E29, wherein said anti-CGRP antibody is for intravenous administration in a dosage of administering 300 mg of said anti-CGRP antibody every 10⁻¹⁴ weeks, preferably every 11-13 weeks, more preferably every 12 weeks.
E32. The anti-CGRP antibody for use of any one of the foregoing embodiments, wherein said patient suffers from medication overuse headache.
E33. The anti-CGRP antibody for use of any one of the foregoing embodiments wherein, prior to administration of said anti-CGRP antibody, the patient exhibits between 1-10 migraine attacks per month in the month or in the 3 months prior to administration.
E34. The anti-CGRP antibody for use of any one of the foregoing embodiments wherein, prior to administration of said anti-CGRP antibody, the patient exhibits between 2-8 migraine attacks per month in the month or in the 3 months prior to administration.
E35. The anti-CGRP antibody for use of any one of the foregoing embodiments wherein, prior to administration of said anti-CGRP antibody, the patient exhibits between 3-7 migraine attacks per month in the month or in the 3 months prior to administration.
E36. The anti-CGRP antibody for use of any one of the foregoing embodiments wherein, prior to administration of said anti-CGRP antibody, the patient exhibits less than 25 headache days per month in the month or in the 3 months prior to administration.
E37. The anti-CGRP antibody for use of any one of the foregoing embodiments wherein, prior to administration of said anti-CGRP antibody, the patient exhibits less than 20 headache days per month in the month or in the 3 months prior to administration.
E38. The anti-CGRP antibody for use of any one of the foregoing embodiments wherein, prior to administration of said anti-CGRP antibody, the patient exhibits less than 15 headache days per month in the month or in the 3 months prior to administration.
E39. The anti-CGRP antibody for use of any one of the foregoing embodiments wherein, prior to administration of said anti-CGRP antibody, the patient exhibits less than 10 headache days per month in the month or in the 3 months prior to administration.
E40. The anti-CGRP antibody for use of any one of the foregoing embodiments wherein said patient was diagnosed with migraine at least 10 years prior to the administration of said anti-CGRP antibody.
E41. The anti-CGRP antibody for use of any one of the foregoing embodiments wherein said patient was diagnosed with migraine at least 15 years prior to the administration of said anti-CGRP antibody.
E42. The anti-CGRP antibody for use of any one of the foregoing embodiments wherein said patient was diagnosed with migraine at least 18 or at least 19 years prior to the administration of said anti-CGRP antibody.
E43. The anti-CGRP antibody for use of any one of the foregoing embodiments, wherein said patient has a reduction in the number of migraine days by at least 50% in the one month period after being administered said antibody relative to the baseline number of migraine days experienced by that patient prior to the administration of said anti-CGRP antibody.
E44. The anti-CGRP antibody for use of any one of the foregoing embodiments, wherein said patient has a reduction in the number of migraine days by at least 75% in the one month period after being administered said antibody relative to the baseline number of migraine days experienced by that patient prior to the administration of said anti-CGRP antibody.
E45. The anti-CGRP antibody for use of any one of the foregoing embodiments, wherein said patient has a reduction in the number of migraine days by 100% in the one month period after being administered said antibody relative to the baseline number of migraine days experienced by that patient prior to the administration of said anti-CGRP antibody.
E46. The anti-CGRP antibody for use of any one of the foregoing embodiments, wherein said patient has a reduction in the number of migraine days by at least 50% in the 12 week period after being administered said antibody relative to the baseline number of migraine days experienced by that patient prior to the administration of said anti-CGRP antibody.
E47. The anti-CGRP antibody for use of any one of the foregoing embodiments, wherein said patient has a reduction in the number of migraine days by at least 75% in the 12 week period after being administered said antibody relative to the baseline number of migraine days experienced by that patient prior to the administration of said anti-CGRP antibody.
E48. The anti-CGRP antibody for use of any one of the foregoing embodiments, wherein said patient has a reduction in the number of migraine days by 100% in the 12 week period after being administered said antibody relative to the baseline number of migraine days experienced by that patient prior to the administration of said anti-CGRP antibody.
E49. The anti-CGRP antibody for use of any one of the foregoing embodiments, wherein said use comprises administering a second dose of said anti-CGRP antibody to said patient about 10⁻¹⁴ weeks, preferably 11-13 weeks, more preferably about 12 weeks or about 3 months after the administration of said anti-CGRP antibody.
E50. The anti-CGRP antibody for use of any one of the foregoing embodiments, wherein said use comprises administering about 100 mg, about 125 mg, about 150 mg, about 175 mg, about 200 mg, about 225 mg, about 250 mg, about 275 mg, or about 300 mg of said anti-CGRP antibody.
E51. The anti-CGRP antibody for use of any one of the foregoing embodiments, wherein said anti-CGRP antibody is aglycosylated or if glycosylated only contains only mannose residues.
E52. The anti-CGRP antibody for use of any one of the foregoing embodiments, wherein said anti-CGRP antibody consists of the light chain polypeptide of SEQ ID NO: 221 and the heavy chain polypeptide of SEQ ID NO: 201 or SEQ ID NO: 566.
E53. The anti-CGRP antibody for use of any one of the foregoing embodiments, wherein said anti-CGRP antibody consists of the light chain polypeptide encoded by SEQ ID NO: 231 and the heavy chain polypeptide encoded by SEQ ID NO: 211 or SEQ ID NO: 567.
E54. The anti-CGRP antibody for use of any one of embodiments E7-E53, wherein said rescue medication comprises one or more of a triptan, non-opioid analgesics (such as paracetamol (acetaminophen), acetylsalicylic acid (aspirin), another NSAID, or another non-opioid analgesic), a combination of two or more drug classes, wherein said triptan use optionally comprises use of one or more of sumatriptan, zolmitriptan, naratriptan, rizatriptan, eletriptan, almotriptan, and frovatriptan, and/or wherein said opioid use optionally comprises use of one or more of oxycodone, tramadol, butorphanol, morphine, codeine, and hydrocodone, and/or wherein said combination of two or more drug class optionally comprises two drugs with analgesic effects (for example, paracetamol and codeine) or acting as adjuvants (for example, caffeine), optionally wherein said combination-analgesics combine non-opioid analgesic includes at least one opioid (such as tramadol, butorphanol, morphine, codeine, hydrocodone, or any combination thereof), barbiturate such as butalbital and/or caffeine.
E55. The anti-CGRP antibody for use of any one of the foregoing embodiments, wherein said headache or said migraine is diagnosed according to the third edition of the International Classification of Headache Disorders.
E56. The anti-CGRP antibody for use of any of any one of the foregoing embodiments, wherein said anti-CGRP antibody is expressed in or obtained by expression in Pichia pastoris.
E57. The anti-CGRP antibody for use of any of any one of embodiments E1-E55, wherein said anti-CGRP antibody is expressed in or obtained by expression in CHO cells.
E58. The anti-CGRP antibody for use of any one of the foregoing embodiments, wherein said anti-CGRP antibody or anti-CGRP antibody fragment is comprised in a formulation comprising or consisting of histidine (L-histidine), sorbitol, polysorbate 80, and water.
E59. The anti-CGRP antibody for use of embodiment E58, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−10% of said values, and having a pH of 5.8 or within +/−10% of said value.
E60. The anti-CGRP antibody for use of embodiment E58, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−5% of said values, and/or having a pH of 5.8 or within +/−5% of said value.
E61. The anti-CGRP antibody for use of embodiment E58, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−1% of said values, and/or having a pH of 5.8 or within +/−1% of said value.
E62. The anti-CGRP antibody for use of embodiment E58, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−0.5% of said values, and/or having a pH of 5.8 or within +/−0.5% of said value.
E63. The anti-CGRP antibody for use of embodiment E58, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−0.1% of said values, and/or having a pH of 5.8 or within +/−0.1% of said value.
E64. An anti-CGRP antibody for use in treating chronic migraine, wherein said antibody is for intravenous administration in a first dosage comprising between about 100 mg and about 300 mg of an anti-CGRP antibody, wherein said anti-CGRP antibody comprises the light chain polypeptide of SEQ ID NO: 221 and the heavy chain polypeptide of SEQ ID NO: 201 or SEQ ID NO: 566, wherein in the first 24 hours after administration of said first dosage the patient exhibits at least a 50% reduction in migraine prevalence.
E65. An anti-CGRP antibody for use in treating chronic migraine, wherein said antibody is for intravenous administration in a first dosage comprising between about 100 mg and about 300 mg of an anti-CGRP antibody, wherein said anti-CGRP antibody comprises the light chain polypeptide of SEQ ID NO: 221 and the heavy chain polypeptide of SEQ ID NO: 201 or SEQ ID NO: 566, wherein on the first day following the day of administration the patient exhibits at least a 50% reduction in migraine prevalence.
E66. The anti-CGRP antibody for use of embodiment E64 or embodiment E65, wherein the first dosage of said anti-CGRP antibody is 100 mg.
E67. The anti-CGRP antibody for use of embodiment E64 or embodiment E65, wherein the first dosage of said anti-CGRP antibody is 300 mg.
E68. The anti-CGRP antibody for use of embodiment E64, E65, or E66, wherein said anti-CGRP antibody is for intravenous administration in a dosage of 100 mg of said anti-CGRP antibody every 10⁻¹⁴ weeks, preferably 11-13 weeks, more preferably every 12 weeks.
E69. The anti-CGRP antibody for use of embodiment E64, E65, or E67, wherein said anti-CGRP antibody is for intravenous administration in a dosage of 300 mg of said anti-CGRP antibody every 10⁻¹⁴ weeks, preferably 11-13 weeks, more preferably every 12 weeks.
E70. The anti-CGRP antibody for use of any one of embodiments E64-E69 wherein, prior to said first dosage, the patient exhibits between about 10 and about 22 migraine days per month.
E71. The anti-CGRP antibody for use of any one of embodiments E64-E70 wherein, prior to said first dosage, the patient exhibits between about 13 and about 19 migraine days per month.
E72. The anti-CGRP antibody for use of any one of embodiments E64-E71 wherein, prior to said first dosage, the patient exhibits about 16 migraine days per month.
E73. The anti-CGRP antibody for use of any one of embodiments E64-E72 wherein, prior to said first dosage, the patient exhibits between about 14 and about 27 headache days per month.
E74. The anti-CGRP antibody for use of any one of embodiments E64-E73 wherein, prior to said first dosage, the patient exhibits between about 17 and about 24 headache days per month.
E75. The anti-CGRP antibody for use of any one of embodiments E64-E74 wherein, prior to said first dosage, the patient exhibits about 20 or about 21 headache days per month.
E76. The anti-CGRP antibody for use of any one of embodiments E64-E75 wherein said patient was diagnosed with migraine at least 10 years prior to said first dosage.
E77. The anti-CGRP antibody for use of any one of embodiments E64-E76 wherein said patient was diagnosed with migraine at least 15 years prior to said first dosage.
E78. The anti-CGRP antibody for use of any one of embodiments E64-E77 wherein said patient was diagnosed with migraine at least 18 or at least 19 years prior to said first dosage.
E79. The anti-CGRP antibody for use of any one of embodiments E64-E78 wherein said patient was diagnosed with chronic migraine at least 5 years prior to said first dosage.
E80. The anti-CGRP antibody for use of any one of embodiments E64-E79 wherein said patient was diagnosed with chronic migraine at least 8 years prior to said first dosage.
E81. The anti-CGRP antibody for use of any one of embodiments E64-E80 wherein said patient was diagnosed with chronic migraine at least 11 or at least 12 years prior to said first dosage.
E82. The anti-CGRP antibody for use of any one of embodiments E64-E81 wherein said anti-CGRP antibody is for administration of said first dosage when the patient has a headache.
E83. The anti-CGRP antibody for use of any one of embodiments E64-E82 wherein said anti-CGRP antibody is for administration of said first dosage when the patient has a migraine.
E84. The anti-CGRP antibody for use of any one of embodiments E64-E83 wherein said anti-CGRP antibody is for administration of said first dosage when the patient has a migraine with aura.
E85. The anti-CGRP antibody for use of any one of embodiments E64-E84, wherein said patient has a reduction in the number of migraine days by at least 50% in the one month period after being administered said first dose relative to the baseline number of migraine days experienced by that patient prior to said first dose.
E86. The anti-CGRP antibody for use of any one of embodiments E64-E85, wherein said patient has a reduction in the number of migraine days by at least 75% in the one month period after being administered said first dose relative to the baseline number of migraine days experienced by that patient prior to said first dose.
E87. The anti-CGRP antibody for use of any one of embodiments E64-E86, wherein said patient has a reduction in the number of migraine days by 100% in the one month period after being administered said first dose relative to the baseline number of migraine days experienced by that patient prior to said first dose.
E88. The anti-CGRP antibody for use of any one of embodiments E64-E87, wherein said patient has a reduction in the number of migraine days by at least 50% in the 12 week period after being administered said first dose relative to the baseline number of migraine days experienced by that patient prior to said first dose.
E89. The anti-CGRP antibody for use of any one of embodiments E64-E88, wherein said patient has a reduction in the number of migraine days by at least 75% in the 12 week period after being administered said first dose relative to the baseline number of migraine days experienced by that patient prior to said first dose.
E90. The anti-CGRP antibody for use of any one of embodiments E64-E89, wherein said patient has a reduction in the number of migraine days by 100% in the 12 week period after being administered said first dose relative to the baseline number of migraine days experienced by that patient prior to said first dose.
E91. The anti-CGRP antibody for use of any one of embodiments E64-E90, further comprising administering a second dose of said anti-CGRP antibody to said patient about 12 weeks or about 3 months after said first dose.
E92. The anti-CGRP antibody for use of any one of embodiments E64-E91, wherein said first dose comprises about 100 mg, about 125 mg, about 150 mg, about 175 mg, about 200 mg, about 225 mg, about 250 mg, about 275 mg, or about 300 mg of said anti-CGRP antibody.
E93. The anti-CGRP antibody for use of any one of embodiments E64-E92, wherein said anti-CGRP antibody or antibody fragment is aglycosylated or if glycosylated only contains only mannose residues.
E94. The anti-CGRP antibody for use of any one of embodiments E64-E93, wherein said anti-CGRP antibody consists of the light chain polypeptide of the light chain polypeptide of SEQ ID NO: 221 and the heavy chain polypeptide of SEQ ID NO: 201 or SEQ ID NO: 566.
E95. The anti-CGRP antibody for use of any of any one of embodiments E64-E94, wherein said anti-CGRP antibody is expressed in or obtained by expression in Pichia pastoris.
E96. The anti-CGRP antibody for use of any of any one of embodiments E64-E94, wherein said anti-CGRP antibody is expressed in or obtained by expression in CHO cells.
E97. The anti-CGRP antibody for use of any one of embodiments E64-E96, wherein said anti-CGRP antibody or anti-CGRP antibody fragment is comprised in a formulation comprising or consisting of histidine (L-histidine), sorbitol, polysorbate 80, and water.
E98. The anti-CGRP antibody for use of embodiment E97, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−10% of said values, and having a pH of 5.8 or within +/−10% of said value.
E99. The anti-CGRP antibody for use of embodiment E97, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−5% of said values, and/or having a pH of 5.8 or within +/−5% of said value.
E100. The anti-CGRP antibody for use of embodiment E97, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−1% of said values, and/or having a pH of 5.8 or within +/−1% of said value.
E101. The anti-CGRP antibody for use of embodiment E97, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−0.5% of said values, and/or having a pH of 5.8 or within +/−0.5% of said value.
E102. The anti-CGRP antibody for use of embodiment E97, wherein said formulation comprises or consists of, per 1 mL volume, 100 mg anti-CGRP antibody, 3.1 mg L-Histidine, 40.5 mg Sorbitol, and 0.15 mg Polysorbate 80, or having amounts of each constituent within +/−0.1% of said values, and/or having a pH of 5.8 or within +/−0.1% of said value.

### EXAMPLES

The following examples are provided in order to illustrate the invention, but are not to be construed as limiting the scope of the claims in any way.

### Example 1

Preparation of Antibodies that Bind CGRP

The preparation of exemplary anti-CGRP antibodies Ab1-Ab14 having the sequences in FIGS. 1A-12 is disclosed in commonly owned PCT Application WO/2012/162243, published on Nov. 29, 2012, the contents of which are incorporated by reference herein. This application exemplifies synthesis of these antibodies in Pichia pastoris cells. The present Applicant further contemplates synthesis of anti-CGRP antibodies Ab1-Ab14, and Ab6 in particular in CHO cells.

### Example 2

### Human Clinical Study Evaluating the Safety and Efficacy of an Anti-CGRP Antibody According to the Invention

### Clinical Treatment Protocol

The humanized anti-CGRP IgG1 antibody identified herein as Ab6 was assessed in human subjects for its ability to inhibit, alleviate or prevent the number of, duration, and/or the intensity of migraine episodes. The Ab6 antibody contains the V_{L} and light chain polypeptides respectively in SEQ ID NO: 222 and SEQ ID NO: 221, and contains the VH and heavy chain polypeptides respectively in SEQ ID NO: 202 and SEQ ID NO: 201. This antibody comprises an IgG1 constant region that contains a mutation in the heavy chain constant region (replacement of asparagine residue at position 297 with an alanine residue which substantially eliminates glycosylation and lytic activity (see U.S. Pat. No. 5,624,821).

Specifically, the clinical efficacy of the Ab6 antibody was tested in a placebo controlled double-blind, randomized study. The individuals in the study were all selected based on specific criteria. Particularly all were diagnosed as migraine sufferers at ≤50 years of age (ICHD-II, 2004 Section 1), and further had a history of migraine ≥12 months with ≥5 and ≤14 migraine days in each 28 day period in the 3 months prior to screening.

Further, all of the individuals in the study used acute migraine medications ≤14 days per 28 day period and, within those days, ≤10 days of triptan use per 28 day period in the 3 months prior to screening and the 28 day period of completion of eDiary prior to randomization.

Table 1 summarizes the demographic characteristics of the study population.

TABLE 1
Baseline Demographics and Clinical Characteristics

| | Placebo iv | Ab6 1000 mg iv |
|---|---|---|
| Characteristic | (n = 82) | (n = 81) |
| Mean ± SD Age (years) | 39.0 (9.6) | 38.6 (10.8) |
| Mean ± SD Weight (kg) | 75.4 (14.4) | 75.0 (16.5) |
| Female Gender | 66 (80%) | 67 (83%) |
| Race: | | |
| Caucasian | 66 (80.5%) | 66 (81.5%) |
| African American | 9 (11.0%) | 10 (12.4%) |
| Asian | 3 (3.7%) | 4 (5.0%) |
| Other | 4 (4.8%) | 1 (1.1%) |
| Baseline (per 28 days): | | |
| Mean ± SD Migraine Days | 8.8 (2.7) | 8.4 (2.1) |
| Mean ± SD Migraine Episodes | 6.7 (2.4) | 6.0 (2.2) |
| Mean ± SD Headache Frequency | 9.6 (2.8) | 9.2 (2.6) |
| Mean ± SD Migraine Hours | 72.2 (51.0) | 80.1 (49.1) |
| Mean ± SD HIT-6 Score | 64.5 (4.44) | 63.8 (5.21) |
| Mean ± SD MSQ RFP Score | 49.0 (17.9) | 49.5 (21.2) |
| Mean ± SD MSQ RFR Score | 61.9 (22.7) | 63.9 (24.0) |
| Mean ± SD MSQ EF Score | 59.5 (22.9) | 59.8 (27.0) |

Throughout the study all of the individuals were required to record their migraine status daily using an e-diary. In the e-diary the subjects in the study were required to record the number of migraine days/month, migraine episodes/month, migraine hours/month, migraine severity, and the use of any abortive medicine such as triptans.

In addition, the study participants were required to use the e-diary to record their migraine status in the 28 day period prior to treatment with antibody or placebo in order to establish a migraine day/hour/episode baseline per month. Also, this allowed the subjects in the study to become familiar with the use of the e-diary.

After the 28-day run-in the subjects in the study were broken into two groups, each including 80 subjects (FIG. 17). In the first group, i.e., the antibody treatment group, (n=80) each subject in the group was administered intravenously a single 1000 mg dose of Ab6. In the second group (n=80), i.e., the placebo group, each of the subjects was given an intravenous injection containing only the aqueous antibody carrier solution.

The individuals in the treated and placebo groups were assessed in the 24 weeks post-dose administration. Initially, a 12 week interim analysis was conducted. Subsequent to the 12 week interm analysis, a refined analysis was conducted. This refined analysis potentially included, for example, addition or removal of patient data in accord with the study protocol, e.g., updating data that had not been fully loaded from the e-diaries. This refinement resulted in slight changes but did not alter the overall conclusions.

The efficacy of the antibody versus the placebo was assessed in part based on the recorded data in the e-diary entries. For example, this analysis included a comparison of the number of recorded migraine days/month, migraine episodes/month, migraine hours/month in the subjects in the treated versus the placebo group. The percentage of responders in each group (i.e., the subjects with 50%, 75%, and 100% reduction in migraine days) in both groups was also compared.

In addition, the responses of the Ab6- and placebo-treated subjects in both groups to MSQ and HIT-6 questionnaires are to be evaluated and compared. MSQ is a frequently utilized disease-specific tool to assess the impact of migraine on health-related quality of life (HRQL). MSQ comprises a 16-item Migraine-Specific Quality-of-Life Questionnaire (Version 1.0), which was developed by Glaxo Wellcome Inc. MSQ is hypothesized to measure 3 parameters: (i) Role Function-Restrictive; (ii) Role Function-Preventive; and (iii) Emotional Function.

The HIT-6 or functional impact (also called the Headache Impact Test or HIT-6) similarly is a well known tool for assessing migraine intensity. This test uses six questions to capture the impact of headache and its treatment on an individual's functional health and well-being.

Also, the pharmacokinetic (PK) properties of the CGRP antibody and immunogenicity are to be assessed in the Ab6 antibody treated subjects.

### Clinical Results and Analysis

The results of this human clinical trial and analysis through week 12 in the treated subjects are summarized in the Table 2 below.

TABLE 2
Responder analysis for migraine days

| Time | % reduction | Placebo | Ab6 1000 mg | |
|---|---|---|---|---|
| period | migraine days | iv | iv | P value |
| Week 1-4 | | n = 80 | n = 75 | |
| | 50 | 40 (50.0) | 58 (77.3) | p = 0.0005 |
| | 75 | 19 (23.8) | 39 (52.0) | p = 0.0005 |
| | 100 | 4 (5.0) | 21 (28.0) | p = 0.0001 |
| Week 5-8 | | n = 80 | n = 78 | |
| | 50 | 43 (53.8) | 59 (75.6) | p = 0.0048 |
| | 75 | 28 (35.0) | 35 (44.9) | p = 0.2555 |
| | 100 | 12 (15.0) | 21 (26.9) | p = 0.0791 |
| Week 9-12 | | n = 77 | n = 72 | |
| | 50 | 51 (66.2) | 54 (75.0) | p = 0.2827 |
| | 75 | 24 (31.2) | 38 (52.8) | p = 0.0083 |
| | 100 | 13 (16.9) | 29 (40.3) | p = 0.0019 |

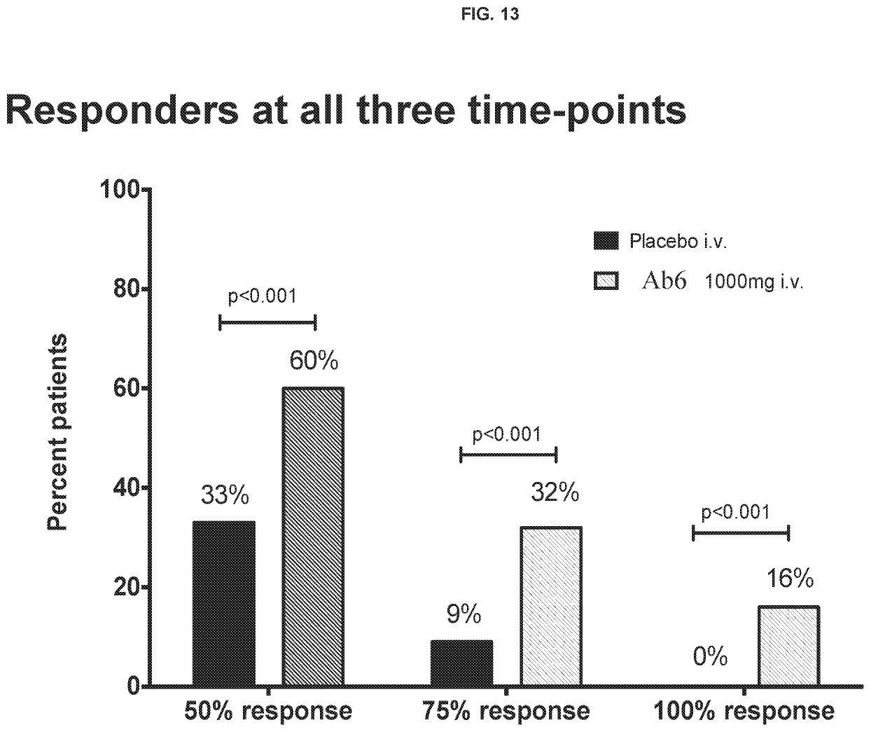

In addition, the results of the clinical study were compared based on the number of responders in the treatment and placebo groups. As shown in FIG. 13 the number of subjects who showed a 50, 75 or 100% reduction in migraine days for each month of the interim period were compared in the treatment and placebo groups. As shown in the figure, 60% of the Ab6-treated group had at least 50% reduction in headache days, 31% of the Ab6-treated group had at least 75% reduction in headache days and 15% of the Ab6 treated group had 100% reduction in headache days.

By contrast, 33% of the placebo-treated group had at least 50% reduction in headache days, 9% of the placebo-treated group had at least 75% reduction in headache days, and 0% (none) of the placebo-treated group had 100% reduction in headache days.

These results clearly show that the reduction in the number of migraine days was much greater in the Ab6-treated group. But for the significant placebo effect, the difference in these numbers would have been more pronounced. (Elevated placebo effect is not surprising as the phenomenon is often very high for migraine and other neurological drugs).

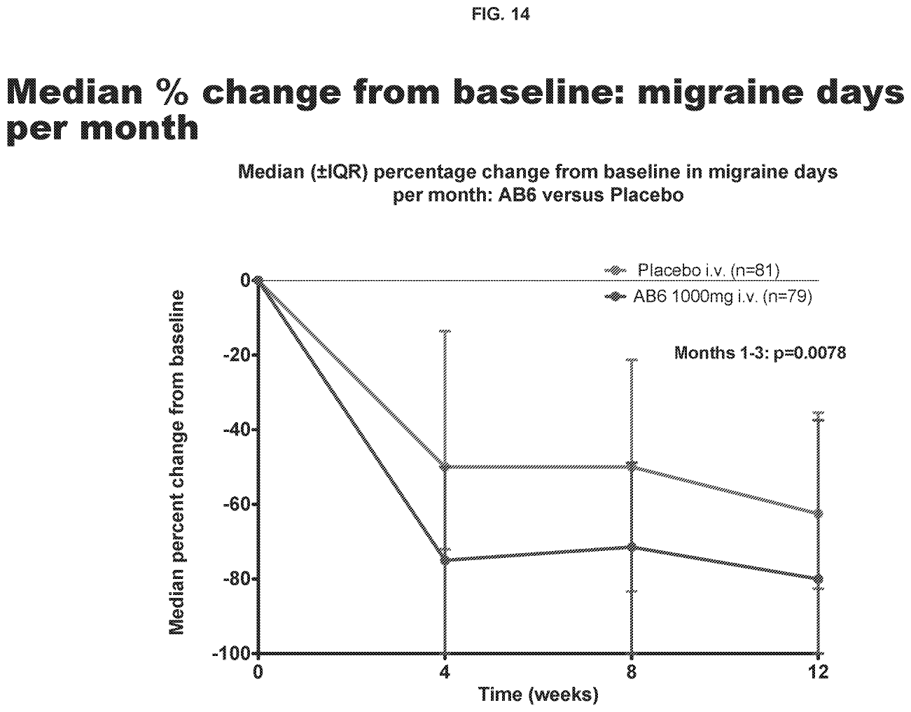

In addition, the % change from baseline in the number of migraine days per month in the placebo and Ab6-treated group was compared. As shown in FIG. 14, the median (±QR) % change from baseline in the number of migraine days per month in the placebo and Ab6-treated group was compared for the 2 groups during the 12 weeks post-treatment. These results which are statistically significant (p=0.0078) clearly show the Ab6-treated group had a much greater reduction in the number of headache days per month compared to baseline than the placebo-treated group.

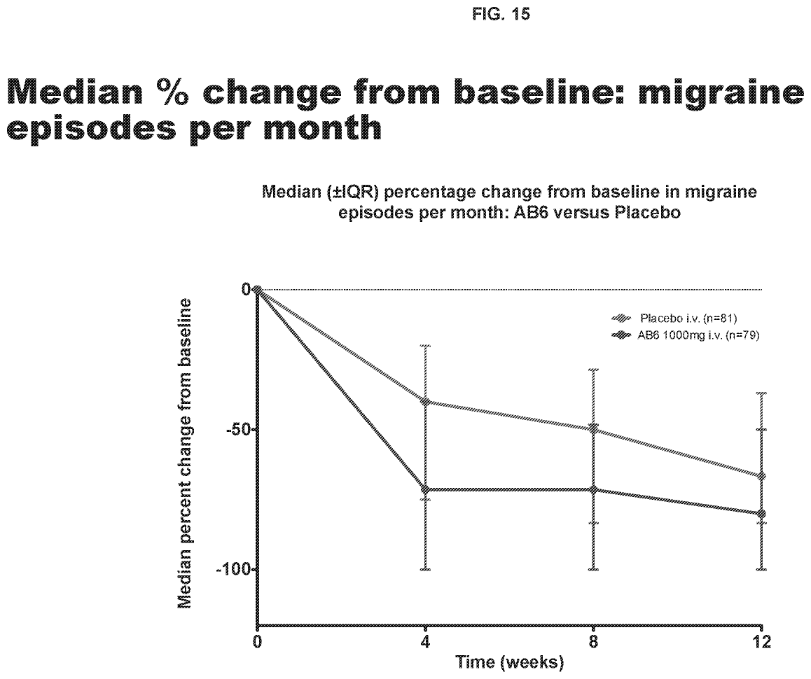

Also, the % change from baseline in the number of migraine episodes per month in the placebo and Ab6-treated group was compared. As shown in FIG. 15 the median (±QR) % change from baseline in the number of migraine episodes per month in the placebo and Ab6-treated group was compared during the 12 weeks post-treatment. These results indicate that the Ab6-treated group had a significantly greater reduction in the number of migraine episodes per month compared to baseline than the placebo-treated group.

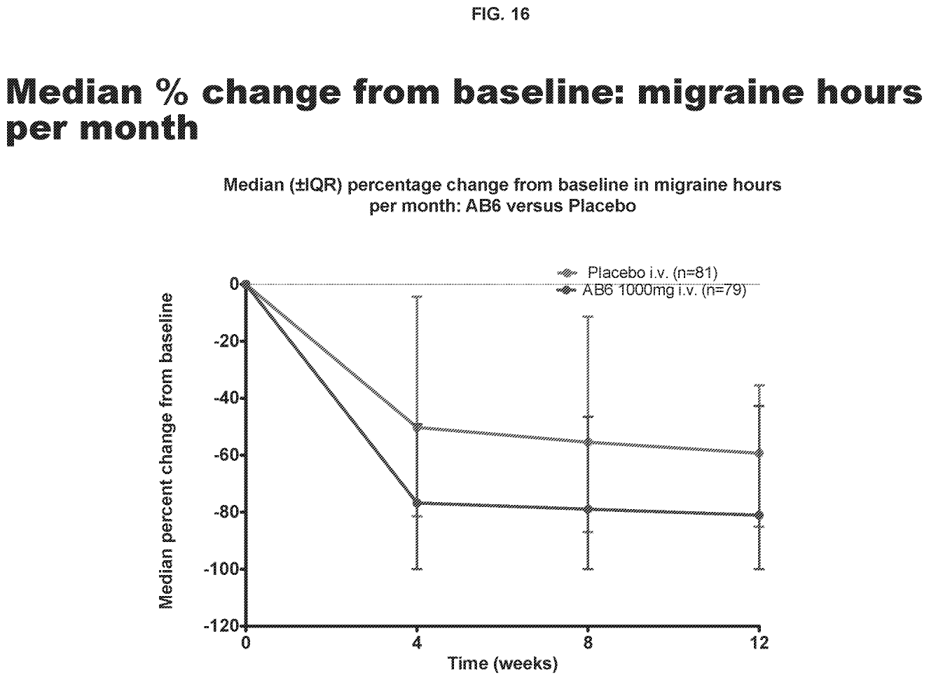

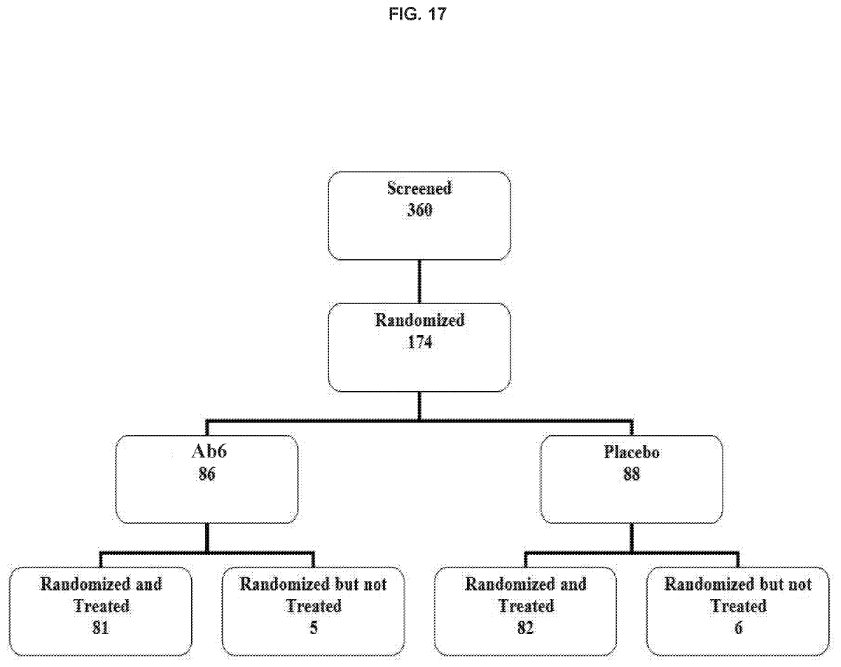

Further, the % change from baseline in the number of migraine hours per month in the placebo and Ab6-treated group was compared. As shown in FIG. 16, the median (±QR) % change from baseline in the number of migraine hours per month in the placebo and Ab6-treated group was compared for the 2 groups during the 12 weeks post-treatment. These results clearly show the Ab6-treated group had a greater reduction in the number of migraine hours per month compared to baseline than the placebo-treated group.

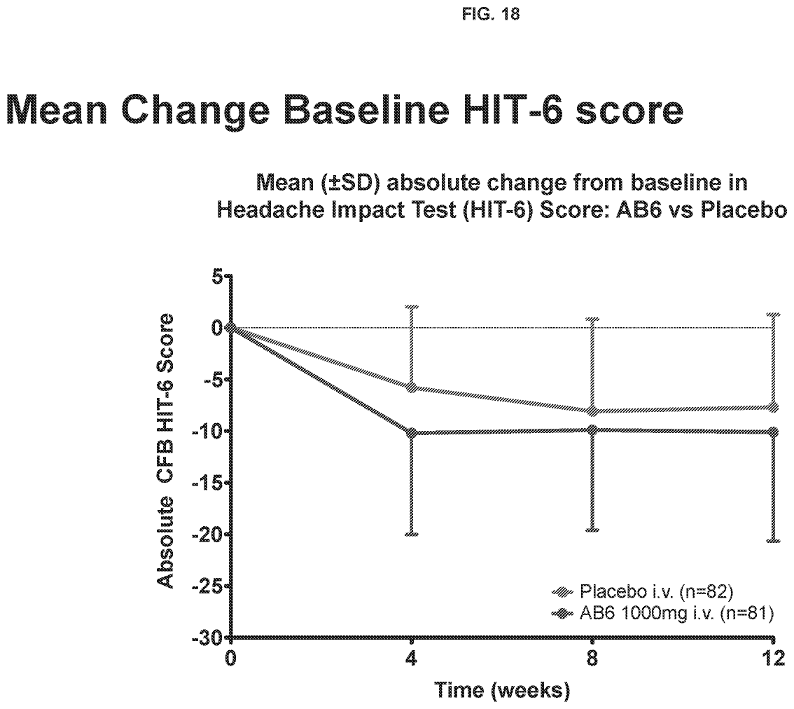

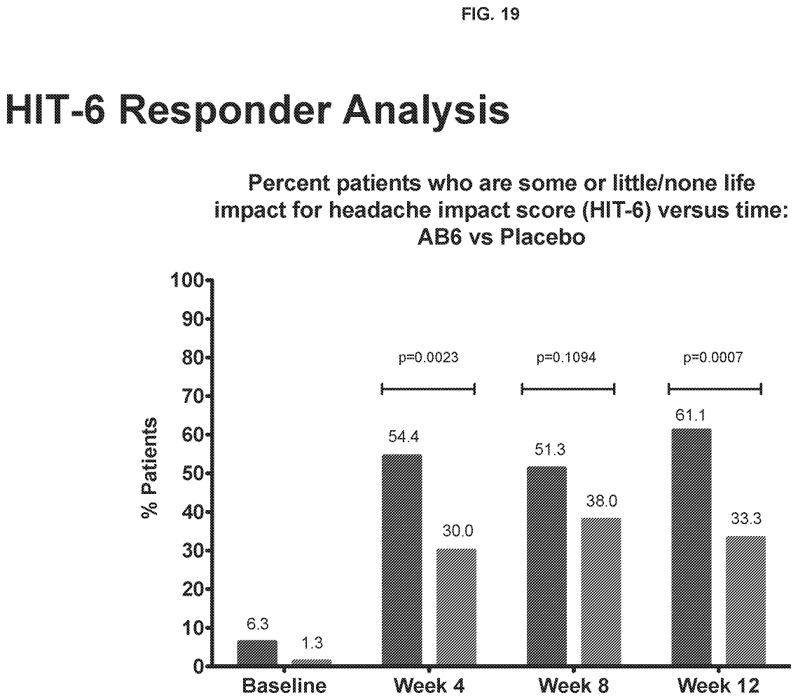

In addition, the HIT-6 results were compared for both groups. As noted, this questionnaire finds well accepted usage in assessing the migraine status of individuals with frequent/chronic migraine. FIG. 18 compares the HIT-6 responder analysis for the Ab6-treated and placebo groups at baseline, week 4 after treatment, week 8 after treatment and week 12 after treatment. The results at each time point reveal that the Ab6-treated group had a statistically significant improvement in the HIT-6 scores relative to the placebo group, i.e., 54.4% for the Ab6-treated compared to 30% for the placebo at week 4 (p=0.0023), 51.3% for the Ab6-treated compared to 38.0% for the placebo at week 8 (p=0.1094) and 61.1% for the Ab6-treated compared to 33.3% for the placebo at week 12 (p=0.0007). FIG. 19 shows the percentage of patients having a HIG-6 score of some or little/none over time in the placebo and Ab6 treatment groups (statistical significance a shown).

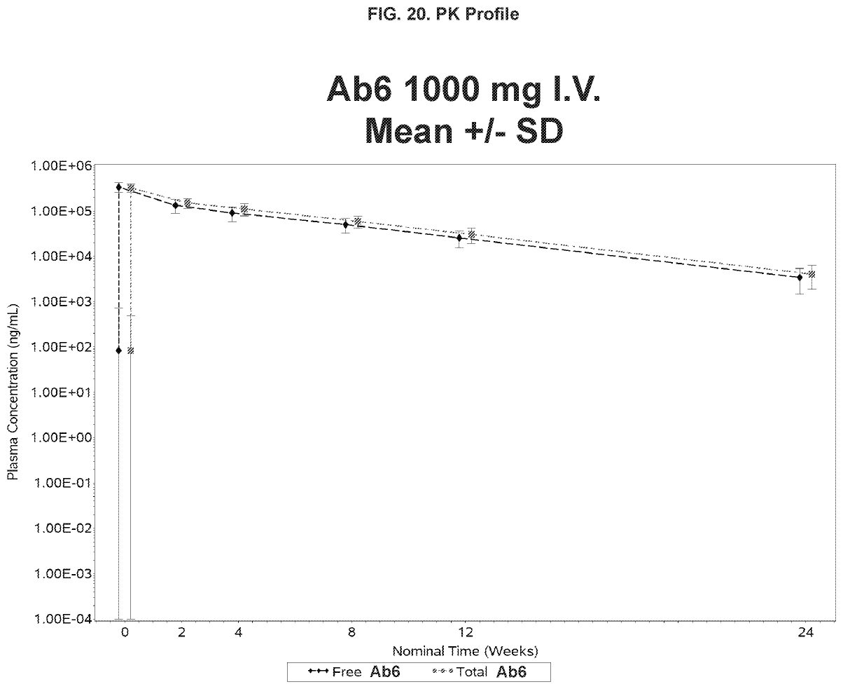

In addition, FIG. 20 contains the pharmacokinetic (PK) profile for Ab6 administered intravenously at a single dosage of 1000 mg in mg/mL over the 24 week period following Ab6 administration.

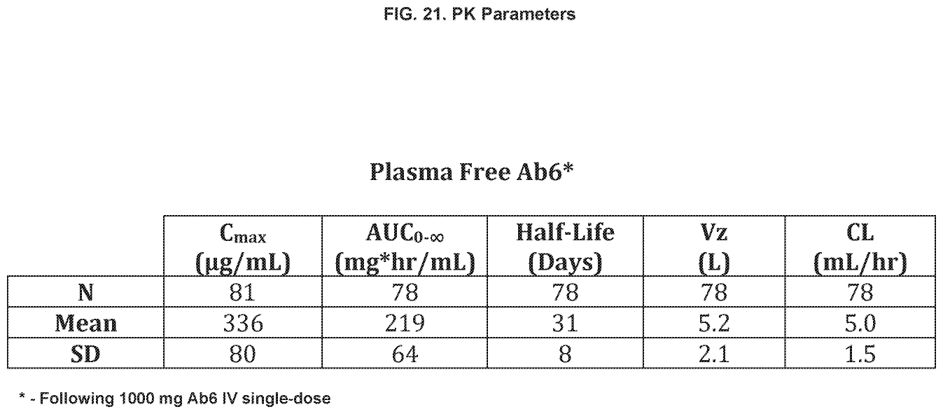

FIG. 21 contains plasma-free pharmacokinetic (PK) parameters N (number of patients), mean, and standard deviation (SD) for a single 1000 mg intravenous dosage of Ab6. The parameters shown in the table and the units are Cₘₐₓ (μg/mL), AUC_{0-∞} (mg*hr/mL), half-life (days), V_{z} (L) and C_{L}(mL/hr).

Further analysis was conducted for patient data between 12-weeks and 24-weeks. The treatment group continued to exhibit decreased migraine days relative to the control group, however, the magnitude of the difference decreased over time. Additionally, the control group exhibited fewer migraine days per month than at baseline. This was thought to result at least in part from “diary fatigue” wherein patients potentially report no migraine on a day in which a migraine actually occurred, in order to avoid the time and effort of answering further queries about the migraine that would result from giving an affirmative answer to the question of whether they had a migraine on a given day.

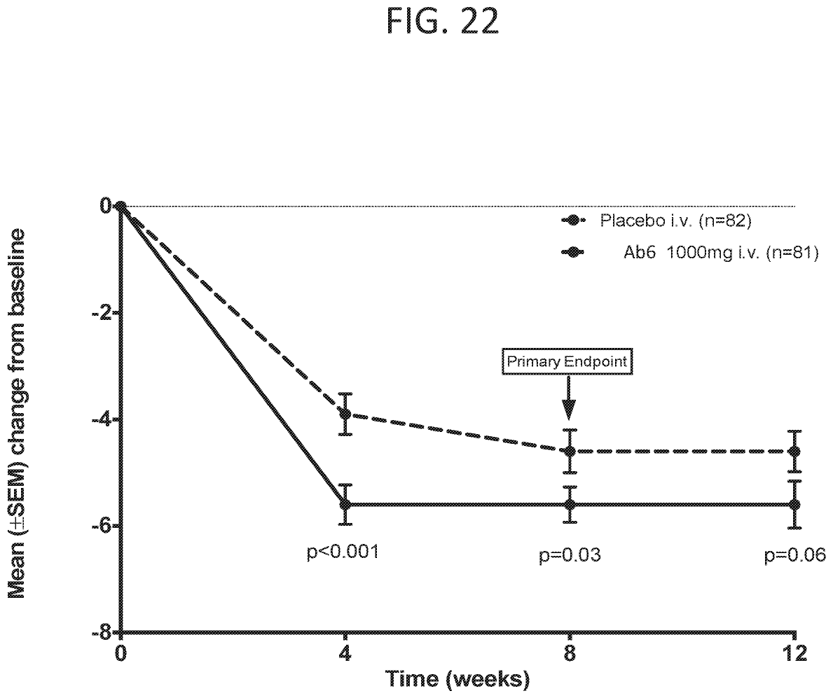

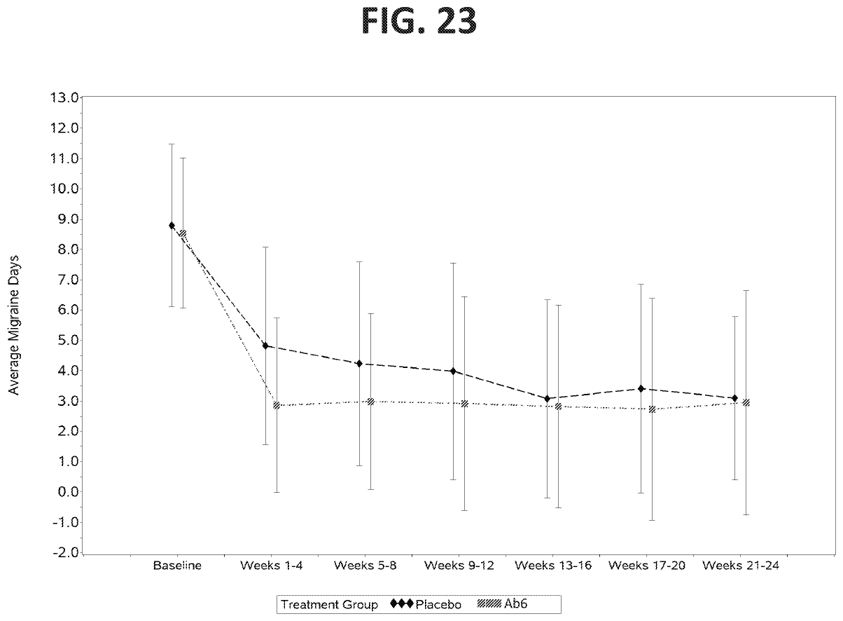

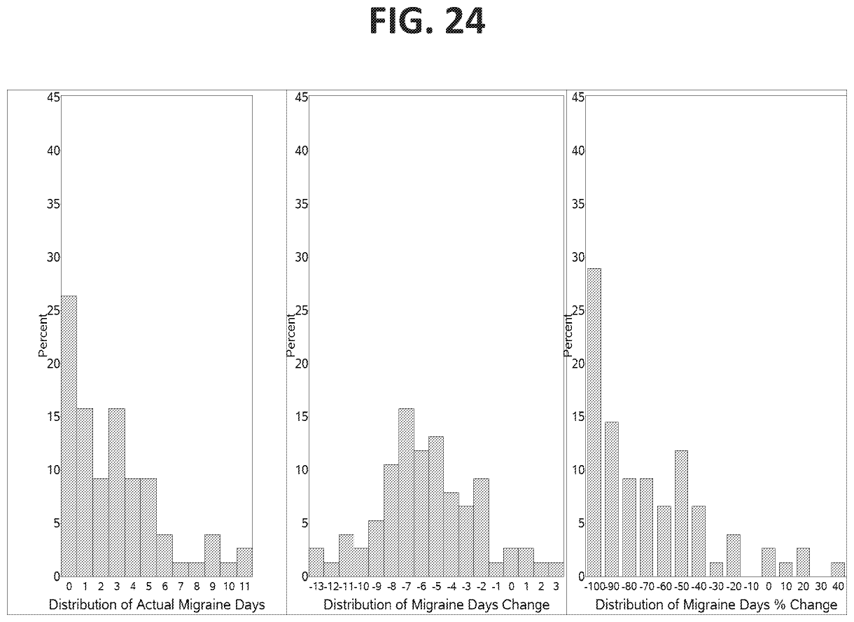

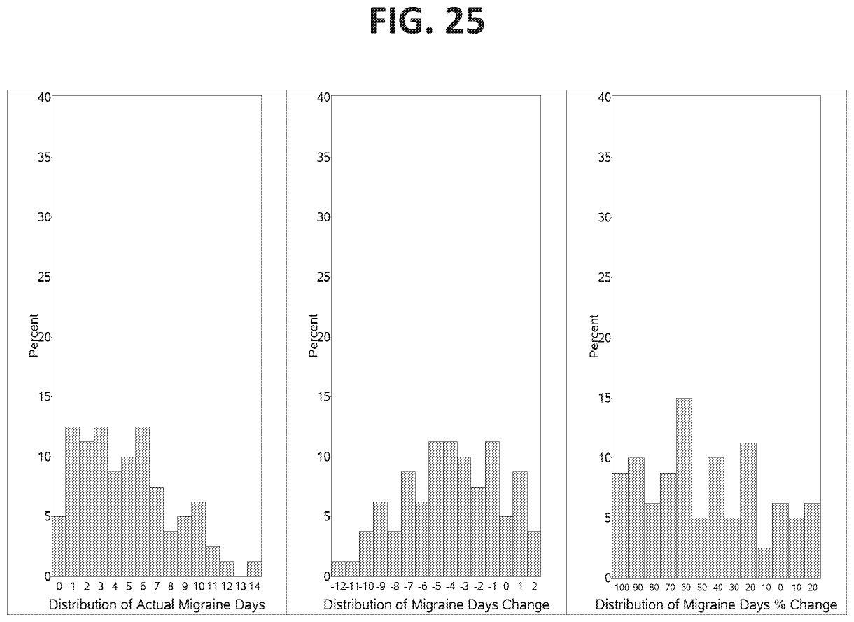

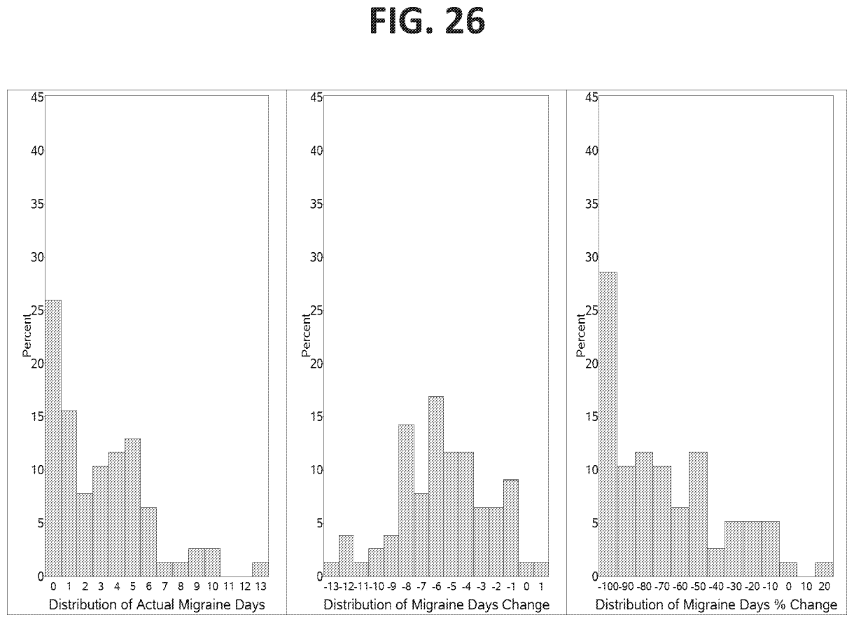

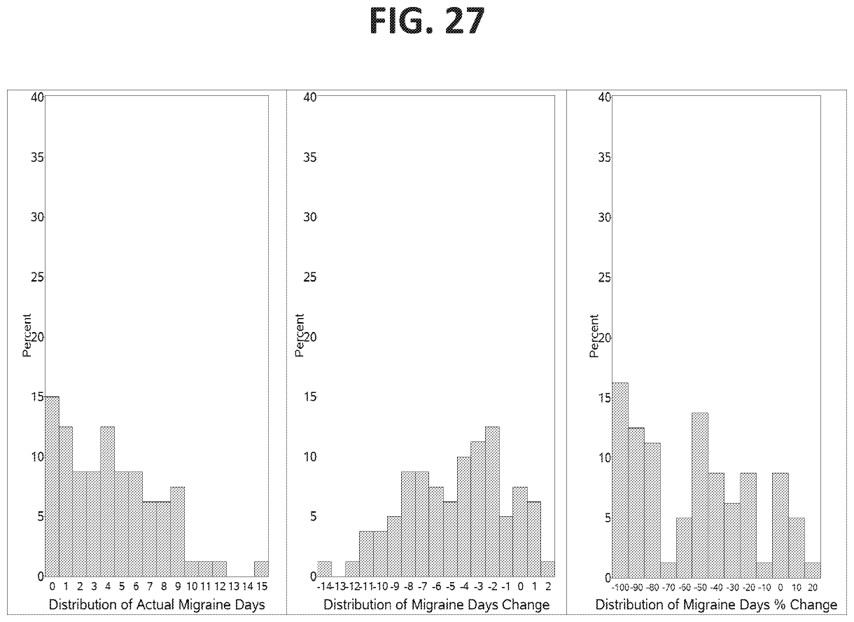

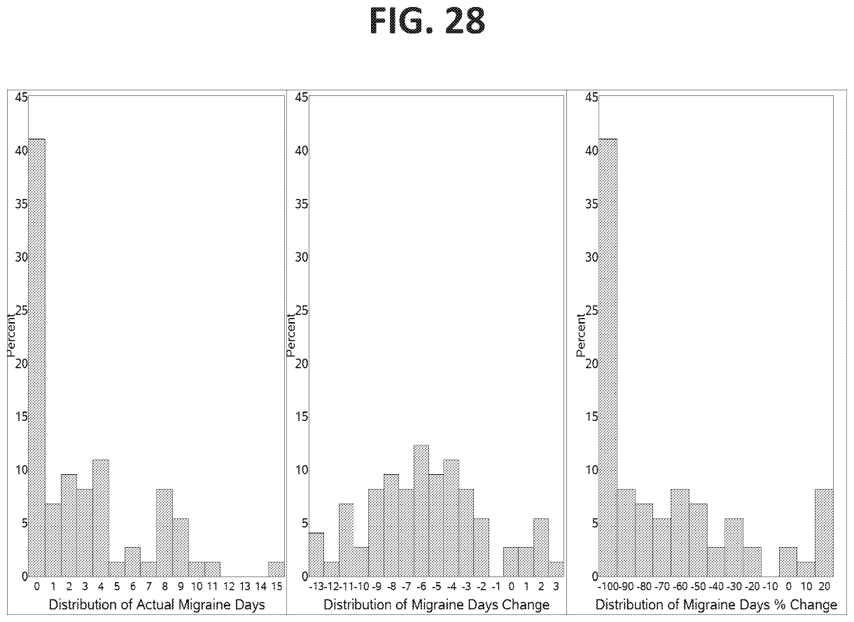

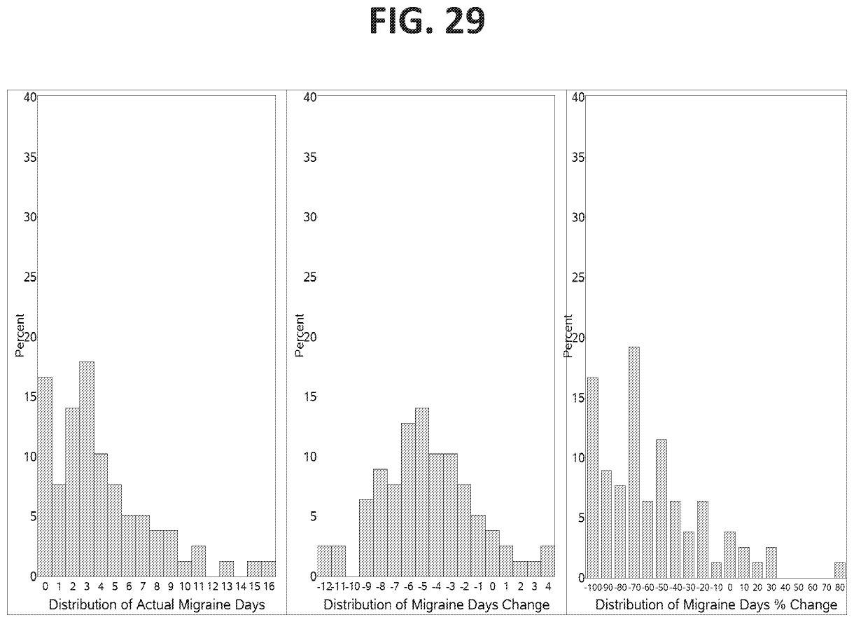

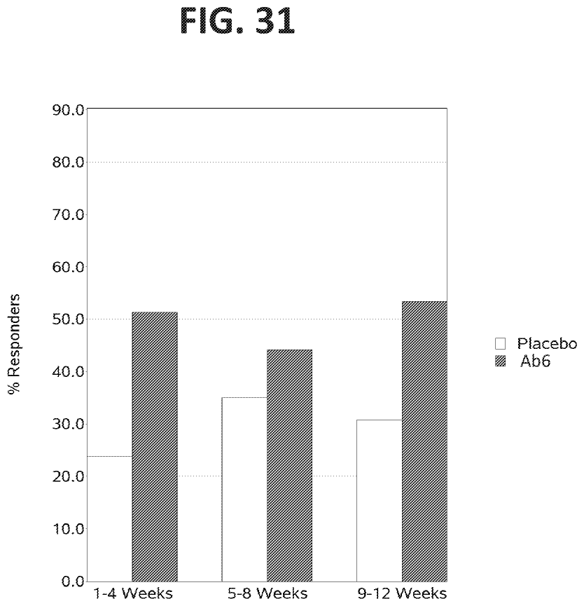

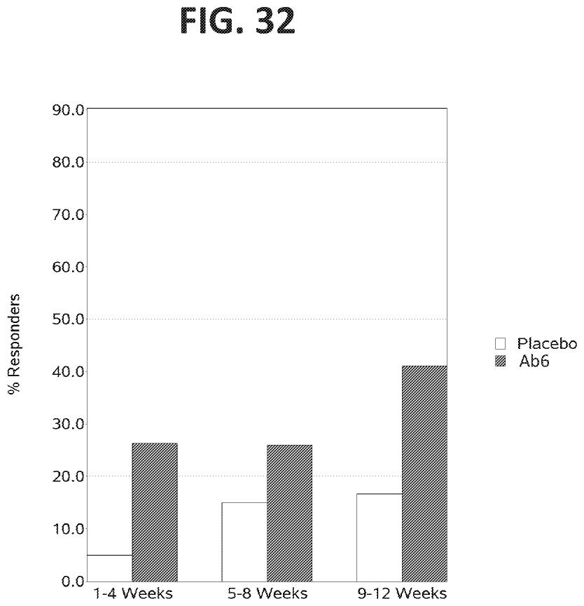

Further analysis of the study results are shown in FIGS. 22-33. These result include analysis of the change (mean+/−SEM) from baseline in migraine days per month for Ab6 (1000 mg i.v.) versus placebo (FIG. 22), change in average migraine days (+/−SD) over time for the full analysis population (FIG. 23). Additionally, shown are the distribution of migraine days actual and change for the Ab6 treatment group during weeks 1-4 (FIG. 24), distribution of migraine days actual and change for the placebo group during weeks 1-4 (FIG. 25), distribution of migraine days actual and change for the Ab6 treatment group during weeks 5-8 (FIG. 26), distribution of migraine days actual and change for the placebo group during weeks 5-8 (FIG. 27), distribution of migraine days actual and change for the Ab6 treatment group during weeks 9-12 (FIG. 28), and distribution of migraine days actual and change for the placebo group during weeks 9-12 (FIG. 29).

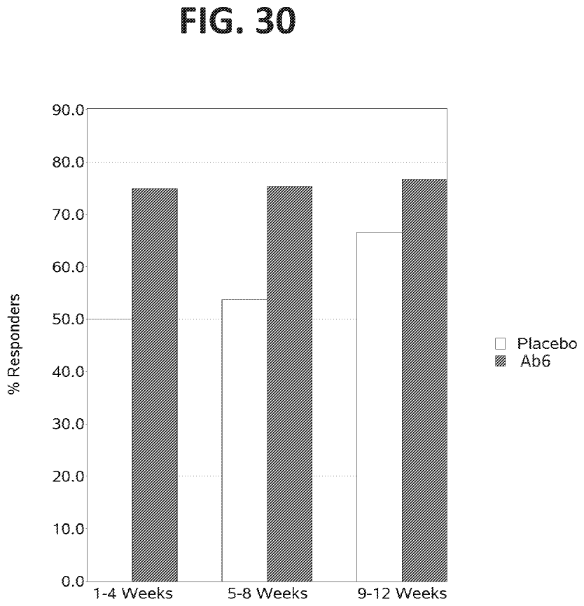

Responder rate analysis was also performed (FIGS. 30-32). These figures respectively show the 50%, 75%, and 100% responder rate for the Ab6 and placebo treatment groups. Subjects with ≥50% reduction in migraine frequency were considered to be a 50% responder. Subjects with ≥75% reduction in migraine frequency were considered to be a 75% responder. Likewise, subjects with 100% reduction in migraine frequency were considered to be a 100% responder.

In FIGS. 22 and 30-32, normalization was applied to visit intervals where eDiaries were completed for 21-27 days by multiplying the observed frequency by the inverse of the completion rate.

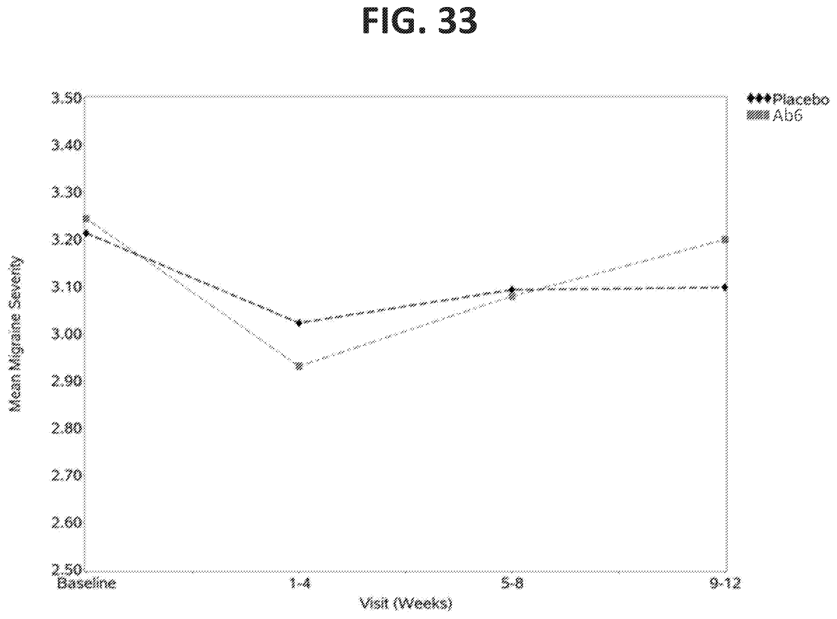

Migraine severity was also analyzed. FIG. 33 shows the mean migraine severity over time for the full analysis population. On the scale used, a mean migraine score of 3 represents “moderate pain.”

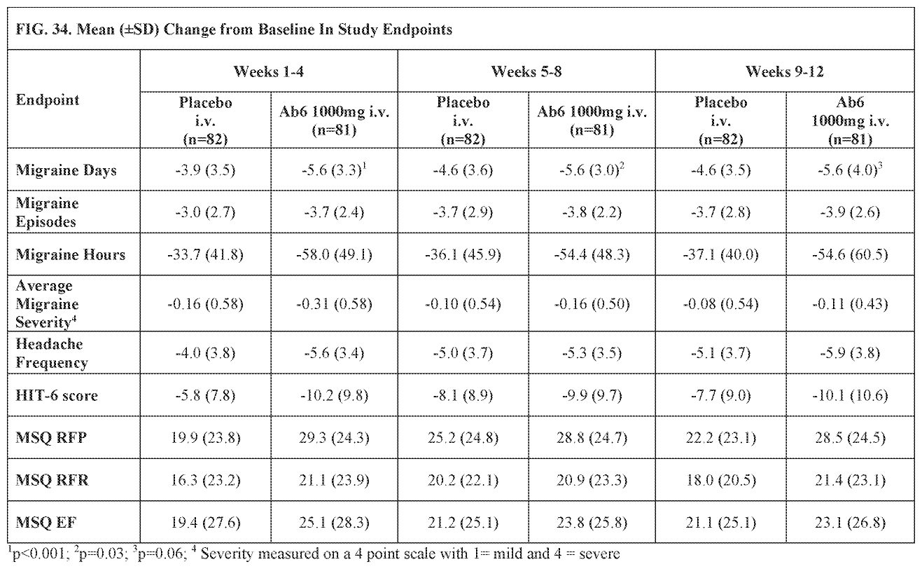

FIG. 34 summarizes the change from baseline in migraine days, migraine episodes, migraine hours, average migraine severity, headache frequency, and outcome measures including the HIT-6 score, MSQ (Migraine Specific Quality of Life Questionnaire) RFP (Role Function-Preventative), MSQ RFR (Role Function-Restrictive), and MSQ EF (Emotional Function).

### Example 3

### Human Clinical Study Evaluating the Safety and Efficacy of an Anti-CGRP Antibody in Chronic Migraine Patients

This example describes a randomized, double-blind, placebo-controlled clinical trial evaluating the safety and efficacy of Ab6 for chronic migraine prevention. In the study, 1,072 patients were randomized to receive Ab6 (300 mg or 100 mg), or placebo administered by infusion once every 12 weeks. To be eligible for the trial, patients must have experienced at least 15 headache days per month, of which at least eight met criteria for migraine. Patients that participated in the trial had an average of 16.1 migraine days per month at baseline. Study endpoints included the mean change from baseline in monthly migraine days, reduction in migraine prevalence at day 1 and over days 1-28, and reduction of at least 50%, 75%, and 100% from baseline in mean monthly migraine days, change from baseline in mean monthly acute migraine-specific medication days, and reductions from baseline in patient-reported impact scores on the Headache Impact Test (HIT-6). The administered antibody, Ab6, is an anti-CGRP antibody consisting of the light chain polypeptide of SEQ ID NO: 221 and heavy chain polypeptide of SEQ ID NO: 201.

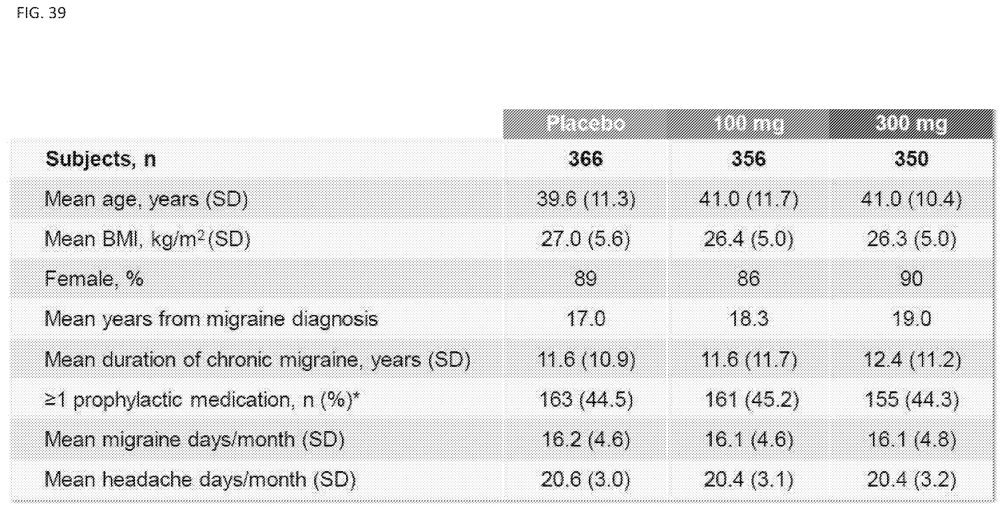

Patient characteristics are summarized in FIG. 39, with separate columns for patients receiving placebo, 100 mg of the antibody, or 300 mg of the antibody. Patients had a mean number of years from migraine diagnosis of between 17.0 and 19.0 years, a mean duration of suffering from chronic migraine of between 11.5 and 12.4 years, and between 44.3% and 45.2% of patients utilized at least one prophylactic medication. At baseline, in both antibody treatment groups the mean number of migraine days per month was 16.1, while for the placebo group, the mean number of migraine days per month was 16.2.

The reduction in a specified percentage (50%, 75%, or 100%) from baseline in mean monthly migraine days refers to the number or percentage of patients in a treatment group that exhibited the given percentage reduction in the number of migraine days per month. For example, a patient exhibiting 16 migraine days per month at baseline would be a 75% responder if the number of migraine days per month was decreased by at least 12 days per month over specified period.

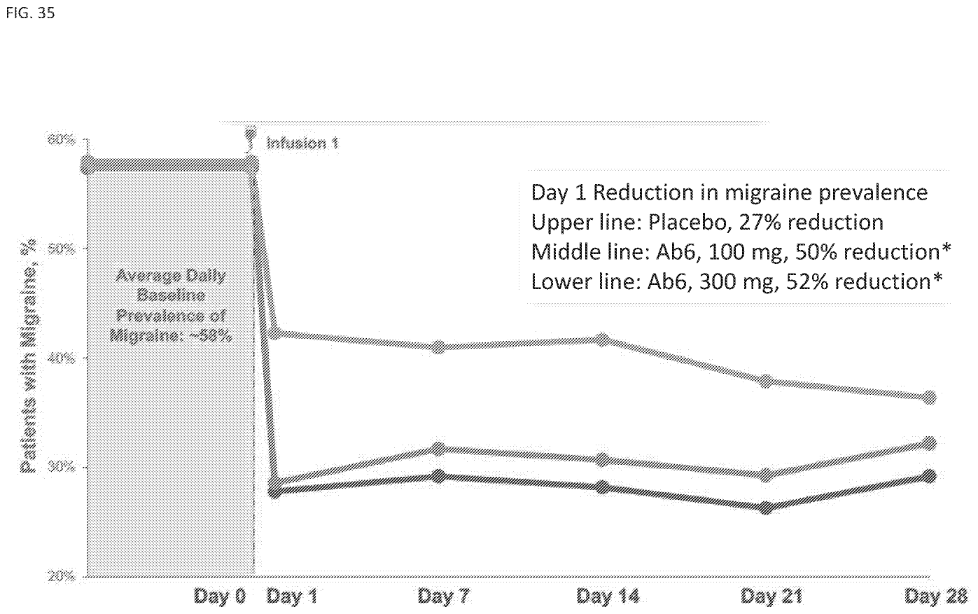

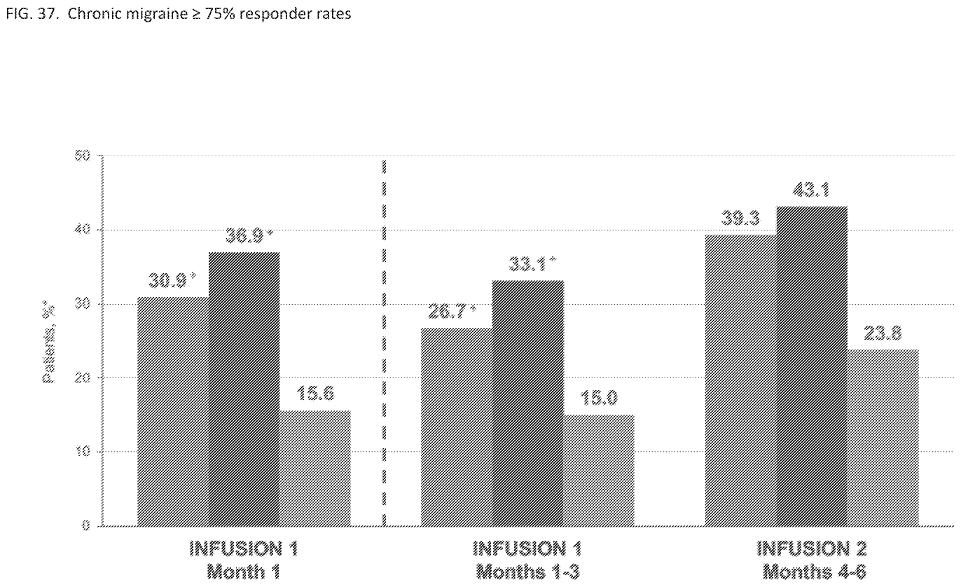

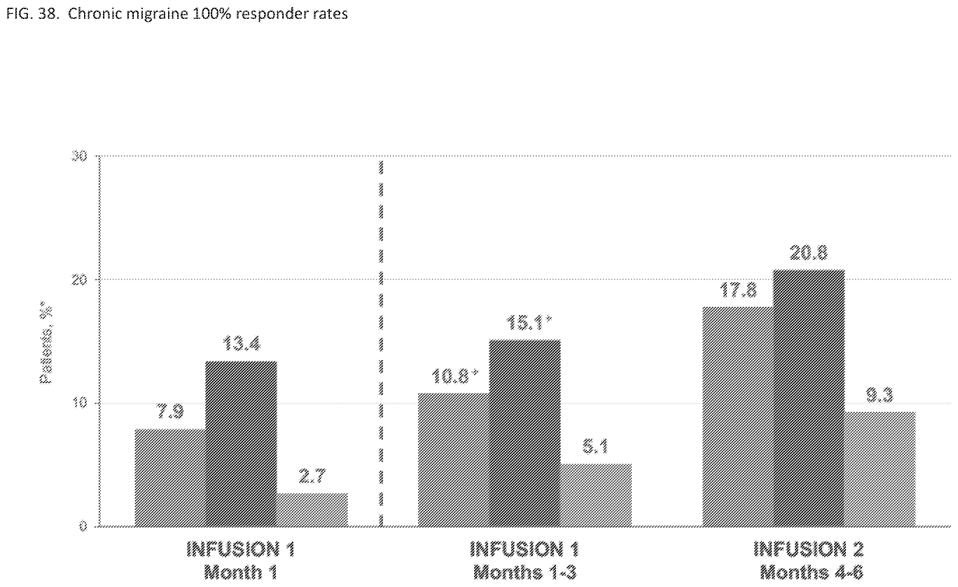

The results are shown in FIGS. 35-39. FIG. 35 shows the percentages of patients with migraine in the 300 mg, 100 mg, and placebo treatment groups at days 1, 7, 14, 21, and 28. The uppermost line shows results for placebo, the lowest line shows results for the 300 mg dosage, and the middle line shows results for the 100 mg dosage.

As shown in FIG. 35, at day 1 the percentage reduction in migraine prevalence was 52% for the 300 mg dosage, 50% at the 100 mg dosage, and 27% for placebo. The decrease was statistically significant compared to the placebo group for both the 100 mg and 300 mg treatment groups.

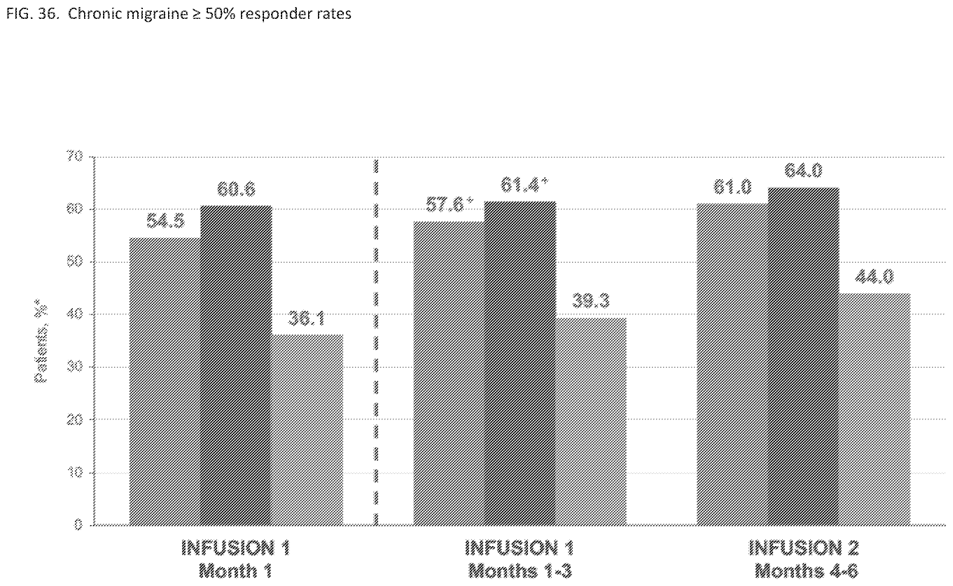

FIGS. 36-38 show the percentage of patients in the 300 mg and 100 mg treatment groups achieving, respectively, 50%, 75%, and 100% reduction in migraine days in month 1, over months 1-3 (after the 1st infusion), and over months 4-5 (after the 2nd infusion). In each graph, the data bars, from left to right, show results for the 100 mg, 300 mg, and placebo groups. Statistical significance is as shown. ++ indicates a statistically significant difference from placebo; + indicates a statistically significant difference from placebo (unadjusted); and § indicates a statistically significant difference from placebo (post hoc).

### Example 4

### Baseline Subgroup Analysis for Human Clinical Studies Evaluating the Safety and Efficacy of an Anti-CGRP Antibody in Chronic or Episodic Migraine Patients

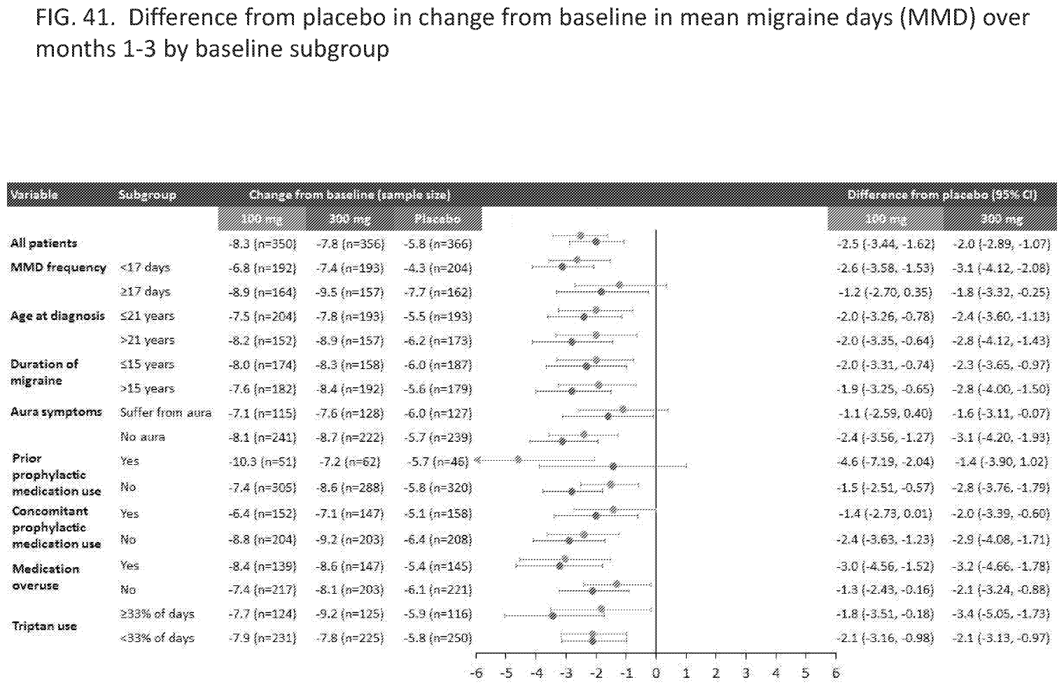

In the study of Chronic Migraine described in Example 3, at intake, each patient was assessed for potential medication overuse headache (MOH). MOH was present in 39.9% (139 patients) in the 100 mg treatment group, 42.0% (147 patients) in the 300 mg treatment group, and 39.6% (145 patients) in the placebo group. Assessment of the treatment outcomes in this patient subset indicated that treatment with the anti-CGRP antibody was efficacious for MOH (FIG. 41). Specifically, in the 100 mg treatment group, mean migraine days per month changed by −3.0 days (95% CI, −4.56 to −1.52 days) in the patients having MOH at baseline, compared to MOH patients receiving placebo. Similarly, in the 300 mg treatment group, mean migraine days per month changed by −3.2 days (95% CI, −4.66 to −1.78 days) in the patients having MOH at baseline, compared to MOH patients receiving placebo. By contrast, for patients without MOH at baseline, in the 100 mg treatment group, mean migraine days per month changed by −1.3 days (95% CI, −2.43 to −0.16 days), compared to patients without MOH at baseline receiving placebo. Likewise, for patients without MOH at baseline in the 300 mg treatment group, mean migraine days per month changed by −2.1 days (95% CI, −3.24 to −0.88 days), compared to patients without MOH at baseline receiving placebo. Efficacy for other subgroups was shown as well, including efficacy for patients with mean migraine day (MMD) frequency less than 17 days or greater than or equal to 17 days, patients with an age at diagnosis of less than or equal to 21 years or greater than 21 years, patients having a duration of migraine of less than or equal to 15 year or greater than 15 years, patients suffering from migraine with aura or migraine with no aura, patients with prior prophylactic medication use or no prior prophylactic medication use, patients with concomitant prophylactic medication use or no concomitant prophylactic medication use, ant patients with triptan use on greater than or equal to 33% of days, or less than 33% of days. In each case, efficacy for each subgroup was shown (FIG. 41).

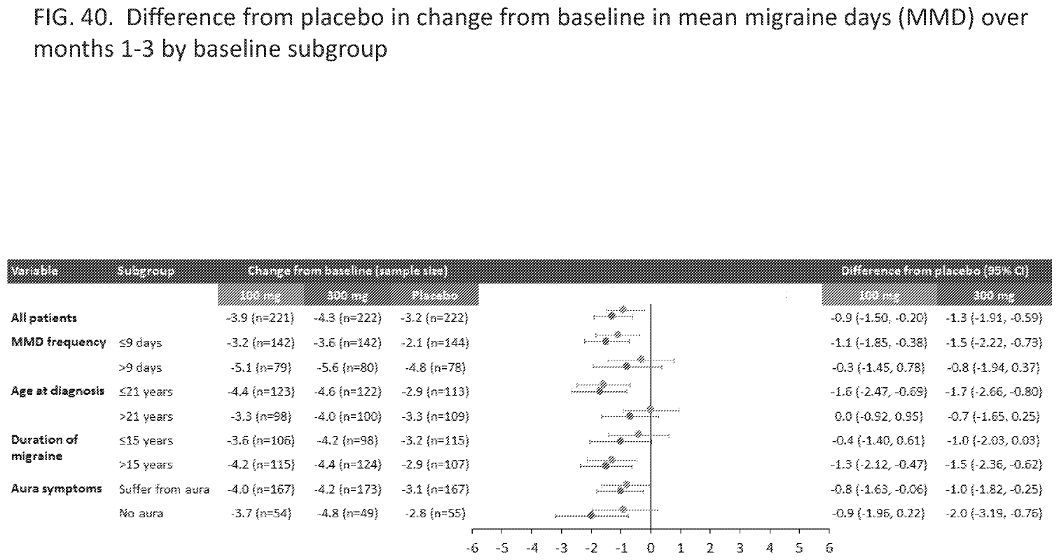

In another human clinical trial of patients with episodic migraine, patients were randomized to receive Ab6 100 mg (n=221), 300 mg (n=222), or placebo (n=222) in a double blind, parallel study. After a 28 day screening period, patients were administered the drug or placebo intravenously every 3 months for 4 total infusions (FIG. 40). Efficacy was shown over months 1-3 for both the 100 mg and 300 mg treatment groups, with a mean change in migraine days of −3.9 for the 100 mg treatment group and −4.3 days for the 300 mg treatment group, compared to −3.2 days for the placebo group. Efficacy for subgroups of patients was also shown, including efficacy for patients with mean migraine day (MMD) frequency less than or equal to 9 days or greater than 9 days, patients with an age at diagnosis of less than or equal to 21 years or greater than 21 years, patients having a duration of migraine of less than or equal to 15 year or greater than 15 years, and patients suffering from migraine with aura or migraine with no aura.

### Example 5

### Effects of Ab6 Treatment on Medication Use in Chronic and Episodic Migraine Patients

During the studies of chronic migraine patients described in Example 3 and episodic migraine patients described in Example 4, patients also recorded use of acute medication in a daily eDiary and were allowed to use acute medication at their own discretion. Acute medications for migraine included ergots, triptans, and analgesics (e.g., NSAIDS, opioids, and caffeine-containing combination analgesics).

For further analysis, patients were stratified by the number of days with acute medication use during the 28-day screening period (1-9 or ≥10 days; “baseline”). Acute medication days were calculated for individual types of acute medications and combined, meaning that if 2 or more types medications were used on the same calendar days, they were counted as separate medication use days. For example, if a patient took an opioid and a triptan on the same day, it counted as 2 days of acute medication use. These analyses included patients with at least 1 acute medication use day during the 28-day baseline screening period.

In both chronic migraine and episodic migraine patients who used acute medication during the 28-day baseline period, Ab6 treatment resulted in greater average reductions in monthly migraine days and acute medication days than placebo as early as Month 1 after dosing, with similar results across 2 dose intervals over 6 months.

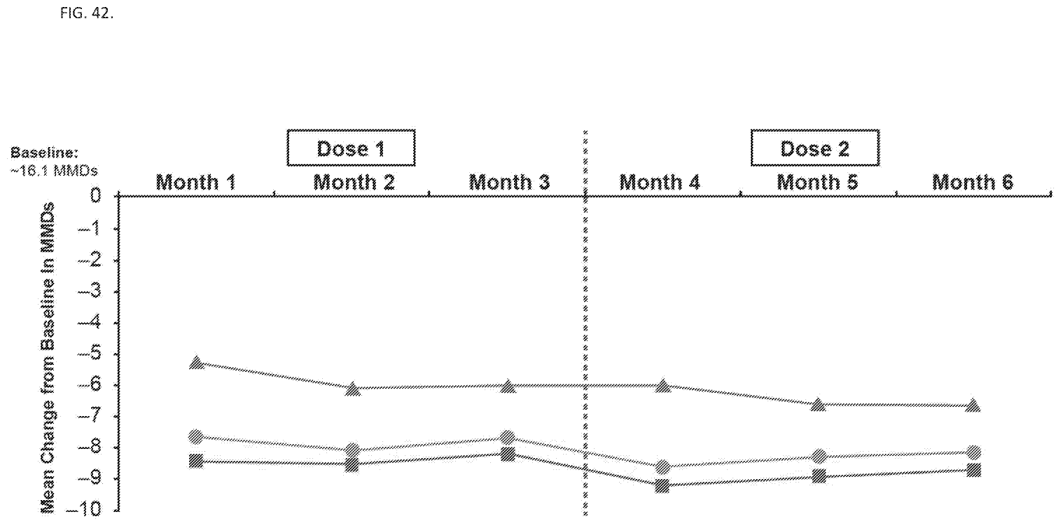

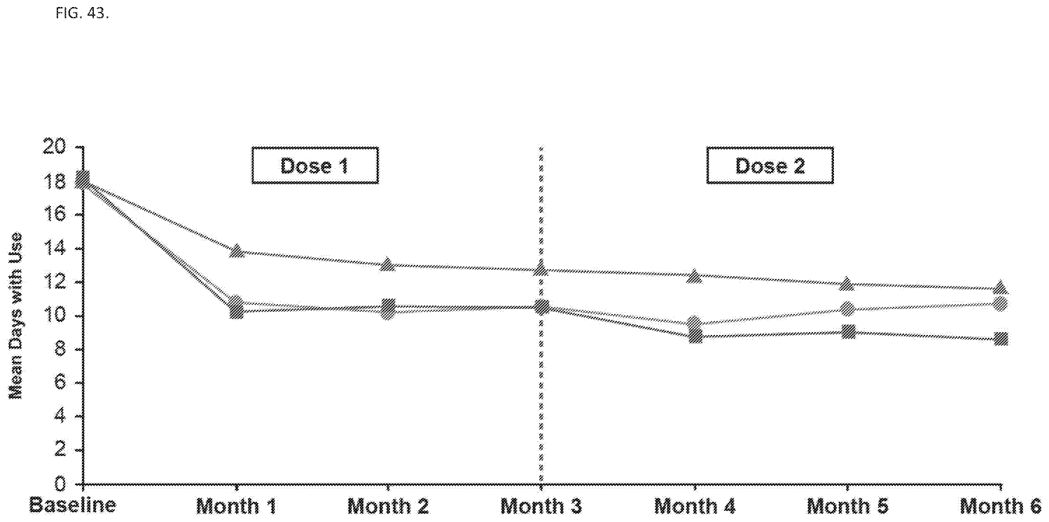

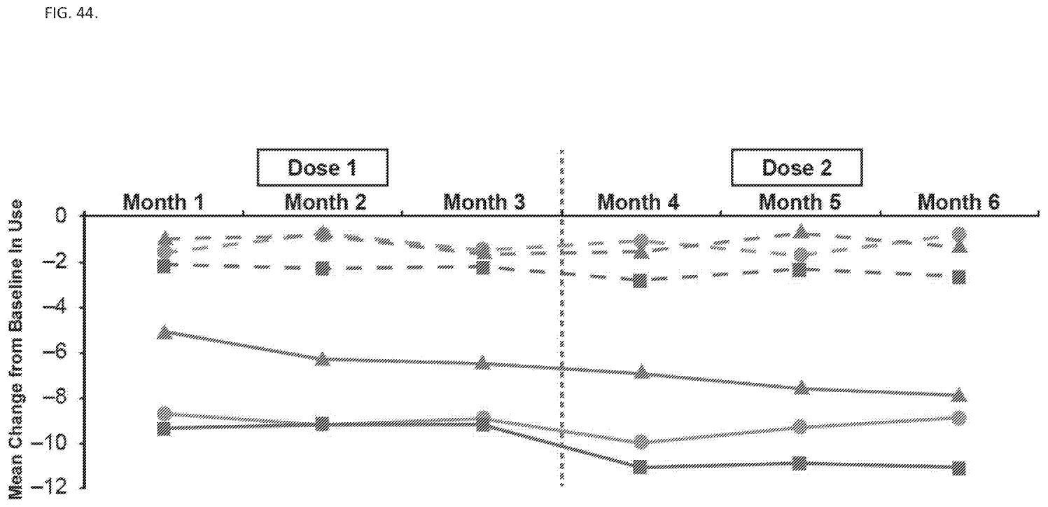

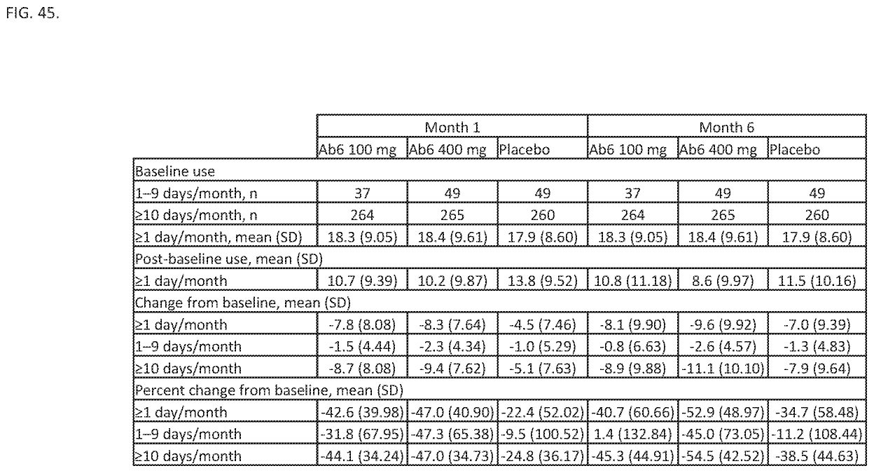

Ab6 consistently demonstrated greater reductions in mean monthly migraine days over 6 months of treatment than placebo in chronic migraine patients taking ≥1 day of acute medication use during baseline (FIG. 42). Chronic migraine patients who had at least one day of acute medication use per month during baseline demonstrated greater decreases in acute medication use than placebo as early as month 1 after treatment and across the entire 6 month treatment period (FIG. 43). In the subgroup of chronic migraine patients who were taking 1-9 days of acute medication during baseline, the change from baseline in days of acute medication use was greater in the 300 mg Ab6 group than placebo across 6 months of treatment (FIG. 44). A clear decrease in medication days per month was observed for patients with at least 10 days of medication use per month at baseline for both Ab6 treatment group compared to placebo over the entire 6 month period. FIG. 45 shows the changes in medication use days at Month 1 and Month 6 in the subgroups of chronic migraine patients with ≥1, 1-9, and ≥10 days of acute medication use at baseline. With the exception of Ab6 100 mg at month 6 in patients with 1-9 days/month of use at baseline, Ab6 demonstrated a greater treatment effect in reducing acute medication use than placebo.

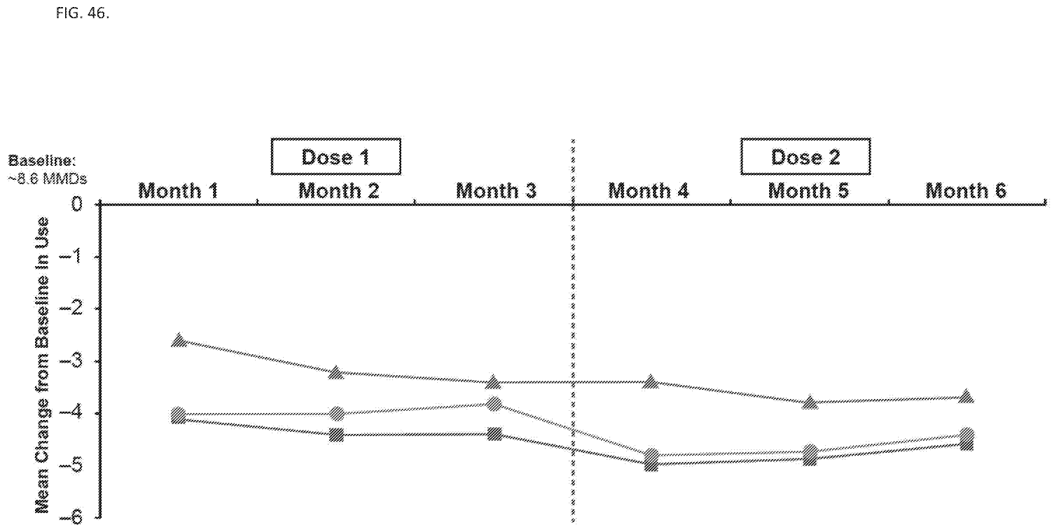

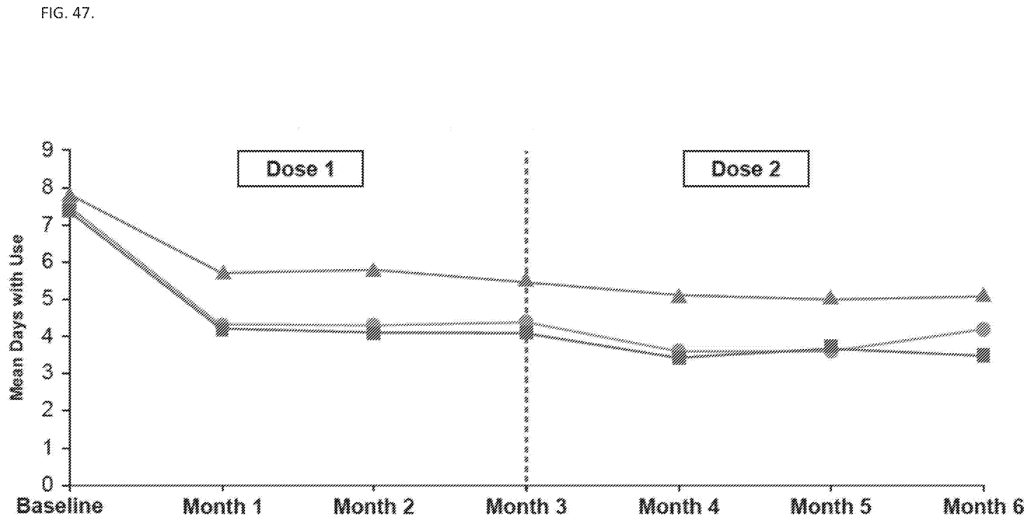

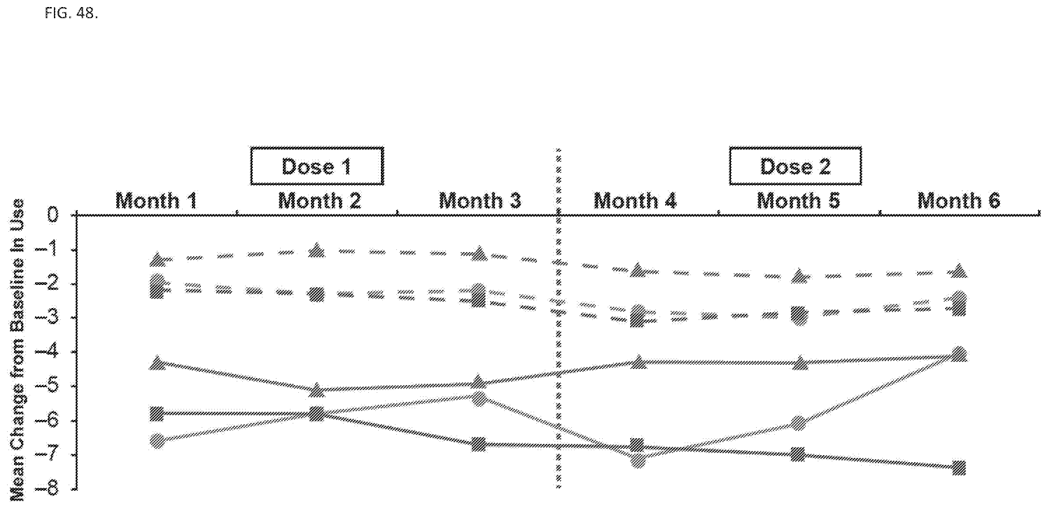

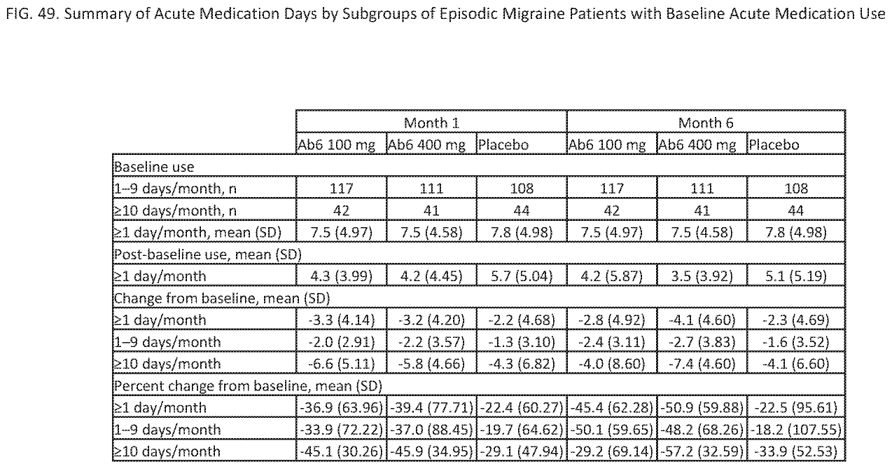

Similarly, across 2 dose intervals over 6 months, episodic migraine patients with one or more days of acute medication use during baseline experienced greater reductions in mean monthly migraine days with Ab6 than Placebo (FIG. 46). Episodic migraine patients who had at least one day of acute medication use per month during baseline demonstrated greater decreases in acute medication use than placebo as early as month 1 after treatment and across the entire 6 month treatment period (FIG. 47). In the subgroup of episodic migraine patients who were taking 1-9 days of acute medication during baseline, the change from baseline in days of acute medication use was greater with Ab6 than placebo across 6 months of treatment (FIG. 48). A similar pattern was observed in the subgroup of patients who were taking ≥10 days of acute medication during baseline, though smaller sample sizes may have contributed to the less consistent pattern over time. FIG. 49 shows the changes in medication use days at Month 1 and Month 6 in the subgroups of episodic migraine patients with ≥1, 1-9, and ≥10 days of acute medication use at baseline. With the exception of Ab6 100 mg at Month 6 in patients with ≥10 days/month of use at baseline, the reduction in acute medication use was greater in the Ab6 treatment groups than placebo.

The results show that both episodic migraine and chronic migraine patients who were at risk for medication-overuse headache (≥10 days/month of acute medication use) demonstrated the greatest reductions in acute medication use, with Ab6 treatment generally resulting in larger decreases in medication use days than placebo.

The most frequently reported acute headache medications in >10% of subjects included Thomapyrin N (44.5%) (a combination of paracetamol, aspirin, and caffeine), ibuprofen (40.6%), sumatriptan (33.6%), paracetamol (acetaminophen) (20.3%), and naproxen sodium (10.2%). The most frequently reported preventive headache medication in >10% of subjects was topiramate (12.5%).

### Example 6

### Efficacy of Anti-CGRP Antibodies in Subjects Experiencing an Acute Attack of Migraine

This example describes a randomized, double-blind, placebo-controlled clinical trial evaluating the safety and efficacy of Ab6 for the acute treatment of migraine. In the study, approximately 450 patients are randomized 1:1 to receive either 100 mg Ab6 or placebo. During a screening period (approx. 1-8 weeks) patients are assessed for migraine frequency and medication use frequency. Eligible patients have a migraine attack frequency of about 4-15 migraine days per month in the 3 months prior to screening. By history, the subject's typical migraine attack, if untreated, would be associated with headache pain of moderate to severe intensity and a most bothersome symptom of nausea, photophobia, or phonophobia. Subjects must be headache free for at least 24 hours prior to onset of a qualifying migraine in order to participate in the trial. On the day of treatment, the patient will travel to the study site and intravenous infusion of 100 mg Ab6 or placebo will commence between about 1-6 hours from the start of the attack. Patients will not have received any other monoclonal antibody (e.g., any CGRP antagonist antibody) within the 6 month period prior to screening.

Co-Primary Endpoints are time to headache pain freedom and time to absence of most bothersome symptom. Co-Key secondary are headache pain freedom at 2 hours and absence of most bothersome symptom at 2 hours. Secondary endpoints are time to headache pain relief, headache pain freedom at 2 hours with sustained headache pain freedom for 24 and 48 hours, use of rescue medication by 24 hours and by 48 hours, absence of photophobia at 2 hours, absence of phonophobia at 2 hours, absence of nausea at 2 hours, change from Baseline in Headache Impact Test (HIT 6) at Week 4, and change from Baseline in Migraine Treatment Optimization Questionnaire-6 (mTOQ-6) at Week 4. Exploratory Endpoints are absence of headache pain at all timepoints other than 2 hours, absence of photophobia at all timepoints other than 2 hours, absence of phonophobia at all timepoints other than 2 hours, absence of nausea at all timepoints other than 2 hours, pain relapse when the subject was headache pain-free at 2 hours, patient Global Impression of Change (PGIC) at Week 4, and time to next migraine. Headache pain is collected on a 4-point scale with 3 being severe, 2 being moderate, 1 being mild, and 0 being no pain. Pain freedom is no pain (0) with the absence of rescue medication (note that in the trial rescue medication is not to be used for 2 hours post completion of infusion in order to separate the effects of the antibody from the rescue medication, however, in the course of normal use, rescue medication optionally may be used; any use of rescue medication is collected as data).

Statistical analysis is performed to determine significance of the difference in endpoints between patients receiving Ab6 or placebo, including the time to pain freedom and time to absence of most bothersome symptom, and each of the other aforementioned endpoints.

Use of rescue medication refers to any intervention (medical or device) provided to the subject to provide relief of migraine. In the study this should not be provided sooner than 2 hours following completion of the study drug administration in order to separate the effects of the antibody from the effects of said rescue medication, however, rescue medication is not contraindicated. The proportion of subjects requiring rescue medication use is summarized in the study. Acute rescue medication includes any medication to treat migraine or migraine associated symptoms, e.g., triptans, analgesics such as non-opioids or opioids/narcotics, acetaminophen, NSAIDS, combination medications such as EXCEDRIN® or EXCEDRIN MIGRAINE®, antiemetic medications, ergotamines, ergot derivatives, etc.

Absence of Migraine-Associated Symptoms (Photophobia, Phonophobia and Nausea) refers to the absence or presence of each of the aforementioned migraine-associated symptoms, as reported by the subject. The proportion of subjects absent the symptoms, with no administration of rescue medication, is summarized in the study.

Headache Impact Test (HIT-6) is assessed as the change from baseline of the total score, and is summarized and compared between treatment groups in the study.

Migraine Treatment Optimization Questionnaire-6 (mTOQ-6) is assessed as the change from baseline of the total score and is summarized and compared between the treatment groups in the study.

Time to Headache Pain Relief is assessed as the first time point post completion of infusion at which the subject reports relief of pain meaning their headache pain has gone from moderate or severe (2 or 3) to mild or no pain (1 or 0) with no administration of rescue medication.

Pain Relapse is assessed as the occurrence of headache of any severity within 48 hours of drug administration for a patient who has no headache pain (0) at 2 hours. The proportion of subjects with recurrence of headache pain of any severity is summarized in the study.

The study shows that Ab6 is effective and safe for acute migraine treatment.

What is claimed is:

1-102. (canceled)

103. A method of acute treatment of migraine in a patient in need thereof, the method comprising intravenously administering to the patient:
(A) 100 mg or 300 mg of a monoclonal antibody comprising:
(a) a variable light chain polypeptide comprising light chain complementary determining region (CDR) 1, 2, and 3 polypeptide sequences of SEQ ID NO: 224, SEQ ID NO: 226, and SEQ ID NO: 228, respectively, and
(b) a variable heavy chain polypeptide comprising heavy chain CDR 1, 2, and 3 polypeptide sequences of SEQ ID NO: 204, SEQ ID NO: 206, and SEQ ID NO: 208, respectively; and
(B) one or more acute migraine medications,
wherein the administering is followed by preventive treatment once every three months using 100 mg or 300 mg of said monoclonal antibody.

104. The method according to claim 103, wherein the one or more acute migraine medications comprise triptan, an analgesic such as non-opioids or opioids/narcotics, acetaminophen, an NSAID, a combination medication, an ergotamine, and/or an ergot derivative.

105. The method according to claim 104, wherein the patient suffers from chronic migraine.

106. The method according to claim 105, wherein the 100 mg or 300 mg of the monoclonal antibody is administered to the patient once every three months, for at least six months.

107. The method according to claim 104, wherein the patient suffers from episodic migraine.

108. The method according to claim 107, wherein the 100 mg or 300 mg of the monoclonal antibody is administered to the patient once every three months, for at least twelve months.

109. The method according to claim 104, wherein the monoclonal antibody comprises a human IgG1 constant region and is produced in Chinese hamster ovary (CHO) cells.

110. The method according to claim 104, wherein the monoclonal antibody comprises a human IgG1 constant region and is produced in Pichia pastoris yeast cells.

111. The method according to claim 104, wherein the monoclonal antibody is a humanized IgG1 monoclonal antibody.

112. The method according to claim 104, wherein:
(A) the variable light chain polypeptide comprises the amino acid sequence of SEQ ID NO: 222; and
(B) the variable heavy chain polypeptide comprises the amino acid sequence of SEQ ID NO: 202.

113. The method according to claim 112, wherein the monoclonal antibody is produced in CHO cells.

114. The method according to claim 112, wherein the monoclonal antibody further comprises the heavy chain constant region polypeptide sequence of SEQ ID NO: 565.

115. The method according to claim 104, wherein the monoclonal antibody comprises:
(A) a light chain polypeptide comprising the amino acid sequence of SEQ ID NO: 221; and
(B) a heavy chain polypeptide comprising the amino acid sequence of SEQ ID NO: 201.

116. The method according to claim 104, wherein the monoclonal antibody comprises:
(A) a light chain polypeptide comprising the amino acid sequence of SEQ ID NO: 221; and
(B) a heavy chain polypeptide comprising the amino acid sequence of SEQ ID NO: 566.

117. The method according to claim 104, wherein the patient experiences:
(i) a reduction in the number of migraine days per month in the one-month period following the administration of the monoclonal antibody, relative to the number of migraine days per month experienced by the patient during the previous one-month period prior to said administration;
(ii) a reduction in the number of migraine days per month in the three-month period following the administration of the monoclonal antibody, relative to the baseline number of migraine days per month experienced by the patient prior to said administration, wherein said baseline number of migraine days per month is determined over a period of one month or longer; and/or
(iii) a reduction in migraine prevalence in the one-month period after being administered the monoclonal antibody, relative to the patient's migraine prevalence during the previous one-month period prior to said administration.

118. A method of reducing the occurrence of migraine in a patient in need thereof, the method comprising intravenously administering to the patient, once every three months, 100 mg or 300 mg of a monoclonal antibody comprising:
(A) a variable light chain polypeptide comprising light chain complementary determining region (CDR) 1, 2, and 3 polypeptide sequences of SEQ ID NO: 224, SEQ ID NO: 226, and SEQ ID NO: 228, respectively, and
(B) a variable heavy chain polypeptide comprising heavy chain CDR 1, 2, and 3 polypeptide sequences of SEQ ID NO: 204, SEQ ID NO: 206, and SEQ ID NO: 208, respectively.

119. The method according to claim 118, wherein the patient suffers from chronic migraine.

120. The method according to claim 119, wherein the 100 mg or 300 mg of the monoclonal antibody is intravenously administered to the patient once every three months, for at least six months.

121. The method according to claim 118, wherein the patient suffers from episodic migraine.

122. The method according to claim 121, wherein the 100 mg or 300 mg of the monoclonal antibody is intravenously administered to the patient once every three months, for at least twelve months.

123. The method according to claim 118, wherein the monoclonal antibody comprises a human IgG1 constant region and is produced in CHO cells.

124. The method according to claim 118, wherein the monoclonal antibody comprises a human IgG1 constant region and is produced in Pichia pastoris yeast cells.

125. The method according to claim 118, wherein the monoclonal antibody is a humanized IgG1 monoclonal antibody.

126. The method according to claim 118, wherein:
(A) the variable light chain polypeptide comprises the amino acid sequence of SEQ ID NO: 222; and
(B) the variable heavy chain polypeptide comprises the amino acid sequence of SEQ ID NO: 202.

127. The method according to claim 126, wherein the monoclonal antibody is produced in CHO cells.

128. The method according to claim 126, wherein the monoclonal antibody further comprises the heavy chain constant region polypeptide sequence of SEQ ID NO: 565.

129. The method according to claim 118, wherein the monoclonal antibody comprises:
(A) a light chain polypeptide comprising the amino acid sequence of SEQ ID NO: 221; and
(B) a heavy chain polypeptide comprising the amino acid sequence of SEQ ID NO: 201.

130. The method according to claim 118, wherein the monoclonal antibody comprises:
(A) a light chain polypeptide comprising the amino acid sequence of SEQ ID NO: 221; and
(B) a heavy chain polypeptide comprising the amino acid sequence of SEQ ID NO: 566.

131. The method according to claim 118, wherein the patient experiences:
(i) a reduction in the number of migraine days per month in the one-month period following the administration of the monoclonal antibody, relative to the number of migraine days per month experienced by the patient during the previous one-month period prior to said administration;
(ii) a reduction in the number of migraine days per month in the three-month period following the administration of the monoclonal antibody, relative to the baseline number of migraine days per month experienced by the patient prior to said administration, wherein said baseline number of migraine days per month is determined over a period of one month or longer; and/or
(iii) a reduction in migraine prevalence in the one-month period following the administration of the monoclonal antibody, relative to the patient's migraine prevalence during the previous one-month period prior to said administration.

132. A method of preventing migraine in a patient in need thereof, the method comprising intravenously administering to the patient, once every three months, 100 mg or 300 mg of a humanized monoclonal antibody comprising:
(a) a variable light chain polypeptide comprising light chain complementary determining region (CDR) 1, 2, and 3 polypeptide sequences of SEQ ID NO: 224, SEQ ID NO: 226, and SEQ ID NO: 228, respectively, and
(b) a variable heavy chain polypeptide comprising the amino acid sequence of SEQ ID NO: 202,
optionally wherein the variable light chain comprises the amino acid sequence of SEQ ID NO: 222,
further optionally wherein the monoclonal antibody comprises:
(a) a light chain polypeptide comprising the amino acid sequence of SEQ ID NO: 221; and
(b) a heavy chain polypeptide comprising the amino acid sequence of SEQ ID NO: 201 or 566.


SEQUENCE LISTING (567 sequences)

SEQ ID NO: 1 - protein, 439 aa, synthetic construct
  REGION 1..439: note = Engineered antibody sequence
QSLEESGGRLVTPGTPLTLTCTVSGLDLSSYYMQWVRQAPGKGLEWIGVIGINDNTYYASWAKGRFTISRASSTTVDLKMTSLTTEDTATYFCARGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 109 aa, synthetic construct
  REGION 1..109: note = Engineered antibody sequence
QSLEESGGRLVTPGTPLTLTCTVSGLDLSSYYMQWVRQAPGKGLEWIGVIGINDNTYYASWAKGRFTISRASSTTVDLKMTSLTTEDTATYFCARGDIWGPGTLVTVSS

SEQ ID NO: 3 - protein, 29 aa, synthetic construct
  REGION 1..29: note = Engineered antibody sequence
QSLEESGGRLVTPGTPLTLTCTVSGLDLS

SEQ ID NO: 4 - protein, 5 aa, synthetic construct
  REGION 1..5: note = Engineered antibody sequence
SYYMQ

SEQ ID NO: 5 - protein, 14 aa, synthetic construct
  REGION 1..14: note = Engineered antibody sequence
WVRQAPGKGLEWIG

SEQ ID NO: 6 - protein, 16 aa, synthetic construct
  REGION 1..16: note = Engineered antibody sequence
VIGINDNTYYASWAKG

SEQ ID NO: 7 - protein, 31 aa, synthetic construct
  REGION 1..31: note = Engineered antibody sequence
RFTISRASSTTVDLKMTSLTTEDTATYFCAR

SEQ ID NO: 8 - skipped

SEQ ID NO: 9 - protein, 11 aa, synthetic construct
  REGION 1..11: note = Engineered antibody sequence
WGPGTLVTVSS

SEQ ID NO: 10 - protein, 330 aa, synthetic construct
  REGION 1..330: note = Engineered antibody sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1320 bp, synthetic construct
  misc_feature 1..1320: note = Engineered antibody sequence
  source 1..1320: mol_type = other DNA
cagtcgctggaggagtccgggggtcgcctggtcacgcctgggacacccctgacactcacctgcacagtctctggactcgacctcagtagctactacatgcaatgggtccgccaggctccagggaaggggctggaatggatcggagtcattggtattaatgataacacatactacgcgagctgggcgaaaggccgattcaccatctccagagcctcgtcgaccacggtggatctgaaaatgaccagtctgacaaccgaggacacggccacctatttctgtgccagaggggacatctggggcccaggcaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - DNA, 327 bp, synthetic construct
  misc_feature 1..327: note = Engineered antibody sequence
  source 1..327: mol_type = other DNA
cagtcgctggaggagtccgggggtcgcctggtcacgcctgggacacccctgacactcacctgcacagtctctggactcgacctcagtagctactacatgcaatgggtccgccaggctccagggaaggggctggaatggatcggagtcattggtattaatgataacacatactacgcgagctgggcgaaaggccgattcaccatctccagagcctcgtcgaccacggtggatctgaaaatgaccagtctgacaaccgaggacacggccacctatttctgtgccagaggggacatctggggcccaggcaccctcgtcaccgtctcgagc

SEQ ID NO: 13 - DNA, 87 bp, synthetic construct
  misc_feature 1..87: note = Engineered antibody sequence
  source 1..87: mol_type = other DNA
cagtcgctggaggagtccgggggtcgcctggtcacgcctgggacacccctgacactcacctgcacagtctctggactcgacctcagt

SEQ ID NO: 14 - DNA, 15 bp, synthetic construct
  misc_feature 1..15: note = Engineered antibody sequence
  source 1..15: mol_type = other DNA
agctactacatgcaa

SEQ ID NO: 15 - DNA, 42 bp, synthetic construct
  misc_feature 1..42: note = Engineered antibody sequence
  source 1..42: mol_type = other DNA
tgggtccgccaggctccagggaaggggctggaatggatcgga

SEQ ID NO: 16 - DNA, 48 bp, synthetic construct
  misc_feature 1..48: note = Engineered antibody sequence
  source 1..48: mol_type = other DNA
gtcattggtattaatgataacacatactacgcgagctgggcgaaaggc

SEQ ID NO: 17 - DNA, 93 bp, synthetic construct
  misc_feature 1..93: note = Engineered antibody sequence
  source 1..93: mol_type = other DNA
cgattcaccatctccagagcctcgtcgaccacggtggatctgaaaatgaccagtctgacaaccgaggacacggccacctatttctgtgccaga

SEQ ID NO: 18 - skipped

SEQ ID NO: 19 - DNA, 33 bp, synthetic construct
  misc_feature 1..33: note = Engineered antibody sequence
  source 1..33: mol_type = other DNA
tggggcccaggcaccctcgtcaccgtctcgagc

SEQ ID NO: 20 - DNA, 993 bp, synthetic construct
  misc_feature 1..993: note = Engineered antibody sequence
  source 1..993: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 21 - protein, 219 aa, synthetic construct
  REGION 1..219: note = Engineered antibody sequence
QVLTQTASPVSAAVGSTVTINCQASQSVYDNNYLAWYQQKPGQPPKQLIYSTSTLASGVSSRFKGSGSGTQFTLTISDLECADAATYYCLGSYDCSSGDCFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 113 aa, synthetic construct
  REGION 1..113: note = Engineered antibody sequence
QVLTQTASPVSAAVGSTVTINCQASQSVYDNNYLAWYQQKPGQPPKQLIYSTSTLASGVSSRFKGSGSGTQFTLTISDLECADAATYYCLGSYDCSSGDCFVFGGGTEVVVKR

SEQ ID NO: 23 - protein, 22 aa, synthetic construct
  REGION 1..22: note = Engineered antibody sequence
QVLTQTASPVSAAVGSTVTINC

SEQ ID NO: 24 - protein, 13 aa, synthetic construct
  REGION 1..13: note = Engineered antibody sequence
QASQSVYDNNYLA

SEQ ID NO: 25 - protein, 15 aa, synthetic construct
  REGION 1..15: note = Engineered antibody sequence
WYQQKPGQPPKQLIY

SEQ ID NO: 26 - protein, 7 aa, synthetic construct
  REGION 1..7: note = Engineered antibody sequence
STSTLAS

SEQ ID NO: 27 - protein, 32 aa, synthetic construct
  REGION 1..32: note = Engineered antibody sequence
GVSSRFKGSGSGTQFTLTISDLECADAATYYC

SEQ ID NO: 28 - protein, 13 aa, synthetic construct
  REGION 1..13: note = Engineered antibody sequence
LGSYDCSSGDCFV

SEQ ID NO: 29 - protein, 11 aa, synthetic construct
  REGION 1..11: note = Engineered antibody sequence
FGGGTEVVVKR

SEQ ID NO: 30 - protein, 106 aa, synthetic construct
  REGION 1..106: note = Engineered antibody sequence
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 660 bp, synthetic construct
  misc_feature 1..660: note = Engineered antibody sequence
  source 1..660: mol_type = other DNA
caagtgctgacccagactgcatcccccgtgtctgcagctgtgggaagcacagtcaccatcaattgccaggccagtcagagtgtttatgataacaactacctagcctggtatcagcagaaaccagggcagcctcccaagcaactgatctattctacatccactctggcatctggggtctcatcgcggttcaaaggcagtggatctgggacacagttcactctcaccatcagcgacctggagtgtgccgatgctgccacttactactgtctaggcagttatgattgtagtagtggtgattgttttgttttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 32 - DNA, 339 bp, synthetic construct
  misc_feature 1..339: note = Engineered antibody sequence
  source 1..339: mol_type = other DNA
caagtgctgacccagactgcatcccccgtgtctgcagctgtgggaagcacagtcaccatcaattgccaggccagtcagagtgtttatgataacaactacctagcctggtatcagcagaaaccagggcagcctcccaagcaactgatctattctacatccactctggcatctggggtctcatcgcggttcaaaggcagtggatctgggacacagttcactctcaccatcagcgacctggagtgtgccgatgctgccacttactactgtctaggcagttatgattgtagtagtggtgattgttttgttttcggcggagggaccgaggtggtggtcaaacgt

SEQ ID NO: 33 - DNA, 66 bp, synthetic construct
  misc_feature 1..66: note = Engineered antibody sequence
  source 1..66: mol_type = other DNA
caagtgctgacccagactgcatcccccgtgtctgcagctgtgggaagcacagtcaccatcaattgc

SEQ ID NO: 34 - DNA, 39 bp, synthetic construct
  misc_feature 1..39: note = Engineered antibody sequence
  source 1..39: mol_type = other DNA
caggccagtcagagtgtttatgataacaactacctagcc

SEQ ID NO: 35 - DNA, 45 bp, synthetic construct
  misc_feature 1..45: note = Engineered antibody sequence
  source 1..45: mol_type = other DNA
tggtatcagcagaaaccagggcagcctcccaagcaactgatctat

SEQ ID NO: 36 - DNA, 21 bp, synthetic construct
  misc_feature 1..21: note = Engineered antibody sequence
  source 1..21: mol_type = other DNA
tctacatccactctggcatct

SEQ ID NO: 37 - DNA, 96 bp, synthetic construct
  misc_feature 1..96: note = Engineered antibody sequence
  source 1..96: mol_type = other DNA
ggggtctcatcgcggttcaaaggcagtggatctgggacacagttcactctcaccatcagcgacctggagtgtgccgatgctgccacttactactgt

SEQ ID NO: 38 - DNA, 39 bp, synthetic construct
  misc_feature 1..39: note = Engineered antibody sequence
  source 1..39: mol_type = other DNA
ctaggcagttatgattgtagtagtggtgattgttttgtt

SEQ ID NO: 39 - DNA, 33 bp, synthetic construct
  misc_feature 1..33: note = Engineered antibody sequence
  source 1..33: mol_type = other DNA
ttcggcggagggaccgaggtggtggtcaaacgt

SEQ ID NO: 40 - DNA, 321 bp, synthetic construct
  misc_feature 1..321: note = Engineered antibody sequence
  source 1..321: mol_type = other DNA
acggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 41 - protein, 441 aa, synthetic construct
  REGION 1..441: note = Engineered antibody sequence
EVQLVESGGGLVQPGGSLRLSCAVSGLDLSSYYMQWVRQAPGKGLEWVGVIGINDNTYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 111 aa, synthetic construct
  REGION 1..111: note = Engineered antibody sequence
EVQLVESGGGLVQPGGSLRLSCAVSGLDLSSYYMQWVRQAPGKGLEWVGVIGINDNTYYASWAKGRFTISRDNSKTTVYLQMNSLRAEDTAVYFCARGDIWGQGTLVTVSS

SEQ ID NO: 43 - protein, 30 aa, synthetic construct
  REGION 1..30: note = Engineered antibody sequence
EVQLVESGGGLVQPGGSLRLSCAVSGLDLS

SEQ ID NO: 44 - protein, 5 aa, synthetic construct
  REGION 1..5: note = Engineered antibody sequence
SYYMQ

SEQ ID NO: 45 - protein, 14 aa, synthetic construct
  REGION 1..14: note = Engineered antibody sequence
WVRQAPGKGLEWVG

SEQ ID NO: 46 - protein, 16 aa, synthetic construct
  REGION 1..16: note = Engineered antibody sequence
VIGINDNTYYASWAKG

SEQ ID NO: 47 - protein, 32 aa, synthetic construct
  REGION 1..32: note = Engineered antibody sequence
RFTISRDNSKTTVYLQMNSLRAEDTAVYFCAR

SEQ ID NO: 48 - skipped

SEQ ID NO: 49 - protein, 11 aa, synthetic construct
  REGION 1..11: note = Engineered antibody sequence
WGQGTLVTVSS

SEQ ID NO: 50 - protein, 330 aa, synthetic construct; identical to SEQ ID NO: 10
  REGION 1..330: note = Engineered antibody sequence

SEQ ID NO: 51 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggactcgacctcagtagctactacatgcaatgggtccgtcaggctccagggaaggggctggagtgggtcggagtcattggtatcaatgataacacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 52 - DNA, 333 bp, synthetic construct
  misc_feature 1..333: note = Engineered antibody sequence
  source 1..333: mol_type = other DNA
gaggtgcagcttgtggagtctgggggaggcttggtccagcctggggggtccctgagactctcctgtgcagtctctggactcgacctcagtagctactacatgcaatgggtccgtcaggctccagggaaggggctggagtgggtcggagtcattggtatcaatgataacacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagc

SEQ ID NO: 53 - DNA, 90 bp, synthetic construct
  misc_feature 1..90: note = Engineered antibody sequence
  source 1..90: mol_type = other DNA
gaggtgcagcttgtggagtctgggggaggcttggtccagcctggggggtccctgagactctcctgtgcagtctctggactcgacctcagt

SEQ ID NO: 54 - DNA, 15 bp, synthetic construct
  misc_feature 1..15: note = Engineered antibody sequence
  source 1..15: mol_type = other DNA
agctactacatgcaa

SEQ ID NO: 55 - DNA, 42 bp, synthetic construct
  misc_feature 1..42: note = Engineered antibody sequence
  source 1..42: mol_type = other DNA
tgggtccgtcaggctccagggaaggggctggagtgggtcgga

SEQ ID NO: 56 - DNA, 48 bp, synthetic construct
  misc_feature 1..48: note = Engineered antibody sequence
  source 1..48: mol_type = other DNA
gtcattggtatcaatgataacacatactacgcgagctgggcgaaaggc

SEQ ID NO: 57 - DNA, 96 bp, synthetic construct
  misc_feature 1..96: note = Engineered antibody sequence
  source 1..96: mol_type = other DNA
cgattcaccatctccagagacaattccaagaccacggtgtatcttcaaatgaacagcctgagagctgaggacactgctgtgtatttctgtgctaga

SEQ ID NO: 58 - skipped

SEQ ID NO: 59 - DNA, 33 bp, synthetic construct
  misc_feature 1..33: note = Engineered antibody sequence
  source 1..33: mol_type = other DNA
tggggccaagggaccctcgtcaccgtctcgagc

SEQ ID NO: 60 - DNA, 993 bp, synthetic construct; identical to SEQ ID NO: 20
  misc_feature 1..993: note = Engineered antibody sequence
  source 1..993: mol_type = other DNA

SEQ ID NO: 61 - protein, 219 aa, synthetic construct
  REGION 1..219: note = Engineered antibody sequence
QVLTQSPSSLSASVGDRVTINCQASQSVYDNNYLAWYQQKPGKVPKQLIYSTSTLASGVPSRFSGSGSGTDFTLTISSLQPEDVATYYCLGSYDCSSGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 113 aa, synthetic construct
  REGION 1..113: note = Engineered antibody sequence
QVLTQSPSSLSASVGDRVTINCQASQSVYDNNYLAWYQQKPGKVPKQLIYSTSTLASGVPSRFSGSGSGTDFTLTISSLQPEDVATYYCLGSYDCSSGDCFVFGGGTKVEIKR

SEQ ID NO: 63 - protein, 22 aa, synthetic construct
  REGION 1..22: note = Engineered antibody sequence
QVLTQSPSSLSASVGDRVTINC

SEQ ID NO: 64 - protein, 13 aa, synthetic construct
  REGION 1..13: note = Engineered antibody sequence
QASQSVYDNNYLA

SEQ ID NO: 65 - protein, 15 aa, synthetic construct
  REGION 1..15: note = Engineered antibody sequence
WYQQKPGKVPKQLIY

SEQ ID NO: 66 - protein, 7 aa, synthetic construct
  REGION 1..7: note = Engineered antibody sequence
STSTLAS

SEQ ID NO: 67 - protein, 32 aa, synthetic construct
  REGION 1..32: note = Engineered antibody sequence
GVPSRFSGSGSGTDFTLTISSLQPEDVATYYC

SEQ ID NO: 68 - protein, 13 aa, synthetic construct
  REGION 1..13: note = Engineered antibody sequence
LGSYDCSSGDCFV

SEQ ID NO: 69 - protein, 11 aa, synthetic construct
  REGION 1..11: note = Engineered antibody sequence
FGGGTKVEIKR

SEQ ID NO: 70 - protein, 106 aa, synthetic construct; identical to SEQ ID NO: 30
  REGION 1..106: note = Engineered antibody sequence

SEQ ID NO: 71 - DNA, 660 bp, synthetic construct
  misc_feature 1..660: note = Engineered antibody sequence
  source 1..660: mol_type = other DNA
caagtgctgacccagtctccatcctccctgtctgcatctgtaggagacagagtcaccatcaattgccaggccagtcagagtgtttatgataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctaggcagttatgattgtagtagtggtgattgttttgttttcggcggaggaaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 72 - DNA, 339 bp, synthetic construct
  misc_feature 1..339: note = Engineered antibody sequence
  source 1..339: mol_type = other DNA
caagtgctgacccagtctccatcctccctgtctgcatctgtaggagacagagtcaccatcaattgccaggccagtcagagtgtttatgataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctaggcagttatgattgtagtagtggtgattgttttgttttcggcggaggaaccaaggtggaaatcaaacgt

SEQ ID NO: 73 - DNA, 66 bp, synthetic construct
  misc_feature 1..66: note = Engineered antibody sequence
  source 1..66: mol_type = other DNA
caagtgctgacccagtctccatcctccctgtctgcatctgtaggagacagagtcaccatcaattgc

SEQ ID NO: 74 - DNA, 39 bp, synthetic construct; identical to SEQ ID NO: 34
  misc_feature 1..39: note = Engineered antibody sequence
  source 1..39: mol_type = other DNA

SEQ ID NO: 75 - DNA, 45 bp, synthetic construct
  misc_feature 1..45: note = Engineered antibody sequence
  source 1..45: mol_type = other DNA
tggtatcagcagaaaccagggaaagttcctaagcaactgatctat

SEQ ID NO: 76 - DNA, 21 bp, synthetic construct
  misc_feature 1..21: note = Engineered antibody sequence
  source 1..21: mol_type = other DNA
tctacatccactctggcatct

SEQ ID NO: 77 - DNA, 96 bp, synthetic construct
  misc_feature 1..96: note = Engineered antibody sequence
  source 1..96: mol_type = other DNA
ggggtcccatctcgtttcagtggcagtggatctgggacagatttcactctcaccatcagcagcctgcagcctgaagatgttgcaacttattactgt

SEQ ID NO: 78 - DNA, 39 bp, synthetic construct; identical to SEQ ID NO: 38
  misc_feature 1..39: note = Engineered antibody sequence
  source 1..39: mol_type = other DNA

SEQ ID NO: 79 - DNA, 33 bp, synthetic construct
  misc_feature 1..33: note = Engineered antibody sequence
  source 1..33: mol_type = other DNA
ttcggcggaggaaccaaggtggaaatcaaacgt

SEQ ID NO: 80 - DNA, 321 bp, synthetic construct; identical to SEQ ID NO: 40
  misc_feature 1..321: note = Engineered antibody sequence
  source 1..321: mol_type = other DNA

SEQ ID NO: 81 - protein, 441 aa, synthetic construct
  REGION 1..441: note = Engineered antibody sequence
EVQLVESGGGLVQPGGSLRLSCAVSGLDLSSYYMQWVRQAPGKGLEWVGVIGINDNTYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 111 aa, synthetic construct; identical to SEQ ID NO: 42
  REGION 1..111: note = Engineered antibody sequence

SEQ ID NO: 83 - protein, 30 aa, synthetic construct; identical to SEQ ID NO: 43
  REGION 1..30: note = Engineered antibody sequence

SEQ ID NO: 84 - protein, 5 aa, synthetic construct
  REGION 1..5: note = Engineered antibody sequence
SYYMQ

SEQ ID NO: 85 - protein, 14 aa, synthetic construct
  REGION 1..14: note = Engineered antibody sequence
WVRQAPGKGLEWVG

SEQ ID NO: 86 - protein, 16 aa, synthetic construct
  REGION 1..16: note = Engineered antibody sequence
VIGINDNTYYASWAKG

SEQ ID NO: 87 - protein, 32 aa, synthetic construct; identical to SEQ ID NO: 47
  REGION 1..32: note = Engineered antibody sequence

SEQ ID NO: 88 - skipped

SEQ ID NO: 89 - protein, 11 aa, synthetic construct
  REGION 1..11: note = Engineered antibody sequence
WGQGTLVTVSS

SEQ ID NO: 90 - protein, 330 aa, synthetic construct
  REGION 1..330: note = Engineered antibody sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggactcgacctcagtagctactacatgcaatgggtccgtcaggctccagggaaggggctggagtgggtcggagtcattggtatcaatgataacacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 92 - DNA, 333 bp, synthetic construct; identical to SEQ ID NO: 52
  misc_feature 1..333: note = Engineered antibody sequence
  source 1..333: mol_type = other DNA

SEQ ID NO: 93 - DNA, 90 bp, synthetic construct; identical to SEQ ID NO: 53
  misc_feature 1..90: note = Engineered antibody sequence
  source 1..90: mol_type = other DNA

SEQ ID NO: 94 - DNA, 15 bp, synthetic construct
  misc_feature 1..15: note = Engineered antibody sequence
  source 1..15: mol_type = other DNA
agctactacatgcaa

SEQ ID NO: 95 - DNA, 42 bp, synthetic construct; identical to SEQ ID NO: 55
  misc_feature 1..42: note = Engineered antibody sequence
  source 1..42: mol_type = other DNA

SEQ ID NO: 96 - DNA, 48 bp, synthetic construct; identical to SEQ ID NO: 56
  misc_feature 1..48: note = Engineered antibody sequence
  source 1..48: mol_type = other DNA

SEQ ID NO: 97 - DNA, 96 bp, synthetic construct; identical to SEQ ID NO: 57
  misc_feature 1..96: note = Engineered antibody sequence
  source 1..96: mol_type = other DNA

SEQ ID NO: 98 - skipped

SEQ ID NO: 99 - DNA, 33 bp, synthetic construct; identical to SEQ ID NO: 59
  misc_feature 1..33: note = Engineered antibody sequence
  source 1..33: mol_type = other DNA

SEQ ID NO: 100 - DNA, 993 bp, synthetic construct
  misc_feature 1..993: note = Engineered antibody sequence
  source 1..993: mol_type = other DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 101 - protein, 219 aa, synthetic construct; identical to SEQ ID NO: 61
  REGION 1..219: note = Engineered antibody sequence

SEQ ID NO: 102 - protein, 113 aa, synthetic construct; identical to SEQ ID NO: 62
  REGION 1..113: note = Engineered antibody sequence

SEQ ID NO: 103 - protein, 22 aa, synthetic construct
  REGION 1..22: note = Engineered antibody sequence
QVLTQSPSSLSASVGDRVTINC

SEQ ID NO: 104 - protein, 13 aa, synthetic construct
  REGION 1..13: note = Engineered antibody sequence
QASQSVYDNNYLA

SEQ ID NO: 105 - protein, 15 aa, synthetic construct
  REGION 1..15: note = Engineered antibody sequence
WYQQKPGKVPKQLIY

SEQ ID NO: 106 - protein, 7 aa, synthetic construct
  REGION 1..7: note = Engineered antibody sequence
STSTLAS

SEQ ID NO: 107 - protein, 32 aa, synthetic construct; identical to SEQ ID NO: 67
  REGION 1..32: note = Engineered antibody sequence

SEQ ID NO: 108 - protein, 13 aa, synthetic construct
  REGION 1..13: note = Engineered antibody sequence
LGSYDCSSGDCFV

SEQ ID NO: 109 - protein, 11 aa, synthetic construct
  REGION 1..11: note = Engineered antibody sequence
FGGGTKVEIKR

SEQ ID NO: 110 - protein, 106 aa, synthetic construct; identical to SEQ ID NO: 30
  REGION 1..106: note = Engineered antibody sequence

SEQ ID NO: 111 - DNA, 660 bp, synthetic construct; identical to SEQ ID NO: 71
  misc_feature 1..660: note = Engineered antibody sequence
  source 1..660: mol_type = other DNA

SEQ ID NO: 112 - DNA, 339 bp, synthetic construct; identical to SEQ ID NO: 72
  misc_feature 1..339: note = Engineered antibody sequence
  source 1..339: mol_type = other DNA

SEQ ID NO: 113 - DNA, 66 bp, synthetic construct; identical to SEQ ID NO: 73
  misc_feature 1..66: note = Engineered antibody sequence
  source 1..66: mol_type = other DNA

SEQ ID NO: 114 - DNA, 39 bp, synthetic construct; identical to SEQ ID NO: 34
  misc_feature 1..39: note = Engineered antibody sequence
  source 1..39: mol_type = other DNA

SEQ ID NO: 115 - DNA, 45 bp, synthetic construct; identical to SEQ ID NO: 75
  misc_feature 1..45: note = Engineered antibody sequence
  source 1..45: mol_type = other DNA

SEQ ID NO: 116 - DNA, 21 bp, synthetic construct
  misc_feature 1..21: note = Engineered antibody sequence
  source 1..21: mol_type = other DNA
tctacatccactctggcatct

SEQ ID NO: 117 - DNA, 96 bp, synthetic construct; identical to SEQ ID NO: 77
  misc_feature 1..96: note = Engineered antibody sequence
  source 1..96: mol_type = other DNA

SEQ ID NO: 118 - DNA, 39 bp, synthetic construct; identical to SEQ ID NO: 38
  misc_feature 1..39: note = Engineered antibody sequence
  source 1..39: mol_type = other DNA

SEQ ID NO: 119 - DNA, 33 bp, synthetic construct; identical to SEQ ID NO: 79
  misc_feature 1..33: note = Engineered antibody sequence
  source 1..33: mol_type = other DNA

SEQ ID NO: 120 - DNA, 321 bp, synthetic construct; identical to SEQ ID NO: 40
  misc_feature 1..321: note = Engineered antibody sequence
  source 1..321: mol_type = other DNA

SEQ ID NO: 121 - protein, 439 aa, synthetic construct
  REGION 1..439: note = Engineered antibody sequence
QSLEESGGRLVTPGTPLTLTCSVSGIDLSGYYMNWVRQAPGKGLEWIGVIGINGATYYASWAKGRFTISKTSSTTVDLKMTSLTTEDTATYFCARGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 109 aa, synthetic construct
  REGION 1..109: note = Engineered antibody sequence
QSLEESGGRLVTPGTPLTLTCSVSGIDLSGYYMNWVRQAPGKGLEWIGVIGINGATYYASWAKGRFTISKTSSTTVDLKMTSLTTEDTATYFCARGDIWGPGTLVTVSS

SEQ ID NO: 123 - protein, 29 aa, synthetic construct
  REGION 1..29: note = Engineered antibody sequence
QSLEESGGRLVTPGTPLTLTCSVSGIDLS

SEQ ID NO: 124 - protein, 5 aa, synthetic construct
  REGION 1..5: note = Engineered antibody sequence
GYYMN

SEQ ID NO: 125 - protein, 14 aa, synthetic construct
  REGION 1..14: note = Engineered antibody sequence
WVRQAPGKGLEWIG

SEQ ID NO: 126 - protein, 16 aa, synthetic construct
  REGION 1..16: note = Engineered antibody sequence
VIGINGATYYASWAKG

SEQ ID NO: 127 - protein, 31 aa, synthetic construct
  REGION 1..31: note = Engineered antibody sequence
RFTISKTSSTTVDLKMTSLTTEDTATYFCAR

SEQ ID NO: 128 - skipped

SEQ ID NO: 129 - protein, 11 aa, synthetic construct
  REGION 1..11: note = Engineered antibody sequence
WGPGTLVTVSS

SEQ ID NO: 130 - protein, 330 aa, synthetic construct; identical to SEQ ID NO: 10
  REGION 1..330: note = Engineered antibody sequence

SEQ ID NO: 131 - DNA, 1320 bp, synthetic construct
  misc_feature 1..1320: note = Engineered antibody sequence
  source 1..1320: mol_type = other DNA
cagtcgctggaggagtccgggggtcgcctggtcacgcctgggacacccctgacactcacctgttccgtctctggcatcgacctcagtggctactacatgaactgggtccgccaggctccagggaaggggctggaatggatcggagtcattggtattaatggtgccacatactacgcgagctgggcgaaaggccgattcaccatctccaaaacctcgtcgaccacggtggatctgaaaatgaccagtctgacaaccgaggacacggccacctatttctgtgccagaggggacatctggggcccgggcaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 132 - DNA, 327 bp, synthetic construct
  misc_feature 1..327: note = Engineered antibody sequence
  source 1..327: mol_type = other DNA
cagtcgctggaggagtccgggggtcgcctggtcacgcctgggacacccctgacactcacctgttccgtctctggcatcgacctcagtggctactacatgaactgggtccgccaggctccagggaaggggctggaatggatcggagtcattggtattaatggtgccacatactacgcgagctgggcgaaaggccgattcaccatctccaaaacctcgtcgaccacggtggatctgaaaatgaccagtctgacaaccgaggacacggccacctatttctgtgccagaggggacatctggggcccgggcaccctcgtcaccgtctcgagc

SEQ ID NO: 133 - DNA, 87 bp, synthetic construct
  misc_feature 1..87: note = Engineered antibody sequence
  source 1..87: mol_type = other DNA
cagtcgctggaggagtccgggggtcgcctggtcacgcctgggacacccctgacactcacctgttccgtctctggcatcgacctcagt

SEQ ID NO: 134 - DNA, 15 bp, synthetic construct
  misc_feature 1..15: note = Engineered antibody sequence
  source 1..15: mol_type = other DNA
ggctactacatgaac

SEQ ID NO: 135 - DNA, 42 bp, synthetic construct; identical to SEQ ID NO: 15
  misc_feature 1..42: note = Engineered antibody sequence
  source 1..42: mol_type = other DNA

SEQ ID NO: 136 - DNA, 48 bp, synthetic construct
  misc_feature 1..48: note = Engineered antibody sequence
  source 1..48: mol_type = other DNA
gtcattggtattaatggtgccacatactacgcgagctgggcgaaaggc

SEQ ID NO: 137 - DNA, 93 bp, synthetic construct
  misc_feature 1..93: note = Engineered antibody sequence
  source 1..93: mol_type = other DNA
cgattcaccatctccaaaacctcgtcgaccacggtggatctgaaaatgaccagtctgacaaccgaggacacggccacctatttctgtgccaga

SEQ ID NO: 138 - skipped

SEQ ID NO: 139 - DNA, 33 bp, synthetic construct
  misc_feature 1..33: note = Engineered antibody sequence
  source 1..33: mol_type = other DNA
tggggcccgggcaccctcgtcaccgtctcgagc

SEQ ID NO: 140 - DNA, 993 bp, synthetic construct; identical to SEQ ID NO: 20
  misc_feature 1..993: note = Engineered antibody sequence
  source 1..993: mol_type = other DNA

SEQ ID NO: 141 - protein, 219 aa, synthetic construct
  REGION 1..219: note = Engineered antibody sequence
QVLTQTPSPVSAAVGSTVTINCQASQSVYHNTYLAWYQQKPGQPPKQLIYDASTLASGVPSRFSGSGSGTQFTLTISGVQCNDAAAYYCLGSYDCTNGDCFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 142 - protein, 113 aa, synthetic construct
  REGION 1..113: note = Engineered antibody sequence
QVLTQTPSPVSAAVGSTVTINCQASQSVYHNTYLAWYQQKPGQPPKQLIYDASTLASGVPSRFSGSGSGTQFTLTISGVQCNDAAAYYCLGSYDCTNGDCFVFGGGTEVVVKR

SEQ ID NO: 143 - protein, 22 aa, synthetic construct
  REGION 1..22: note = Engineered antibody sequence
QVLTQTPSPVSAAVGSTVTINC

SEQ ID NO: 144 - protein, 13 aa, synthetic construct
  REGION 1..13: note = Engineered antibody sequence
QASQSVYHNTYLA

SEQ ID NO: 145 - protein, 15 aa, synthetic construct
  REGION 1..15: note = Engineered antibody sequence
WYQQKPGQPPKQLIY

SEQ ID NO: 146 - protein, 7 aa, synthetic construct
  REGION 1..7: note = Engineered antibody sequence
DASTLAS

SEQ ID NO: 147 - protein, 32 aa, synthetic construct
  REGION 1..32: note = Engineered antibody sequence
GVPSRFSGSGSGTQFTLTISGVQCNDAAAYYC

SEQ ID NO: 148 - protein, 13 aa, synthetic construct
  REGION 1..13: note = Engineered antibody sequence
LGSYDCTNGDCFV

SEQ ID NO: 149 - protein, 11 aa, synthetic construct
  REGION 1..11: note = Engineered antibody sequence
FGGGTEVVVKR

SEQ ID NO: 150 - protein, 106 aa, synthetic construct; identical to SEQ ID NO: 30
  REGION 1..106: note = Engineered antibody sequence

SEQ ID NO: 151 - DNA, 660 bp, synthetic construct
  misc_feature 1..660: note = Engineered antibody sequence
  source 1..660: mol_type = other DNA
caagtgctgacccagactccatcccccgtgtctgcagctgtgggaagcacagtcaccatcaattgccaggccagtcagagtgtttatcataacacctacctggcctggtatcagcagaaaccagggcagcctcccaaacaactgatctatgatgcatccactctggcgtctggggtcccatcgcggttcagcggcagtggatctgggacacagttcactctcaccatcagcggcgtgcagtgtaacgatgctgccgcttactactgtctgggcagttatgattgtactaatggtgattgttttgttttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 152 - DNA, 339 bp, synthetic construct
  misc_feature 1..339: note = Engineered antibody sequence
  source 1..339: mol_type = other DNA
caagtgctgacccagactccatcccccgtgtctgcagctgtgggaagcacagtcaccatcaattgccaggccagtcagagtgtttatcataacacctacctggcctggtatcagcagaaaccagggcagcctcccaaacaactgatctatgatgcatccactctggcgtctggggtcccatcgcggttcagcggcagtggatctgggacacagttcactctcaccatcagcggcgtgcagtgtaacgatgctgccgcttactactgtctgggcagttatgattgtactaatggtgattgttttgttttcggcggagggaccgaggtggtggtcaaacgt

SEQ ID NO: 153 - DNA, 66 bp, synthetic construct
  misc_feature 1..66: note = Engineered antibody sequence
  source 1..66: mol_type = other DNA
caagtgctgacccagactccatcccccgtgtctgcagctgtgggaagcacagtcaccatcaattgc

SEQ ID NO: 154 - DNA, 39 bp, synthetic construct
  misc_feature 1..39: note = Engineered antibody sequence
  source 1..39: mol_type = other DNA
caggccagtcagagtgtttatcataacacctacctggcc

SEQ ID NO: 155 - DNA, 45 bp, synthetic construct
  misc_feature 1..45: note = Engineered antibody sequence
  source 1..45: mol_type = other DNA
tggtatcagcagaaaccagggcagcctcccaaacaactgatctat

SEQ ID NO: 156 - DNA, 21 bp, synthetic construct
  misc_feature 1..21: note = Engineered antibody sequence
  source 1..21: mol_type = other DNA
gatgcatccactctggcgtct

SEQ ID NO: 157 - DNA, 96 bp, synthetic construct
  misc_feature 1..96: note = Engineered antibody sequence
  source 1..96: mol_type = other DNA
ggggtcccatcgcggttcagcggcagtggatctgggacacagttcactctcaccatcagcggcgtgcagtgtaacgatgctgccgcttactactgt

SEQ ID NO: 158 - DNA, 39 bp, synthetic construct
  misc_feature 1..39: note = Engineered antibody sequence
  source 1..39: mol_type = other DNA
ctgggcagttatgattgtactaatggtgattgttttgtt

SEQ ID NO: 159 - DNA, 33 bp, synthetic construct; identical to SEQ ID NO: 39
  misc_feature 1..33: note = Engineered antibody sequence
  source 1..33: mol_type = other DNA

SEQ ID NO: 160 - DNA, 321 bp, synthetic construct; identical to SEQ ID NO: 40
  misc_feature 1..321: note = Engineered antibody sequence
  source 1..321: mol_type = other DNA

SEQ ID NO: 161 - protein, 441 aa, synthetic construct
  REGION 1..441: note = Engineered antibody sequence
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 111 aa, synthetic construct
  REGION 1..111: note = Engineered antibody sequence
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSS

SEQ ID NO: 163 - protein, 30 aa, synthetic construct
  REGION 1..30: note = Engineered antibody sequence
EVQLVESGGGLVQPGGSLRLSCAVSGIDLS

SEQ ID NO: 164 - protein, 5 aa, synthetic construct
  REGION 1..5: note = Engineered antibody sequence
GYYMN

SEQ ID NO: 165 - protein, 14 aa, synthetic construct
  REGION 1..14: note = Engineered antibody sequence
WVRQAPGKGLEWVG

SEQ ID NO: 166 - protein, 16 aa, synthetic construct
  REGION 1..16: note = Engineered antibody sequence
VIGINGATYYASWAKG

SEQ ID NO: 167 - protein, 32 aa, synthetic construct; identical to SEQ ID NO: 47
  REGION 1..32: note = Engineered antibody sequence

SEQ ID NO: 168 - skipped

SEQ ID NO: 169 - protein, 11 aa, synthetic construct
  REGION 1..11: note = Engineered antibody sequence
WGQGTLVTVSS

SEQ ID NO: 170 - protein, 330 aa, synthetic construct; identical to SEQ ID NO: 10
  REGION 1..330: note = Engineered antibody sequence

SEQ ID NO: 171 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcgacctcagtggctactacatgaactgggtccgtcaggctccagggaaggggctggagtgggtcggagtcattggtattaatggtgccacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 172 - DNA, 333 bp, synthetic construct
  misc_feature 1..333: note = Engineered antibody sequence
  source 1..333: mol_type = other DNA
gaggtgcagcttgtggagtctgggggaggcttggtccagcctggggggtccctgagactctcctgtgcagtctctggaatcgacctcagtggctactacatgaactgggtccgtcaggctccagggaaggggctggagtgggtcggagtcattggtattaatggtgccacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagc

SEQ ID NO: 173 - DNA, 90 bp, synthetic construct
  misc_feature 1..90: note = Engineered antibody sequence
  source 1..90: mol_type = other DNA
gaggtgcagcttgtggagtctgggggaggcttggtccagcctggggggtccctgagactctcctgtgcagtctctggaatcgacctcagt

SEQ ID NO: 174 - DNA, 15 bp, synthetic construct
  misc_feature 1..15: note = Engineered antibody sequence
  source 1..15: mol_type = other DNA
ggctactacatgaac

SEQ ID NO: 175 - DNA, 42 bp, synthetic construct; identical to SEQ ID NO: 55
  misc_feature 1..42: note = Engineered antibody sequence
  source 1..42: mol_type = other DNA

SEQ ID NO: 176 - DNA, 48 bp, synthetic construct; identical to SEQ ID NO: 136
  misc_feature 1..48: note = Engineered antibody sequence
  source 1..48: mol_type = other DNA

SEQ ID NO: 177 - DNA, 96 bp, synthetic construct; identical to SEQ ID NO: 57
  misc_feature 1..96: note = Engineered antibody sequence
  source 1..96: mol_type = other DNA

SEQ ID NO: 178 - skipped

SEQ ID NO: 179 - DNA, 33 bp, synthetic construct; identical to SEQ ID NO: 59
  misc_feature 1..33: note = Engineered antibody sequence
  source 1..33: mol_type = other DNA

SEQ ID NO: 180 - DNA, 993 bp, synthetic construct; identical to SEQ ID NO: 20
  misc_feature 1..993: note = Engineered antibody sequence
  source 1..993: mol_type = other DNA

SEQ ID NO: 181 - protein, 219 aa, synthetic construct
  REGION 1..219: note = Engineered antibody sequence
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 113 aa, synthetic construct
  REGION 1..113: note = Engineered antibody sequence
QVLTQSPSSLSASVGDRVTINCQASQSVYHNTYLAWYQQKPGKVPKQLIYDASTLASGVPSRFSGSGSGTDFTLTISSLQPEDVATYYCLGSYDCTNGDCFVFGGGTKVEIKR

SEQ ID NO: 183 - protein, 22 aa, synthetic construct
  REGION 1..22: note = Engineered antibody sequence
QVLTQSPSSLSASVGDRVTINC

SEQ ID NO: 184 - protein, 13 aa, synthetic construct
  REGION 1..13: note = Engineered antibody sequence
QASQSVYHNTYLA

SEQ ID NO: 185 - protein, 15 aa, synthetic construct
  REGION 1..15: note = Engineered antibody sequence
WYQQKPGKVPKQLIY

SEQ ID NO: 186 - protein, 7 aa, synthetic construct
  REGION 1..7: note = Engineered antibody sequence
DASTLAS

SEQ ID NO: 187 - protein, 32 aa, synthetic construct; identical to SEQ ID NO: 67
  REGION 1..32: note = Engineered antibody sequence

SEQ ID NO: 188 - protein, 13 aa, synthetic construct
  REGION 1..13: note = Engineered antibody sequence
LGSYDCTNGDCFV

SEQ ID NO: 189 - protein, 11 aa, synthetic construct
  REGION 1..11: note = Engineered antibody sequence
FGGGTKVEIKR

SEQ ID NO: 190 - protein, 106 aa, synthetic construct; identical to SEQ ID NO: 30
  REGION 1..106: note = Engineered antibody sequence

SEQ ID NO: 191 - DNA, 660 bp, synthetic construct
  misc_feature 1..660: note = Engineered antibody sequence
  source 1..660: mol_type = other DNA
caagtgctgacccagtctccatcctccctgtctgcatctgtaggagacagagtcaccatcaattgccaggccagtcagagtgtttatcataacacctacctggcctggtatcagcagaaaccagggaaagttcctaagcaactgatctatgatgcatccactctggcatctggggtcccatctcgtttcagtggcagtggatctgggacagatttcactctcaccatcagcagcctgcagcctgaagatgttgcaacttattactgtctgggcagttatgattgtactaatggtgattgttttgttttcggcggaggaaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 192 - DNA, 339 bp, synthetic construct
  misc_feature 1..339: note = Engineered antibody sequence
  source 1..339: mol_type = other DNA
caagtgctgacccagtctccatcctccctgtctgcatctgtaggagacagagtcaccatcaattgccaggccagtcagagtgtttatcataacacctacctggcctggtatcagcagaaaccagggaaagttcctaagcaactgatctatgatgcatccactctggcatctggggtcccatctcgtttcagtggcagtggatctgggacagatttcactctcaccatcagcagcctgcagcctgaagatgttgcaacttattactgtctgggcagttatgattgtactaatggtgattgttttgttttcggcggaggaaccaaggtggaaatcaaacgt

SEQ ID NO: 193 - DNA, 66 bp, synthetic construct; identical to SEQ ID NO: 73
  misc_feature 1..66: note = Engineered antibody sequence
  source 1..66: mol_type = other DNA

SEQ ID NO: 194 - DNA, 39 bp, synthetic construct; identical to SEQ ID NO: 154
  misc_feature 1..39: note = Engineered antibody sequence
  source 1..39: mol_type = other DNA

SEQ ID NO: 195 - DNA, 45 bp, synthetic construct; identical to SEQ ID NO: 75
  misc_feature 1..45: note = Engineered antibody sequence
  source 1..45: mol_type = other DNA

SEQ ID NO: 196 - DNA, 21 bp, synthetic construct
  misc_feature 1..21: note = Engineered antibody sequence
  source 1..21: mol_type = other DNA
gatgcatccactctggcatct

SEQ ID NO: 197 - DNA, 96 bp, synthetic construct; identical to SEQ ID NO: 77
  misc_feature 1..96: note = Engineered antibody sequence
  source 1..96: mol_type = other DNA

SEQ ID NO: 198 - DNA, 39 bp, synthetic construct; identical to SEQ ID NO: 158
  misc_feature 1..39: note = Engineered antibody sequence
  source 1..39: mol_type = other DNA

SEQ ID NO: 199 - DNA, 33 bp, synthetic construct; identical to SEQ ID NO: 79
  misc_feature 1..33: note = Engineered antibody sequence
  source 1..33: mol_type = other DNA

SEQ ID NO: 200 - DNA, 321 bp, synthetic construct; identical to SEQ ID NO: 40
  misc_feature 1..321: note = Engineered antibody sequence
  source 1..321: mol_type = other DNA

SEQ ID NO: 201 - protein, 441 aa, synthetic construct
  REGION 1..441: note = Engineered antibody sequence
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 111 aa, synthetic construct; identical to SEQ ID NO: 162
  REGION 1..111: note = Engineered antibody sequence

SEQ ID NO: 203 - protein, 30 aa, synthetic construct; identical to SEQ ID NO: 163
  REGION 1..30: note = Engineered antibody sequence

SEQ ID NO: 204 - protein, 5 aa, synthetic construct
  REGION 1..5: note = Engineered antibody sequence
GYYMN

SEQ ID NO: 205 - protein, 14 aa, synthetic construct
  REGION 1..14: note = Engineered antibody sequence
WVRQAPGKGLEWVG

SEQ ID NO: 206 - protein, 16 aa, synthetic construct
  REGION 1..16: note = Engineered antibody sequence
VIGINGATYYASWAKG

SEQ ID NO: 207 - protein, 32 aa, synthetic construct; identical to SEQ ID NO: 47
  REGION 1..32: note = Engineered antibody sequence

SEQ ID NO: 208 - skipped

SEQ ID NO: 209 - protein, 11 aa, synthetic construct
  REGION 1..11: note = Engineered antibody sequence
WGQGTLVTVSS

SEQ ID NO: 210 - protein, 330 aa, synthetic construct; identical to SEQ ID NO: 90
  REGION 1..330: note = Engineered antibody sequence

SEQ ID NO: 211 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcgacctcagtggctactacatgaactgggtccgtcaggctccagggaaggggctggagtgggtcggagtcattggtattaatggtgccacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 212 - DNA, 333 bp, synthetic construct; identical to SEQ ID NO: 172
  misc_feature 1..333: note = Engineered antibody sequence
  source 1..333: mol_type = other DNA

SEQ ID NO: 213 - DNA, 90 bp, synthetic construct; identical to SEQ ID NO: 173
  misc_feature 1..90: note = Engineered antibody sequence
  source 1..90: mol_type = other DNA

SEQ ID NO: 214 - DNA, 15 bp, synthetic construct
  misc_feature 1..15: note = Engineered antibody sequence
  source 1..15: mol_type = other DNA
ggctactacatgaac

SEQ ID NO: 215 - DNA, 42 bp, synthetic construct; identical to SEQ ID NO: 55
  misc_feature 1..42: note = Engineered antibody sequence
  source 1..42: mol_type = other DNA

SEQ ID NO: 216 - DNA, 48 bp, synthetic construct; identical to SEQ ID NO: 136
  misc_feature 1..48: note = Engineered antibody sequence
  source 1..48: mol_type = other DNA

SEQ ID NO: 217 - DNA, 96 bp, synthetic construct; identical to SEQ ID NO: 57
  misc_feature 1..96: note = Engineered antibody sequence
  source 1..96: mol_type = other DNA

SEQ ID NO: 218 - skipped

SEQ ID NO: 219 - DNA, 33 bp, synthetic construct; identical to SEQ ID NO: 59
  misc_feature 1..33: note = Engineered antibody sequence
  source 1..33: mol_type = other DNA

SEQ ID NO: 220 - DNA, 993 bp, synthetic construct; identical to SEQ ID NO: 100
  misc_feature 1..993: note = Engineered antibody sequence
  source 1..993: mol_type = other DNA

SEQ ID NO: 221 - protein, 219 aa, synthetic construct; identical to SEQ ID NO: 181
  REGION 1..219: note = Engineered antibody sequence

SEQ ID NO: 222 - protein, 113 aa, synthetic construct; identical to SEQ ID NO: 182
  REGION 1..113: note = Engineered antibody sequence

SEQ ID NO: 223 - protein, 22 aa, synthetic construct
  REGION 1..22: note = Engineered antibody sequence
QVLTQSPSSLSASVGDRVTINC

SEQ ID NO: 224 - protein, 13 aa, synthetic construct
  REGION 1..13: note = Engineered antibody sequence
QASQSVYHNTYLA

SEQ ID NO: 225 - protein, 15 aa, synthetic construct
  REGION 1..15: note = Engineered antibody sequence
WYQQKPGKVPKQLIY

SEQ ID NO: 226 - protein, 7 aa, synthetic construct
  REGION 1..7: note = Engineered antibody sequence
DASTLAS

SEQ ID NO: 227 - protein, 32 aa, synthetic construct; identical to SEQ ID NO: 67
  REGION 1..32: note = Engineered antibody sequence

SEQ ID NO: 228 - protein, 13 aa, synthetic construct
  REGION 1..13: note = Engineered antibody sequence
LGSYDCTNGDCFV

SEQ ID NO: 229 - protein, 11 aa, synthetic construct
  REGION 1..11: note = Engineered antibody sequence
FGGGTKVEIKR

SEQ ID NO: 230 - protein, 106 aa, synthetic construct; identical to SEQ ID NO: 30
  REGION 1..106: note = Engineered antibody sequence

SEQ ID NO: 231 - DNA, 660 bp, synthetic construct; identical to SEQ ID NO: 191
  misc_feature 1..660: note = Engineered antibody sequence
  source 1..660: mol_type = other DNA

SEQ ID NO: 232 - DNA, 339 bp, synthetic construct; identical to SEQ ID NO: 192
  misc_feature 1..339: note = Engineered antibody sequence
  source 1..339: mol_type = other DNA

SEQ ID NO: 233 - DNA, 66 bp, synthetic construct; identical to SEQ ID NO: 73
  misc_feature 1..66: note = Engineered antibody sequence
  source 1..66: mol_type = other DNA

SEQ ID NO: 234 - DNA, 39 bp, synthetic construct; identical to SEQ ID NO: 154
  misc_feature 1..39: note = Engineered antibody sequence
  source 1..39: mol_type = other DNA

SEQ ID NO: 235 - DNA, 45 bp, synthetic construct; identical to SEQ ID NO: 75
  misc_feature 1..45: note = Engineered antibody sequence
  source 1..45: mol_type = other DNA

SEQ ID NO: 236 - DNA, 21 bp, synthetic construct
  misc_feature 1..21: note = Engineered antibody sequence
  source 1..21: mol_type = other DNA
gatgcatccactctggcatct

SEQ ID NO: 237 - DNA, 96 bp, synthetic construct; identical to SEQ ID NO: 77
  misc_feature 1..96: note = Engineered antibody sequence
  source 1..96: mol_type = other DNA

SEQ ID NO: 238 - DNA, 39 bp, synthetic construct; identical to SEQ ID NO: 158
  misc_feature 1..39: note = Engineered antibody sequence
  source 1..39: mol_type = other DNA

SEQ ID NO: 239 - DNA, 33 bp, synthetic construct; identical to SEQ ID NO: 79
  misc_feature 1..33: note = Engineered antibody sequence
  source 1..33: mol_type = other DNA

SEQ ID NO: 240 - DNA, 321 bp, synthetic construct; identical to SEQ ID NO: 40
  misc_feature 1..321: note = Engineered antibody sequence
  source 1..321: mol_type = other DNA

SEQ ID NO: 241 - protein, 440 aa, synthetic construct
  REGION 1..440: note = Engineered antibody sequence
QEQLKESGGRLVTPGTSLTLTCTVSGIDLSNHYMQWVRQAPGKGLEWIGVVGINGRTYYASWAKGRFTISRTSSTTVDLKMTRLTTEDTATYFCARGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 110 aa, synthetic construct
  REGION 1..110: note = Engineered antibody sequence
QEQLKESGGRLVTPGTSLTLTCTVSGIDLSNHYMQWVRQAPGKGLEWIGVVGINGRTYYASWAKGRFTISRTSSTTVDLKMTRLTTEDTATYFCARGDIWGPGTLVTVSS

SEQ ID NO: 243 - protein, 30 aa, synthetic construct
  REGION 1..30: note = Engineered antibody sequence
QEQLKESGGRLVTPGTSLTLTCTVSGIDLS

SEQ ID NO: 244 - protein, 5 aa, synthetic construct
  REGION 1..5: note = Engineered antibody sequence
NHYMQ

SEQ ID NO: 245 - protein, 14 aa, synthetic construct
  REGION 1..14: note = Engineered antibody sequence
WVRQAPGKGLEWIG

SEQ ID NO: 246 - protein, 16 aa, synthetic construct
  REGION 1..16: note = Engineered antibody sequence
VVGINGRTYYASWAKG

SEQ ID NO: 247 - protein, 31 aa, synthetic construct
  REGION 1..31: note = Engineered antibody sequence
RFTISRTSSTTVDLKMTRLTTEDTATYFCAR

SEQ ID NO: 248 - skipped

SEQ ID NO: 249 - protein, 11 aa, synthetic construct
  REGION 1..11: note = Engineered antibody sequence
WGPGTLVTVSS

SEQ ID NO: 250 - protein, 330 aa, synthetic construct; identical to SEQ ID NO: 10
  REGION 1..330: note = Engineered antibody sequence

SEQ ID NO: 251 - DNA, 1323 bp, synthetic construct
  misc_feature 1..1323: note = Engineered antibody sequence
  source 1..1323: mol_type = other DNA
caggagcagctgaaggagtccgggggtcgcctggtcacgcctgggacatccctgacactcacctgcaccgtctctggaatcgacctcagtaaccactacatgcaatgggtccgccaggctccagggaaggggctggagtggatcggagtcgttggtattaatggtcgcacatactacgcgagctgggcgaaaggccgattcaccatctccagaacctcgtcgaccacggtggatctgaaaatgaccaggctgacaaccgaggacacggccacctatttctgtgccagaggggacatctggggcccaggcaccctggtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 252 - DNA, 330 bp, synthetic construct
  misc_feature 1..330: note = Engineered antibody sequence
  source 1..330: mol_type = other DNA
caggagcagctgaaggagtccgggggtcgcctggtcacgcctgggacatccctgacactcacctgcaccgtctctggaatcgacctcagtaaccactacatgcaatgggtccgccaggctccagggaaggggctggagtggatcggagtcgttggtattaatggtcgcacatactacgcgagctgggcgaaaggccgattcaccatctccagaacctcgtcgaccacggtggatctgaaaatgaccaggctgacaaccgaggacacggccacctatttctgtgccagaggggacatctggggcccaggcaccctggtcaccgtctcgagc

SEQ ID NO: 253 - DNA, 90 bp, synthetic construct
  misc_feature 1..90: note = Engineered antibody sequence
  source 1..90: mol_type = other DNA
caggagcagctgaaggagtccgggggtcgcctggtcacgcctgggacatccctgacactcacctgcaccgtctctggaatcgacctcagt

SEQ ID NO: 254 - DNA, 15 bp, synthetic construct
  misc_feature 1..15: note = Engineered antibody sequence
  source 1..15: mol_type = other DNA
aaccactacatgcaa

SEQ ID NO: 255 - DNA, 42 bp, synthetic construct
  misc_feature 1..42: note = Engineered antibody sequence
  source 1..42: mol_type = other DNA
tgggtccgccaggctccagggaaggggctggagtggatcgga

SEQ ID NO: 256 - DNA, 48 bp, synthetic construct
  misc_feature 1..48: note = Engineered antibody sequence
  source 1..48: mol_type = other DNA
gtcgttggtattaatggtcgcacatactacgcgagctgggcgaaaggc

SEQ ID NO: 257 - DNA, 93 bp, synthetic construct
  misc_feature 1..93: note = Engineered antibody sequence
  source 1..93: mol_type = other DNA
cgattcaccatctccagaacctcgtcgaccacggtggatctgaaaatgaccaggctgacaaccgaggacacggccacctatttctgtgccaga

SEQ ID NO: 258 - skipped

SEQ ID NO: 259 - DNA, 33 bp, synthetic construct
  misc_feature 1..33: note = Engineered antibody sequence
  source 1..33: mol_type = other DNA
tggggcccaggcaccctggtcaccgtctcgagc

SEQ ID NO: 260 - DNA, 993 bp, synthetic construct; identical to SEQ ID NO: 20
  misc_feature 1..993: note = Engineered antibody sequence
  source 1..993: mol_type = other DNA

SEQ ID NO: 261 - protein, 219 aa, synthetic construct
  REGION 1..219: note = Engineered antibody sequence
QVLTQTASPVSAAVGSTVTINCQASQSVYNYNYLAWYQQKPGQPPKQLIYSTSTLASGVSSRFKGSGSGTQFTLTISDVQCDDAATYYCLGSYDCSTGDCFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - protein, 113 aa, synthetic construct
  REGION 1..113: note = Engineered antibody sequence
QVLTQTASPVSAAVGSTVTINCQASQSVYNYNYLAWYQQKPGQPPKQLIYSTSTLASGVSSRFKGSGSGTQFTLTISDVQCDDAATYYCLGSYDCSTGDCFVFGGGTEVVVKR

SEQ ID NO: 263 - protein, 22 aa, synthetic construct
  REGION 1..22: note = Engineered antibody sequence
QVLTQTASPVSAAVGSTVTINC

SEQ ID NO: 264 - protein, 13 aa, synthetic construct
  REGION 1..13: note = Engineered antibody sequence
QASQSVYNYNYLA

SEQ ID NO: 265 - protein, 15 aa, synthetic construct
  REGION 1..15: note = Engineered antibody sequence
WYQQKPGQPPKQLIY

SEQ ID NO: 266 - protein, 7 aa, synthetic construct
  REGION 1..7: note = Engineered antibody sequence
STSTLAS

SEQ ID NO: 267 - protein, 32 aa, synthetic construct
  REGION 1..32: note = Engineered antibody sequence
GVSSRFKGSGSGTQFTLTISDVQCDDAATYYC

SEQ ID NO: 268 - protein, 13 aa, synthetic construct
  REGION 1..13: note = Engineered antibody sequence
LGSYDCSTGDCFV

SEQ ID NO: 269 - protein, 11 aa, synthetic construct
  REGION 1..11: note = Engineered antibody sequence
FGGGTEVVVKR

SEQ ID NO: 270 - protein, 106 aa, synthetic construct; identical to SEQ ID NO: 30
  REGION 1..106: note = Engineered antibody sequence

SEQ ID NO: 271 - DNA, 660 bp, synthetic construct
  misc_feature 1..660: note = Engineered antibody sequence
  source 1..660: mol_type = other DNA
caagtgctgacccagactgcatcccccgtgtctgcagctgtgggaagcacagtcaccatcaattgccaggccagtcagagtgtttataattacaactaccttgcctggtatcagcagaaaccagggcagcctcccaagcaactgatctattctacatccactctggcatctggggtctcatcgcgattcaaaggcagtggatctgggacacagttcactctcaccatcagcgacgtgcagtgtgacgatgctgccacttactactgtctaggcagttatgactgtagtactggtgattgttttgttttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 272 - DNA, 339 bp, synthetic construct
  misc_feature 1..339: note = Engineered antibody sequence
  source 1..339: mol_type = other DNA
caagtgctgacccagactgcatcccccgtgtctgcagctgtgggaagcacagtcaccatcaattgccaggccagtcagagtgtttataattacaactaccttgcctggtatcagcagaaaccagggcagcctcccaagcaactgatctattctacatccactctggcatctggggtctcatcgcgattcaaaggcagtggatctgggacacagttcactctcaccatcagcgacgtgcagtgtgacgatgctgccacttactactgtctaggcagttatgactgtagtactggtgattgttttgttttcggcggagggaccgaggtggtggtcaaacgt

SEQ ID NO: 273 - DNA, 66 bp, synthetic construct; identical to SEQ ID NO: 33
  misc_feature 1..66: note = Engineered antibody sequence
  source 1..66: mol_type = other DNA

SEQ ID NO: 274 - DNA, 39 bp, synthetic construct
  misc_feature 1..39: note = Engineered antibody sequence
  source 1..39: mol_type = other DNA
caggccagtcagagtgtttataattacaactaccttgcc

SEQ ID NO: 275 - DNA, 45 bp, synthetic construct; identical to SEQ ID NO: 35
  misc_feature 1..45: note = Engineered antibody sequence
  source 1..45: mol_type = other DNA

SEQ ID NO: 276 - DNA, 21 bp, synthetic construct
  misc_feature 1..21: note = Engineered antibody sequence
  source 1..21: mol_type = other DNA
tctacatccactctggcatct

SEQ ID NO: 277 - DNA, 96 bp, synthetic construct
  misc_feature 1..96: note = Engineered antibody sequence
  source 1..96: mol_type = other DNA
ggggtctcatcgcgattcaaaggcagtggatctgggacacagttcactctcaccatcagcgacgtgcagtgtgacgatgctgccacttactactgt

SEQ ID NO: 278 - DNA, 39 bp, synthetic construct
  misc_feature 1..39: note = Engineered antibody sequence
  source 1..39: mol_type = other DNA
ctaggcagttatgactgtagtactggtgattgttttgtt

SEQ ID NO: 279 - DNA, 33 bp, synthetic construct; identical to SEQ ID NO: 39
  misc_feature 1..33: note = Engineered antibody sequence
  source 1..33: mol_type = other DNA

SEQ ID NO: 280 - DNA, 321 bp, synthetic construct; identical to SEQ ID NO: 40
  misc_feature 1..321: note = Engineered antibody sequence
  source 1..321: mol_type = other DNA

SEQ ID NO: 281 - protein, 441 aa, synthetic construct
  REGION 1..441: note = Engineered antibody sequence
EVQLVESGGGLVQPGGSLRLSCAVSGIDLSNHYMQWVRQAPGKGLEWVGVVGINGRTYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 111 aa, synthetic construct
  REGION 1..111: note = Engineered antibody sequence
EVQLVESGGGLVQPGGSLRLSCAVSGIDLSNHYMQWVRQAPGKGLEWVGVVGINGRTYYASWAKGRFTISRDNSKTTVYLQMNSLRAEDTAVYFCARGDIWGQGTLVTVSS

SEQ ID NO: 283 - protein, 30 aa, synthetic construct; identical to SEQ ID NO: 163
  REGION 1..30: note = Engineered antibody sequence

SEQ ID NO: 284 - protein, 5 aa, synthetic construct
  REGION 1..5: note = Engineered antibody sequence
NHYMQ

SEQ ID NO: 285 - protein, 14 aa, synthetic construct
  REGION 1..14: note = Engineered antibody sequence
WVRQAPGKGLEWVG

SEQ ID NO: 286 - protein, 16 aa, synthetic construct
  REGION 1..16: note = Engineered antibody sequence
VVGINGRTYYASWAKG

SEQ ID NO: 287 - protein, 32 aa, synthetic construct; identical to SEQ ID NO: 47
  REGION 1..32: note = Engineered antibody sequence

SEQ ID NO: 288 - skipped

SEQ ID NO: 289 - protein, 11 aa, synthetic construct
  REGION 1..11: note = Engineered antibody sequence
WGQGTLVTVSS

SEQ ID NO: 290 - protein, 330 aa, synthetic construct; identical to SEQ ID NO: 10
  REGION 1..330: note = Engineered antibody sequence

SEQ ID NO: 291 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcgacctcagtaaccactacatgcaatgggtccgtcaggctccagggaaggggctggagtgggtcggagtcgttggtatcaatggtcgcacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 292 - DNA, 333 bp, synthetic construct
  misc_feature 1..333: note = Engineered antibody sequence
  source 1..333: mol_type = other DNA
gaggtgcagcttgtggagtctgggggaggcttggtccagcctggggggtccctgagactctcctgtgcagtctctggaatcgacctcagtaaccactacatgcaatgggtccgtcaggctccagggaaggggctggagtgggtcggagtcgttggtatcaatggtcgcacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagc

SEQ ID NO: 293 - DNA, 90 bp, synthetic construct; identical to SEQ ID NO: 173
  misc_feature 1..90: note = Engineered antibody sequence
  source 1..90: mol_type = other DNA

SEQ ID NO: 294 - DNA, 15 bp, synthetic construct
  misc_feature 1..15: note = Engineered antibody sequence
  source 1..15: mol_type = other DNA
aaccactacatgcaa

SEQ ID NO: 295 - DNA, 42 bp, synthetic construct; identical to SEQ ID NO: 55
  misc_feature 1..42: note = Engineered antibody sequence
  source 1..42: mol_type = other DNA

SEQ ID NO: 296 - DNA, 48 bp, synthetic construct
  misc_feature 1..48: note = Engineered antibody sequence
  source 1..48: mol_type = other DNA
gtcgttggtatcaatggtcgcacatactacgcgagctgggcgaaaggc

SEQ ID NO: 297 - DNA, 96 bp, synthetic construct; identical to SEQ ID NO: 57
  misc_feature 1..96: note = Engineered antibody sequence
  source 1..96: mol_type = other DNA

SEQ ID NO: 298 - skipped

SEQ ID NO: 299 - DNA, 33 bp, synthetic construct; identical to SEQ ID NO: 59
  misc_feature 1..33: note = Engineered antibody sequence
  source 1..33: mol_type = other DNA

SEQ ID NO: 300 - DNA, 993 bp, synthetic construct; identical to SEQ ID NO: 20
  misc_feature 1..993: note = Engineered antibody sequence
  source 1..993: mol_type = other DNA

SEQ ID NO: 301 - protein, 219 aa, synthetic construct
  REGION 1..219: note = Engineered antibody sequence
QVLTQSPSSLSASVGDRVTINCQASQSVYNYNYLAWYQQKPGKVPKQLIYSTSTLASGVPSRFSGSGSGTDFTLTISSLQPEDVATYYCLGSYDCSTGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 302 - protein, 113 aa, synthetic construct
  REGION 1..113: note = Engineered antibody sequence
QVLTQSPSSLSASVGDRVTINCQASQSVYNYNYLAWYQQKPGKVPKQLIYSTSTLASGVPSRFSGSGSGTDFTLTISSLQPEDVATYYCLGSYDCSTGDCFVFGGGTKVEIKR

SEQ ID NO: 303 - protein, 22 aa, synthetic construct
  REGION 1..22: note = Engineered antibody sequence
QVLTQSPSSLSASVGDRVTINC

SEQ ID NO: 304 - protein, 13 aa, synthetic construct
  REGION 1..13: note = Engineered antibody sequence
QASQSVYNYNYLA

SEQ ID NO: 305 - protein, 15 aa, synthetic construct
  REGION 1..15: note = Engineered antibody sequence
WYQQKPGKVPKQLIY

SEQ ID NO: 306 - protein, 7 aa, synthetic construct
  REGION 1..7: note = Engineered antibody sequence
STSTLAS

SEQ ID NO: 307 - protein, 32 aa, synthetic construct; identical to SEQ ID NO: 67
  REGION 1..32: note = Engineered antibody sequence

SEQ ID NO: 308 - protein, 13 aa, synthetic construct
  REGION 1..13: note = Engineered antibody sequence
LGSYDCSTGDCFV

SEQ ID NO: 309 - protein, 11 aa, synthetic construct
  REGION 1..11: note = Engineered antibody sequence
FGGGTKVEIKR

SEQ ID NO: 310 - protein, 106 aa, synthetic construct; identical to SEQ ID NO: 30
  REGION 1..106: note = Engineered antibody sequence

SEQ ID NO: 311 - DNA, 660 bp, synthetic construct
  misc_feature 1..660: note = Engineered antibody sequence
  source 1..660: mol_type = other DNA
caagtgctgacccagtctccatcctccctgtctgcatctgtaggagacagagtcaccatcaattgccaggccagtcagagtgtttacaattacaactaccttgcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtactggtgattgttttgttttcggcggaggaaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 312 - DNA, 339 bp, synthetic construct
  misc_feature 1..339: note = Engineered antibody sequence
  source 1..339: mol_type = other DNA
caagtgctgacccagtctccatcctccctgtctgcatctgtaggagacagagtcaccatcaattgccaggccagtcagagtgtttacaattacaactaccttgcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtactggtgattgttttgttttcggcggaggaaccaaggtggaaatcaaacgt

SEQ ID NO: 313 - DNA, 66 bp, synthetic construct; identical to SEQ ID NO: 73
  misc_feature 1..66: note = Engineered antibody sequence
  source 1..66: mol_type = other DNA

SEQ ID NO: 314 - DNA, 39 bp, synthetic construct
  misc_feature 1..39: note = Engineered antibody sequence
  source 1..39: mol_type = other DNA
caggccagtcagagtgtttacaattacaactaccttgcc

SEQ ID NO: 315 - DNA, 45 bp, synthetic construct; identical to SEQ ID NO: 75
  misc_feature 1..45: note = Engineered antibody sequence
  source 1..45: mol_type = other DNA

SEQ ID NO: 316 - DNA, 21 bp, synthetic construct
  misc_feature 1..21: note = Engineered antibody sequence
  source 1..21: mol_type = other DNA
tctacatccactctggcatct

SEQ ID NO: 317 - DNA, 96 bp, synthetic construct; identical to SEQ ID NO: 77
  misc_feature 1..96: note = Engineered antibody sequence
  source 1..96: mol_type = other DNA

SEQ ID NO: 318 - DNA, 39 bp, synthetic construct
  misc_feature 1..39: note = Engineered antibody sequence
  source 1..39: mol_type = other DNA
ctgggcagttatgattgtagtactggtgattgttttgtt

SEQ ID NO: 319 - DNA, 33 bp, synthetic construct; identical to SEQ ID NO: 79
  misc_feature 1..33: note = Engineered antibody sequence
  source 1..33: mol_type = other DNA

SEQ ID NO: 320 - DNA, 321 bp, synthetic construct; identical to SEQ ID NO: 40
  misc_feature 1..321: note = Engineered antibody sequence
  source 1..321: mol_type = other DNA

SEQ ID NO: 321 - protein, 439 aa, synthetic construct
  REGION 1..439: note = Engineered antibody sequence
QSLEESGGRLVTPGTPLTLTCTVSGIGLSSYYMQWVRQSPGRGLEWIGVIGSDGKTYYATWAKGRFTISKTSSTTVDLRMASLTTEDTATYFCTRGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 109 aa, synthetic construct
  REGION 1..109: note = Engineered antibody sequence
QSLEESGGRLVTPGTPLTLTCTVSGIGLSSYYMQWVRQSPGRGLEWIGVIGSDGKTYYATWAKGRFTISKTSSTTVDLRMASLTTEDTATYFCTRGDIWGPGTLVTVSS

SEQ ID NO: 323 - protein, 29 aa, synthetic construct
  REGION 1..29: note = Engineered antibody sequence
QSLEESGGRLVTPGTPLTLTCTVSGIGLS

SEQ ID NO: 324 - protein, 5 aa, synthetic construct
  REGION 1..5: note = Engineered antibody sequence
SYYMQ

SEQ ID NO: 325 - protein, 14 aa, synthetic construct
  REGION 1..14: note = Engineered antibody sequence
WVRQSPGRGLEWIG

SEQ ID NO: 326 - protein, 16 aa, synthetic construct
  REGION 1..16: note = Engineered antibody sequence
VIGSDGKTYYATWAKG

SEQ ID NO: 327 - protein, 31 aa, synthetic construct
  REGION 1..31: note = Engineered antibody sequence
RFTISKTSSTTVDLRMASLTTEDTATYFCTR

SEQ ID NO: 328 - skipped

SEQ ID NO: 329 - protein, 11 aa, synthetic construct
  REGION 1..11: note = Engineered antibody sequence
WGPGTLVTVSS

SEQ ID NO: 330 - protein, 330 aa, synthetic construct; identical to SEQ ID NO: 10
  REGION 1..330: note = Engineered antibody sequence

SEQ ID NO: 331 - DNA, 1320 bp, synthetic construct
  misc_feature 1..1320: note = Engineered antibody sequence
  source 1..1320: mol_type = other DNA
cagtcgctggaggagtccgggggtcgcctggtcacgcctgggacacccctgacactcacctgcacagtctctggaatcggcctcagtagctactacatgcagtgggtccgccagtctccagggagggggctggaatggatcggagtcattggtagtgatggtaagacatactacgcgacctgggcgaaaggccgattcaccatctccaagacctcgtcgaccacggtggatctgagaatggccagtctgacaaccgaggacacggccacctatttctgtaccagaggggacatctggggcccggggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 332 - DNA, 327 bp, synthetic construct
  misc_feature 1..327: note = Engineered antibody sequence
  source 1..327: mol_type = other DNA
cagtcgctggaggagtccgggggtcgcctggtcacgcctgggacacccctgacactcacctgcacagtctctggaatcggcctcagtagctactacatgcagtgggtccgccagtctccagggagggggctggaatggatcggagtcattggtagtgatggtaagacatactacgcgacctgggcgaaaggccgattcaccatctccaagacctcgtcgaccacggtggatctgagaatggccagtctgacaaccgaggacacggccacctatttctgtaccagaggggacatctggggcccggggaccctcgtcaccgtctcgagc

SEQ ID NO: 333 - DNA, 87 bp, synthetic construct
  misc_feature 1..87: note = Engineered antibody sequence
  source 1..87: mol_type = other DNA
cagtcgctggaggagtccgggggtcgcctggtcacgcctgggacacccctgacactcacctgcacagtctctggaatcggcctcagt

SEQ ID NO: 334 - DNA, 15 bp, synthetic construct
  misc_feature 1..15: note = Engineered antibody sequence
  source 1..15: mol_type = other DNA
agctactacatgcag

SEQ ID NO: 335 - DNA, 42 bp, synthetic construct
  misc_feature 1..42: note = Engineered antibody sequence
  source 1..42: mol_type = other DNA
tgggtccgccagtctccagggagggggctggaatggatcgga

SEQ ID NO: 336 - DNA, 48 bp, synthetic construct
  misc_feature 1..48: note = Engineered antibody sequence
  source 1..48: mol_type = other DNA
gtcattggtagtgatggtaagacatactacgcgacctgggcgaaaggc

SEQ ID NO: 337 - DNA, 93 bp, synthetic construct
  misc_feature 1..93: note = Engineered antibody sequence
  source 1..93: mol_type = other DNA
cgattcaccatctccaagacctcgtcgaccacggtggatctgagaatggccagtctgacaaccgaggacacggccacctatttctgtaccaga

SEQ ID NO: 338 - skipped

SEQ ID NO: 339 - DNA, 33 bp, synthetic construct
  misc_feature 1..33: note = Engineered antibody sequence
  source 1..33: mol_type = other DNA
tggggcccggggaccctcgtcaccgtctcgagc

SEQ ID NO: 340 - DNA, 993 bp, synthetic construct; identical to SEQ ID NO: 20
  misc_feature 1..993: note = Engineered antibody sequence
  source 1..993: mol_type = other DNA

SEQ ID NO: 341 - protein, 219 aa, synthetic construct
  REGION 1..219: note = Engineered antibody sequence
QVLTQTPSPVSAAVGSTVTINCQASQNVYNNNYLAWYQQKPGQPPKQLIYSTSTLASGVSSRFRGSGSGTQFTLTISDVQCDDAATYYCLGSYDCSRGDCFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 342 - protein, 113 aa, synthetic construct
  REGION 1..113: note = Engineered antibody sequence
QVLTQTPSPVSAAVGSTVTINCQASQNVYNNNYLAWYQQKPGQPPKQLIYSTSTLASGVSSRFRGSGSGTQFTLTISDVQCDDAATYYCLGSYDCSRGDCFVFGGGTEVVVKR

SEQ ID NO: 343 - protein, 22 aa, synthetic construct
  REGION 1..22: note = Engineered antibody sequence
QVLTQTPSPVSAAVGSTVTINC

SEQ ID NO: 344 - protein, 13 aa, synthetic construct
  REGION 1..13: note = Engineered antibody sequence
QASQNVYNNNYLA

SEQ ID NO: 345 - protein, 15 aa, synthetic construct
  REGION 1..15: note = Engineered antibody sequence
WYQQKPGQPPKQLIY

SEQ ID NO: 346 - protein, 7 aa, synthetic construct
  REGION 1..7: note = Engineered antibody sequence
STSTLAS

SEQ ID NO: 347 - protein, 32 aa, synthetic construct
  REGION 1..32: note = Engineered antibody sequence
GVSSRFRGSGSGTQFTLTISDVQCDDAATYYC

SEQ ID NO: 348 - protein, 13 aa, synthetic construct
  REGION 1..13: note = Engineered antibody sequence
LGSYDCSRGDCFV

SEQ ID NO: 349 - protein, 11 aa, synthetic construct
  REGION 1..11: note = Engineered antibody sequence
FGGGTEVVVKR

SEQ ID NO: 350 - protein, 106 aa, synthetic construct; identical to SEQ ID NO: 30
  REGION 1..106: note = Engineered antibody sequence

SEQ ID NO: 351 - DNA, 660 bp, synthetic construct
  misc_feature 1..660: note = Engineered antibody sequence
  source 1..660: mol_type = other DNA
caagtgctgacccagactccatcccccgtgtctgcagctgtgggaagcacagtcaccatcaattgccaggccagtcagaatgtttataataacaactacctagcctggtatcagcagaaaccagggcagcctcccaagcaactgatctattctacgtccactctggcatctggggtctcatcgcgattcagaggcagtggatctgggacacagttcactctcaccatcagcgacgtgcagtgtgacgatgctgccacttactactgtctaggcagttatgattgtagtcgtggtgattgttttgttttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 352 - DNA, 339 bp, synthetic construct
  misc_feature 1..339: note = Engineered antibody sequence
  source 1..339: mol_type = other DNA
caagtgctgacccagactccatcccccgtgtctgcagctgtgggaagcacagtcaccatcaattgccaggccagtcagaatgtttataataacaactacctagcctggtatcagcagaaaccagggcagcctcccaagcaactgatctattctacgtccactctggcatctggggtctcatcgcgattcagaggcagtggatctgggacacagttcactctcaccatcagcgacgtgcagtgtgacgatgctgccacttactactgtctaggcagttatgattgtagtcgtggtgattgttttgttttcggcggagggaccgaggtggtggtcaaacgt

SEQ ID NO: 353 - DNA, 66 bp, synthetic construct; identical to SEQ ID NO: 153
  misc_feature 1..66: note = Engineered antibody sequence
  source 1..66: mol_type = other DNA

SEQ ID NO: 354 - DNA, 39 bp, synthetic construct
  misc_feature 1..39: note = Engineered antibody sequence
  source 1..39: mol_type = other DNA
caggccagtcagaatgtttataataacaactacctagcc

SEQ ID NO: 355 - DNA, 45 bp, synthetic construct; identical to SEQ ID NO: 35
  misc_feature 1..45: note = Engineered antibody sequence
  source 1..45: mol_type = other DNA

SEQ ID NO: 356 - DNA, 21 bp, synthetic construct
  misc_feature 1..21: note = Engineered antibody sequence
  source 1..21: mol_type = other DNA
tctacgtccactctggcatct

SEQ ID NO: 357 - DNA, 96 bp, synthetic construct
  misc_feature 1..96: note = Engineered antibody sequence
  source 1..96: mol_type = other DNA
ggggtctcatcgcgattcagaggcagtggatctgggacacagttcactctcaccatcagcgacgtgcagtgtgacgatgctgccacttactactgt

SEQ ID NO: 358 - DNA, 39 bp, synthetic construct
  misc_feature 1..39: note = Engineered antibody sequence
  source 1..39: mol_type = other DNA
ctaggcagttatgattgtagtcgtggtgattgttttgtt

SEQ ID NO: 359 - DNA, 33 bp, synthetic construct; identical to SEQ ID NO: 39
  misc_feature 1..33: note = Engineered antibody sequence
  source 1..33: mol_type = other DNA

SEQ ID NO: 360 - DNA, 321 bp, synthetic construct; identical to SEQ ID NO: 40
  misc_feature 1..321: note = Engineered antibody sequence
  source 1..321: mol_type = other DNA

SEQ ID NO: 361 - protein, 441 aa, synthetic construct
  REGION 1..441: note = Engineered antibody sequence
EVQLVESGGGLVQPGGSLRLSCAVSGIGLSSYYMQWVRQAPGKGLEWVGVIGSDGKTYYATWAKGRFTISRDNSKTTVYLQMNSLRAEDTAVYFCTRGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 362 - protein, 111 aa, synthetic construct
  REGION 1..111: note = Engineered antibody sequence
EVQLVESGGGLVQPGGSLRLSCAVSGIGLSSYYMQWVRQAPGKGLEWVGVIGSDGKTYYATWAKGRFTISRDNSKTTVYLQMNSLRAEDTAVYFCTRGDIWGQGTLVTVSS

SEQ ID NO: 363 - protein, 30 aa, synthetic construct
  REGION 1..30: note = Engineered antibody sequence
EVQLVESGGGLVQPGGSLRLSCAVSGIGLS

SEQ ID NO: 364 - protein, 5 aa, synthetic construct
  REGION 1..5: note = Engineered antibody sequence
SYYMQ

SEQ ID NO: 365 - protein, 14 aa, synthetic construct
  REGION 1..14: note = Engineered antibody sequence
WVRQAPGKGLEWVG

SEQ ID NO: 366 - protein, 16 aa, synthetic construct
  REGION 1..16: note = Engineered antibody sequence
VIGSDGKTYYATWAKG

SEQ ID NO: 367 - protein, 32 aa, synthetic construct
  REGION 1..32: note = Engineered antibody sequence
RFTISRDNSKTTVYLQMNSLRAEDTAVYFCTR

SEQ ID NO: 368 - skipped

SEQ ID NO: 369 - protein, 11 aa, synthetic construct
  REGION 1..11: note = Engineered antibody sequence
WGQGTLVTVSS

SEQ ID NO: 370 - protein, 330 aa, synthetic construct; identical to SEQ ID NO: 10
  REGION 1..330: note = Engineered antibody sequence

SEQ ID NO: 371 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcggcctcagtagctactacatgcaatgggtccgtcaggctccagggaaggggctggagtgggtcggagtcattggtagtgatggtaagacatactacgcgacctgggcgaaaggccgattcaccatctccagagacaattccaagaccacggtgtatcttcaaatgaacagcctgagagctgaggacactgctgtgtatttctgtaccagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 372 - DNA, 333 bp, synthetic construct
  misc_feature 1..333: note = Engineered antibody sequence
  source 1..333: mol_type = other DNA
gaggtgcagcttgtggagtctgggggaggcttggtccagcctggggggtccctgagactctcctgtgcagtctctggaatcggcctcagtagctactacatgcaatgggtccgtcaggctccagggaaggggctggagtgggtcggagtcattggtagtgatggtaagacatactacgcgacctgggcgaaaggccgattcaccatctccagagacaattccaagaccacggtgtatcttcaaatgaacagcctgagagctgaggacactgctgtgtatttctgtaccagaggggacatctggggccaagggaccctcgtcaccgtctcgagc

SEQ ID NO: 373 - DNA, 90 bp, synthetic construct
  misc_feature 1..90: note = Engineered antibody sequence
  source 1..90: mol_type = other DNA
gaggtgcagcttgtggagtctgggggaggcttggtccagcctggggggtccctgagactctcctgtgcagtctctggaatcggcctcagt

SEQ ID NO: 374 - DNA, 15 bp, synthetic construct
  misc_feature 1..15: note = Engineered antibody sequence
  source 1..15: mol_type = other DNA
agctactacatgcaa

SEQ ID NO: 375 - DNA, 42 bp, synthetic construct; identical to SEQ ID NO: 55
  misc_feature 1..42: note = Engineered antibody sequence
  source 1..42: mol_type = other DNA

SEQ ID NO: 376 - DNA, 48 bp, synthetic construct; identical to SEQ ID NO: 336
  misc_feature 1..48: note = Engineered antibody sequence
  source 1..48: mol_type = other DNA

SEQ ID NO: 377 - DNA, 96 bp, synthetic construct
  misc_feature 1..96: note = Engineered antibody sequence
  source 1..96: mol_type = other DNA
cgattcaccatctccagagacaattccaagaccacggtgtatcttcaaatgaacagcctgagagctgaggacactgctgtgtatttctgtaccaga

SEQ ID NO: 378 - skipped

SEQ ID NO: 379 - DNA, 33 bp, synthetic construct; identical to SEQ ID NO: 59
  misc_feature 1..33: note = Engineered antibody sequence
  source 1..33: mol_type = other DNA

SEQ ID NO: 380 - DNA, 993 bp, synthetic construct; identical to SEQ ID NO: 20
  misc_feature 1..993: note = Engineered antibody sequence
  source 1..993: mol_type = other DNA

SEQ ID NO: 381 - protein, 219 aa, synthetic construct
  REGION 1..219: note = Engineered antibody sequence
QVLTQSPSSLSASVGDRVTINCQASQNVYNNNYLAWYQQKPGKVPKQLIYSTSTLASGVPSRFSGSGSGTDFTLTISSLQPEDVATYYCLGSYDCSRGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 382 - protein, 113 aa, synthetic construct
  REGION 1..113: note = Engineered antibody sequence
QVLTQSPSSLSASVGDRVTINCQASQNVYNNNYLAWYQQKPGKVPKQLIYSTSTLASGVPSRFSGSGSGTDFTLTISSLQPEDVATYYCLGSYDCSRGDCFVFGGGTKVEIKR

SEQ ID NO: 383 - protein, 22 aa, synthetic construct
  REGION 1..22: note = Engineered antibody sequence
QVLTQSPSSLSASVGDRVTINC

SEQ ID NO: 384 - protein, 13 aa, synthetic construct
  REGION 1..13: note = Engineered antibody sequence
QASQNVYNNNYLA

SEQ ID NO: 385 - protein, 15 aa, synthetic construct
  REGION 1..15: note = Engineered antibody sequence
WYQQKPGKVPKQLIY

SEQ ID NO: 386 - protein, 7 aa, synthetic construct
  REGION 1..7: note = Engineered antibody sequence
STSTLAS

SEQ ID NO: 387 - protein, 32 aa, synthetic construct; identical to SEQ ID NO: 67
  REGION 1..32: note = Engineered antibody sequence

SEQ ID NO: 388 - protein, 13 aa, synthetic construct
  REGION 1..13: note = Engineered antibody sequence
LGSYDCSRGDCFV

SEQ ID NO: 389 - protein, 11 aa, synthetic construct
  REGION 1..11: note = Engineered antibody sequence
FGGGTKVEIKR

SEQ ID NO: 390 - protein, 106 aa, synthetic construct; identical to SEQ ID NO: 30
  REGION 1..106: note = Engineered antibody sequence

SEQ ID NO: 391 - DNA, 660 bp, synthetic construct
  misc_feature 1..660: note = Engineered antibody sequence
  source 1..660: mol_type = other DNA
caagtgctgacccagtctccatcctccctgtctgcatctgtaggagacagagtcaccatcaattgccaggccagtcagaatgtttacaataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtcgtggtgattgttttgttttcggcggaggaaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 392 - DNA, 339 bp, synthetic construct
  misc_feature 1..339: note = Engineered antibody sequence
  source 1..339: mol_type = other DNA
caagtgctgacccagtctccatcctccctgtctgcatctgtaggagacagagtcaccatcaattgccaggccagtcagaatgtttacaataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtcgtggtgattgttttgttttcggcggaggaaccaaggtggaaatcaaacgt

SEQ ID NO: 393 - DNA, 66 bp, synthetic construct; identical to SEQ ID NO: 73
  misc_feature 1..66: note = Engineered antibody sequence
  source 1..66: mol_type = other DNA

SEQ ID NO: 394 - DNA, 39 bp, synthetic construct
  misc_feature 1..39: note = Engineered antibody sequence
  source 1..39: mol_type = other DNA
caggccagtcagaatgtttacaataacaactacctagcc

SEQ ID NO: 395 - DNA, 45 bp, synthetic construct; identical to SEQ ID NO: 75
  misc_feature 1..45: note = Engineered antibody sequence
  source 1..45: mol_type = other DNA

SEQ ID NO: 396 - DNA, 21 bp, synthetic construct
  misc_feature 1..21: note = Engineered antibody sequence
  source 1..21: mol_type = other DNA
tctacatccactctggcatct

SEQ ID NO: 397 - DNA, 96 bp, synthetic construct; identical to SEQ ID NO: 77
  misc_feature 1..96: note = Engineered antibody sequence
  source 1..96: mol_type = other DNA

SEQ ID NO: 398 - DNA, 39 bp, synthetic construct
  misc_feature 1..39: note = Engineered antibody sequence
  source 1..39: mol_type = other DNA
ctgggcagttatgattgtagtcgtggtgattgttttgtt

SEQ ID NO: 399 - DNA, 33 bp, synthetic construct; identical to SEQ ID NO: 79
  misc_feature 1..33: note = Engineered antibody sequence
  source 1..33: mol_type = other DNA

SEQ ID NO: 400 - DNA, 321 bp, synthetic construct; identical to SEQ ID NO: 40
  misc_feature 1..321: note = Engineered antibody sequence
  source 1..321: mol_type = other DNA

SEQ ID NO: 401 - protein, 439 aa, synthetic construct
  REGION 1..439: note = Engineered antibody sequence
QSLEESGGRLVTPGGSLTLTCTVSGIDVTNYYMQWVRQAPGKGLEWIGVIGVNGKRYYASWAKGRFTISKTSSTTVDLKMTSLTTEDTATYFCARGDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 402 - protein, 109 aa, synthetic construct
  REGION 1..109: note = Engineered antibody sequence
QSLEESGGRLVTPGGSLTLTCTVSGIDVTNYYMQWVRQAPGKGLEWIGVIGVNGKRYYASWAKGRFTISKTSSTTVDLKMTSLTTEDTATYFCARGDIWGPGTLVTVSS

SEQ ID NO: 403 - protein, 29 aa, synthetic construct
  REGION 1..29: note = Engineered antibody sequence
QSLEESGGRLVTPGGSLTLTCTVSGIDVT

SEQ ID NO: 404 - protein, 5 aa, synthetic construct
  REGION 1..5: note = Engineered antibody sequence
NYYMQ

SEQ ID NO: 405 - protein, 14 aa, synthetic construct
  REGION 1..14: note = Engineered antibody sequence
WVRQAPGKGLEWIG

SEQ ID NO: 406 - protein, 16 aa, synthetic construct
  REGION 1..16: note = Engineered antibody sequence
VIGVNGKRYYASWAKG

SEQ ID NO: 407 - protein, 31 aa, synthetic construct; identical to SEQ ID NO: 127
  REGION 1..31: note = Engineered antibody sequence

SEQ ID NO: 408 - skipped

SEQ ID NO: 409 - protein, 11 aa, synthetic construct
  REGION 1..11: note = Engineered antibody sequence
WGPGTLVTVSS

SEQ ID NO: 410 - protein, 330 aa, synthetic construct; identical to SEQ ID NO: 10
  REGION 1..330: note = Engineered antibody sequence

SEQ ID NO: 411 - DNA, 1320 bp, synthetic construct
  misc_feature 1..1320: note = Engineered antibody sequence
  source 1..1320: mol_type = other DNA
cagtcgctggaggagtccgggggtcgcctggtcacgcctggaggatccctgacactcacctgcacagtctctggaatcgacgtcactaactactatatgcaatgggtccgccaggctccagggaaggggctggaatggatcggagtcattggtgtgaatggtaagagatactacgcgagctgggcgaaaggccgattcaccatctccaaaacctcgtcgaccacggtggatctgaaaatgaccagtctgacaaccgaggacacggccacctatttctgtgccagaggcgacatctggggcccggggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 412 - DNA, 327 bp, synthetic construct
  misc_feature 1..327: note = Engineered antibody sequence
  source 1..327: mol_type = other DNA
cagtcgctggaggagtccgggggtcgcctggtcacgcctggaggatccctgacactcacctgcacagtctctggaatcgacgtcactaactactatatgcaatgggtccgccaggctccagggaaggggctggaatggatcggagtcattggtgtgaatggtaagagatactacgcgagctgggcgaaaggccgattcaccatctccaaaacctcgtcgaccacggtggatctgaaaatgaccagtctgacaaccgaggacacggccacctatttctgtgccagaggcgacatctggggcccggggaccctcgtcaccgtctcgagc

SEQ ID NO: 413 - DNA, 87 bp, synthetic construct
  misc_feature 1..87: note = Engineered antibody sequence
  source 1..87: mol_type = other DNA
cagtcgctggaggagtccgggggtcgcctggtcacgcctggaggatccctgacactcacctgcacagtctctggaatcgacgtcact

SEQ ID NO: 414 - DNA, 15 bp, synthetic construct
  misc_feature 1..15: note = Engineered antibody sequence
  source 1..15: mol_type = other DNA
aactactatatgcaa

SEQ ID NO: 415 - DNA, 42 bp, synthetic construct; identical to SEQ ID NO: 15
  misc_feature 1..42: note = Engineered antibody sequence
  source 1..42: mol_type = other DNA

SEQ ID NO: 416 - DNA, 48 bp, synthetic construct
  misc_feature 1..48: note = Engineered antibody sequence
  source 1..48: mol_type = other DNA
gtcattggtgtgaatggtaagagatactacgcgagctgggcgaaaggc

SEQ ID NO: 417 - DNA, 93 bp, synthetic construct; identical to SEQ ID NO: 137
  misc_feature 1..93: note = Engineered antibody sequence
  source 1..93: mol_type = other DNA

SEQ ID NO: 418 - skipped

SEQ ID NO: 419 - DNA, 33 bp, synthetic construct; identical to SEQ ID NO: 339
  misc_feature 1..33: note = Engineered antibody sequence
  source 1..33: mol_type = other DNA

SEQ ID NO: 420 - DNA, 993 bp, synthetic construct; identical to SEQ ID NO: 20
  misc_feature 1..993: note = Engineered antibody sequence
  source 1..993: mol_type = other DNA

SEQ ID NO: 421 - protein, 219 aa, synthetic construct
  REGION 1..219: note = Engineered antibody sequence
QVLTQTASPVSPAVGSTVTINCRASQSVYYNNYLAWYQQKPGQPPKQLIYSTSTLASGVSSRFKGSGSGTQFTLTISDVQCDDAATYYCLGSYDCSNGDCFVFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 422 - protein, 113 aa, synthetic construct
  REGION 1..113: note = Engineered antibody sequence
QVLTQTASPVSPAVGSTVTINCRASQSVYYNNYLAWYQQKPGQPPKQLIYSTSTLASGVSSRFKGSGSGTQFTLTISDVQCDDAATYYCLGSYDCSNGDCFVFGGGTEVVVKR

SEQ ID NO: 423 - protein, 22 aa, synthetic construct
  REGION 1..22: note = Engineered antibody sequence
QVLTQTASPVSPAVGSTVTINC

SEQ ID NO: 424 - protein, 13 aa, synthetic construct
  REGION 1..13: note = Engineered antibody sequence
RASQSVYYNNYLA

SEQ ID NO: 425 - protein, 15 aa, synthetic construct
  REGION 1..15: note = Engineered antibody sequence
WYQQKPGQPPKQLIY

SEQ ID NO: 426 - protein, 7 aa, synthetic construct
  REGION 1..7: note = Engineered antibody sequence
STSTLAS

SEQ ID NO: 427 - protein, 32 aa, synthetic construct; identical to SEQ ID NO: 267
  REGION 1..32: note = Engineered antibody sequence

SEQ ID NO: 428 - protein, 13 aa, synthetic construct
  REGION 1..13: note = Engineered antibody sequence
LGSYDCSNGDCFV

SEQ ID NO: 429 - protein, 11 aa, synthetic construct
  REGION 1..11: note = Engineered antibody sequence
FGGGTEVVVKR

SEQ ID NO: 430 - protein, 106 aa, synthetic construct; identical to SEQ ID NO: 30
  REGION 1..106: note = Engineered antibody sequence

SEQ ID NO: 431 - DNA, 660 bp, synthetic construct
  misc_feature 1..660: note = Engineered antibody sequence
  source 1..660: mol_type = other DNA
caggtgctgacccagactgcatcccccgtgtctccagctgtgggaagcacagtcaccatcaattgccgggccagtcagagtgtttattataacaactacctagcctggtatcagcagaaaccagggcagcctcccaagcaactgatctattctacatccactctggcatctggggtctcatcgcggttcaaaggcagtggatctgggacacagttcactctcaccatcagcgacgtgcagtgtgacgatgctgccacttactactgtctaggcagttatgattgtagtaatggtgattgttttgttttcggcggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 432 - DNA, 339 bp, synthetic construct
  misc_feature 1..339: note = Engineered antibody sequence
  source 1..339: mol_type = other DNA
caggtgctgacccagactgcatcccccgtgtctccagctgtgggaagcacagtcaccatcaattgccgggccagtcagagtgtttattataacaactacctagcctggtatcagcagaaaccagggcagcctcccaagcaactgatctattctacatccactctggcatctggggtctcatcgcggttcaaaggcagtggatctgggacacagttcactctcaccatcagcgacgtgcagtgtgacgatgctgccacttactactgtctaggcagttatgattgtagtaatggtgattgttttgttttcggcggagggaccgaggtggtggtcaaacgt

SEQ ID NO: 433 - DNA, 66 bp, synthetic construct
  misc_feature 1..66: note = Engineered antibody sequence
  source 1..66: mol_type = other DNA
caggtgctgacccagactgcatcccccgtgtctccagctgtgggaagcacagtcaccatcaattgc

SEQ ID NO: 434 - DNA, 39 bp, synthetic construct
  misc_feature 1..39: note = Engineered antibody sequence
  source 1..39: mol_type = other DNA
cgggccagtcagagtgtttattataacaactacctagcc

SEQ ID NO: 435 - DNA, 45 bp, synthetic construct; identical to SEQ ID NO: 35
  misc_feature 1..45: note = Engineered antibody sequence
  source 1..45: mol_type = other DNA

SEQ ID NO: 436 - DNA, 21 bp, synthetic construct
  misc_feature 1..21: note = Engineered antibody sequence
  source 1..21: mol_type = other DNA
tctacatccactctggcatct

SEQ ID NO: 437 - DNA, 96 bp, synthetic construct
  misc_feature 1..96: note = Engineered antibody sequence
  source 1..96: mol_type = other DNA
ggggtctcatcgcggttcaaaggcagtggatctgggacacagttcactctcaccatcagcgacgtgcagtgtgacgatgctgccacttactactgt

SEQ ID NO: 438 - DNA, 39 bp, synthetic construct
  misc_feature 1..39: note = Engineered antibody sequence
  source 1..39: mol_type = other DNA
ctaggcagttatgattgtagtaatggtgattgttttgtt

SEQ ID NO: 439 - DNA, 33 bp, synthetic construct; identical to SEQ ID NO: 39
  misc_feature 1..33: note = Engineered antibody sequence
  source 1..33: mol_type = other DNA

SEQ ID NO: 440 - DNA, 321 bp, synthetic construct; identical to SEQ ID NO: 40
  misc_feature 1..321: note = Engineered antibody sequence
  source 1..321: mol_type = other DNA

SEQ ID NO: 441 - protein, 441 aa, synthetic construct
  REGION 1..441: note = Engineered antibody sequence
EVQLVESGGGLVQPGGSLRLSCAVSGIDVTNYYMQWVRQAPGKGLEWVGVIGVNGKRYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 442 - protein, 111 aa, synthetic construct
  REGION 1..111: note = Engineered antibody sequence
EVQLVESGGGLVQPGGSLRLSCAVSGIDVTNYYMQWVRQAPGKGLEWVGVIGVNGKRYYASWAKGRFTISRDNSKTTVYLQMNSLRAEDTAVYFCARGDIWGQGTLVTVSS

SEQ ID NO: 443 - protein, 30 aa, synthetic construct
  REGION 1..30: note = Engineered antibody sequence
EVQLVESGGGLVQPGGSLRLSCAVSGIDVT

SEQ ID NO: 444 - protein, 5 aa, synthetic construct
  REGION 1..5: note = Engineered antibody sequence
NYYMQ

SEQ ID NO: 445 - protein, 14 aa, synthetic construct
  REGION 1..14: note = Engineered antibody sequence
WVRQAPGKGLEWVG

SEQ ID NO: 446 - protein, 16 aa, synthetic construct
  REGION 1..16: note = Engineered antibody sequence
VIGVNGKRYYASWAKG

SEQ ID NO: 447 - protein, 32 aa, synthetic construct; identical to SEQ ID NO: 47
  REGION 1..32: note = Engineered antibody sequence

SEQ ID NO: 448 - skipped

SEQ ID NO: 449 - protein, 11 aa, synthetic construct
  REGION 1..11: note = Engineered antibody sequence
WGQGTLVTVSS

SEQ ID NO: 450 - protein, 330 aa, synthetic construct; identical to SEQ ID NO: 10
  REGION 1..330: note = Engineered antibody sequence

SEQ ID NO: 451 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcgacgtcactaactactacatgcaatgggtccgtcaggctccagggaaggggctggagtgggtcggagtcattggtgtgaatggtaagagatactacgcgagctgggcgaaaggccgattcaccatctccagagacaattccaagaccacggtgtatcttcaaatgaacagcctgagagctgaggacactgctgtgtatttctgtgccagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 452 - DNA, 333 bp, synthetic construct
  misc_feature 1..333: note = Engineered antibody sequence
  source 1..333: mol_type = other DNA
gaggtgcagcttgtggagtctgggggaggcttggtccagcctggggggtccctgagactctcctgtgcagtctctggaatcgacgtcactaactactacatgcaatgggtccgtcaggctccagggaaggggctggagtgggtcggagtcattggtgtgaatggtaagagatactacgcgagctgggcgaaaggccgattcaccatctccagagacaattccaagaccacggtgtatcttcaaatgaacagcctgagagctgaggacactgctgtgtatttctgtgccagaggggacatctggggccaagggaccctcgtcaccgtctcgagc

SEQ ID NO: 453 - DNA, 90 bp, synthetic construct
  misc_feature 1..90: note = Engineered antibody sequence
  source 1..90: mol_type = other DNA
gaggtgcagcttgtggagtctgggggaggcttggtccagcctggggggtccctgagactctcctgtgcagtctctggaatcgacgtcact

SEQ ID NO: 454 - DNA, 15 bp, synthetic construct
  misc_feature 1..15: note = Engineered antibody sequence
  source 1..15: mol_type = other DNA
aactactacatgcaa

SEQ ID NO: 455 - DNA, 42 bp, synthetic construct; identical to SEQ ID NO: 55
  misc_feature 1..42: note = Engineered antibody sequence
  source 1..42: mol_type = other DNA

SEQ ID NO: 456 - DNA, 48 bp, synthetic construct; identical to SEQ ID NO: 416
  misc_feature 1..48: note = Engineered antibody sequence
  source 1..48: mol_type = other DNA

SEQ ID NO: 457 - DNA, 96 bp, synthetic construct
  misc_feature 1..96: note = Engineered antibody sequence
  source 1..96: mol_type = other DNA
cgattcaccatctccagagacaattccaagaccacggtgtatcttcaaatgaacagcctgagagctgaggacactgctgtgtatttctgtgccaga

SEQ ID NO: 458 - skipped

SEQ ID NO: 459 - DNA, 33 bp, synthetic construct; identical to SEQ ID NO: 59
  misc_feature 1..33: note = Engineered antibody sequence
  source 1..33: mol_type = other DNA

SEQ ID NO: 460 - DNA, 993 bp, synthetic construct; identical to SEQ ID NO: 20
  misc_feature 1..993: note = Engineered antibody sequence
  source 1..993: mol_type = other DNA

SEQ ID NO: 461 - protein, 219 aa, synthetic construct
  REGION 1..219: note = Engineered antibody sequence
QVLTQSPSSLSASVGDRVTINCRASQSVYYNNYLAWYQQKPGKVPKQLIYSTSTLASGVPSRFSGSGSGTDFTLTISSLQPEDVATYYCLGSYDCSNGDCFVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 462 - protein, 113 aa, synthetic construct
  REGION 1..113: note = Engineered antibody sequence
QVLTQSPSSLSASVGDRVTINCRASQSVYYNNYLAWYQQKPGKVPKQLIYSTSTLASGVPSRFSGSGSGTDFTLTISSLQPEDVATYYCLGSYDCSNGDCFVFGGGTKVEIKR

SEQ ID NO: 463 - protein, 22 aa, synthetic construct
  REGION 1..22: note = Engineered antibody sequence
QVLTQSPSSLSASVGDRVTINC

SEQ ID NO: 464 - protein, 13 aa, synthetic construct
  REGION 1..13: note = Engineered antibody sequence
RASQSVYYNNYLA

SEQ ID NO: 465 - protein, 15 aa, synthetic construct
  REGION 1..15: note = Engineered antibody sequence
WYQQKPGKVPKQLIY

SEQ ID NO: 466 - protein, 7 aa, synthetic construct
  REGION 1..7: note = Engineered antibody sequence
STSTLAS

SEQ ID NO: 467 - protein, 32 aa, synthetic construct; identical to SEQ ID NO: 67
  REGION 1..32: note = Engineered antibody sequence

SEQ ID NO: 468 - protein, 13 aa, synthetic construct
  REGION 1..13: note = Engineered antibody sequence
LGSYDCSNGDCFV

SEQ ID NO: 469 - protein, 11 aa, synthetic construct
  REGION 1..11: note = Engineered antibody sequence
FGGGTKVEIKR

SEQ ID NO: 470 - protein, 106 aa, synthetic construct; identical to SEQ ID NO: 30
  REGION 1..106: note = Engineered antibody sequence

SEQ ID NO: 471 - DNA, 660 bp, synthetic construct
  misc_feature 1..660: note = Engineered antibody sequence
  source 1..660: mol_type = other DNA
caagtgctgacccagtctccatcctccctgtctgcatctgtaggagacagagtcaccatcaattgccgggccagtcagagtgtttactataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtaatggtgattgttttgttttcggcggaggaaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 472 - DNA, 339 bp, synthetic construct
  misc_feature 1..339: note = Engineered antibody sequence
  source 1..339: mol_type = other DNA
caagtgctgacccagtctccatcctccctgtctgcatctgtaggagacagagtcaccatcaattgccgggccagtcagagtgtttactataacaactacctagcctggtatcagcagaaaccagggaaagttcctaagcaactgatctattctacatccactctggcatctggggtcccatctcgtttcagtggcagtggatctgggacagatttcactctcaccatcagcagcctgcagcctgaagatgttgcaacttattactgtctgggcagttatgattgtagtaatggtgattgttttgttttcggcggaggaaccaaggtggaaatcaaacgt

SEQ ID NO: 473 - DNA, 66 bp, synthetic construct; identical to SEQ ID NO: 73
  misc_feature 1..66: note = Engineered antibody sequence
  source 1..66: mol_type = other DNA

SEQ ID NO: 474 - DNA, 39 bp, synthetic construct
  misc_feature 1..39: note = Engineered antibody sequence
  source 1..39: mol_type = other DNA
cgggccagtcagagtgtttactataacaactacctagcc

SEQ ID NO: 475 - DNA, 45 bp, synthetic construct; identical to SEQ ID NO: 75
  misc_feature 1..45: note = Engineered antibody sequence
  source 1..45: mol_type = other DNA

SEQ ID NO: 476 - DNA, 21 bp, synthetic construct
  misc_feature 1..21: note = Engineered antibody sequence
  source 1..21: mol_type = other DNA
tctacatccactctggcatct

SEQ ID NO: 477 - DNA, 96 bp, synthetic construct; identical to SEQ ID NO: 77
  misc_feature 1..96: note = Engineered antibody sequence
  source 1..96: mol_type = other DNA

SEQ ID NO: 478 - DNA, 39 bp, synthetic construct
  misc_feature 1..39: note = Engineered antibody sequence
  source 1..39: mol_type = other DNA
ctgggcagttatgattgtagtaatggtgattgttttgtt

SEQ ID NO: 479 - DNA, 33 bp, synthetic construct; identical to SEQ ID NO: 79
  misc_feature 1..33: note = Engineered antibody sequence
  source 1..33: mol_type = other DNA

SEQ ID NO: 480 - DNA, 321 bp, synthetic construct; identical to SEQ ID NO: 40
  misc_feature 1..321: note = Engineered antibody sequence
  source 1..321: mol_type = other DNA

SEQ ID NO: 481 - protein, 441 aa, synthetic construct
  REGION 1..441: note = Engineered antibody sequence
QSVEESGGGLVQPEGSLTLTCTASGFDFSSNAMWWVRQAPGKGLEWIGCIYNGDGSTYYASWVNGRFSISKTSSTTVTLQLNSLTVADTATYYCARDLDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 482 - protein, 111 aa, synthetic construct
  REGION 1..111: note = Engineered antibody sequence
QSVEESGGGLVQPEGSLTLTCTASGFDFSSNAMWWVRQAPGKGLEWIGCIYNGDGSTYYASWVNGRFSISKTSSTTVTLQLNSLTVADTATYYCARDLDLWGPGTLVTVSS

SEQ ID NO: 483 - protein, 29 aa, synthetic construct
  REGION 1..29: note = Engineered antibody sequence
QSVEESGGGLVQPEGSLTLTCTASGFDFS

SEQ ID NO: 484 - protein, 5 aa, synthetic construct
  REGION 1..5: note = Engineered antibody sequence
SNAMW

SEQ ID NO: 485 - protein, 14 aa, synthetic construct
  REGION 1..14: note = Engineered antibody sequence
WVRQAPGKGLEWIG

SEQ ID NO: 486 - protein, 17 aa, synthetic construct
  REGION 1..17: note = Engineered antibody sequence
CIYNGDGSTYYASWVNG

SEQ ID NO: 487 - protein, 31 aa, synthetic construct
  REGION 1..31: note = Engineered antibody sequence
RFSISKTSSTTVTLQLNSLTVADTATYYCAR

SEQ ID NO: 488 - protein, 4 aa, synthetic construct
  REGION 1..4: note = Engineered antibody sequence
DLDL

SEQ ID NO: 489 - protein, 11 aa, synthetic construct
  REGION 1..11: note = Engineered antibody sequence
WGPGTLVTVSS

SEQ ID NO: 490 - protein, 330 aa, synthetic construct; identical to SEQ ID NO: 10
  REGION 1..330: note = Engineered antibody sequence

SEQ ID NO: 491 - DNA, 1326 bp, synthetic construct
  misc_feature 1..1326: note = Engineered antibody sequence
  source 1..1326: mol_type = other DNA
cagtcggtggaggagtccgggggaggcctggtccagcctgagggatccctgacactcacctgcacagcctctggattcgacttcagtagcaatgcaatgtggtgggtccgccaggctccagggaaggggctggagtggatcggatgcatttacaatggtgatggcagcacatactacgcgagctgggtgaatggccgattctccatctccaaaacctcgtcgaccacggtgactctgcaactgaatagtctgacagtcgcggacacggccacgtattattgtgcgagagatcttgacttgtggggcccgggcaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 492 - DNA, 333 bp, synthetic construct
  misc_feature 1..333: note = Engineered antibody sequence
  source 1..333: mol_type = other DNA
cagtcggtggaggagtccgggggaggcctggtccagcctgagggatccctgacactcacctgcacagcctctggattcgacttcagtagcaatgcaatgtggtgggtccgccaggctccagggaaggggctggagtggatcggatgcatttacaatggtgatggcagcacatactacgcgagctgggtgaatggccgattctccatctccaaaacctcgtcgaccacggtgactctgcaactgaatagtctgacagtcgcggacacggccacgtattattgtgcgagagatcttgacttgtggggcccgggcaccctcgtcaccgtctcgagc

SEQ ID NO: 493 - DNA, 87 bp, synthetic construct
  misc_feature 1..87: note = Engineered antibody sequence
  source 1..87: mol_type = other DNA
cagtcggtggaggagtccgggggaggcctggtccagcctgagggatccctgacactcacctgcacagcctctggattcgacttcagt

SEQ ID NO: 494 - DNA, 15 bp, synthetic construct
  misc_feature 1..15: note = Engineered antibody sequence
  source 1..15: mol_type = other DNA
agcaatgcaatgtgg

SEQ ID NO: 495 - DNA, 42 bp, synthetic construct; identical to SEQ ID NO: 255
  misc_feature 1..42: note = Engineered antibody sequence
  source 1..42: mol_type = other DNA

SEQ ID NO: 496 - DNA, 51 bp, synthetic construct
  misc_feature 1..51: note = Engineered antibody sequence
  source 1..51: mol_type = other DNA
tgcatttacaatggtgatggcagcacatactacgcgagctgggtgaatggc

SEQ ID NO: 497 - DNA, 93 bp, synthetic construct
  misc_feature 1..93: note = Engineered antibody sequence
  source 1..93: mol_type = other DNA
cgattctccatctccaaaacctcgtcgaccacggtgactctgcaactgaatagtctgacagtcgcggacacggccacgtattattgtgcgaga

SEQ ID NO: 498 - DNA, 12 bp, synthetic construct
  misc_feature 1..12: note = Engineered antibody sequence
  source 1..12: mol_type = other DNA
gatcttgacttg

SEQ ID NO: 499 - DNA, 33 bp, synthetic construct; identical to SEQ ID NO: 139
  misc_feature 1..33: note = Engineered antibody sequence
  source 1..33: mol_type = other DNA

SEQ ID NO: 500 - DNA, 993 bp, synthetic construct; identical to SEQ ID NO: 20
  misc_feature 1..993: note = Engineered antibody sequence
  source 1..993: mol_type = other DNA

SEQ ID NO: 501 - protein, 219 aa, synthetic construct
  REGION 1..219: note = Engineered antibody sequence
AIVMTQTPSSKSVPVGDTVTINCQASESLYNNNALAWFQQKPGQPPKRLIYDASKLASGVPSRFSGGGSGTQFTLTISGVQCDDAATYYCGGYRSDSVDGVAFAGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 502 - protein, 113 aa, synthetic construct
  REGION 1..113: note = Engineered antibody sequence
AIVMTQTPSSKSVPVGDTVTINCQASESLYNNNALAWFQQKPGQPPKRLIYDASKLASGVPSRFSGGGSGTQFTLTISGVQCDDAATYYCGGYRSDSVDGVAFAGGTEVVVKR

SEQ ID NO: 503 - protein, 23 aa, synthetic construct
  REGION 1..23: note = Engineered antibody sequence
AIVMTQTPSSKSVPVGDTVTINC

SEQ ID NO: 504 - protein, 13 aa, synthetic construct
  REGION 1..13: note = Engineered antibody sequence
QASESLYNNNALA

SEQ ID NO: 505 - protein, 15 aa, synthetic construct
  REGION 1..15: note = Engineered antibody sequence
WFQQKPGQPPKRLIY

SEQ ID NO: 506 - protein, 7 aa, synthetic construct
  REGION 1..7: note = Engineered antibody sequence
DASKLAS

SEQ ID NO: 507 - protein, 32 aa, synthetic construct
  REGION 1..32: note = Engineered antibody sequence
GVPSRFSGGGSGTQFTLTISGVQCDDAATYYC

SEQ ID NO: 508 - protein, 12 aa, synthetic construct
  REGION 1..12: note = Engineered antibody sequence
GGYRSDSVDGVA

SEQ ID NO: 509 - protein, 11 aa, synthetic construct
  REGION 1..11: note = Engineered antibody sequence
FAGGTEVVVKR

SEQ ID NO: 510 - protein, 106 aa, synthetic construct; identical to SEQ ID NO: 30
  REGION 1..106: note = Engineered antibody sequence

SEQ ID NO: 511 - DNA, 660 bp, synthetic construct
  misc_feature 1..660: note = Engineered antibody sequence
  source 1..660: mol_type = other DNA
gccatcgtgatgacccagactccatcttccaagtctgtccctgtgggagacacagtcaccatcaattgccaggccagtgagagtctttataataacaacgccttggcctggtttcagcagaaaccagggcagcctcccaagcgcctgatctatgatgcatccaaactggcatctggggtcccatcgcggttcagtggcggtgggtctgggacacagttcactctcaccatcagtggcgtgcagtgtgacgatgctgccacttactactgtggaggctacagaagtgatagtgttgatggtgttgctttcgccggagggaccgaggtggtggtcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 512 - DNA, 339 bp, synthetic construct
  misc_feature 1..339: note = Engineered antibody sequence
  source 1..339: mol_type = other DNA
gccatcgtgatgacccagactccatcttccaagtctgtccctgtgggagacacagtcaccatcaattgccaggccagtgagagtctttataataacaacgccttggcctggtttcagcagaaaccagggcagcctcccaagcgcctgatctatgatgcatccaaactggcatctggggtcccatcgcggttcagtggcggtgggtctgggacacagttcactctcaccatcagtggcgtgcagtgtgacgatgctgccacttactactgtggaggctacagaagtgatagtgttgatggtgttgctttcgccggagggaccgaggtggtggtcaaacgt

SEQ ID NO: 513 - DNA, 69 bp, synthetic construct
  misc_feature 1..69: note = Engineered antibody sequence
  source 1..69: mol_type = other DNA
gccatcgtgatgacccagactccatcttccaagtctgtccctgtgggagacacagtcaccatcaattgc

SEQ ID NO: 514 - DNA, 39 bp, synthetic construct
  misc_feature 1..39: note = Engineered antibody sequence
  source 1..39: mol_type = other DNA
caggccagtgagagtctttataataacaacgccttggcc

SEQ ID NO: 515 - DNA, 45 bp, synthetic construct
  misc_feature 1..45: note = Engineered antibody sequence
  source 1..45: mol_type = other DNA
tggtttcagcagaaaccagggcagcctcccaagcgcctgatctat

SEQ ID NO: 516 - DNA, 21 bp, synthetic construct
  misc_feature 1..21: note = Engineered antibody sequence
  source 1..21: mol_type = other DNA
gatgcatccaaactggcatct

SEQ ID NO: 517 - DNA, 96 bp, synthetic construct
  misc_feature 1..96: note = Engineered antibody sequence
  source 1..96: mol_type = other DNA
ggggtcccatcgcggttcagtggcggtgggtctgggacacagttcactctcaccatcagtggcgtgcagtgtgacgatgctgccacttactactgt

SEQ ID NO: 518 - DNA, 36 bp, synthetic construct
  misc_feature 1..36: note = Engineered antibody sequence
  source 1..36: mol_type = other DNA
ggaggctacagaagtgatagtgttgatggtgttgct

SEQ ID NO: 519 - DNA, 33 bp, synthetic construct
  misc_feature 1..33: note = Engineered antibody sequence
  source 1..33: mol_type = other DNA
ttcgccggagggaccgaggtggtggtcaaacgt

SEQ ID NO: 520 - DNA, 321 bp, synthetic construct; identical to SEQ ID NO: 40
  misc_feature 1..321: note = Engineered antibody sequence
  source 1..321: mol_type = other DNA

SEQ ID NO: 521 - protein, 441 aa, synthetic construct
  REGION 1..441: note = Engineered antibody sequence
EVQLVESGGGLVQPGGSLRLSCAVSGIGLSSYYMQWVRQAPGKGLEWVGVIGSDGKTYYATWAKGRFTISRDNSKTTVYLQMNSLRAEDTAVYFCTRGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 522 - protein, 111 aa, synthetic construct; identical to SEQ ID NO: 362
  REGION 1..111: note = Engineered antibody sequence

SEQ ID NO: 523 - protein, 30 aa, synthetic construct; identical to SEQ ID NO: 363
  REGION 1..30: note = Engineered antibody sequence

SEQ ID NO: 524 - protein, 5 aa, synthetic construct
  REGION 1..5: note = Engineered antibody sequence
SYYMQ

SEQ ID NO: 525 - protein, 14 aa, synthetic construct
  REGION 1..14: note = Engineered antibody sequence
WVRQAPGKGLEWVG

SEQ ID NO: 526 - protein, 16 aa, synthetic construct
  REGION 1..16: note = Engineered antibody sequence
VIGSDGKTYYATWAKG

SEQ ID NO: 527 - protein, 32 aa, synthetic construct; identical to SEQ ID NO: 367
  REGION 1..32: note = Engineered antibody sequence

SEQ ID NO: 528 - skipped

SEQ ID NO: 529 - protein, 11 aa, synthetic construct
  REGION 1..11: note = Engineered antibody sequence
WGQGTLVTVSS

SEQ ID NO: 530 - protein, 330 aa, synthetic construct; identical to SEQ ID NO: 90
  REGION 1..330: note = Engineered antibody sequence

SEQ ID NO: 531 - DNA, 1326 bp, synthetic construct
  misc_feature 1..1326: note = Engineered antibody sequence
  source 1..1326: mol_type = other DNA
gaggtgcagcttgtggagtctgggggaggcttggtccagcctggggggtccctgagactctcctgtgcagtctctggaatcggcctcagtagctactacatgcaatgggtccgtcaggctccagggaaggggctggagtgggtcggagtcattggtagtgatggtaagacatactacgcgacctgggcgaaaggccgattcaccatctccagagacaattccaagaccacggtgtatcttcaaatgaacagcctgagagctgaggacactgctgtgtatttctgtaccagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 532 - DNA, 333 bp, synthetic construct; identical to SEQ ID NO: 372
  misc_feature 1..333: note = Engineered antibody sequence
  source 1..333: mol_type = other DNA

SEQ ID NO: 533 - DNA, 90 bp, synthetic construct; identical to SEQ ID NO: 373
  misc_feature 1..90: note = Engineered antibody sequence
  source 1..90: mol_type = other DNA

SEQ ID NO: 534 - DNA, 15 bp, synthetic construct
  misc_feature 1..15: note = Engineered antibody sequence
  source 1..15: mol_type = other DNA
agctactacatgcaa

SEQ ID NO: 535 - DNA, 42 bp, synthetic construct; identical to SEQ ID NO: 55
  misc_feature 1..42: note = Engineered antibody sequence
  source 1..42: mol_type = other DNA

SEQ ID NO: 536 - DNA, 48 bp, synthetic construct; identical to SEQ ID NO: 336
  misc_feature 1..48: note = Engineered antibody sequence
  source 1..48: mol_type = other DNA

SEQ ID NO: 537 - DNA, 96 bp, synthetic construct; identical to SEQ ID NO: 377
  misc_feature 1..96: note = Engineered antibody sequence
  source 1..96: mol_type = other DNA

SEQ ID NO: 538 - skipped

SEQ ID NO: 539 - DNA, 33 bp, synthetic construct; identical to SEQ ID NO: 59
  misc_feature 1..33: note = Engineered antibody sequence
  source 1..33: mol_type = other DNA

SEQ ID NO: 540 - DNA, 993 bp, synthetic construct; identical to SEQ ID NO: 100
  misc_feature 1..993: note = Engineered antibody sequence
  source 1..993: mol_type = other DNA

SEQ ID NO: 541 - protein, 219 aa, synthetic construct; identical to SEQ ID NO: 381
  REGION 1..219: note = Engineered antibody sequence

SEQ ID NO: 542 - protein, 113 aa, synthetic construct; identical to SEQ ID NO: 382
  REGION 1..113: note = Engineered antibody sequence

SEQ ID NO: 543 - protein, 22 aa, synthetic construct
  REGION 1..22: note = Engineered antibody sequence
QVLTQSPSSLSASVGDRVTINC

SEQ ID NO: 544 - protein, 13 aa, synthetic construct
  REGION 1..13: note = Engineered antibody sequence
QASQNVYNNNYLA

SEQ ID NO: 545 - protein, 15 aa, synthetic construct
  REGION 1..15: note = Engineered antibody sequence
WYQQKPGKVPKQLIY

SEQ ID NO: 546 - protein, 7 aa, synthetic construct
  REGION 1..7: note = Engineered antibody sequence
STSTLAS

SEQ ID NO: 547 - protein, 32 aa, synthetic construct; identical to SEQ ID NO: 67
  REGION 1..32: note = Engineered antibody sequence

SEQ ID NO: 548 - protein, 13 aa, synthetic construct
  REGION 1..13: note = Engineered antibody sequence
LGSYDCSRGDCFV

SEQ ID NO: 549 - protein, 11 aa, synthetic construct
  REGION 1..11: note = Engineered antibody sequence
FGGGTKVEIKR

SEQ ID NO: 550 - protein, 106 aa, synthetic construct; identical to SEQ ID NO: 30
  REGION 1..106: note = Engineered antibody sequence

SEQ ID NO: 551 - DNA, 660 bp, synthetic construct; identical to SEQ ID NO: 391
  misc_feature 1..660: note = Engineered antibody sequence
  source 1..660: mol_type = other DNA

SEQ ID NO: 552 - DNA, 339 bp, synthetic construct; identical to SEQ ID NO: 392
  misc_feature 1..339: note = Engineered antibody sequence
  source 1..339: mol_type = other DNA

SEQ ID NO: 553 - DNA, 66 bp, synthetic construct; identical to SEQ ID NO: 73
  misc_feature 1..66: note = Engineered antibody sequence
  source 1..66: mol_type = other DNA

SEQ ID NO: 554 - DNA, 39 bp, synthetic construct; identical to SEQ ID NO: 394
  misc_feature 1..39: note = Engineered antibody sequence
  source 1..39: mol_type = other DNA

SEQ ID NO: 555 - DNA, 45 bp, synthetic construct; identical to SEQ ID NO: 75
  misc_feature 1..45: note = Engineered antibody sequence
  source 1..45: mol_type = other DNA

SEQ ID NO: 556 - DNA, 21 bp, synthetic construct
  misc_feature 1..21: note = Engineered antibody sequence
  source 1..21: mol_type = other DNA
tctacatccactctggcatct

SEQ ID NO: 557 - DNA, 96 bp, synthetic construct; identical to SEQ ID NO: 77
  misc_feature 1..96: note = Engineered antibody sequence
  source 1..96: mol_type = other DNA

SEQ ID NO: 558 - DNA, 39 bp, synthetic construct; identical to SEQ ID NO: 398
  misc_feature 1..39: note = Engineered antibody sequence
  source 1..39: mol_type = other DNA

SEQ ID NO: 559 - DNA, 33 bp, synthetic construct; identical to SEQ ID NO: 79
  misc_feature 1..33: note = Engineered antibody sequence
  source 1..33: mol_type = other DNA

SEQ ID NO: 560 - DNA, 321 bp, synthetic construct; identical to SEQ ID NO: 40
  misc_feature 1..321: note = Engineered antibody sequence
  source 1..321: mol_type = other DNA

SEQ ID NO: 561 - protein, 37 aa, Homo sapiens
  REGION 1..37: note = C-term amidated
ACDTATCVTHRLAGLLSRSGGVVKNNFVPTNVGSKAF

SEQ ID NO: 562 - protein, 37 aa, Homo sapiens
  REGION 1..37: note = C-term amidated
ACNTATCVTHRLAGLLSRSGGMVKSNFVPTNVGSKAF

SEQ ID NO: 563 - protein, 106 aa, Homo sapiens; identical to SEQ ID NO: 30

SEQ ID NO: 564 - protein, 330 aa, synthetic construct; identical to SEQ ID NO: 10
  REGION 1..330: note = Engineered antibody sequence

SEQ ID NO: 565 - protein, 329 aa, synthetic construct
  REGION 1..329: note = Engineered antibody sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 566 - protein, 440 aa, synthetic construct
  REGION 1..440: note = Engineered antibody sequence
EVQLVESGGGLVQPGGSLRLSCAVSGIDLSGYYMNWVRQAPGKGLEWVGVIGINGATYYASWAKGRFTISRDNSKTTVYLQMNSLRAEDTAVYFCARGDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDARVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 567 - DNA, 1323 bp, synthetic construct
  misc_feature 1..1323: note = Engineered antibody sequence
  source 1..1323: mol_type = other DNA
gaggtgcagcttgtggagtctgggggaggcttggtccagcctggggggtccctgagactctcctgtgcagtctctggaatcgacctcagtggctactacatgaactgggtccgtcaggctccagggaaggggctggagtgggtcggagtcattggtattaatggtgccacatactacgcgagctgggcgaaaggccgattcaccatctccagagacaattccaagaccacggtgtatcttcaaatgaacagcctgagagctgaggacactgctgtgtatttctgtgctagaggggacatctggggccaagggaccctcgtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacgcgagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga